(19) United States
(12) Patent Application Publication
(10) Pub. No.: US 2016/0346249 A1
(43) Pub. Date: Dec. 1, 2016

(54) USE OF AMITIFADINE, (+)-1-(3,4-DICHLOROPHENYL)-3-AZABICYCLO[3.1.0]HEXANE IN METHODS AND COMPOSITIONS WITH ENHANCED EFFICACY AND REDUCED METABOLIC SIDE EFFECTS AND TOXICITY FOR TREATMENT OF DEPRESSION AND OTHER CENTRAL NERVOUS SYSTEM DISORDERS AND CONDITIONS AFFECTED BY MONOAMINE NEUROTRANSMITTERS

(71) Applicants: Anthony Alexander McKinney, Cambridge, MA (US); Frank Bymaster, Brownsburg, IN (US); Richard Welter, Cambridge, MA (US); Randall Marshall, Cambridge, MA (US)

(72) Inventors: Anthony Alexander McKinney, Cambridge, MA (US); Frank Bymaster, Brownsburg, IN (US); Richard Welter, Cambridge, MA (US); Randall Marshall, Cambridge, MA (US)

(21) Appl. No.: 13/964,024

(22) Filed: Aug. 9, 2013

Related U.S. Application Data
(60) Provisional application No. 61/682,315, filed on Aug. 13, 2012.

(51) Int. Cl. A61K 31/403

(52) U.S. Cl. CPC A61K 31/403

(57) ABSTRACT

The present invention relates to (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane and their use alone or in combination with additional psychotherapeutic compositions in the treatment of conditions affected by monoamine neurotransmitters, including treatment of refractory individuals. (+)-1-(3,4-dichlorophenyl)-3-azabicyclo[3.1.0]hexane compositions are metabolized by either or both MAO-A or cytochrome P450 enzymes and thus are effective in the treatment of individuals with cytochrome P450 polymorphisms or who are taking other medications that affect the cytochrome P450 pathway.

This application claims priority to U.S. Provisional Application No. 61/682,315, filed Aug. 13, 2012, the contents of which are incorporated herein by reference.

### TECHNICAL FIELD

The present invention relates to selective inhibition of the reuptake of monoamine neurotransmitters. Specifically, the present invention relates to compositions comprising (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane and their use in the treatment of conditions affected by monoamine neurotransmitters.

### ADDITIONAL DISCLOSURE

This application includes the additional disclosure of U.S. patent application Ser. No. 13/310,694, filed Dec. 2, 2011, U.S. Provisional patent application Ser. No. 61/662,462, filed Jun. 21, 2012, U.S. Provisional patent application Ser. No. 61/677,453, filed Jul. 30, 2012, U.S. Provisional patent application Ser. No. 61/573,499, filed Sep. 6, 2011, U.S. Continuation patent application Ser. No. 13/366,209, filed Feb. 3, 2012, U.S. patent application Ser. No. 13/335,981, filed Dec. 23, 2011, U.S. patent application Ser. No. 10/466,457, filed Jan. 11, 2002, now U.S. Pat. No. 7,098,229, U.S. patent application Ser. No. 09/753,883, filed Jan. 11, 2011, now U.S. Pat. No. 6,372,919, U.S. Continuation patent application Ser. No. 13/297,452, filed Nov. 16, 2011, and U.S. Continuation patent application Ser. No. 13/366,211, filed Feb. 3, 2012, each of which are incorporated herein by reference in their entirety for all purposes.

### BACKGROUND OF THE INVENTION

Drug development has generally focused on affecting a specific target molecule in order to minimize side effects and increase potency. However, clinical studies of disorders ranging from cancer to schizophrenia have indicated that drugs affecting a variety of targets may be more efficacious (Frantz et al., 2005). In the treatment of depression, the use of combinations of serotonin-norepinephrine reuptake inhibitors (Thase et al., 2001) and combinations of selective serotonin reuptake inhibitors with dopamine and norepinephrine inhibitors can be more effective than administration of a selective serotonin reuptake inhibitor by itself (Trivedi et al., 2006).

Triple reuptake inhibitors selectively inhibit the reuptake of multiple monoamine neurotransmitters. Specifically, they inhibit the reuptake of 5-hydroxytryptamine (serotonin), norepinephrine and dopamine by blocking the action of the serotonin transporter, norepinephrine transporter and dopamine transporter. There are several triple reuptake inhibitors under investigation for use in the treatment of a variety of conditions including depression, anxiety, panic disorder, post-traumatic stress disorder, obsessive compulsive disorder, schizophrenia and allied disorders, addiction, obesity, tic disorders, attention deficit hyperactivity disorder (ADHD), Parkinson's disease, chronic pain and Alzheimer's disease as well as other cognitive impairment disorders. (See, e.g. McMillen et al., 2007; Gardner et al., 2006; Tizzano et al. 2008; Basile et al., 2007).

According to the World Health Organization, depression is the leading cause of disability and the fourth leading contributor to the global burden of disease (World Health Organization, 2012). It affects more than 121 million people worldwide. Two-thirds of patients who are initially prescribed antidepressant medications do not experience a timely remission (Fava et al., 1996). Residual symptoms are associated with an increased risk of relapse, impaired social and occupational functioning, and chronicity (Judd et al., 1998). The risk of a subsequent episode of Major Depressive Disorder increases 10-fold with one prior episode and 14-18 fold after more than one prior episode (Byrne et al., 1998). Maintenance therapy is recommended after an initial treatment response in order to prevent such relapses; nonetheless, it is estimated that between 9-57% of major depression patients relapse during the maintenance period (Byrne et al., 1998). Relapses have been attributed to a variety of sources including noncompliance, tachyphylaxis, drug tolerance, breakthrough depression, antidepressant tolerance, loss of an initial placebo response, and progression of the disease. Additionally, some relapses have been associated with individuals with a cytochrome P450 genetic profile with variable or non-standard activity of one or more cytochrome P450 enzymes or who have been concurrently taking a medication or other substance that modifies the action of one or more of the cytochrome P450 enzymes. Alterations in the action of a cytochrome P450 enzyme responsible for metabolizing a pharmaceutical agent can change both the concentration of the pharmaceutical agent in the body and the length of time in which the pharmaceutical agent remains in an individual's system complicating dosing regimes and decreasing therapeutic efficacy.

The cytochrome P450 family of enzymes is a group of highly structurally polymorphic enzymes primarily found in the liver that generally catalyze the oxidative transformation of both endogenous and exogenous molecules (Meunier, 2004) and metabolize approximately 90% of all drugs (Lynch, 2007). However, the specific allele encoding for a cytochrome P450 enzyme that an individual possesses significantly affects the ability of an individual to metabolize particular pharmaceutical agents. Allelic variation may lead to an individual being an ultra rapid metabolizer, extensive metabolizer, intermediate metabolizer, or poor metabolizer of a particular pharmaceutical agent. For example, CYP2D6, one of the cytochrome P450 enzymes, is believed to be involved in the metabolism of 25% of all drugs currently on the market (Teh et al., 2012) including many beta blockers, antidepressants, and opioids (Bernhard et al. 2006, Abraham et al. 2001). More than 80 alleles of CYP2D6 have been recognized, 20 of which significantly alter the metabolism of drugs that are substrates for this enzyme. 5 to 10 percent of Caucasians and 2 to 7 percent of individuals of African descent have CYP2D6 alleles that result in no enzyme expression and thus are poor metabolizers of drugs dependent on CYP2D6 and 1 to 2 percent of Caucasians and 4.5 percent of individuals of African descent have CYP2D6 alleles that result in over expression of the CYP2D6 enzyme and are thus ultra-rapid metabolizers of drugs dependent on CYP2D6 (Cai, et al., 2006; Crews et al. 2012). Such variations in metabolism can affect dosage and therapeutic levels of many common antidepressants such as fluoxetine, paroxetine, venlafaxine, desipramine, clomipramine, imipramine, nortriptyline, amitriptyline, imipramine, doxepin, duloxetine, trazadone, and mirtazapine, all of which are metabolized by CYP2D6 enzymes. Similarly, one in five Asian persons is a poor metabolizer of drugs dependent on CYP2C19, another member of the cytochrome P450 family which metabolizes pharmaceutical agents such as phenytoin (Dilantin®), phenobarbital, and omeprazole (Prilosec®), (Chong, 2003), citalopram, escitalopram, clomipramine, amitriptyline, sertraline, imipramine, nortriptyline and doxepin among others. Additionally, members of the cytochrome P450 family may be inhibited by frequently used medications such as cimetidine (Tagamet®), diphenydramine (Benadryl®), paroxetine (Paxil®), clarithromycin (Biaxin®), erythromycin, fluoxetine (Prozac®), and ciprofloaxin (Cipro®) or may be induced by compounds such as tobacco or fluconazole (Diflucan®). Thus, standard drug doses may cause adverse effects related to elevated drug serum levels if a person is a poor metabolizer, is inadvertently also taking a cytochrome P450 enzyme inhibitor, or has impaired liver function. Conversely, individuals who are ultrarapid metabolizers or are taking a cytochrome P450 enzyme inducer may have trouble reaching or maintaining therapeutic levels at standard drug doses of compounds metabolized by a cytochrome P450 enzyme.

Many currently used antipsychotics and antidepressants have a narrow therapeutic range, with concentration-dependent adverse effects occurring at concentrations only slightly higher than the dose required for psychiatric effect (Van der Weide et al., 2006). Additionally, some antidepressants such as fluoxetine and paroxtine are inhibitors of one or more cytochrome P450 enzymes, affecting the dosing and usefulness of other medications an individual may be taking (deVane et al. 2006) and potentially generating adverse reactions for other therapeutic agents.

The involvement of cytochrome P450 enzymes in the metabolism of 90% of drugs complicates dosing and decreases the effectiveness and predictability of current medications for the treatment of conditions affected by monoamine neurotransmitters. There is therefore an unmet need for the identification of effective pharmaceuticals which are not primarily processed by the cytochrome p450 pathway. Given the risk of relapse and the global burden of depression, there is additionally an unmet need for the identification of effective pharmaceuticals which may be used in the treatment of depression and other conditions affected by monoamine neurotransmitters, particularly for individuals that were unresponsive to initial therapies or who experience a relapse of their condition.

### SUMMARY OF EXEMPLARY EMBODIMENTS

Provided herein are compositions and methods using an unbalanced triple reuptake inhibitor, (+)-1-(3,4-dichlorophenyl)-3-azabicyclo[3.1.0]hexane, amitifadine, as shown below, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane, for the treatment of mammals, including humans, suffering from signs and symptoms of disorders generally treated with triple reuptake inhibitors including, but not limited to, depression, anxiety, panic disorder, post-traumatic stress disorder, obsessive compulsive disorder, schizophrenia and allied disorders, addiction, obesity, tic disorders, attention deficit hyperactivity disorder (ADHD), Parkinson's disease, chronic pain states, and Alzheimer's disease and other cognitive impairing conditions. Unbalanced as used herein refers to the relative effects of a triple reuptake inhibitor on each of the monoamine transporters. In this case, amitifadine has the most activity against the serotonin transporter, half as much to the norepinephrine transporter and one eighth as much to the dopamine transporter. In contrast, a balanced triple reuptake inhibitor would have similar activity against each of the three monoamine transporters.

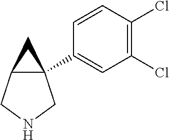 [SMILES: Clc1ccc([C@]23CNC[C@H]2C3)cc1Cl]

### (+)-1-(3,4-dichlorophenyl)-3-azabicyclo[3.1.0]hexane

(+)-1-(3,4-dichlorophenyl)-3-azabicyclo[3.1.0]hexane agents as used herein are substantially free of the corresponding (−) enantiomer, (+1-(3,4-dichlorophenyl)-3-azabicyclo[3.1.0]hexane. In addition to being enantiomeric, (+)-1-(3,4-dichlorophenyl)-3-azabicyclo[3.1.0]hexane exists in at least three polymorphic forms, labeled herein polymorphs A, B and C. The polymorphs may be used in pharmaceutical compositions in combination or in forms that are substantially free of one or more of the other polymorphic forms.

(+)-1-(3,4-dichlorophenyl)-3-azabicyclo[3.1.0]hexane may furthermore be in the form of pharmaceutically acceptable active salts, glycosylated derivatives, metabolites, solvates, hydrates and/or prodrugs. Exemplary metabolites include the lactam, 5-(3,4-dichloro-phenyl)-3-aza-bicyclo[3.1.0]hexan-2-one (EB-10101) and the carbamate, which is subsequently conjugated to form the glucuronide. Additionally, many pharmacologically active organic compounds regularly crystallize incorporating second, foreign molecules, especially solvent molecules, into the crystal structure of the principal pharmacologically active compound to form pseudopolymorphs. When the second molecule is a solvent molecule, the pseudopolymorphs can also be referred to as solvates. Pharmaceutically acceptable forms of (+)-1-(3,4-dichlorophenyl)-3-azabicyclo[3.1.0]hexane may also include inorganic and organic acid addition salts such as hydrochloride salt, i.e. amitifadine HCl.

Additional background information regarding (±)-1-(3,4-dichlorophenyl)-3-azabicyclo[3.1.0]hexane, such as binding studies, may be found, for example, in U.S. Pat. No. 4,435,419, WO/20040466457, WO2007127396, WO02066427, WO2006023659, U.S. patent application Ser. No. 11/740,667, and U.S. Pat. No. 6,372,919, each of which is incorporated herein by reference in their entirety.

Further provided herein are combinatorial compositions and coordinate treatment means using additional or secondary psychotherapeutic agents in combination with (+)-1-(3,4-dichlorophenyl)-3-azabicyclo[3.1.0]hexane agents including (+)-1-(3,4-dichlorophenyl)-3-azabicyclo[3.1.0]hexane, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane. Suitable secondary psychotherapeutic drugs for use in the compositions and methods herein include, but are not limited to, drugs from the general classes of anti-convulsant, mood-stabilizing, anti-psychotic, anxiolytic, benzodiazepines, calcium channel blockers, anti-inflammatories, and antidepressants. (See, e.g., R J. Baldessarini in Goodman & Gilman's The Pharmacological Basis of Therapeutics, 11th Edition, Chapters 17 and 18, McGraw-Hill, 2005 for a review). Exemplary antidepressants include, for example, tri-cyclic antidepressants (TCAs), specific monoamine reuptake inhibitors, selective serotonin reuptake inhibitors, selective norepinephrine or noradrenaline reuptake inhibitors, selective dopamine reuptake inhibitors, norepinephrine-dopamine reuptake inhibitors, serotonin-norepinephrine reuptake inhibitors, multiple monoamine reuptake inhibitors, monoamine oxidase inhibitors, atypical antidepressants, atypical antipsychotics, anticonvulsants, or opiate agonists.

It is shown herein that use of (+)-1-(3,4-dichlorophenyl)-3-azabicyclo[3.1.0]hexane agents ((+)-1-(3,4-dichlorophenyl)-3-azabicyclo[3.1.0]hexane, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) are effective in treating, preventing, alleviating, or moderating disorders affected by monoamine neurotransmitters or biogenic amines, specifically disorders that are alleviated by inhibiting dopamine and/or norepinephrine and/or serotonin reuptake. Such conditions include, but are not limited to, depression, anxiety, panic disorder, post-traumatic stress disorder, obsessive compulsive disorder, schizophrenia and allied disorders, obesity, tic disorders, ADHD, substance abuse disorders, Parkinson's disease, chronic pain states, Alzheimer's disease, and other cognitive impairing conditions. Use of the compositions of the present invention may increase monoamine neurotransmitter levels and/or selectively inhibit reuptake of monoamine neurotransmitters and/or biogenic amines. Additional information regarding (+)-1-(3,4-dichlorophenyl)-3-azabicyclo[3.1.0]hexane may be found in U.S. patent application Ser. No. 13/310,694, filed Dec. 2, 2011, and its predecessor application, U.S. Provisional Patent Application No. 61/419,769, filed Dec. 3, 2010, both of which are incorporated by reference herein in their entirety.

The unbalanced serotonin-norepinephrine-dopamine reuptake inhibition ratio of ˜1:2:8, respectively, of (+)-1-(3,4-dichlorophenyl)-3-azabicyclo[3.1.0]hexane. (Skolnick et al., 2003) allows for higher dosages of (+)-1-(3,4-dichlorophenyl)-3-azabicyclo[3.1.0]hexane to be used without triggering the dopaminergic or norepinephrine side effects such as elevated heart rate, increased blood pressure, gastrointestinal (nausea/vomiting and constipation/diarrhea) effects, dry mouth, insomnia, anxiety, and hypomania seen in similar dosages of balanced triple reuptake inhibitors or unbalanced triple reuptake inhibitors with different inhibition ratios.

Additionally, unlike most anti-depressants, the (+)-1-(3,4-dichlorophenyl)-3-azabicyclo[3.1.0]hexane compositions as described herein are metabolized by both or either the cytochrome P450 enzymes and the monoamine oxidase (MAO) family of enzymes. Metabolism by multiple routes may be a desired property for a drug because it may offer alternative pathways if one or more pathways are altered by drug-induced inhibition or a polymorphic change such as found with CYP2D6 and 2C19 isoforms.

The compositions described herein are also unexpectedly useful in the treatment of individuals who have previously been treated one or more times for disorders affected by monoamine neurotransmitters, particularly depression. (+)-1-(3,4-dichlorophenyl)-3-azabicyclo[3.1.0]hexane agents have shown unexpected efficacy in the treatment of individuals who have been refractory to previous treatments for disorders affected by monoamine neurotransmitters, i.e. individuals that have not responded or have responded in an unsatisfactory manner to at least one other treatment, specifically anti-depressants such as, but not limited to, tri-cyclic antidepressants (TCAs), specific monoamine reuptake inhibitors, selective serotonin reuptake inhibitors (e.g. citalopram), selective norepinephrine or noradrenaline reuptake inhibitors, serotonin-norepinephrine reuptake inhibitors, selective dopamine reuptake inhibitors, norepinephrine-dopamine reuptake inhibitors, multiple monoamine reuptake inhibitors, monoamine oxidase inhibitors, atypical antidepressants, atypical antipsychotics, anticonvulsants, anti-inflammatories or opiate agonists. Individuals may have been refractory to previous treatment(s) for any reason. In some embodiments, refractory individuals may have failed to respond or failed to respond sufficiently to a previous treatment. In one embodiment, a refractory individual may have treatment resistant depression. In other embodiments, a refractory individual may have responded to the initial treatment, but not succeed in entering remission from the maintenance treatment. In further embodiments, refractory individuals may have initially responded to previous treatments but had a relapse. Such a relapse may have occurred while on or off maintenance therapy. In additional embodiments, refractory individuals may be tachyphylactic. In further embodiments, the refractory individual may have developed pharmacokinetic tolerance, i.e. a change in the concentration of the drug acting at its target site, resulting from alterations in absorption, distribution, biotransformation, or elimination of the drug as a result of previous exposure to it (Byrne et al, 1998). In yet another embodiment, refractory individuals may have genetic variants of cytochrome P450 enzymes such as CYP2D6 and CYP2C19 that cause them to process pharmaceutical agents such as anti-depressants in an atypical fashion. Such mutant alleles of cytochrome P450 enzymes may produce phenotypes that cause pharmaceutical agents to not be metabolized, to be metabolized more slowly than average, to be metabolized more rapidly than normal, to be metabolized ultra-rapidly, or some variation thereof. In some embodiments, patients with genetic variants of cytochrome P450 enzymes are identified by one or more diagnostic genotyping tests and then administered amitifadine. In other embodiments, refractory individuals may have impaired liver function or may be taking additional medications that are inhibitors or enhancers of cytochrome P450 enzymes. Such impaired liver function may be for any reason. In some embodiments, impaired liver function may be due to cirrhosis. In some embodiments, impaired liver function may be due to jaundice. In some embodiments, impaired liver function may be due to chronic liver disease. In some embodiments, impaired liver function may be due to previous administration of one or more hepatotoxic medications. In some embodiments, impaired liver function may be due to concurrent administration of one or more hepatotoxic medications. In some embodiments, impaired liver function may be due to sequential administration of one or more hepatotoxic medications. In other embodiments, impaired liver function may be due to alcoholism. In some embodiments, refractory individuals may have been unable to continue taking the medication due to intolerance of the medication including side effects such as, but not limited to, sexual dysfunction, weight gain, insomnia, dry mouth, constipation, nausea and vomiting, dizziness, memory loss, agitation, anxiety, sedation, headache, urinary retention, or abdominal pain. Unsatisfactory or failed responses may be determined by any means generally used, including patient self-reporting, clinical observation and depression rating scales.

The compositions described herein are additionally useful for individuals taking medications for other conditions that preclude the taking of an additional medication that modifies or is processed by the same cytochrome P450 enzymes, e.g. the decongestant phenylephrine or the analgesic hydrocodone should not be taken in combination with the antidepressant duloxetine.

The compositions described herein are also surprisingly useful in the treatment of addiction. The usefulness of amitifadine and amitifadine agents in the treatment of addiction is surprising given that amitifadine more strongly inhibits serotonin and norepinephrine reuptake than dopamine reuptake and substances that trigger dependencies in human beings generally increase the release of dopamine in the nucleus accumbens. (Di Chiara et al., 2004).

Administration of pharmaceutical compositions comprising (+)-1-(3,4-dichlorophenyl)-3-azabicyclo[3.1.0]hexane agents including (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane in effective amounts will be effective to improve an individual's score on a depression rating scale such as, but not limited to, Montgomery Asberg Depression Rating Scale (MADRS), the Hamilton Rating Scale for Depression (HAMD-17), the Clinical Global Impression-Severity Scale (CGI-S) and the Clinical Global Impression-Improvement Scale (CGI-I). In some embodiments, administration of the pharmaceutical compositions contemplated herein will be sufficient to place an individual into remission. Remission may be measured by any of a variety of ways, for example, remission from depression may be determined with a MADRS score of ≦12, HAMD-17 score of ≦7 or CGI-S score of ≦2.

Administration of pharmaceutical compositions comprising (+)-1-(3,4-dichlorophenyl)-3-azabicyclo[3.1.0]hexane agents including (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane in effective amounts will additionally be effective to decrease cravings for addictive substances, improving an individual's score on one or more multidimensional scales such as Questionnaire of Smoking Urges (QSU) developed by Tiffany & Drobes (Br. J. Addict. 86(11):1467-76 (1991)), Measurement of Drug Craving scale (Sayette et al. 2000), Drug History Questionnaire (DHQ), Desires for Drug Questionnaire (Franken et al., Addict. Behav. 27:675-85 (2002)), Heaviness of Smoking Index (HSI), the Fagerstrom Test for Nicotine Dependence (FTND) or the Obsessive-Compulsive Beliefs Questionnaire-87 (OBQ-87), Drinker Inventory of Consequences (DrInC) (Forcehimes et al., Addict Behay. 2007 August; 32(8):1699-704. Epub 2006 Dec. 19. Psychometrics of the Drinker Inventory of Consequences (DrInC)) and Profile of Mood States (POMS) (Nyenhuis et al., J Clin Psychol. 1999 January; 55(1):79-86).

The present invention may be understood more fully by reference to the detailed description and examples which are intended to exemplify non-limiting embodiments of the invention.

### BRIEF DESCRIPTION OF THE DRAWINGS

FIG. 1 is a graph showing a decrease in patients' scores on the Montgomery Asberg Depression Rating Scale when treated with amitifadine (EB-1010) ((+)-1-(3,4-dichlorophenyl)-3-azabicyclo[3.1.0]hexane) in comparison to placebo in a six week double-blind study and one week post-treatment (modified intent-to-treat, n=56) (mixed-effects model repeated measures approach (MMRM) least square (LS) means).

FIG. 2 is a graph showing that treatment with amitifadine (EB-1010) ((+)-1-(3,4-dichlorophenyl)-3-azabicyclo[3.1.0]hexane) resulted in a decrease on the Hamilton Depression Rating Scale (HAM-D) in comparison to placebo in a six week double-blind study and one week post-treatment (modified intent-to-treat, n=56) (MMRM LS means).

FIG. 3 is a graph showing that treatment with amitifadine (EB-1010) ((+)-1-(3,4-dichlorophenyl)-3-azabicyclo[3.1.0]hexane) resulted in a decrease on the Clinical Global Impression-Improvement Scale (CGI-I) in a six week double-blind study and one week post-treatment indicating improvement in the condition of the patients in a six week double-blind study and one week post-treatment (modified intent-to-treat, n=56) (MMRM LS means).

FIG. 4 is a graph showing an improvement in the condition of patients treated with EB-1010 ((+)-1-(3,4-dichlorophenyl)-3-azabicyclo[3.1.0]hexane, amitifadine) in comparison to placebo in a six week double-blind study and one week post-treatment as determined using the Clinical Global Impression-Severity (CGI-S) scale. (Modified intent-to-treat, n=56) (MMRM LS means).

FIG. 5 is a graph showing that treatment with EB-1010 ((+)-1-(3,4-dichlorophenyl)-3-azabicyclo[3.1.0]hexane, amitifadine) resulted in significantly greater remission rates than treatment with placebo as measured by the Clinical Global Impressions-Severity (CGI-S) scale (Last Observation Carried Forward (LOCF)).

FIG. 6 is a graph showing that treatment with EB-1010 ((+)-1-(3,4-dichlorophenyl)-3-azabicyclo[3.1.0]hexane, amitifadine) resulted in statistically significant improvement on the adhedonia factor score of the MADRS compared to placebo in a six week double-blind study and one week post-treatment. (Modified intent-to-treat, n=56) (MMRM LS means).

FIG. 7 is a graph showing that Derogatis Interview for Sexual Functioning-Self Report (DISF-SR) scores stratified by low mean baseline scores versus high mean baseline scores and that there was no difference in those treated with EB-1010 ((+)-1-(3,4-dichlorophenyl)-3-azabicyclo[3.1.0]hexane, amitifadine) or placebo indicating that treatment with EB-1010 is not associated with the emergence of sexual dysfunction that is typically observed with serotonergic and serotonergic combination antidepressants (LOCF).

FIG. 8 is a graph showing the correlation of dose of amitifadine to Cₘₐₓ in humans.

FIG. 9 is a graph showing the correlation of dose of amitifadine to AUC in human subjects.

FIG. 10 is a chart showing the formation of lactam metabolites (EB-10101) in the liver microsome incubation of EB-1010 (amitifadine).

FIG. 11 is LC/(+)ESI MRM chromatograms of human liver microsome incubation of EB-1010 (amitifadine) with NADPH.

FIG. 12 is a chart showing the effect of pre-treated temperature on the formation of lactam metabolites 5-(3,4-dichloro-phenyl)-3-aza-bicyclo[3.1.0]hexan-2-one (EB-10101) (top) and an isomer of 5-(3,4-dichloro-phenyl)-3-aza-bicyclo[3.1.0]hexan-2-one (EB-10101) (bottom) of EB-1010 (amitifadine) when EB-1010 (amitifadine) is incubated with human liver microsomes.

FIG. 13 is a chart showing the effect of clorgyline (top) and selegiline (bottom) on the metabolism of amitifadine (EB-1010) to 5-(3,4-dichloro-phenyl)-3-aza-bicyclo[3.1.0]hexan-2-one (EB-10101) in human liver microsomal and mitochondrial incubations.

FIG. 14A and FIG. 14B are graphs showing the mean plasma concentration (SD) of amitifadine in healthy adult male volunteers following a single oral dose of amitifadine HCl 10, 25, 50, 100, or 150 mg IR Formulation. FIG. 14A is a linear presentation and FIG. 14B is a semi-logarithmic presentation of the mean concentrations.

FIG. 15 is a graph showing the mean AUC_{0-∞} (±SD) of amitifadine in healthy adult male volunteers following a single oral dose of amitifadine HCl 10, 25, 50, 100, or 150 mg IR Formulation.

FIG. 16 is a graph showing the mean Cₘₐₓ (±SD) of amitifadine in healthy adult male volunteers following a single oral dose of amitifadine HCl (Dov 21,947) 10, 25, 50, 100, or 150 mg IR Formulation.

FIG. 17 is two graphs showing mean plasma concentrations of DOV 21,947 (amitifadine) and the Lactam Metabolite Dov 216,298 (5-(3,4-dichloro-phenyl)-3-aza-bicyclo[3.1.0]hexan-2-one) in Healthy Adult Male and Female Subjects following a single oral dose of 300 mg (top) or 450 mg (bottom) of Amitifadine HCL IR Formulation.

FIG. 18 is the MS spectra of amitifadine standard using HPLC method 1.

FIG. 19 is the proposed ion fragmentation of amitifadine.

FIG. 20 is the extracted mass ion chromatograms of amitifadine in 0 and 4 hour human hepatocyte incubations using HPLC method 1. Data shown for each sample are extracted ion of full scans. +ESI: positive ion mode of electrospray ionization is used in this scan.

FIG. 21 is the extracted ion chromatograms of extract of 4 hour human hepatocyte incubation with amitifadine using methods described HPLC method 2. Data shown for each sample are extracted ion of full scans. The +ESI: positive ion mode of electrospray ionization is used in this scan.

FIG. 22 is the extracted mass chromatograms of 4 hour human hepatocyte incubation with amitifadine using HPLC method 1. Data shown for each sample are extracted ion of full scans. The +ESI: positive ion mode of electrospray ionization is used in this scan.

FIG. 23 is the brain to plasma ratio of amitifadine (parent) and EB-10101 (metabolite) determined in rats at various time points. Amitifadine is administered orally at 10 mg/kg po., and concentrations of amitifadine and EB-10101 are determined by a LC-MS/MS method. The mean±SEM plasma levels of amitifadine are 830±360, 570±100, 798±334, and 1233±98 at 0.5, 1, 2 and 4 hours, respectively, and the plasma levels of EB-10101 are 1153±610, 1620±303, 3831±1127, and 8896±794, respectively.

### DETAILED DESCRIPTION OF EXEMPLARY EMBODIMENTS

Described herein is an enantiomer of (±)-1-(3,4-dichlorophenyl)-3-azabicyclo[3.1.0]hexane, amitifadine ((±)-1-(3,4-dichlorophenyl)-3-azabicyclo[3.1.0]hexane) which provides therapeutic efficacy in the treatment of conditions affected by monoamine neurotransmitters including, but not limited to, depression, anxiety, panic disorder, post-traumatic stress disorder, obsessive compulsive disorder, schizophrenia and allied disorders, obesity, tic disorders, addiction, attention deficit hyperactivity disorder (ADHD), Parkinson's disease, chronic pain and Alzheimer's disease or other cognitive impairments. Further described herein are coordinate treatment methods and combined drug compositions, dosage forms, packages, and kits for preventing or treating conditions affected by monoamine neurotransmitters including, but not limited to, depression.

(+)-1-(3,4-dichlorophenyl)-3-azabicyclo[3.1.0]hexane is a triple reuptake inhibitor (TRI), or serotonin-norepinephrine-dopamine reuptake inhibitor (SNDRI). It was previously described in U.S. Pat. No. 6,372,919.

(+)-1-(3,4-dichlorophenyl)-3-azabicyclo[3.1.0]hexane possesses a desirable unbalanced triple monoamine uptake inhibition ratio, with highly potent serotonin reuptake inhibition and lesser norepinephrine and, particularly, dopamine reuptake inhibition in a ratio of ˜1:2:8, respectively (IC50 values of 12, 23, and 96 nM, respectively in human embryonic kidney (HEK) 293 cells expressing the corresponding human recombinant transporters for [3H]serotonin, [3H]norepinephrine, and [3H]dopamine). (Skolnick et al., 2003). There is preclinical evidence in support of the hypothesis that antidepressants that work by enhancing the synaptic availability of serotonin, norepinephrine, and dopamine may be superior to antidepressants that selectively affect only serotonin and/or norepinephrine reuptake. (Skolnick et al., 2003) The lesser dopamine reuptake inhibition is thought to be sufficient to confer a beneficial effect in the treatment of anhedonia (a core symptom presumably due to a mesocorticolimbic dopaminergic hypofunction in major depressive illness) and cognitive dysfunction, while avoiding undesirable effects thought to be triggered by excessive stimulation of dopamine systems, such as hypomania, nausea, insomnia or excessive pleasure seeking behaviors. Additionally, an unbalanced triple reuptake inhibitor may provide a lower side effect profile than a balanced triple reuptake inhibitor and allow for higher concentrations of an unbalanced inhibitor such as (+)-1-(3,4-dichlorophenyl)-3-azabicyclo[3.1.0]hexane to be used without incurring the dopaminergic and/or noradrenergic side effects frequently seen in the use of balanced triple reuptake inhibitors or unbalanced triple reuptake inhibitors that have different inhibition ratios. (+)-1-(3,4-dichlorophenyl)-3-azabicyclo[3.1.0]hexane offers the additional advantage of being metabolized by either or both the monoamine oxidase family of enzymes, and the cytochrome P450 family of enzymes. Metabolism by multiple routes may be a desired property for a drug because it may offer alternative pathways if one or more pathways are altered by drug-induced inhibition or a polymorphic change such as found with CYP2D6 and 2C19 isoforms.

Provided herein are compositions and methods using (+)-1-(3,4-dichlorophenyl)-3-azabicyclo[3.1.0]hexane (EB-1010), amitifadine, as shown below, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane, for the treatment of mammals, including humans, suffering from signs and symptoms of disorders generally treated with triple reuptake inhibitors including, but not limited to, depression, anxiety, panic disorder, post-traumatic stress disorder, obsessive compulsive disorder, schizophrenia and allied disorders, addiction, obesity, tic disorders, attention deficit hyperactivity disorder (ADHD), Parkinson's disease, chronic pain and Alzheimer's disease or other cognitive impairments. (+)-1-(3,4-dichlorophenyl)-3-azabicyclo[3.1.0]hexane is particularly useful in treating depression in those who have been previously treated for a condition affected by monoamine neurotransmitters, specifically those who have failed an initial course of antidepressant therapy, such as selective serotonin reuptake inhibitor therapy, or who have relapsed after an initial course of antidepressant therapy. Additional information regarding (+)-1-(3,4-dichlorophenyl)-3-azabicyclo[3.1.0]hexane may be found in U.S. patent application Ser. No. 13/310,694, filed Dec. 2, 2011, and its predecessor application, U.S. Provisional Patent Application No. 61/419,769, filed Dec. 3, 2010, both of which are incorporated by reference herein in their entirety.

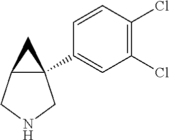 [SMILES: Clc1ccc([C@]23CNC[C@H]2C3)cc1Cl]

### (+)-1-(3,4-dichlorophenyl)-3-azabicyclo[3.1.0]hexane

An efficient means of preparing (+)-1-(3,4-dichlorophenyl)-3-azabicyclo[3.1.0]hexane is described in U.S. patent application Ser. No. 11/740,667, incorporated herein by reference in its entirety. Additional exemplary means of preparing (+)-1-(3,4-dichlorophenyl)-3-azabicyclo[3.1.0]hexane may be found, for example, in U.S. patent application Ser. Nos. 10/920,748; 11/205,956; 12/208,284; 12/428,399; WO20040466457; WO2007127396; WO02066427; WO2006023659; and U.S. Pat. No. 6,372,919, each of which is incorporated herein by reference in its entirety.

Methods for preparing (±)-1-(3,4-dichlorophenyl)-3-azabicyclo[3.1.0]hexane may be found, for example, in U.S. Pat. No. 4,435,419 and U.S. patent application Ser. Nos. 10/920,748; 11/205,956; 12/208,284; 12/428,399 each of which is incorporated herein by reference in their entirety.

As used herein, the term “substantially pure (+)-1-(3,4-dichlorophenyl)-3-azabicyclo[3.1.0]hexane” or “enantiomerically pure (+)-1-(3,4-dichlorophenyl)-3-azabicyclo[3.1.0]hexane” means that the compositions contain more (+)-1-(3,4-dichlorophenyl)-3-azabicyclo[3.1.0]hexane than (−)-1-(3,4-dichlorophenyl)-3-azabicyclo[3.1.0]hexane. Specifically, the compositions refer to an enantiomeric excess greater than 80%, preferably greater than 90%, more preferably greater than 95%, and most preferably greater than 98%, greater than 99% of the (+)-1-(3,4-dichlorophenyl)-3-azabicyclo[3.1.0]hexane, as determined by configuration and/or optical activity. Typically, the compositions contain no more than about 5% w/w of the corresponding (−) enantiomer, more preferably no more than about 2%, more preferably no more than about 1% w/w of the corresponding (−) enantiomer of (+)-1-(3,4-dichlorophenyl)-3-azabicyclo[3.1.0]hexane.

(+)-1-(3,4-dichlorophenyl)-3-azabicyclo[3.1.0]hexane is polymorphic. The present invention comprises the use of one or more polymorphic forms of (+)-1-(3,4-dichlorophenyl)-3-azabicyclo[3.1.0]hexane, specifically forms A, B and C as disclosed in U.S. patent application Ser. Nos. 11/205,956; 12/208,284 and 12/428,399 incorporated herein by reference in their entirety.

Polymorph form A may be characterized as the hemi-hydrate of acid addition salts of (+)-1-(3,4-dichlorophenyl)-3-azabicyclo[3.1.0]hexane. The polymorphs of acid addition salts of (+)-1-(3,4-dichlorophenyl)-3-azabicyclo[3.1.0]hexane may be characterized by their X-ray powder diffraction patterns (XRPD) and/or their Raman spectroscopy peaks. A Bragg-Brentano instrument, which includes the Shimadzu system, used for the X-ray powder diffraction pattern measurements reported herein, gives a systematic peak shift (all peaks can be shifted at a given “° 2θ” angle) which result from sample preparation errors as described in Chen et al., J Pharmaceutical and Biomedical Analysis, 2001; 26, 63. Therefore, any “° 2θ” angle reading of a peak value is subject to an error of about (±) 0.2°.

The following Table 1 shows the values for the relative intensities for peaks of the X-ray powder diffraction pattern of purified polymorph form A of the hydrochloride salt of (+)-1-(3,4-dichlorophenyl)-3-azabicyclo[3.1.0]hexane having a crystal size of from about 10 to 40 microns. With respect to the percent value of relative intensity (I/Io) given in Table 1, Io represents the value of the maximum peak determined by XRPD for the sample for all “° 2θ” angles and I represents the value for the intensity of a peak measured at a given “° 2θ” angle”. The angle “° 2θ” is a diffraction angle which is the angle between the incident X-rays and the diffracted X-rays.

TABLE 1
XRPD Peaks (°2θ) and Relative Intensities (I/Io) for Polymorph Form A
Form A

| °2θ | I/Io |
|---|---|
| 4.55 | 25 |
| 9.10 | 15 |
| 13.65 | 6 |
| 17.14 | 60 |
| 17.85 | 11 |
| 18.24 | 23 |
| 18.49 | 14 |
| 19.27 | 14 |
| 19.62 | 22 |
| 21.74 | 15 |
| 21.96 | 100 |
| 22.24 | 12 |
| 23.01 | 7 |
| 24.52 | 43 |
| 24.79 | 10 |
| 26.74 | 52 |
| 27.44 | 11 |
| 27.63 | 17 |
| 28.36 | 16 |
| 28.48 | 26 |
| 29.00 | 14 |
| 29.20 | 19 |
| 29.40 | 27 |
| 29.57 | 27 |
| 30.24 | 18 |
| 31.01 | 13 |
| 31.62 | 17 |
| 32.20 | 24 |
| 32.93 | 12 |
| 33.42 | 9 |
| 34.24 | 6 |
| 35.08 | 15 |
| 35.65 | 16 |
| 36.31 | 14 |
| 37.11 | 26 |
| 37.78 | 9 |
| 39.85 | 9 |

The following Table 2 shows the relative intensities for peaks of the X-ray powder diffraction pattern of purified polymorph form B of the hydrochloride salt of (+)-1-(3,4-dichlorophenyl)-3-azabicyclo[3.1.0]hexane having a crystal size of from about 10 to 40 microns.

TABLE 2
XRPD Peaks (°2θ) and Relative Intensities (I/Io) for Polymorph Form B
Form B

| °2θ | I/Io |
|---|---|
| 10.50 | 6 |
| 13.34 | 12 |
| 15.58 | 42 |
| 17.12 | 6 |
| 17.36 | 8 |
| 17.52 | 26 |
| 18.21 | 11 |
| 20.40 | 7 |
| 21.35 | 97 |
| 21.61 | 17 |
| 21.93 | 11 |
| 22.64 | 6 |
| 23.04 | 79 |
| 24.09 | 6 |
| 24.52 | 14 |
| 25.43 | 96 |
| 26.24 | 53 |
| 26.36 | 73 |
| 26.75 | 11 |
| 26.88 | 7 |
| 27.44 | 6 |
| 27.94 | 12 |
| 28.36 | 20 |
| 28.54 | 30 |
| 29.39 | 10 |
| 29.72 | 9 |
| 30.07 | 7 |
| 30.58 | 8 |
| 30.72 | 100 |
| 31.07 | 14 |
| 31.38 | 12 |
| 31.55 | 7 |
| 31.78 | 12 |
| 32.14 | 10 |
| 32.31 | 7 |
| 32.80 | 7 |
| 32.95 | 6 |
| 33.45 | 44 |
| 33.74 | 12 |
| 35.25 | 10 |
| 35.40 | 12 |
| 35.58 | 9 |
| 36.75 | 8 |
| 37.55 | 18 |
| 39.01 | 15 |
| 39.22 | 7 |
| 39.37 | 7 |
| 39.86 | 11 |

The following Table 3 shows the values of the relative intensities of the peaks of the X-ray powder diffraction pattern of purified polymorph form C of the hydrochloride salt of (+)-1-(3,4-dichlorophenyl)-3-azabicyclo[3.1.0]hexane having a crystal size of from about 10 to 40 microns.

TABLE 3
XRPD Peaks (°2θ) and Relative Intensities (I/Io) for Polymorph Form C
Form C

| °2θ | I/Io |
|---|---|
| 5.46 | 6 |
| 5.66 | 20 |
| 6.37 | 6 |
| 7.26 | 6 |
| 8.75 | 6 |
| 13.34 | 25 |
| 13.94 | 11 |
| 15.65 | 7 |
| 16.26 | 7 |
| 17.01 | 8 |
| 17.38 | 9 |
| 17.64 | 83 |
| 17.92 | 15 |
| 18.23 | 40 |
| 19.08 | 7 |
| 19.38 | 46 |
| 19.86 | 20 |
| 20.07 | 100 |
| 21.16 | 17 |
| 21.32 | 94 |
| 21.64 | 37 |
| 22.42 | 25 |
| 22.70 | 12 |
| 22.97 | 70 |
| 23.31 | 6 |
| 24.09 | 15 |
| 24.86 | 94 |
| 25.24 | 32 |
| 25.38 | 49 |
| 26.12 | 13 |
| 26.32 | 90 |
| 26.87 | 18 |
| 27.21 | 39 |
| 27.90 | 54 |
| 28.14 | 8 |
| 28.56 | 32 |
| 28.74 | 17 |
| 29.20 | 6 |
| 29.72 | 6 |
| 29.92 | 26 |
| 30.54 | 13 |
| 30.72 | 19 |
| 30.96 | 31 |
| 31.42 | 7 |
| 31.68 | 11 |
| 31.80 | 15 |
| 31.97 | 6 |
| 32.43 | 21 |
| 33.26 | 12 |
| 33.40 | 15 |
| 33.64 | 25 |
| 33.84 | 18 |
| 34.11 | 15 |
| 34.70 | 11 |
| 35.07 | 8 |
| 35.64 | 11 |
| 35.91 | 8 |
| 36.09 | 21 |
| 37.80 | 12 |
| 38.06 | 6 |
| 38.17 | 6 |
| 39.04 | 6 |
| 39.23 | 8 |
| 39.77 | 7 |

There are key major peaks at given angles in these X-ray powder diffraction patterns which are unique to each given polymorph form. These peaks are present in the XRPD patterns of each of the polymorph forms having a crystal size of about 10 to 40 microns. Any of these major peaks, either alone or in any distinguishing combination, are sufficient to distinguish one of the polymorph forms from the other two polymorph forms. For polymorph form A, the “° 2θ” angles of these major peaks which characterize polymorph form A, subject to the error set forth above, are as follows: 17.14; 19.62; 21.96; 24.52; and 26.74. For polymorph form B, the “° 2θ” angles of these major peaks which characterize polymorph form B, subject to the error set forth above, are as follows: 15.58; 17.52; 21.35; 23.04; 25.43; and 30.72. For polymorph form C, the “° 2θ” angles of these major peaks which characterize polymorph form C, subject to the error set forth above, are as follows: 13.34; 17.64; 20.07; 21.32; 22.97; 24.86; 26.32; and 27.90. Any of these major peaks, either alone or in any distinguishing combination, are sufficient to distinguish a polymorph from the other polymorph forms.

Another method of characterizing the three polymorphs of (+)-1-(3,4-dichlorophenyl)-3-azabicyclo[3.1.0]hexane is through Raman spectroscopy. The procedure for carrying out Raman Spectroscopy is described on pages 260-275 of Skoog and West, Principles of Instrumental Analysis (2nd Ed.); Saunders College, Philadelphia (1980).

The Raman spectra peak positions in wavenumbers (cm⁻¹) for polymorph form A, B and C of the hydrochloride salt of (+)-1-(3,4-dichlorophenyl)-3-azabicyclo[3.1.0]hexane are given in Table 4, below.

TABLE 4
Raman Peak Listing for Polymorph Form A, B and C (peaks > 400 cm⁻¹)
Peak Positions In Wavenumbers (cm⁻¹)

| Form A | Form B | Form C | | Form B | Form A |
|---|---|---|---|---|---|
| 436 | 418 | 441 | 1246 | 1245 | 1135 |
| 479 | 446 | 474 | 1266 | 1278 | 1189 |
| 534 | 478 | 532 | 1279 | 1309 | 1229 |
| 549 | 533 | 648 | 1309 | 1343 | 1274 |
| 646 | 648 | 674 | 1343 | 1380 | 1309 |
| 691 | 676 | 690 | 1398 | 1398 | 1338 |
| 680 | 686 | 767 | 1456 | 1456 | 1366 |
| 762 | 767 | 811 | 1471 | 1483 | 1393 |
| 812 | 825 | 826 | 1557 | 1557 | 1453 |
| 836 | 852 | 856 | 1595 | 1593 | 1484 |
| 892 | 895 | 895 | 2900 | 2895 | 1557 |
| 921 | 964 | 970 | 2966 | 2963 | 1597 |
| 959 | 979 | 1031 | 2992 | 2993 | 2890 |
| 982 | 1031 | 1059 | 3048 | 3027 | 2969 |
| 998 | 1054 | 1094 | 3070 | 3066 | 2982 |
| 1030 | 1070 | 1122 | | | 3017 |
| 1056 | 1099 | 1137 | | | 3046 |
| 1099 | 1136 | 1189 | | | 3064 |
| 1122 | 1189 | 1228 | | | |

Table 4 provides the complete patterns of the Raman peak positions with respect to the hydrochloride salts of polymorph forms A, B and C respectively. However, there are certain key peaks within these patterns which are unique to each of the hydrochloride salts of these polymorphs. Any of these key peaks, either alone or in any distinguishing combination, are sufficient to distinguish one of the polymorph forms from the other two polymorph forms. These peak positions, expressed in wavenumbers (cm⁻¹) for the hydrochloride salt of polymorph form A are: 762; 836; 921; 959; 1393; 1597; 2890; 2982; and 3064. The characterizing peak positions expressed in wavenumbers (cm⁻¹) for the hydrochloride salt of polymorph form B are: 1245; 1380; 2963; 2993; 3027; and 3066. The characterizing peak positions expressed in wavenumbers (cm⁻¹) for the hydrochloride salt of polymorph form C are: 1059; 1094; 1266; 1343; 1595; 2900; 2966; and 3070. Any of these key peaks, either alone or in any distinguishing combination, are sufficient to distinguish each polymorph form from the other two polymorph forms.

Polymorph forms A, B and C of (+)-1-(3,4-dichlorophenyl)-3-azabicyclo[3.1.0]hexane, particularly as hydrochloride acid addition salts, can each be prepared substantially free of its other enantiomeric, geometric and polymorphic isomeric forms through re-crystallization of a mixture of the A and B polymorph forms produced in accordance with prior art procedures. Depending upon the particular solvent, conditions and concentrations of materials utilized to re-crystallize the mixture of polymorph forms A and B, one can selectively produce the desired polymorph form of (+)-1-(3,4-dichlorophenyl)-3-azabicyclo[3.1.0]hexane, substantially free of its other enantiomeric, geometric and polymorphic isomers. The term “substantially free” of its other enantiomeric, geometric and polymorphic isomeric forms designates that the crystalline material is at least about 95% by weight pure in that it contains no more than about 5% w/w of its other enantiomeric, geometric and polymorphic isomeric forms.

In preparing polymorph forms A and B substantially free of other polymorph forms, crystallization from a mixture of A and B may be utilized. However, the crystallization technique with regard to producing each of these polymorph forms substantially free of other polymorph forms is different. In preparing polymorph form A, which is the hemi-hydrate of the acid addition salt of (+)-1-(3,4-dichlorophenyl)-3-azabicyclo[3.1.0]hexane, it is best to utilize a solvent medium to dissolve a solid containing polymorph form A such as a mixture of polymorph forms A and B in an organic solvent which contains water. The preferred organic solvents that can be utilized in this procedure include lower alkanol solvents such as methanol, butanol, ethanol or isopropanol as well as other solvents such as acetone, dichloromethane and tetrahydrofuran.

Polymorph form B is the anhydrous form of the acid addition salt of (+)-1-(3,4-dichlorophenyl)-3-azabicyclo[3.1.0]hexane. Polymorph form B of the acid addition salt of (+)-1-(3,4-dichlorophenyl)-3-azabicyclo[3.1.0]hexane can be prepared from a solid containing polymorph form A or a mixture of polymorph forms A and B by dissolving the polymorph form A or the mixture of polymorph forms A and B, preferably as the hydrochloride salt, utilizing anhydrous conditions.

Polymorph form C can be prepared from either polymorph form A or polymorph form B or mixtures thereof. Polymorph form C is prepared by extensive heating of either polymorph form A or polymorph form B, or mixtures thereof, at temperatures of at least 50° C., preferably from 60° C. to 80° C. Heating can be continued until polymorph form C substantially free of other polymorph forms is formed.

The techniques set forth above also allow for the preparation of mixtures of the individual polymorph forms of the acid addition salt of (+)-1-(3,4-dichlorophenyl)-3-azabicyclo[3.1.0]hexane containing specific amounts of each of the polymorphs. In particular, mixtures of polymorph form A and either polymorph form B or polymorph form C; polymorph form B and polymorph form C; and polymorph form A, polymorph form B and polymorph form C can be readily prepared with the desired amounts of each of the polymorphs. Using the techniques set forth above, mixtures containing specific percentages of the individual polymorphic forms of the acid addition salt of (+)-1-(3,4-dichlorophenyl)-3-azabicyclo[3.1.0]hexane can be obtained. For example, mixtures containing from about 10% to about 10-20%, 20-35%, 35-50%, 50-70%, 70-85%, 85-95% and up to 95-99% or greater (by weight) of polymorph form A, with the remainder of the mixture being either or both polymorph form B and polymorph form C, can be prepared. As another example, mixtures containing from about 10% to about 10-20%, 20-35%, 35-50%, 50-70%, 70-85%, 85-95% and up to 95-99% or greater (by weight) of polymorph form B, with the remainder of the mixture being either or both polymorph form A and polymorph form C, can be prepared. As a further example, mixtures containing from about 10% to about 10-20%, 20-35%, 35-50%, 50-70%, 70-85%, 85-95% and up to 95-99% or greater (by weight) of polymorph form C, with the remainder of the mixture being either or both polymorph form A and polymorph form B, can be prepared.

Additionally, many pharmacologically active organic compounds regularly crystallize incorporating second, foreign molecules, especially solvent molecules, into the crystal structure of the principal pharmacologically active compound to form pseudopolymorphs. When the second molecule is a solvent molecule, the pseudopolymorphs can also be referred to as solvates. All of these additional forms of (+)-1-(3,4-dichlorophenyl)-3-azabicyclo[3.1.0]hexane are likewise contemplated for use within the present invention.

The polymorph forms A, B and C of the present invention can be prepared as acid addition salts formed from an acid and the basic nitrogen group of (+)-1-(3,4-dichlorophenyl)-3-azabicyclo[3.1.0]hexane. Suitable acid addition salts are formed from acids, which form non-toxic salts, examples of which are hydrochloride, hydrobromide, hydroiodide, sulphate, hydrogen sulphate, nitrate, phosphate, and hydrogen phosphate. Examples of pharmaceutically acceptable addition salts include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like. The hydrochloride salt formed with hydrochloric acid is an exemplary useful salt.

Additionally contemplated herein are isolation of and use of the metabolites of (+)-1-(3,4-dichlorophenyl)-3-azabicyclo[3.1.0]hexane. Exemplary metabolites include the lactam 5-(3,4-dichloro-phenyl)-3-aza-bicyclo[3.1.0]hexan-2-one (EB-10101) and the carbamate, which is subsequently conjugated to form the glucuronide.

As disclosed herein, (+)-1-(3,4-dichlorophenyl)-3-azabicyclo[3.1.0]hexane agents ((+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) are effective in treating a variety of conditions including, but not limited to, depression, anxiety, panic disorder, post-traumatic stress disorder, obsessive compulsive disorder, schizophrenia and allied disorders, addiction, obesity, tic disorders, Parkinson's disease, ADHD, chronic pain and Alzheimer's disease or other cognitively impairing conditions. Within related aspects of the invention, combinatorial formulations are provided that use substantially pure (+)-1-(3,4-dichlorophenyl)-3-azabicyclo[3.1.0]hexane, or pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane alone or in combination with other psychotherapeutic drugs to modulate, prevent, alleviate, ameliorate, reduce or treat symptoms or conditions influenced by monoamine neurotransmitters or biogenic amines. Subjects amenable to treatment according to the invention include mammalian subjects, including humans, suffering from or at risk for any of a variety of conditions including, but not limited to, depression, anxiety, panic disorder, post-traumatic stress disorder, obsessive compulsive disorder, schizophrenia and allied disorders, obesity, tic disorders, addiction, ADHD, Parkinson's disease, chronic pain and Alzheimer's disease or other cognitively impairing conditions.

(+)-1-(3,4-dichlorophenyl)-3-azabicyclo[3.1.0]hexane or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane may be administered alone or in combination with one or more other psychotherapeutic drugs including, but not limited to, drugs from the general classes of anti-convulsant, mood-stabilizing, anti-psychotic, anxiolytic, benzodiazepines, calcium channel blockers, and antidepressants. (See, e.g., R J. Baldessarini in Goodman & Gilman's The Pharmacological Basis of Therapeutics, 11th Edition, Chapters 17 and 18, McGraw-Hill, 2005 for a review). Additionally, (+)-1-(3,4-dichlorophenyl)-3-azabicyclo[3.1.0]hexane or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane may be administered in combination with anti-inflammatory or other pain relieving medications.

Within the coordinate administration methods of the invention, the secondary therapeutic and/or psychotherapeutic drug is administered concurrently or sequentially with (+)-1-(3,4-dichlorophenyl)-3-azabicyclo[3.1.0]hexane, or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane to treat or prevent one or more symptoms of the targeted disorder. When administered simultaneously, the additional therapeutic and/or psychotherapeutic agent and (+)-1-(3,4-dichlorophenyl)-3-azabicyclo[3.1.0]hexane or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane may be combined in a single composition or combined dosage form. Alternatively, the combinatorially effective additional therapeutic and/or psychotherapeutic drug and (+)-1-(3,4-dichlorophenyl)-3-azabicyclo[3.1.0]hexane agents (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) may be administered at the same time in separate dosage forms. When the coordinate administration is conducted simultaneously or sequentially, the additional therapeutic and/or psychotherapeutic agent and (+)-1-(3,4-dichlorophenyl)-3-azabicyclo[3.1.0]hexane agent may each exert biological activities and therapeutic effects over different time periods, although a distinguishing aspect of all coordinate treatment methods of the invention is that treated subjects exhibit positive therapeutic benefits.

Administration of (+)-1-(3,4-dichlorophenyl)-3-azabicyclo[3.1.0]hexane, a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane or the coordinate treatment method or combinatorial drug composition of the invention to suitable subjects will yield a reduction in one or more target symptom(s) associated with the selected disorder or development of the disorder by at least 2%, 5%, 10%, 20%, 30%, 50% or greater, up to a 75-90%, or 95% or greater, compared to placebo-treated or other suitable control subjects. Comparable levels of efficacy are contemplated for the entire range of disorders described herein, including all contemplated neurological and psychiatric disorders, and related conditions and symptoms, for treatment or prevention using the compositions and methods of the invention. These values for efficacy may be determined by comparing accepted therapeutic indices or clinical values for particular test and control individuals over a course of treatment/study, or more typically by comparing accepted therapeutic indices or clinical values between test and control groups of individuals using standard human clinical trial design and implementation.

As used herein, the terms “prevention” and “preventing,” when referring to a disorder or symptom, refers to a reduction in the risk or likelihood that a mammalian subject will develop said disorder, symptom, condition, or indicator after treatment according to the invention, or a reduction in the risk or likelihood that a mammalian subject will exhibit a recurrence or relapse of said disorder, symptom, condition, or indicator once a subject has been treated according to the invention and cured or restored to a normal state (e.g., placed in remission from a targeted disorder). As used herein, the terms “treatment” or “treating,” when referring to the targeted disorder, refers to inhibiting or reducing the progression, nature, or severity of the subject condition, or delaying the onset of the condition.

In accordance with the invention, compounds disclosed herein, optionally formulated with additional ingredients in a pharmaceutically acceptable composition, are administered to mammalian subjects, for example a human patient, to treat or prevent one or more symptom(s) of a disorder alleviated by inhibiting dopamine reuptake, and/or norepinephrine reuptake, and/or serotonin reuptake. In certain embodiments, “treatment” or “treating” refers to amelioration of one or more symptom(s) of a disorder, whereby the symptom(s) is/are alleviated by inhibiting dopamine and/or norepinephrine and/or serotonin reuptake. In other embodiments, “treatment” or “treating” refers to an amelioration of at least one measurable physical parameter associated with a disorder. In yet another embodiment, “treatment” or “treating” refers to inhibiting or reducing the progression or severity of a disorder (or one or more symptom(s) thereof) alleviated by inhibiting dopamine and/or norepinephrine and/or serotonin reuptake, e.g., as discerned based on physical, physiological, and/or psychological parameters. In additional embodiments, “treatment” or “treating” refers to delaying the onset of a disorder (or one or more symptom(s) thereof) alleviated by inhibiting dopamine and/or norepinephrine and/or serotonin reuptake.

An “effective amount,” “therapeutic amount,” “therapeutically effective amount,” or “effective dose” of (+)-1-(3,4-dichlorophenyl)-3-azabicyclo[3.1.0]hexane (amitifadine) agent (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) and/or an additional psychotherapeutic agent as used herein means an effective amount or dose of the active compound as described herein sufficient to elicit a desired pharmacological or therapeutic effect in a human subject. In the case of antidepressant therapeutic agents, these terms most often refer to a measurable, statistically significant reduction in an occurrence, frequency, or severity of one or more symptom(s) of a specified disorder, including any combination of neurological and/or psychological symptoms, diseases, or conditions, associated with or caused by the targeted disorder and/or reduction in the development of depression in a target population.

Therapeutic efficacy can alternatively be demonstrated by a decrease in the frequency or severity of symptoms associated with the treated condition or disorder, or by altering the nature, occurrence, recurrence, or duration of symptoms associated with the treated condition or disorder. In this context, “effective amounts,” “therapeutic amounts,” “therapeutically effective amounts,” and “effective doses” of additional psychotherapeutic drugs and (+)-1-(3,4-dichlorophenyl)-3-azabicyclo[3.1.0]hexane agents (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) within the invention can be readily determined by ordinarily skilled artisans following the teachings of this disclosure and employing tools and methods generally known in the art, often based on routine clinical or patient-specific factors.

Efficacy of the coordinate treatment methods and drug compositions of the invention will often be determined by use of conventional patient surveys or clinical scales to measure clinical indices of disorders in subjects. The methods and compositions of the invention will yield a reduction in one or more scores or selected values generated from such surveys or scales completed by test subjects (indicating for example an incidence or severity of a selected disorder), by at least 5%, 10%, 20%, 30%, 50% or greater, up to a 75-90%, or 95% compared to correlative scores or values observed for control subjects treated with placebo or other suitable control treatment. In at risk populations, the methods and compositions of the invention will yield a stable or minimally variable change in one or more scores or selected values generated from such surveys or scales completed by test subjects. More detailed data regarding efficacy of the methods and compositions of the invention can be determined using alternative clinical trial designs.

Useful patient surveys and clinical scales for comparative measurement of clinical indices of psychiatric disorders in subjects treated using the methods and compositions of the invention can include any of a variety of widely used and well known surveys and clinical scales. Among these useful tools are the Mini International Neuropsychiatric Interview© (MINI) (Sheehan et al., 1998); Clinical Global Impression scale (CGI) (Guy, W., ECDEU Assessment Manual for Psychopharmacology, DHEW Publication No. (ADM) 76-338, rev. 1976); Clinical Global Impression Severity of Illness (CGI-S) (Guy, 1976); Clinical Global Impression Improvement (CGI-I) (Guy, et al. 1976); Beck Depression Inventory (BDI) (Beck, 2006); Revised Hamilton Rating Scale for Depression (RHRSD) (Warren, 1994); Major Depressive Inventory (MDI) (Olsen et al. 2003); and Children's Depression Index (CDI) (Kovacs, et al. 1981); Hamilton Depression Rating Scale© (HDRS) (Hamilton, M., J. Neurol. Neurosurg. Psychiatr. 23:56-62, 1960; Hamilton, M., Br. J. Soc. Clin. Psychol. 6:278-296, 1967); Montgomery-Asberg Depression Rating Scale© (MADRS) (Montgomery and Asberg, 1979); Beck Scale for Suicide Ideation® (BSS) (Beck and Steer, 1991 Columbia-Suicide Severity Rating Scale© (C-SSRS) or Columbia Classification Algorithm of Suicide Assessment© (C CASA) (Posner, K, et al., 2007); Sheehan-Suicidality Tracking Scale© (S-SST) (Coric et al., 2009); Beck Hopelessness Scale© (BHS) (Beck, Steer, 1988); Geriatric Depression Scale (GDS) (Yesavage, J. A. et al., J. Psychiatr. Res. 17:37-49, 1983); and the HAM-D scale for depression (Hamilton, 1960).

Useful patient surveys and clinical scales for comparative measurement of cravings associated with addictive or compulsive behaviors (e.g., an urge or desire to smoke or to use drugs of abuse) include one or more multidimensional scales such as the Questionnaire of Smoking Urges (QSU) developed by Tiffany & Drobes (Br. J. Addict. 86(11):1467-76 (1991)) which assesses the subject's desire to smoke and his or her expectancies of both positive and negative reinforcement from smoking and intention to smoke. Other questionnaires or indices useful for assessing cravings for nicotine or smoking can include the Measurement of Drug Craving scale (Sayette et al. 2000), Drug History Questionnaire (DHQ), Desires for Drug Questionnaire (Franken et al., Addict. Behay. 27:675-85 (2002)), Heaviness of Smoking Index (HSI), the Fagerstrom Test for Nicotine Dependence (FTND), Drinker Inventory of Consequences (DrInC) (Forcehimes et al., Addict Behay. 2007 August; 32(8):1699-704. Epub 2006 Dec. 19. Psychometrics of the Drinker Inventory of Consequences (DrInC)) and Profile of Mood States (POMS) (Nyenhuis et al., J Clin Psychol. 1999 January; 55(1):79-86). In some cases, cravings or impulses associated with addictive and/or compulsive behaviors or behavioral modification protocols can be assessed using the Obsessive-Compulsive Beliefs Questionnaire-87 (OBQ-87).

The methods and compositions of the invention will yield a reduction in one or more scores or values generated from these clinical surveys (using any single scale or survey, or any combination of one or more of the surveys described above) by at least 10%, 20%, 30%, 50% or greater, up to a 75-90%, or 95% compared to correlative scores or values observed for control subjects treated with placebo or other suitable control treatment. In prophylactic treatment, the methods and compositions of the invention will yield a stabilization or diminished change in the scores or values generated from these clinical surveys.

In some embodiments, administration of the pharmaceutical compositions contemplated herein will be sufficient to place an individual into remission for a condition, specifically depression. The length of time defined as remission varies by author but is generally accepted as 4 to 6 months after successful treatment (Byrne, et al., 1998). Remission from depression may be measured by any of a variety of ways, for example with patient surveys and clinical scales. An indication of remission, for example would be scores on the MADRS≦12, HAMD-17≦7 or CGI-S≦2.

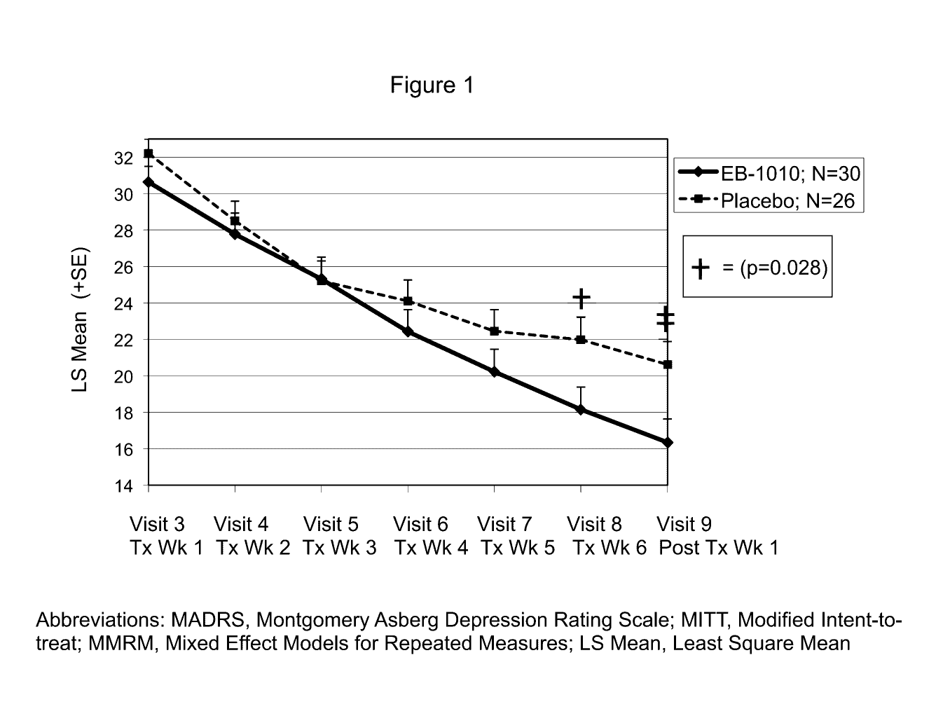

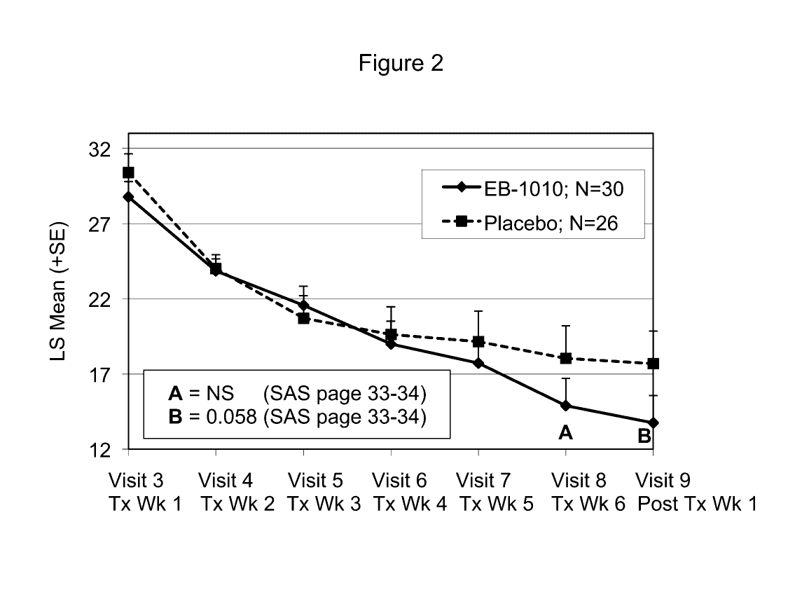

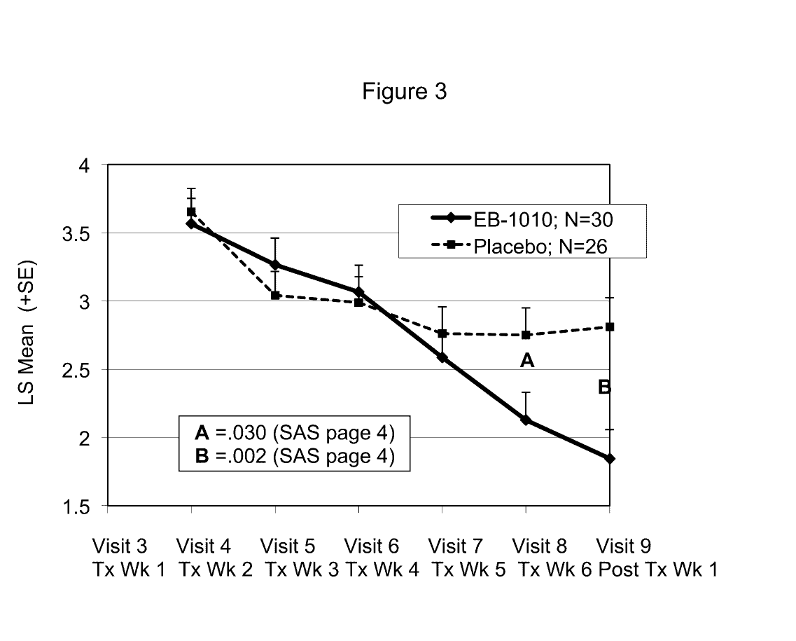

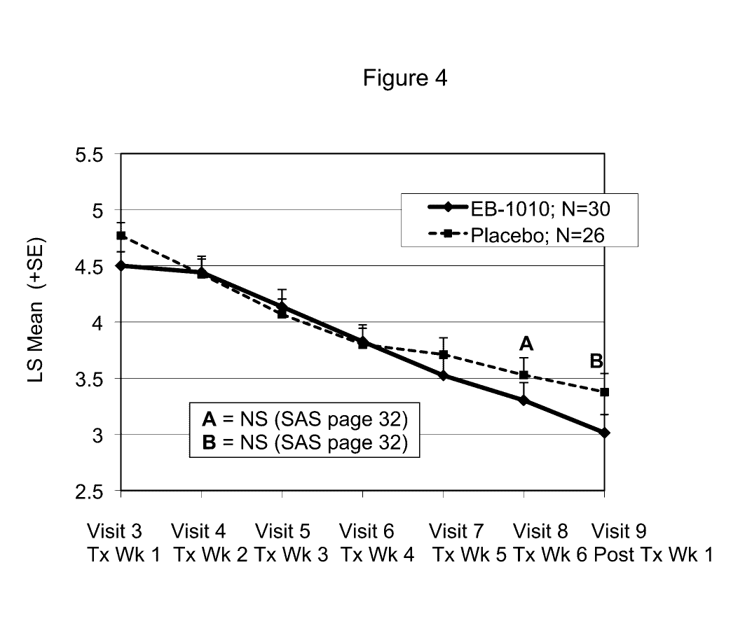

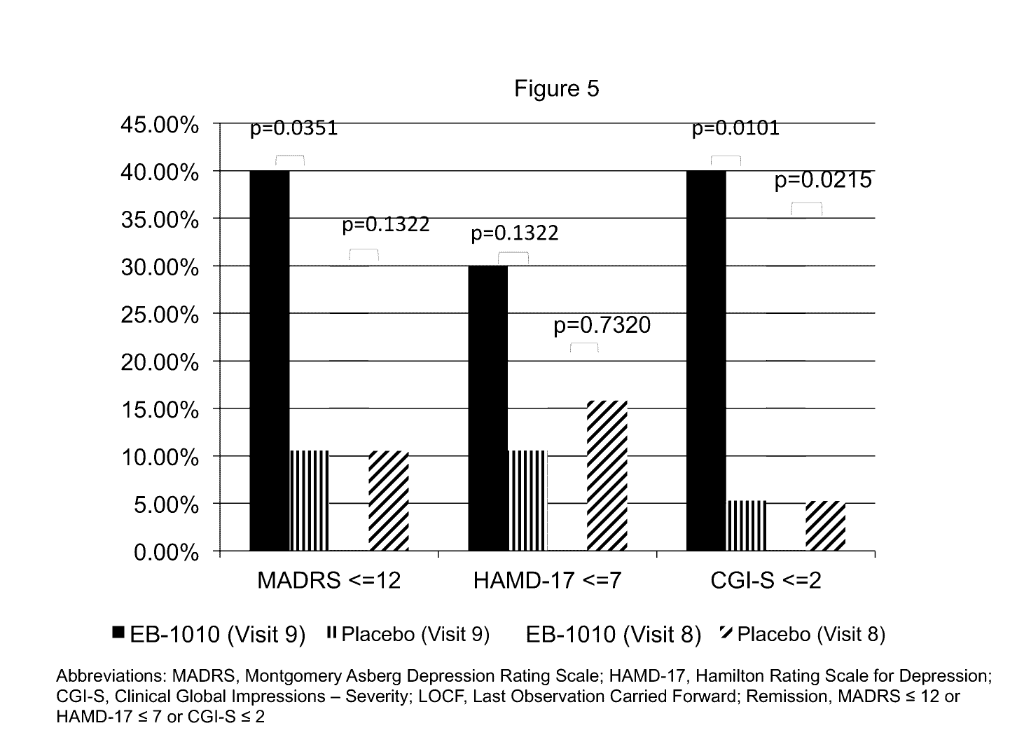

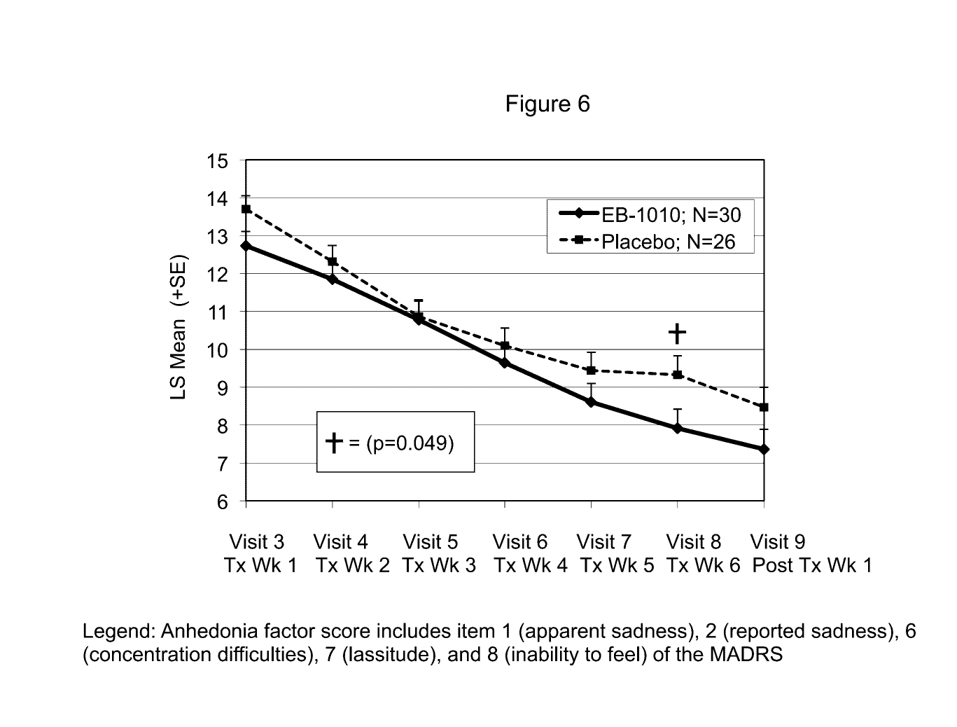

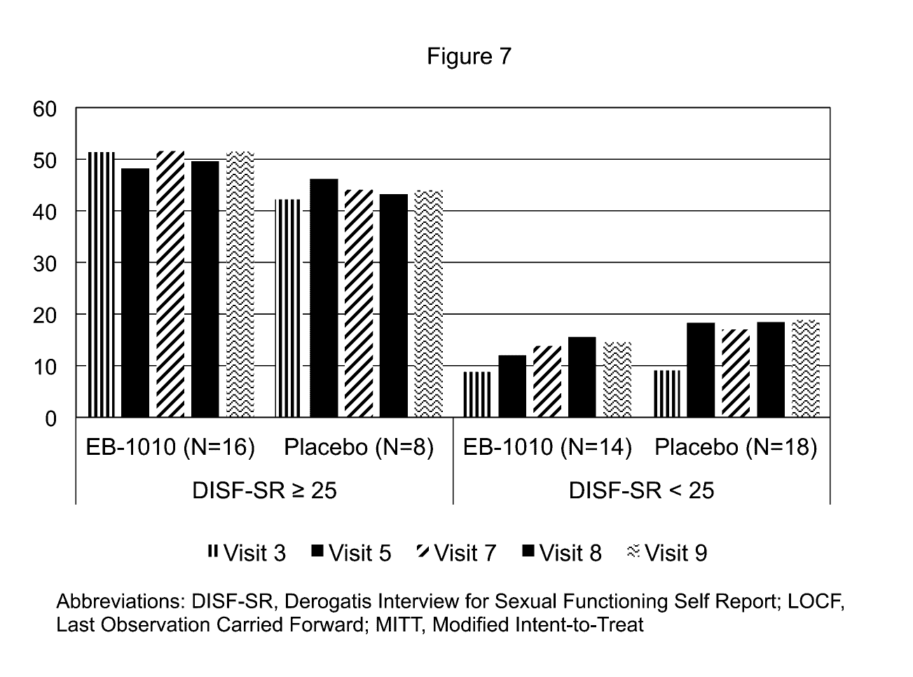

As shown in the figures and the examples below, administration of (+)-1-(3,4-dichlorophenyl)-3-azabicyclo[3.1.0]hexane in comparison to placebo in a six-week double blind study significantly decreased the depression levels in patients as measured using the Montgomery-Asberg Depression rating scale (FIG. 1, data analyzed using the mixed model for repeated measures least square means (MMRM LS)), the Hamilton Depression Rating Scale (FIG. 2, data analyzed using the mixed model for repeated measures LS means), Clinical Global Impression Improvement (CGI-I) (FIG. 3, data analyzed using the mixed model for repeated measures LS means (MMRM LS), and the Clinical Global Impression Severity of Illness (CGI-S) (FIG. 4, data analyzed using the mixed model for repeated measures LS means). Treatment with amitifadine also resulted in significantly greater remission rates than treatment with placebo (FIG. 5, as measured by the Clinical Global Impressions-Severity (CGI-S) scale using Last Observation Carried Forward (LOCF)) and statistically significant improvement on the anhedonia factor score of the MADRS compared to placebo (FIG. 6, data analyzed using the mixed model for repeated measures LS means (MMRM LS). Additionally, treatment with (+)-1-(3,4-dichlorophenyl)-3-azabicyclo[3.1.0]hexane showed no difference in comparison with placebo in evaluation of sexual dysfunction (FIG. 7, data analyzed using the last observation carried forward method (LOCF), indicating that (+)-1-(3,4-dichlorophenyl)-3-azabicyclo[3.1.0]hexane is not associated with emergence of sexual dysfunction. These results demonstrate surprising efficacy in comparison to other triple reuptake inhibitors. For example, SEP-225289, a triple reuptake inhibitor that underwent Phase II clinical testing by Sepracor, did not meet the primary efficacy endpoint compared to placebo, which was a reduction in symptoms of depression following eight weeks of treatment, as assessed using the clinician-rated, 17-item HAM-D scale (Sepracor Press Release, Jul. 1, 2009). Similarly, GSK372475, a balanced triple reuptake inhibitor in development by GlaxoSmithKline, also failed to demonstrate a significant benefit in comparison to placebo. (Graff, Ole et al. 2009).

Additionally, the unbalanced reuptake inhibition profile of (+)-1-(3,4-dichlorophenyl)-3-azabicyclo[3.1.0]hexane allows for higher doses of (+)-1-(3,4-dichlorophenyl)-3-azabicyclo[3.1.0]hexane to be used without incurring the side effects that limit the effectiveness of balanced triple reuptake inhibitors such as GSK372475. In contrast to GSK372475, (+)-1-(3,4-dichlorophenyl)-3-azabicyclo[3.1.0]hexane is well tolerated and has a similar adverse event profile as placebo. (See, Example XLVIII and Graff, et al. 2009). (+)-1-(3,4-dichlorophenyl)-3-azabicyclo[3.1.0]hexane use also did not lead to the noradrenergic side effects such as significantly elevated heart rate and increased systolic and diastolic blood pressure seen with GSK37425 (See Tables 52 and 53 and Graff, 2009) or dopaminergic side effects such as nausea, vomiting, and hypomania.

The SEP-22589 inhibition profile for 5-HT, NE and DA (IC50's, SEP-289: 15, 4 and 3 nM (Schrieber, 2009)) is about equipotent for norepinephrine and dopamine reuptake inhibition and less potent for serotonin reuptake inhibition, leading to higher rates of noradrenergic or dopaminergic side effects than similar anti-depressant effective amounts of (+)-1-(3,4-dichlorophenyl)-3-azabicyclo[3.1.0]hexane.

The use of (+)-1-(3,4-dichlorophenyl)-3-azabicyclo[3.1.0]hexane will have substantially fewer dopaminergic or noradrenergic side effects than use of similar doses of balanced triple reuptake inhibitors. The use of substantially pure (+)-1-(3,4-dichlorophenyl)-3-azabicyclo[3.1.0]hexane will reduce adverse effects including side effects by 1%, 3%, 10%, 20%, 30%, 50% or greater, up to a 75%, 80%, 90%, or 95% or greater over use of a balanced triple reuptake inhibitor. Additionally, the use of (+)-1-(3,4-dichlorophenyl)-3-azabicyclo[3.1.0]hexane will have fewer dopaminergic or noradrenergic side effects than triple reuptake inhibitors with higher rates of inhibition for dopamine or noradrenaline reuptake. Thus, the use of substantially pure (+)-1-(3,4-dichlorophenyl)-3-azabicyclo[3.1.0]hexane will allow relatively greater reuptake inhibition of the 5-HT (serotonin) transporter, less of the NE (norepinephrine) transporter and even less of the DA (dopamine) transporter which allows maximal improvement of psychiatric symptoms while reducing adverse dopaminergic or noradrenergic effects including side effects by 1%, 3%, 10%, 20%, 30%, 50% or greater, up to a 75%, 80%, 90%, or 95% or greater over use of unbalanced triple reuptake inhibitors with higher rates of inhibition for dopamine or noradrenaline reuptake inhibitors.

The use of (+)-1-(3,4-dichlorophenyl)-3-azabicyclo[3.1.0]hexane will result in reuptake inhibition of the 5-HT transporter in individuals of about 10%, 15%, 20%, 30%, 50% or greater, up to a 75%, 80%, 90%, or 95% or greater than reuptake inhibition of the NE transporter or the DA transporter. In some embodiments reuptake inhibition of the 5HT transporter will be more than about 100% greater than reuptake inhibition of the DA or NE transporter in a particular individual. In some embodiments, reuptake inhibition of the 5-HT transporter will be two, three, four, five, six, seven or eight fold greater than the reuptake inhibition of the DA transporter. In other embodiments, reuptake inhibition of the 5-HT transporter will be one and half or twice that of the NE transporter. Reuptake inhibition of the NE transporter may be about 10%, 15%, 20%, 30%, 50% or greater, up to a 75%, 80%, 90%, or 95% or greater than reuptake inhibition of the DA transporter. In some embodiments, reuptake inhibition of the NE transporter may be two, three or four times greater than the reuptake inhibition of the DA transporter.

The use of (+)-1-(3,4-dichlorophenyl)-3-azabicyclo[3.1.0]hexane will result in binding of the 5-HT transporter in individuals at levels of about 10%, 15%, 20%, 30%, 50% or greater, up to a 75%, 80%, 90%, or 95% or greater than binding of the NE transporter or the DA transporter. In some embodiments, binding of the 5-HT transporter will be more than about 100% greater than the binding of the NE transporter or the DA transporter. In some embodiments, binding of the 5-HT transporter will be two, three, four, five, six, seven or eight fold greater than the binding of the DA transporter. In other embodiments, binding of the 5-HT transporter will be one and half or twice that of the NE transporter. Binding of the NE transporter may be about 10%, 15%, 20%, 30%, 50% or greater, up to a 75%, 80%, 90%, or 95% or greater than binding of the DA transporter in treated individuals. In some embodiments, binding of the NE transporter may be two, three or four times greater than binding of the DA transporter in an individual. The relative binding as determined by Kᵢ of 5-HT may be slightly higher, substantially higher, or significantly higher than the binding of the DA transporter or NE transporter alone or in combination.

Response to a pharmaceutical agent is related to the concentration of the agent in plasma and at its target site. Therapeutic failure may take the form of either suboptimal response (inadequate concentration at target site due to rapid metabolism) or toxicity (high concentrations due to slow metabolism). Metabolic conversion of drugs in an individual represents one of the important pharmacokinetic factors contributing to the therapeutic effect as well as the intensity and frequency of adverse effects (\check{Z}ourková et al., 2003). For example, the CYP2D6 enzyme encoded by cytochrome P450 2D6 genes plays the primary role in metabolizing antidepressants such as fluoxetine, imipramine, doxepin, duloxetine, trazodone, and mirtazapine. However, the CYP2D6 enzyme shows the largest phenotypical variability among the CYPs with more than 80 recognized alleles, 20 of which significantly alter the metabolism of drugs that are substrates for this enzyme. The wild-type CYP2D6*1 allele of the CYP2D6 gene determines the appropriate enzyme activity and is designated extensive metabolizer (EM) phenotype. EMs are the most common allele among Caucasians. The % of Caucasians with ultrarapid, extensive, intermediate, and poor metabolizer phenotypes are 1-2%, 77-92%, 2-11%, and 5-10%, respectively (Crews et al., 2012). Thus, 8-23% of Caucasians have a mutant allele of the CYP2D6 gene that creates a phenotype with altered CYP2D6 activity.

Phenotypic variability may cause differences in metabolism of drugs processed by CYP2D6 to vary from poor (little or no CYP2D6 function) to ultra-rapid (greater than normal function) complicating dosing and effectiveness of pharmaceutical agents. This variability is particularly relevant since the cytochrome P450 2D6 gene encodes the CYP2D6 enzyme which plays a primary role in the metabolism of many of the important antidepressants including fluoxetine, paroxetine, venlafaxine, desipramine, clomipramine, imipramine, nortriptyline, amitriptyline, imipramine, doxepin, duloxetine, trazodone, and mirtazapine. Similarly, the CYP2C19 enzyme which metabolizes the antidepressants citalopram, escitalopram, clomipramine, amitriptyline, sertraline, imipramine, nortriptyline and doxepin has 25 known variant alleles and may also have phenotapic variability from poor metabolizer, extensive metabolizer, rapid metabolizer and ultra-rapid metabolizer. CYP (cytochrome P450) polymorphisms are responsible for the development of a significant number of adverse drug reactions and have been estimated to account for at least 100,000 deaths and costs of 100 billion dollars a year in the U.S. alone. (Ingleman-Sundberg, 2004) Individuals who are rapid or ultra-rapid metabolizers of these psychotropic medications could be subject to a loss of antidepressant activity, whereas individuals who are poor metabolizers may become intolerant due to side effects. (Byrne et al., 1998; Mrazek et al., 2010; Lobello et al., 2010; and Parker 2011). Additionally, many currently used antipsychotics and antidepressants have a narrow therapeutic range, with concentration dependent adverse effects occurring at concentrations only slightly higher than the dose required for psychiatric effect (Van der Weide et al., 2006). The use of compositions such as amitifadine which is metabolized to a major extent by the monoamine oxidase-A system as well as a CYP enzyme would decrease the effect of CYP enzyme inhibition and drug-drug interactions that may be a factor in decreased responsiveness to a particular agent, thus yielding more predictability and effectiveness in dosing. Furthermore, the use of compositions such as amitifadine allows for easier and more predictable treatment of individuals who are being treated for multiple conditions, many of which would be treated by medications that use or modify cytochrome P450 enzyme activity.

(+)-1-(3,4-dichlorophenyl)-3-azabicyclo[3.1.0]hexane, amitifadine, is currently believed to be metabolized by both or either cytochrome P450 enzymes or monoamine oxidases (MAO). MAO are a family of flavin-adenosine-dinucleotide (FAD) continuing enzymes found bound to the outer membrane of mitochondria in most cell types in the body that catalyze the oxidation of monoamines. They are found throughout the body, both within the central nervous system (neurons and glia) and outside the central nervous system (liver, gastrointestinal tract, placenta, and platelets).

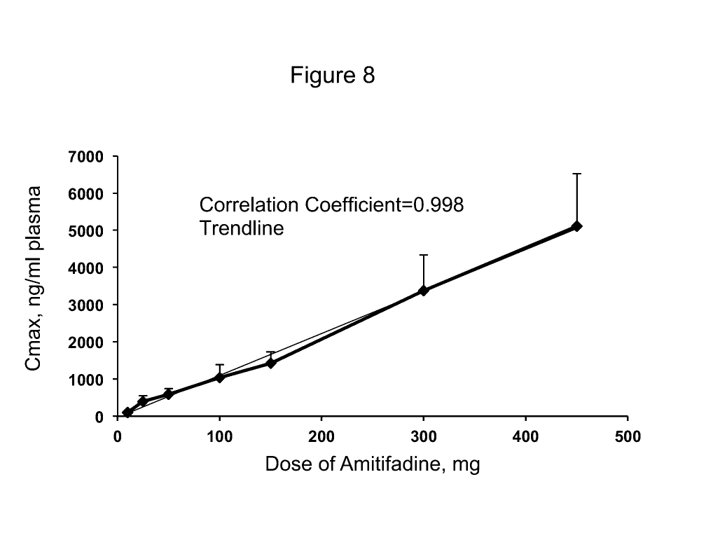

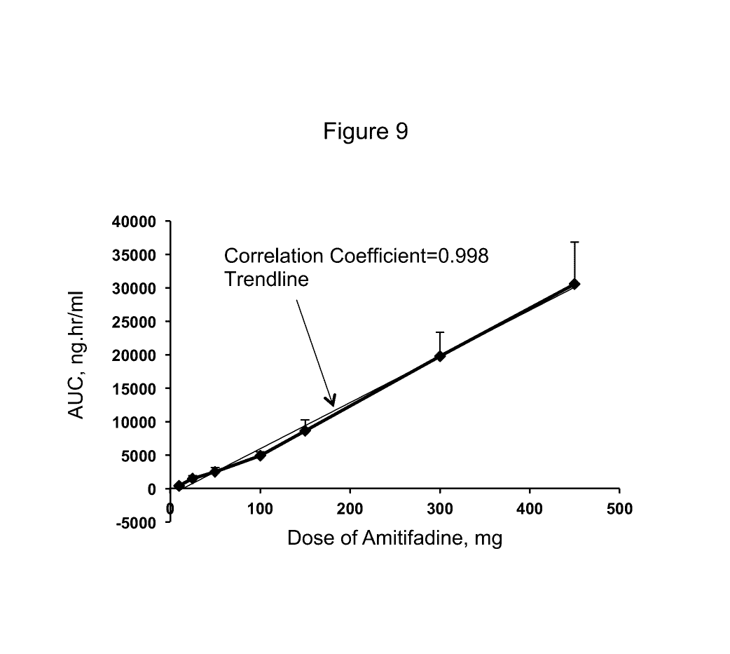

Multiple dose studies of amitifadine in animals and humans have shown linear pharmacokinetics with amitifadine, indicating no saturation of the metabolic system. The overall effect is to maintain a consistent and predictable metabolic route that does not appear to saturate (See FIGS. 8 and 9). The correlation coefficient in humans between dose and Cₘₐₓ or dose and AUC is highly correlated with a coefficient of 0.998 for both. The variability of Cₘₐₓ and AUC was ≦30% at all doses indicating predictable pharmacokinetics and low variation among subjects. This predictable plasma level allows for more controlled dosing and resultant better efficacy as well as reduced adverse events.

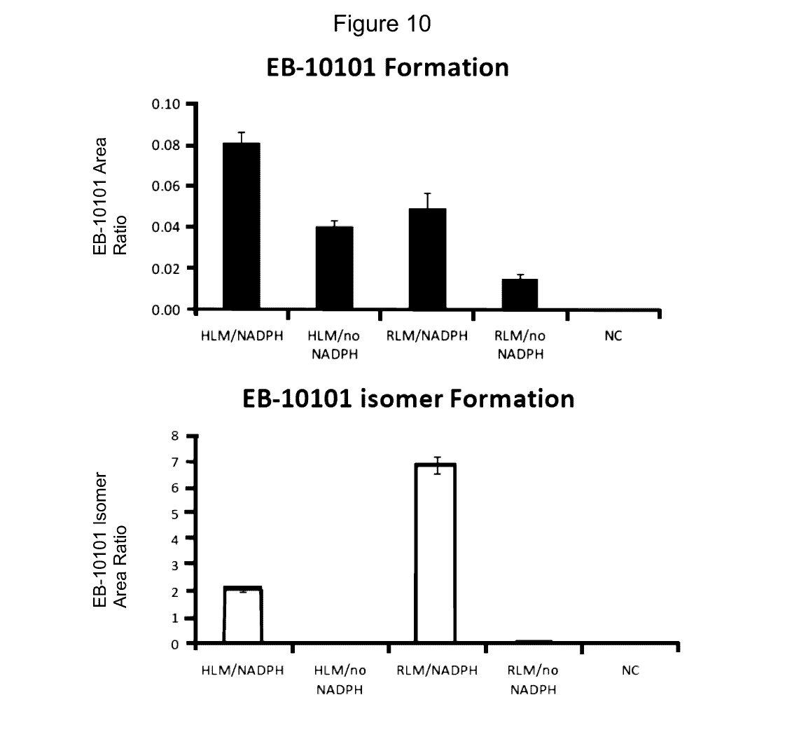

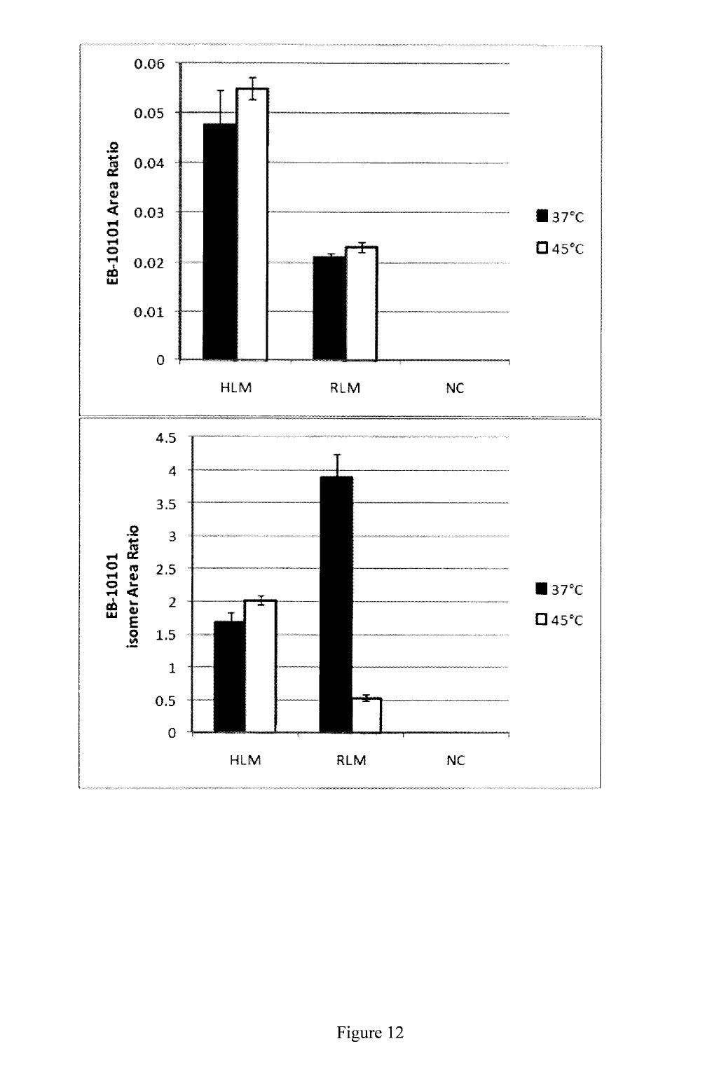

The major metabolite ((5-(3,4-dichloro-phenyl)-3-aza-bicyclo[3.1.0]hexan-2-one (EB-10101)) of amitifadine is a lactam. In human liver microsomes, as shown in Example XXXVII, the formation of the lactam from amitifadine is partially NADPH-dependent—a required co-factor for CYP and flavin mono-oxygenease enzymes (FMO). The amount of the lactam formed in the presence of NADPH is ˜2-fold higher than that in the absence of NADPH (FIG. 10), meaning about 50% of the lactam is formed by enzymes other than CYPs or FMOs. The formation of the lactam was not mediated by FMOs since the formation of the lactam in human liver microsomes was independent of elevated pre-incubation temperature Example XXXVIII (FIG. 12). Pre-incubation of microsomes at 45° inactivates FMO, but not CYP enzymes. The results indicate that the formation of the lactam is not FMO-mediated.

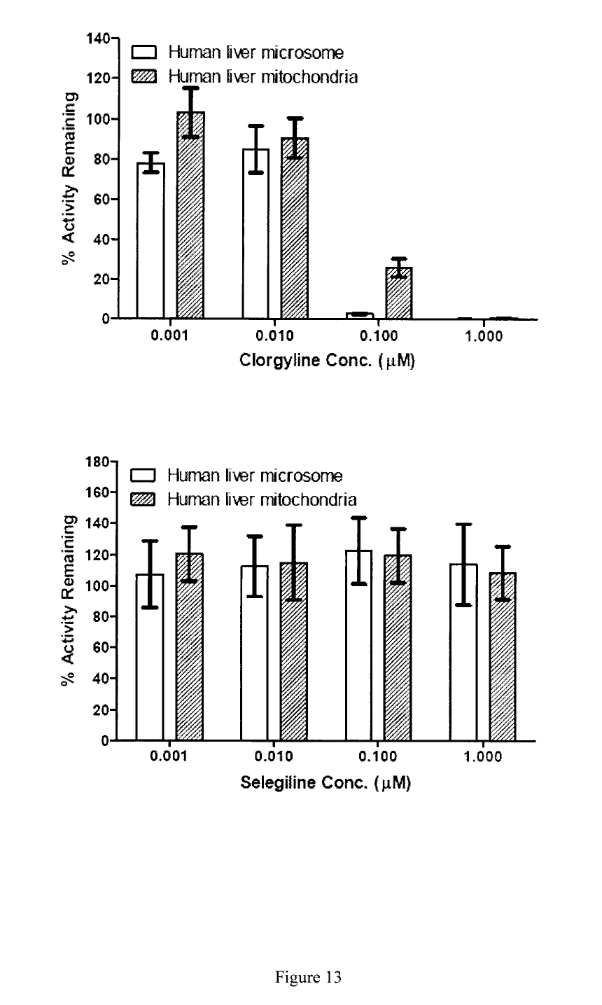

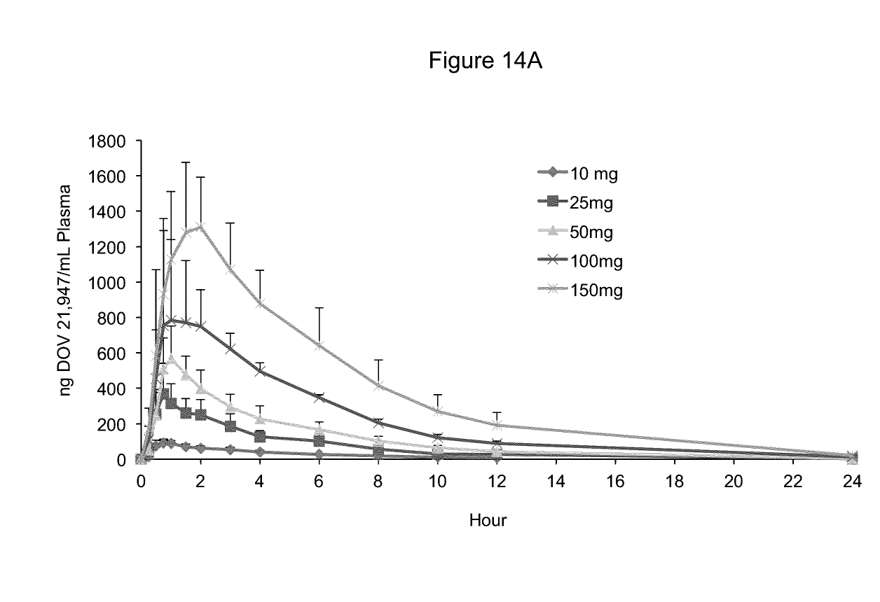

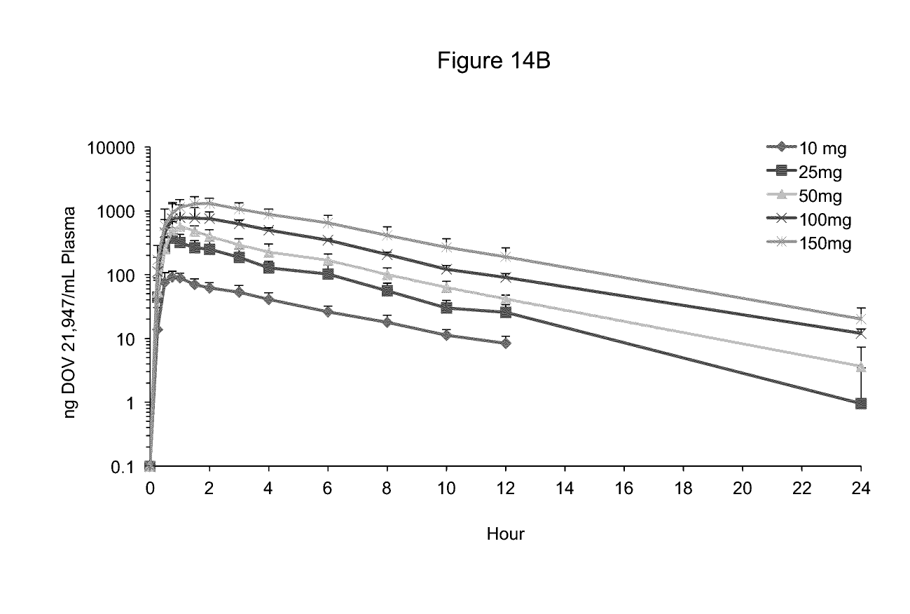

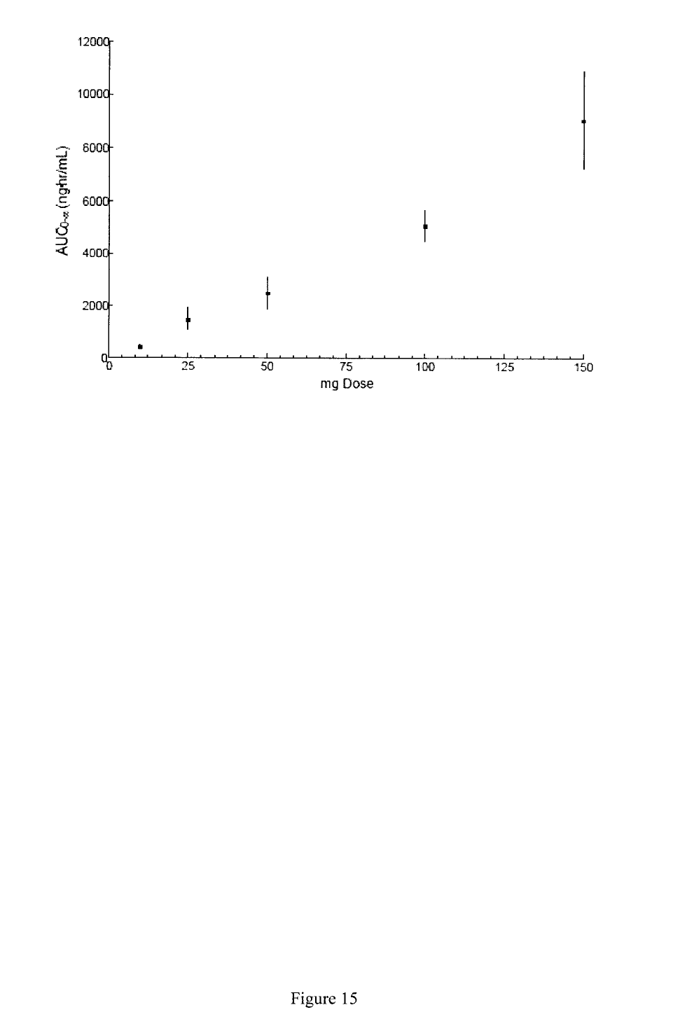

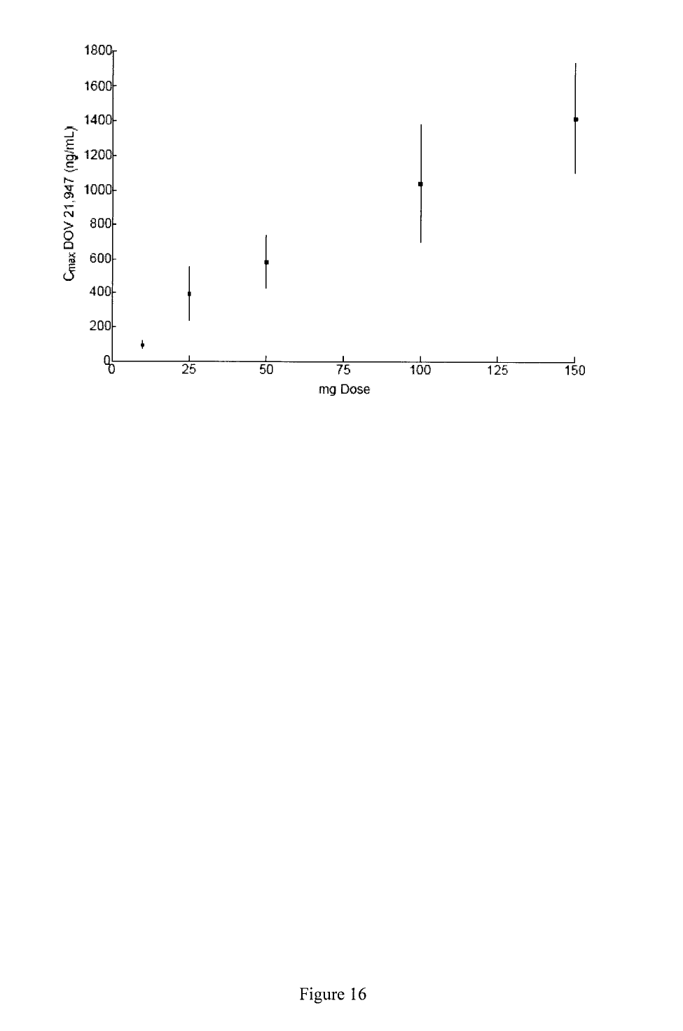

Additional experiments were undertaken to determine if the lactam from the preferred compound was formed by action of MAO enzymes. As shown in Example XXXIX and FIG. 13, the formation of the lactam in incubations with human liver microsomes or mitochondria was inhibited by the MAO-A inhibitor, clorgyline. The formation rates of the lactam decreased with increasing clorgyline concentrations. In addition, the formation of the lactam in human recombinant MAO-A enzyme incubations confirmed that the formation was MAO-A mediated (Example XL). The formation of the lactam in human liver microsomes or mitochondria incubations was not inhibited by the MAO-B inhibitor, selegiline. The formation rates of the lactam remained unchanged with increasing selegiline concentrations. The absence of the formation of the lactam in the human recombinant MAO-B enzyme incubations confirmed that the formation was not MAO-B mediated. In contrast, bicifadine is metabolized to a lactam exclusively by MAO-B.

(+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane are useful for treating or preventing endogenous disorders alleviated by inhibiting dopamine and/or norepinephrine and/or serotonin reuptake. Such disorders include, but are not limited to, attention-deficit disorder, depression, anxiety disorder, panic disorder, post-traumatic stress disorder, obsessive compulsive disorder, schizophrenia and allied disorders, anxiety, obesity, tic disorders, Parkinson's disease, chronic pain, attention deficit hyperactivity disorder (ADHD), addictive and substance abuse disorders, Alzheimer's disease and other cognitively impairing conditions.

Disorders alleviated by inhibiting dopamine and/or norepinephrine and/or serotonin reuptake are not limited to the specific disorders described herein, and the compositions and methods of the invention will be understood or readily ascertained to provide effective treatment agents for treating and/or preventing a wide range of additional disorders and associated symptoms. For example, the compounds of the invention will provide promising candidates for treatment and/or prevention of depression, attention deficit hyperactivity disorder and related symptoms, as well as forms and symptoms of alcohol abuse, drug abuse, cognitive disorders, obsessive compulsive behaviors, learning disorders, reading problems, gambling addiction, manic symptoms, phobias, panic attacks, oppositional defiant behavior, conduct disorder, academic problems in school, smoking, abnormal sexual behaviors, schizoid behaviors, somatization, depression, sleep disorders, general anxiety, stuttering, and tic disorders (See, for example, U.S. Pat. No. 6,132,724). Additional disorders contemplated for treatment employing the compositions and methods of the invention are described, for example, in the Quick Reference to the Diagnostic Criteria From DSM-IV ((Diagnostic and Statistical Manual of Mental Disorders, Fourth Edition), The American Psychiatric Association, Washington, D.C., 2000, 358 pages.) Cognitive disorders for treatment and/or prevention according to the invention, include, but are not limited to, Attention-Deficit/Hyperactivity Disorder, Predominately Inattentive Type; Attention-Deficit/Hyperactivity Disorder, Predominately Hyperactivity-Impulsive Type; Attention-Deficit/Hyperactivity Disorder, Combined Type; Attention-Deficit/Hyperactivity Disorder not otherwise specified (NOS); Conduct Disorder; Oppositional Defiant Disorder; and Disruptive Behavior Disorder not otherwise specified (NOS). Depressive disorders amenable for treatment and/or prevention according to the invention include, but are not limited to, Major Depressive Disorder, Recurrent; Dysthymic Disorder; Depressive Disorder not otherwise specified (NOS); and Major Depressive Disorder, Single Episode. Addictive disorders amenable for treatment and/or prevention employing the methods and compositions of the invention include, but are not limited to, eating disorders, impulse control disorders, alcohol-related disorders, nicotine-related disorders, amphetamine-related disorders, cannabis-related disorders, cocaine-related disorders, hallucinogen use disorders, inhalant-related disorders, and opioid-related disorders, all of which are further sub-classified as listed below. Substance abuse disorders include, but are not limited to alcohol-related disorders, nicotine-related disorders, Amphetamine-related disorders, cannabis-related disorders, cocaine-related disorders, hallucinogen-use disorders, inhalant-related disorders, and opioid-related disorders. For example, alcohol-related disorders include, but are not limited to, Alcohol-Induced Psychotic Disorder, with delusions; Alcohol Abuse; Alcohol Intoxication; Alcohol Withdrawal; Alcohol Intoxication Delirium; Alcohol Withdrawal Delirium; Alcohol-Induced Persisting Dementia; Alcohol-Induced Persisting Amnestic Disorder; Alcohol Dependence; Alcohol-Induced Psychotic Disorder, with hallucinations; Alcohol-Induced Mood Disorder; Alcohol-Induced Anxiety Disorder; Alcohol-Induced Sexual Dysfunction; Alcohol-Induced Sleep Disorders; Alcohol-Related Disorders not otherwise specified (NOS); Alcohol Intoxication; and Alcohol Withdrawal. Amphetamine-related disorders include, but are not limited to, Amphetamine Dependence, Amphetamine Abuse, Amphetamine Intoxication, Amphetamine Withdrawal, Amphetamine Intoxication Delirium, Amphetamine-Induced Psychotic Disorder with delusions, Amphetamine-Induced Psychotic Disorders with hallucinations, Amphetamine-Induced Mood Disorder, Amphetamine-Induced Anxiety Disorder, Amphetamine-Induced Sexual Dysfunction, Amphetamine-Induced Sleep Disorder, Amphetamine Related Disorder not otherwise specified (NOS), Amphetamine Intoxication, and Amphetamine Withdrawal. Cannabis-related disorders include, but are not limited to, Cannabis Dependence; Cannabis Abuse; Cannabis Intoxication; Cannabis Intoxication Delirium; Cannabis-Induced Psychotic Disorder, with delusions; Cannabis-Induced Psychotic Disorder with hallucinations; Cannabis-Induced Anxiety Disorder; Cannabis Related Disorder not otherwise specified (NOS); and Cannabis Intoxication. Cocaine-related disorders include, but are not limited to, Cocaine Dependence, Cocaine Abuse, Cocaine Intoxication, Cocaine Withdrawal, Cocaine Intoxication Delirium, Cocaine-Induced Psychotic Disorder with delusions, Cocaine-Induced Psychotic Disorders with hallucinations, Cocaine-Induced Mood Disorder, Cocaine-Induced Anxiety Disorder, Cocaine-Induced Sexual Dysfunction, Cocaine-Induced Sleep Disorder, Cocaine Related Disorder not otherwise specified (NOS), Cocaine Intoxication, and Cocaine Withdrawal. Hallucinogen-use disorders include, but are not limited to, Hallucinogen Dependence, Hallucinogen Abuse, Hallucinogen Intoxication, Hallucinogen Withdrawal, Hallucinogen Intoxication Delirium, Hallucinogen-Induced Psychotic Disorder with delusions, Hallucinogen-Induced Psychotic Disorders with hallucinations, Hallucinogen-Induced Mood Disorder, Hallucinogen-Induced Anxiety Disorder, Hallucinogen-Induced Sexual Dysfunction, Hallucinogen-Induced Sleep Disorder, Hallucinogen Related Disorder not otherwise specified (NOS), Hallucinogen Intoxication, and Hallucinogen Persisting Perception Disorder (Flashbacks) Inhalant-related disorders include, but are not limited to, Inhalant Dependence; Inhalant Abuse; Inhalant Intoxication; Inhalant Intoxication Delirium; Inhalant-Induced Psychotic Disorder, with delusions; Inhalant-Induced Psychotic Disorder with hallucinations; Inhalant-Induced Anxiety Disorder; Inhalant Related Disorder not otherwise specified (NOS); and Inhalant Intoxication. Opioid-related disorders include, but are not limited to, Opioid Dependence, Opioid Abuse, Opioid Intoxication, Opioid Intoxication Delirium, Opioid-Induced Psychotic Disorder with delusions, Opioid-Induced Psychotic Disorder with hallucinations, Opioid-Induced Anxiety Disorder, Opioid Related Disorder not otherwise specified (NOS), Opioid Intoxication, and Opioid Withdrawal.

By virtue of their multiple reuptake inhibitor activity, the novel compounds of the present invention are thus useful in a wide range of veterinary and human medical applications, in particular for treating and/or preventing a wide array of disorders and/or associated symptom(s) alleviated by inhibiting dopamine and/or norepinephrine and/or serotonin reuptake. The unbalanced serotonin-norepinephrine-dopamine reuptake inhibition ratio of ˜1:2:8, respectively of (+)-1-(3,4-dichlorophenyl)-3-azabicyclo[3.1.0]hexane (Skolnick et al., 2003) provides several advantages in comparison to a balanced triple reuptake inhibitor and allows for higher dosages of (+)-1-(3,4-dichlorophenyl)-3-azabicyclo[3.1.0]hexane to be used without triggering the dopaminergic or norepinephrine side effects such as elevated heart rate, increased blood pressure, nausea, vomiting, insomnia and hypomania seen in similar dosages of balanced triple reuptake inhibitors.

Additionally, unlike most anti-depressants which are metabolized entirely or predominately by cytochrome P450 enzymes, the (+)-1-(3,4-dichlorophenyl)-3-azabicyclo[3.1.0]hexane compositions as described herein are metabolized by both or either the cytochrome P450 enzymes and the monoamine oxidase (MAO) family of enzymes. The metabolism of the (+)-1-(3,4-dichlorophenyl)-3-azabicyclo[3.1.0]hexane compositions as described herein by the MAO enzymes decreases the likelihood of drug-drug interactions through the cytochrome P450 pathway and increases the effectiveness of such compositions in individuals with liver impairments.

Furthermore, the compositions of the present invention are effective in the treatment of those who have been previously treated for disorders affected by monoamine neurotransmitters such as depression. The compositions are additionally effective in the treatment of those who have had refractory experiences with prior treatments, i.e. individuals who have not responded, responded insufficiently, relapsed, or been unable to tolerate previous treatment(s) or who have otherwise responded in an unsatisfactory manner to other medications affecting monoamine neurotransmitters such as anti-depressants including, but not limited to, tri-cyclic antidepressants (TCAs), specific monoamine reuptake inhibitors, selective serotonin reuptake inhibitors, selective norepinephrine or noradrenaline reuptake inhibitors, selective dopamine reuptake inhibitors, serotonin-norepinephrine reuptake inhibitors, norepinephrine-dopamine reuptake inhibitors, multiple monoamine reuptake inhibitors, monoamine oxidase inhibitors, atypical antidepressants, atypical antipsychotics, anticonvulsants, or opiate agonists. Individuals may have been refractory to previous treatment(s) for any reason. In some embodiments, refractory individuals may have failed to respond or failed to respond sufficiently to a previous treatment. In one embodiment, a refractory individual may have treatment resistant depression. In other embodiments, a refractory individual may have responded to the initial treatment, but not succeed in entering remission from the treatment. In other embodiments, refractory individuals may have initially responded to previous treatments but had a relapse. Such a relapse may have occurred while on or off maintenance therapy. In additional embodiments, refractory individuals may be tachyphylactic. In further embodiments, the refractory individual may have developed pharmacokinetic tolerance, i.e. a change in the concentration of the drug acting at its target site, resulting from alterations in absorption, distribution, biotransformation, or elimination of the drug as a result of previous exposure to it. (Byrne et al, 1998). In yet another embodiment, refractory individuals may have genetic variants of cytochrome P450 enzymes that cause them to process pharmaceutical agents such as antidepressants in an atypical fashion. Such allelic variants may cause pharmaceutical agents to not be metabolized, to be metabolized more slowly than average, be metabolized more rapidly, be metabolized ultra-rapidly, or some variation thereof. In other embodiments, refractory individuals may have impaired liver function or may be taking additional medications that are inhibitors or enhancers of cytochrome P450 enzymes. Such impaired liver function may be for any reason. In some embodiments, impaired liver function may be due to cirrhosis. In some embodiments, impaired liver function may be due to jaundice. In some embodiments, impaired liver function may be due to chronic liver disease. In some embodiments, impaired liver function may be due to previous administration of one or more hepatotoxic medications. In some embodiments, impaired liver function may be due to concurrent administration of one or more hepatotoxic medications. In some embodiments, impaired liver function may be due to sequential administration of one or more hepatotoxic medications. In other embodiments, impaired liver function may be due to alcoholism. In some embodiments, refractory individuals may have been unable to continue taking the medication due to intolerance of the medication including side effects such as, but not limited to, sexual dysfunction, weight gain, insomnia, dry mouth, constipation, nausea and vomiting, dizziness, memory loss, agitation, anxiety, sedation, headache, urinary retention, or abdominal pain.

More than 70% of individuals on antidepressants are concurrently on an additional medication. (Preskorn S H. J Prac Psych Behav Hlth. 1998; 4:37-40.) With cytochrome P450 enzymes metabolizing approximately 90% of all drugs (Lynch, 2007), there is a high potential for drug-drug interactions. The compositions described herein are therefore additionally useful for individuals taking medications for other conditions that preclude the taking of an additional medication that modifies or is processed by the same cytochrome P450 enzymes, e.g. the decongestant phenylephrine or the analgesic hydrocodone should not be taken in combination with the antidepressant duloxetine.

Within additional aspects of the invention, combinatorial formulations and coordinate administration methods are provided which employ an effective amount of a (+)-1-(3,4-dichlorophenyl)-3-azabicyclo[3.1.0]hexane (or a pharmaceutically effective salt, solvate, hydrate, polymorph, or prodrug thereof), and one or more additional active agent(s) that is/are combinatorially formulated or coordinately administered with the compound of the invention—yielding a combinatorial formulation or coordinate administration method that is effective to modulate, alleviate, treat or prevent a targeted disorder, or one or more symptom(s) thereof, in a mammalian subject. Exemplary combinatorial formulations and coordinate treatment methods in this context comprise a therapeutic compound of the invention in combination with one or more additional or adjunctive treatment agents or methods for treating the targeted disorder or symptom(s), for example one or more antidepressant or anxiolytic agent(s) and/or therapeutic method(s).

In related embodiments of the invention, the compounds disclosed herein can be used in combination therapy with at least one other therapeutic agent or method. In this context, compounds of the invention can be administered concurrently or sequentially with administration of a second therapeutic agent, for example a second agent that acts to treat or prevent the same, or different, disorder or symptom(s) for which the compound of the invention is administered. The compound of the invention and the second therapeutic and/or psychotherapeutic agent can be combined in a single composition or administered in different compositions. The second therapeutic and/or psychotherapeutic agent may also be effective for treating and/or preventing a disorder or associated symptom(s) by inhibiting dopamine and/or norepinephrine and/or serotonin reuptake. The coordinate administration may be done simultaneously or sequentially in either order, and there may be a time period while only one or both (or all) active therapeutic agents, individually and/or collectively, exert their biological activities and therapeutic effects. A distinguishing aspect of all such coordinate treatment methods is that the compound of the invention exerts at least some detectable therapeutic activity toward alleviating or preventing the targeted disorder or symptom(s), as described herein, and/or elicit a favorable clinical response, which may or may not be in conjunction with a secondary clinical response provided by the secondary therapeutic agent. Often, the coordinate administration of a compound of the invention with a secondary therapeutic agent as contemplated herein will yield an enhanced therapeutic response beyond the therapeutic response elicited by either or both the compound of the invention and/or secondary therapeutic agent alone.

In one embodiment, combination therapy involves alternating between administering a compound of the present invention and a second therapeutic agent (i.e., alternating therapy regimens between the two drugs, e.g., at one week, one month, three month, six month, or one year intervals). Alternating drug regimens in this context will often reduce or even eliminate adverse side effects, such as toxicity, that may attend long-term administration of one or both drugs alone.

In certain embodiments of the invention, the additional psychotherapeutic agent is an antidepressant drug which may include, for example, any species within the broad families of tri-cyclic antidepressants (TCAs) including, but not limited to, amitriptyline, amoxapine, amineptine, doxepin, maprotiline, nortyptyline, protryptyline, trimipramine, imipramine, clomipramine, or desipramine; specific monoamine reuptake inhibitors such as selective serotonin reuptake inhibitors (SSRIs) including, but not limited to, escitalopram, fluoxetine, fluvoxamine, sertraline, citalopram, vilazodone, and paroxetine; and selective norepinephrine or noradrenaline reuptake inhibitors including but not limited to, reboxetine, atomoxetine, amedalin, CP-39,332, daledalin, edivoxetine (ly-2216684), esreboxetine, lortalamine, mazindol, nisoxetine, talopram, talsupram, tandamine, viloxazine; selective dopamine reuptake inhibitors; multiple monoamine reuptake inhibitors, e.g., reuptake inhibitors that inhibit both serotonin and norepinephrine reuptake (SNRIs) including, but not limited to, venlafaxine, duloxetine, milnacipran, desvenlafaxine, and SEP-227162; or inhibit both norepinephrine and dopamine (NDRIs), including but not limited to bupropion, amineptine, prolintane, dexmethylphenidate, desoxypipradrol, difemetorex, diphenylprlinol, fencamfamine, fencamine, lefetamine, mesocarb, methylenedioxypyrovalerone, methylphenidate, nomifensine, pyrovalerone, tametraline or pipradrol; or those that inhibit both serotonin and dopamine; monoamine oxidase inhibitors (MAOIs); and indeterminate (atypical) antidepressants. The additional psychotherapeutic agent may additionally include atypical antipsychotics including, but not limited to, aripiprazole, ziprasidone, risperidone, quetiepine, asenapine, or olanzapine; or anticonvulsants including, but not limited to, gabapentin, pregabalin, lamotrigine, carbamazepine, oxcarbazepine, valproate, levetriacetam, and topiramate. Additional psychotherapeutic agents may additionally include opiate agonists including, but not limited to, buprenorphine, methadone and levo-α-acetylmethadol (LAAM). Exemplary anxiolytics include, but are not limited to, benzodiazepines such as, but not limited to, alprazolam, chlordiazepoxide, clonazepam, diazepam, etizolam, lorazepam and oxazepam; selective serotonin reuptake inhibitors; azapirones such as, but not limited to, buspirone, tandospirone, and gepirone; barbiturates, hydroxyzine, and pregabalin.

In other embodiments of combinatorial formulations and coordinate treatment methods provided herein, the secondary psychotherapeutic agent is an anti-attention-deficit-disorder treatment agent. Examples of useful anti-attention-deficit-disorder agents for use in these embodiments include, but are not limited to, bupropion; methylphenidate; dextroamphetamine and other amphetamines; atomoxetine; tricyclic antidepressants, such as imipramine, desipramine, and nortriptyline; and psychostimulants, such as pemoline and deanol.

In additional embodiments of combinatorial formulations and coordinate treatment methods provided herein, the secondary psychotherapeutic agent is an anti-addictive-disorder or anti-substance abuse agent. Examples of useful anti-addictive-disorder agents include, but are not limited to, tricyclic antidepressants; glutamate antagonists, such as ketamine HCl, dextromethorphan, dextrorphan tartrate and dizocilpine (MK801); degrading enzymes, such as anesthetics and aspartate antagonists; GABA agonists, such as baclofen and muscimol HBr; reuptake blockers; degrading enzyme blockers; glutamate agonists, such as D-cycloserine, carboxyphenylglycine, and L-glutamic acid; aspartate agonists; GABA antagonists such as gabazine (SR-95531), saclofen, bicuculline, picrotoxin, and (+) apomorphine HCl; and dopamine antagonists, such as spiperone HCl, haloperidol, and (−) sulpiride; anti-alcohol agents including, but not limited to, baclofen, clomethiazole, acamprosate, topiramate, disulfiram and naltrexone; anti-nicotine agents including but not limited to, clonidine; anti-opiate agents including, but not limited to, methadone, clonidine, lofexidine, levomethadyl acetate HCl, naltrexone, and buprenorphine; anti-cocaine agents including, but not limited to, desipramine, amantadine, and buprenorphine; anti-lysergic acid diethylamide (“anti-LSD”) agent including but not limited to, diazepam; anti-1-(1-phenylcyclohexyl)piperidine (“anti-PCP”) agent including, but not limited to, haloperidol.

In other embodiments of combinatorial formulations and coordinate treatment methods provided herein, the secondary therapeutic agent is an appetite suppressant. Examples of useful appetite suppressants include, but are not limited to, fenfluramine, phenylpropanolamine, bupropion, and mazindol.

In yet additional embodiments of combinatorial formulations and coordinate treatment methods provided herein, the secondary therapeutic agent is an anti-Parkinson's-disease agent. Examples of useful anti-Parkinson's-disease agents include, but are not limited to dopamine precursors, such as levodopa, L-phenylalanine, and L-tyrosine; neuroprotective agents; dopamine agonists; dopamine reuptake inhibitors; anticholinergics such as amantadine and memantine; and 1,3,5-trisubstituted adamantanes, such as 1-amino-3,5-dimethyl-adamantane. (See, U.S. Pat. No. 4,122,193)

In further embodiments of combinatorial formulations and coordinate treatment methods provided herein, the secondary therapeutic agent is an anti-inflammatory agent. Examples of useful anti-inflammatory agents include, but are not limited to celecoxib, ibuprofen, ketoprofen, naproxen sodium, piroxicam, sulindac, aspirin, and nabumetone.

Suitable routes of administration for a (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention for mammalian subjects may range from about 5 mg to about 1800 mg, about 10 mg to about 1800 mg, 25 mg to about 1800 mg, about 50 mg to about 1000 mg, about 75 mg to about 900 mg, about 100 mg to about 750 mg, or about 150 mg to about 500 mg. In certain embodiments, the effective dosage will be selected within narrower ranges of, for example, about 5 mg to about 10 mg, 10 mg to about 25 mg, about 30 mg to about 50 mg, about 10 mg to about 300 mg, about 25 mg to about 300 mg, about 75 mg to about 100 mg, about 100 mg to about 250 mg, or about 250 mg to about 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In exemplary embodiments, dosages of about 10 mg to about 25 mg, about 30 mg to about 50 mg, about 25 mg to about 150, about 75 mg to about 100 mg, about 100 mg to about 250 mg, or about 250 mg to about 500 mg, are administered one, two, three, or four times per day. In more detailed embodiments, dosages of about 50-75 mg, about 100-200 mg, about 250-400 mg, or about 400-600 mg are administered once or twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing, and mode of delivery of compositions of the invention comprising an effective amount of a (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for the compounds of the invention will ordinarily be selected to approximate a minimal dosing regimen that is necessary and sufficient to substantially prevent or alleviate one or more symptom(s) of a neurological or psychiatric condition in the subject, as described herein. Thus, following administration of a (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention according to the formulations and methods herein, test subjects will exhibit a 10%, 20%, 30%, 50% or greater reduction, up to a 75-90%, or 95% or greater, reduction, in one or more symptoms associated with a targeted monoamine neurotransmitter influenced disorder or other neurological or psychiatric condition, compared to placebo-treated or other suitable control subjects.

Pharmaceutical dosage forms of a compound of the present invention may optionally include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

Pharmaceutical dosage forms of (+)-1-(3,4-dichlorophenyl)-3-azabicyclo[3.1.0]hexane may include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like.

Within various combinatorial or coordinate treatment methods of the invention, the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) may each be administered by any of a variety of delivery routes and modes, which may be the same or different for each agent.

An additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets and capsules, which may be prepared by any conventional method of preparing pharmaceutical oral unit dosage forms can be utilized in preparing oral unit dosage forms. Oral unit dosage forms, such as tablets or capsules, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum, and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate. Such rapidly acting dosage forms would be useful, for example, in the prevention or treatment of acute episodes of mania.

The additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention can be prepared and administered in any of a variety of inhalation or nasal delivery forms known in the art. Devices capable of depositing aerosolized formulations of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of depression. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a compound of the present invention, and any additional active or inactive ingredient(s).

Intranasal delivery permits the passage of active compounds of the invention into the blood stream directly after administering an effective amount of the compound to the nose, without requiring the product to be deposited in the lung. In addition, intranasal delivery can achieve direct, or enhanced, delivery of the active additional psychotherapeutic compound and/or (+)-1-(3,4-dichlorophenyl)-3-azabicyclo[3.1.0]hexane to the central nervous system. In these and other embodiments, intranasal administration of the compounds of the invention may be advantageous for treating disorders influenced by monoamine neurotransmitters, by providing for rapid absorption and delivery.

For intranasal and pulmonary administration, a liquid aerosol formulation will often contain an active compound of the invention combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional compositions and methods of the invention are provided for topical administration of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the present invention. Topical compositions may comprise a compound of the present invention and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound of the present invention dissolved or dispersed in water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours.

Yet additional formulations of a compound of the present invention are provided for parenteral administration, including aqueous and non-aqueous sterile injection solutions which may optionally contain anti-oxidants, buffers, bacteriostats and/or solutes which render the formulation isotonic with the blood of the mammalian subject; aqueous and non-aqueous sterile suspensions which may include suspending agents and/or thickening agents; dispersions; and emulsions. The formulations may be presented in unit-dose or multi-dose containers. Pharmaceutically acceptable formulations and ingredients will typically be sterile or readily sterilizable, biologically inert, and easily administered. Parenteral preparations typically contain buffering agents and preservatives, and may be lyophilized for reconstitution at the time of administration.

Parental formulations may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

Within exemplary compositions and dosage forms of the invention, the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) for treating disorders disclosed herein is/are administered in an extended release or sustained release formulation. In these formulations, the sustained release composition of the formulation provides therapeutically effective plasma levels of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) over a sustained delivery period of approximately 8 hours or longer, or over a sustained delivery period of approximately 18 hours or longer, up to a sustained delivery period of approximately 24 hours or longer.

In exemplary embodiments, the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is/are combined with a sustained release vehicle, matrix, binder, or coating material. As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings.

In one aspect, the current invention comprises an oral sustained release dosage composition for administering an additional psychotherapeutic compound and/or (+)-1-(3,4-dichlorophenyl)-3-azabicyclo[3.1.0]hexane agent (including (+)-1-(3,4-dichlorophenyl)-3-azabicyclo[3.1.0]hexane and pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) according to the invention. In a related aspect, the invention comprises a method of reducing one or more side effects that attend administration of an oral dosage form of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) compound by employing a sustained release formulation. Within these methods, an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is provided in a sustained release oral dosage form and the dosage form is introduced into a gastrointestinal tract of a mammalian subject presenting with a disorder amenable to treatment using the subject therapeutic drug, by having the subject swallow the dosage form. The method further includes releasing the active additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in a sustained, delayed, gradual or modified release delivery mode into the gastrointestinal tract (e.g., the intestinal lumen) of the subject over a period of hours, during which the active additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) reach(es), and is/are sustained at, therapeutic concentration(s) in a blood plasma, tissue, organ or other target site of activity (e.g., a central nervous system tissue, fluid or compartment) in the patient. When following this method, the side effect profile of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is less than a side effect profile of an equivalent dose of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) administered in an immediate release oral dosage form.

In certain embodiments, the additional psychotherapeutic compound and/or (+)-1-(3,4-dichlorophenyl)-3-azabicyclo[3.1.0]hexane is/are released from the sustained release compositions and dosage forms of the invention and delivered into the blood plasma or other target site of activity in the subject at a sustained therapeutic level over a period of at least about 6 hours, often over a period of at least about 8 hours, at least about 12 hours, or at least about 18 hours, and in other embodiments over a period of about 24 hours or greater. By sustained therapeutic level is meant a plasma concentration level of at least a lower end of a therapeutic dosage range as exemplified herein. In more detailed embodiments of the invention, the sustained release compositions and dosage forms will yield a therapeutic level of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) following administration to a mammalian subject in a desired dosage amount (e.g., 5, 10, 25, 50, 100, 200, 300, 400, 600, or 800 mg) that yields a minimum plasma concentration of at least a lower end of a therapeutic dosage range as exemplified herein over a period of at least about 6 hours, at least about 8 hours, at least about 12 hours, at least about 18 hours, or up to 24 hours or longer. In alternate embodiments of the invention, the sustained release compositions and dosage forms will yield a therapeutic level of additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) following administration to a mammalian subject in a desired dosage amount (e.g., 5, 10, 25, 50, 100, 200, 400, 600, or 800 mg) that yields a minimum plasma concentration that is known to be associated with clinical efficacy, over a period of at least about 6 hours, at least about 8 hours, at least about 12 hours, at least about 18 hours, or up to 24 hours or longer.

In certain embodiments, the active additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is/are released from the compositions and dosage forms of the invention and delivered into the blood plasma or other target site of activity in the subject (including, but not limited to, areas of the brain such as the thalamus, striatum, ventral tegmental area, cortical areas, hippocampus, hypothalamus, or nucleus accumbens) in a sustained release profile characterized in that from about 0% to 20% of the active compound is released and delivered (as determined, e.g., by measuring blood plasma levels) within in 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

In more detailed embodiments of the invention, compositions and oral dosage forms of an additional psychotherapeutic compound and/or (+)-1-(3,4-dichlorophenyl)-3-azabicyclo[3.1.0]hexane agents are provided, wherein the compositions and dosage forms, after ingestion, provide a curve of concentration of the additional psychotherapeutic compound and/or (+)-1-(3,4-dichlorophenyl)-3-azabicyclo[3.1.0]hexane agents over time, the curve having an area under the curve (AUC) which is approximately proportional to the dose of the additional psychotherapeutic compound and/or (+)-1-(3,4-dichlorophenyl)-3-azabicyclo[3.1.0]hexane agents administered, and a maximum concentration (Cₘₐₓ) that is proportional to the dose of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) administered.

In other detailed embodiments, the Cₘₐₓ of the active additional psychotherapeutic compound and/or (+)-1-(3,4-dichlorophenyl)-3-azabicyclo[3.1.0]hexane agents provided after oral delivery of a composition or dosage form of the invention is less than about 80%, often less than about 75%, in some embodiments less than about 60%, or 50%, of a Cₘₐₓ obtained after administering an equivalent dose of the active compound in an immediate release oral dosage form.

Within exemplary embodiments of the invention, the compositions and dosage forms containing the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) and a sustained release vehicle, matrix, binder, or coating will yield sustained delivery of the active compound such that, following administration of the composition or dosage form to a mammalian treatment subject, the Cₘₐₓ of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in the treatment subject is less than about 80% of a Cₘₐₓ provided in a control subject after administration of the same amount of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in an immediate release formulation.

As used herein, the term “immediate release dosage form” refers to a dosage form of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) wherein the active compound readily dissolves upon contact with a liquid physiological medium, for example phosphate buffered saline (PBS) or natural or artificial gastric fluid. In certain embodiments, an immediate release formulation will be characterized in that at least 70% of the active compound will be dissolved within a half hour after the dosage form is contacted with a liquid physiological medium. In alternate embodiments, at least 80%, 85%, 90% or more, or up to 100%, of the active compound in an immediate release dosage form will dissolve within a half hour following contact of the dosage form with a liquid physiological medium in an art-accepted in vitro dissolution assay. These general characteristics of an immediate release dosage form will often relate to powdered or granulated compositions of an additional psychotherapeutic compound and/or (+)-1-(3,4-dichlorophenyl)-3-azabicyclo[3.1.0]hexane agents in a capsulated dosage form, for example in a gelatin-encapsulated dosage form, where dissolution will often be relatively immediate after dissolution/failure of the gelatin capsule. In alternate embodiments, the immediate release dosage form may be provided in the form of a compressed tablet, granular preparation, powder, or even liquid dosage form, in which cases the dissolution profile will often be even more immediate (e.g., wherein at least 85%-95% of the active compound is dissolved within a half hour).

In additional embodiments of the invention, an immediate release dosage form will include compositions wherein the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is not admixed, bound, coated or otherwise associated with a formulation component that substantially impedes in vitro or in vivo dissolution and/or in vivo bioavailability of the active compound. Within certain embodiments, the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) will be provided in an immediate release dosage form that does not contain significant amounts of a sustained release vehicle, matrix, binder or coating material. In this context, the term “significant amounts of a sustained release vehicle, matrix, binder or coating material” is not intended to exclude any amount of such materials, but an amount sufficient to impede in vitro or in vivo dissolution of an additional psychotherapeutic compound and/or (+)-1-(3,4-dichlorophenyl)-3-azabicyclo[3.1.0]hexane agents in a formulation containing such materials by at least 5%, often at least 10%, and up to at least 15%-20% compared to dissolution of the additional psychotherapeutic compound and/or (+)-1-(3,4-dichlorophenyl)-3-azabicyclo[3.1.0]hexane agents when provided in a composition that is essentially free of such materials.

In alternate embodiments of the invention, an immediate release dosage form of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) may be any dosage form comprising the active compound which fits the FDA Biopharmaceutics Classification System (BCS) Guidance definition (see, e.g., http://www.fda.gov/cder/OPS/BCS_guidance.htm) of a “high solubility substance in a rapidly dissolving formulation.” In exemplary embodiments, an immediate release formulation of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) formulation according to this aspect of the invention will exhibit rapid dissolution characteristics according to BCS Guidance parameters, such that at least approximately 85% of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in the formulation will go into a test solution within about 30 minutes at pH 1, pH 4.5, and pH 6.8.

The compositions, dosage forms and methods of the invention thus include novel tools for coordinate treatment of disorders involving monoamine neurotransmitters by providing for sustained release and/or sustained delivery of the additional psychotherapeutic agent and/or (+)-1-(3,4-dichlorophenyl)-3-azabicyclo[3.1.0]hexane agents. As used herein, “sustained release” and “sustained delivery” are evinced by a sustained, delayed, extended, or modified, in vitro or in vivo dissolution rate, in vivo release and/or delivery rate, and/or in vivo pharmacokinetic value(s) or profile.

The sustained release dosage forms of the present invention can take any form as long as one or more of the dissolution, release, delivery and/or pharmacokinetic property(ies) identified above are satisfied. Within illustrative embodiments, the composition or dosage form can comprise an additional psychotherapeutic compound and/or (+)-1-(3,4-dichlorophenyl)-3-azabicyclo[3.1.0]hexane agents combined with any one or combination of: a drug-releasing polymer, matrix, bead, microcapsule, or other solid drug-releasing vehicle; drug-releasing tiny timed-release pills or mini-tablets; compressed solid drug delivery vehicle; controlled release binder; multi-layer tablet or other multi-layer or multi-component dosage form; drug-releasing lipid; drug-releasing wax; and a variety of other sustained drug release materials as contemplated herein, or formulated in an osmotic dosage form.

The present invention thus provides a broad range of sustained release compositions and dosage forms comprising an additional psychotherapeutic agent and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane), which in certain embodiments are adapted for providing sustained release of the active compound(s) following, e.g., oral administration. Sustained release vehicles, matrices, binders and coatings for use in accordance with the invention include any biocompatible sustained release material which is inert to the active agent and which is capable of being physically combined, admixed, or incorporated with the active compound. Useful sustained release materials may be dissolved, degraded, disintegrated, and/or metabolized slowly under physiological conditions following delivery (e.g., into a gastrointestinal tract of a subject, or following contact with gastric fluids or other bodily fluids). Useful sustained release materials are typically non-toxic and inert when contacted with fluids and tissues of mammalian subjects, and do not trigger significant adverse side effects such as irritation, immune response, inflammation, or the like. They are typically metabolized into metabolic products which are biocompatible and easily eliminated from the body.

In certain embodiments, sustained release polymeric materials are employed as the sustained release vehicle, matrix, binder, or coating (see, e.g., “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, N.Y. (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105, each incorporated herein by reference). Within exemplary embodiments, useful polymers for co-formulating with the additional psychotherapeutic compound and/or (+)-1-(3,4-dichlorophenyl)-3-azabicyclo[3.1.0]hexane agents to yield a sustained release composition or dosage form include, but are not limited to, ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

Additional polymeric materials for use as sustained release vehicles, matrices, binders, or coatings within the compositions and dosage forms of the invention include, but are not limited to, additional cellulose ethers, e.g., as described in Alderman, Int. J. Pharm. Tech. & Prod. Mfr., 1984, 5(3) 1-9 (incorporated herein by reference). Other useful polymeric materials and matrices are derived from copolymeric and homopolymeric polyesters having hydrolysable ester linkages. A number of these are known in the art to be biodegradable and to lead to degradation products having no or low toxicity. Exemplary polymers in this context include polyglycolic acids (PGAs) and polylactic acids (PLAs), poly(DL-lactic acid-co-glycolic acid)(DL PLGA), poly(D-lactic acid-coglycolic acid)(D PLGA) and poly(L-lactic acid-co-glycolic acid)(L PLGA). Other biodegradable or bioerodible polymers for use within the invention include such polymers as poly(ε-caprolactone), poly(ε-aprolactone-CO-lactic acid), poly(ε-aprolactone-CO-glycolic acid), poly(β-hydroxy butyric acid), poly(alkyl-2-cyanoacrilate), hydrogels such as poly(hydroxyethyl methacrylate), polyamides, poly-amino acids (e.g., poly-L-leucine, poly-glutamic acid, poly-L-aspartic acid, and the like), poly (ester ureas), poly (2-hydroxyethyl DL-aspartamide), polyacetal polymers, polyorthoesters, polycarbonates, polymaleamides, polysaccharides, and copolymers thereof. Methods for preparing pharmaceutical formulations using these polymeric materials are generally known to those skilled in the art (see, e.g., Sustained and Controlled Release Drug Delivery Systems, J. R. Robinson, ed., Marcel Dekker, Inc., New York, 1978, incorporated herein by reference).

In other embodiments of the invention, the compositions and dosage forms comprise an additional psychotherapeutic compound and/or (+)-1-(3,4-dichlorophenyl)-3-azabicyclo[3.1.0]hexane agents coated on a polymer substrate. The polymer can be an erodible or a nonerodible polymer. The coated substrate may be folded onto itself to provide a bilayer polymer drug dosage form. For example, the additional psychotherapeutic compound and/or (+)-1-(3,4-dichlorophenyl)-3-azabicyclo[3.1.0]hexane agents can be coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the bioerodible dosage form erodes at a controlled rate to dispense the active compound over a sustained release period. Representative biodegradable polymers for use in this and other aspects of the invention can be selected from, for example, biodegradable poly(amides), poly (amino acids), poly(esters), poly(lactic acid), poly(glycolic acid), poly(carbohydrate), poly(orthoester), poly (orthocarbonate), poly(acetyl), poly(anhydrides), biodegradable poly(dehydropyrans), and poly(dioxinones) which are known in the art (see, e.g., Rosoff, Controlled Release of Drugs, Chap. 2, pp. 53-95 (1989); and U.S. Pat. Nos. 3,811,444; 3,962,414; 4,066,747, 4,070,347; 4,079,038; and 4,093,709, each incorporated herein by reference).

In another embodiment of the invention, the dosage form comprises an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) loaded into a polymer that releases the drug(s) by diffusion through a polymer, or by flux through pores or by rupture of a polymer matrix. The drug delivery polymeric dosage form comprises the active compound contained in or on the polymer. The dosage form comprises at least one exposed surface at the beginning of dose delivery. The non-exposed surface, when present, can be coated with a pharmaceutically acceptable material impermeable to the passage of a drug. The dosage form may be manufactured by procedures known in the art, for example by blending a pharmaceutically acceptable carrier like polyethylene glycol, with a pre-determined dose of the active compound(s) at an elevated temperature (e.g., 37° C.), and adding it to a silastic medical grade elastomer with a cross-linking agent, for example, octanoate, followed by casting in a mold. The step is repeated for each optional successive layer. The system is allowed to set for 1 hour, to provide the dosage form. Representative polymers for manufacturing such sustained release dosage forms include, but are not limited to, olefin, and vinyl polymers, addition polymers, condensation polymers, carbohydrate polymers, and silicon polymers as represented by polyethylene, polypropylene, polyvinyl acetate, polymethylacrylate, polyisobutylmethacrylate, poly alginate, polyamide and polysilicon. These polymers and procedures for manufacturing them have been described in the art (see, e.g., Coleman et al., Polymers 1990, 31, 1187-1231; Roerdink et al., Drug Carrier Systems 1989, 9, 57-10; Leong et al., Adv. Drug Delivery Rev. 1987, 1, 199-233; and Roff et al., Handbook of Common Polymers 1971, CRC Press; U.S. Pat. No. 3,992,518).

In other embodiments of the invention, the compositions and dosage forms comprise an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) incorporated with or contained in beads that on dissolution or diffusion release the active compound over an extended period of hours, for example over a period of at least 6 hours, over a period of at least 8 hours, over a period of at least 12 hours, or over a period of up to 24 hours or longer. The drug-releasing beads may have a central composition or core comprising an additional psychotherapeutic compound and/or (+)-1-(3,4-dichlorophenyl)-3-azabicyclo[3.1.0]hexane agents and a pharmaceutically acceptable carrier, along with one or more optional excipients such as a lubricants, antioxidants, dispersants, and buffers. The beads may be medical preparations with a diameter of about 1 to 2 mm. In exemplary embodiments they are formed of non-cross-linked materials to enhance their discharge from the gastrointestinal tract. The beads may be coated with a release rate-controlling polymer that gives a timed release pharmacokinetic profile. In alternate embodiments the beads may be manufactured into a tablet for therapeutically effective drug administration. The beads can be made into matrix tablets by direct compression of a plurality of beads coated with, for example, an acrylic resin and blended with excipients such as hydroxypropylmethyl cellulose. The manufacture and processing of beads for use within the invention is described in the art (see, e.g., Lu, Int. J. Pharm., 1994, 112, 117-124; Pharmaceutical Sciences by Remington, 14^{th} ed, pp. 1626-1628 (1970); Fincher, J. Pharm. Sci. 1968, 57, 1825-1835; and U.S. Pat. No. 4,083,949, each incorporated by reference) as has the manufacture of tablets (Pharmaceutical Sciences, by Remington, 17^{th} Ed, Ch. 90, pp 1603-1625, 1985, incorporated herein by reference).

In another embodiment of the invention, the dosage form comprises a plurality of tiny pills or mini-tablets. The tiny pills or mini-tablets provide a number of individual doses for providing various time doses for achieving a sustained-release drug delivery profile over an extended period of time up to 24 hours. The tiny pills or mini-tablets may comprise a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, and a hydrophilic colloid. The hydrophilic polymer may be formed into a plurality (e.g., 4 to 50) tiny pills or mini-tablet, wherein each tiny pill or mini-tablet comprises a pre-determined dose of the additional psychotherapeutic agent and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane), e.g., a dose of about 10 ng, 0.5 mg, 1 mg, 1.2 mg, 1.4 mg, 1.6 mg, 5.0 mg etc. The tiny pills and mini-tablets may further comprise a release rate-controlling wall of 0.001 up to 10 mm thickness to provide for timed release of the active compound. Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills and mini-tablets are known in the art (see, e.g., U.S. Pat. Nos. 4,434,153; 4,721,613; 4,853,229; 2,996,431; 3,139,383 and 4,752,470, each incorporated herein by reference). The tiny pills and mini-tablets may further comprise a blend of particles, which may include particles of different sizes and/or release properties, and the particles may be contained in a hard gelatin or non-gelatin capsule or soft gelatin capsule.

In yet another embodiment of the invention, drug-releasing lipid matrices can be used to formulate therapeutic compositions and dosage forms comprising an additional psychotherapeutic agent and/or (+)-1-(3,4-dichlorophenyl)-3-azabicyclo[3.1.0]hexane agents. In one exemplary embodiment, solid microparticles of the active compound are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700 (each incorporated herein by reference). The lipid-coated particles can optionally be compressed to form a tablet. Another controlled release lipid-based matrix material which is suitable for use in the sustained release compositions and dosage forms of the invention comprises polyglycolized glycerides, e.g., as described in Roussin et al., U.S. Pat. No. 6,171,615 (incorporated herein by reference).

In other embodiments of the invention, drug-releasing waxes can be used for producing sustained release compositions and dosage forms comprising an additional psychotherapeutic agent and/or (+)-1-(3,4-dichlorophenyl)-3-azabicyclo[3.1.0]hexane agents. Examples of suitable sustained drug-releasing waxes include, but are not limited to, carnauba wax, candedilla wax, esparto wax, ouricury wax, hydrogenated vegetable oil, bees wax, paraffin, ozokerite, castor wax, and mixtures thereof (see, e.g., Cain et al., U.S. Pat. No. 3,402,240; Shtohryn et al. U.S. Pat. No. 4,820,523; and Walters, U.S. Pat. No. 4,421,736, each incorporated herein by reference).

In still another embodiment, osmotic delivery systems are used for sustained release delivery of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) (see, e.g., Verma et al., Drug Dev. Ind. Pharm., 2000, 26:695-708, incorporated herein by reference). In one exemplary embodiment, the osmotic delivery system is an OROS® system (Alza Corporation, Mountain View, Calif.) and is adapted for oral sustained release delivery of drugs (see, e.g., U.S. Pat. No. 3,845,770; and U.S. Pat. No. 3,916,899, each incorporated herein by reference).

In another embodiment of the invention, the dosage form comprises an osmotic dosage form, which comprises a semi-permeable wall that surrounds a therapeutic composition comprising the additional psychotherapeutic agent and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane). In use within a patient, the osmotic dosage form comprising a homogenous composition imbibes fluid through the semipermeable wall into the dosage form in response to the concentration gradient across the semipermeable wall. The therapeutic composition in the dosage form develops osmotic energy that causes the therapeutic composition to be administered through an exit from the dosage form over a prolonged period of time up to 24 hours (or even in some cases up to 30 hours) to provide controlled and sustained prodrug release. These delivery platforms can provide an essentially zero order delivery profile as opposed to the spiked profiles of immediate release formulations.

In alternate embodiments of the invention, the dosage form comprises another osmotic dosage form comprising a wall surrounding a compartment, the wall comprising a semipermeable polymeric composition permeable to the passage of fluid and substantially impermeable to the passage of the active compound present in the compartment, a drug-containing layer composition in the compartment, a hydrogel push layer composition in the compartment comprising an osmotic formulation for imbibing and absorbing fluid for expanding in size for pushing the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) composition layer from the dosage form, and at least one passageway in the wall for releasing the drug composition. This osmotic system delivers the active compound by imbibing fluid through the semipermeable wall at a fluid imbibing rate determined by the permeability of the semipermeable wall and the osmotic pressure across the semipermeable wall causing the push layer to expand, thereby delivering the active compound through the exit passageway to a patient over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 molecular weight which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer may comprise a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose), an osmagent, e.g., selected from the group consisting of sodium chloride, potassium chloride, potassium acid phosphate, tartaric acid, citric acid, raffinose, magnesium sulfate, magnesium chloride, urea, inositol, sucrose, glucose and sorbitol, and other agents such a hydroxypropylalkylcellulose of 9,000 to 225,000 average-number molecular weight (e.g., hydroxypropylethylcellulose, hydroxypropypentylcellulose, hydroxypropylmethylcellulose, or hydropropylbutylcellulose), ferric oxide, antioxidants (e.g., ascorbic acid, butylated hydroxyanisole, butylatedhydroxyquinone, butylhydroxyanisol, hydroxycoumarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine), and/or lubricants (e.g., calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid).

In the osmotic dosage forms, the semipermeable wall comprises a composition that is permeable to the passage of fluid and impermeable to passage of the additional psychotherapeutic agent and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane). The wall is nontoxic and comprises a polymer selected from the group consisting of a cellulose acylate, cellulose diacylate, cellulose triacylate, cellulose acetate, cellulose diacetate and cellulose triacetate. The wall typically comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether (e.g., hydroxypropylcellulose or a hydroxypropylalkycellulose such as hydroxypropylmethylcellulose). The total weight percent of all components comprising the wall is equal to 100 wt %. The internal compartment comprises the drug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of drug to a patient over time. The dosage form comprises a passageway in the wall that connects the exterior of the dosage form with the internal compartment. The osmotic powered dosage form delivers the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) from the dosage form to the patient at a zero order rate of release over a period of up to about 24 hours. As used herein, the expression “passageway” comprises means and methods suitable for the metered release of an additional psychotherapeutic compound and/or (+)-1-(3,4-dichlorophenyl)-3-azabicyclo[3.1.0]hexane agents from the compartment of an osmotic dosage form. The exit means comprises at least one passageway, including orifice, bore, aperture, pore, porous element, hollow fiber, capillary tube, channel, porous overlay, or porous element that provides for the osmotic controlled release of the active compound. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in U.S. Pat. Nos. 3,845,770; 3,916,899; 4,063,064; 4,088,864; 4,816,263; 4,200,098; and 4,285,987 (each incorporated herein by reference).

Within other aspects of the invention, microparticle, microcapsule, and/or microsphere drug delivery technologies can be employed to provide sustained release delivery of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) within the compositions, dosage forms and methods of the invention. A variety of methods is known by which an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) can be encapsulated in the form of microparticles, for example using by encapsulating the active compound within a biocompatible, biodegradable wall-forming material (e.g., a polymer)—to provide sustained or delayed release of the active compound. In these methods, the active compound is typically dissolved, dispersed, or emulsified in a solvent containing the wall forming material. Solvent is then removed from the microparticles to form the finished microparticle product. Examples of conventional microencapsulation processes are disclosed, e.g., in U.S. Pat. Nos. 3,737,337; 4,389,330; 4,652,441; 4,917,893; 4,677,191; 4,728,721; 5,407,609; 5,650,173; 5,654,008; and 6,544,559 (each incorporated herein by reference). These documents disclose methods that can be readily implemented to prepare microparticles containing an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in a sustained release formulation according to the invention. As explained, for example, in U.S. Pat. No. 5,650,173, by appropriately selecting the polymeric materials, a microparticle formulation can be made in which the resulting microparticles exhibit both diffusional release and biodegradation release properties. For a diffusional mechanism of release, the active agent is released from the microparticles prior to substantial degradation of the polymer. The active agent can also be released from the microparticles as the polymeric excipient erodes. In addition, U.S. Pat. No. 6,596,316 (incorporated herein by reference) discloses methods for preparing microparticles having a selected release profile for fine tuning a release profile of an active agent from the microparticles.

In another embodiment of the invention, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric coatings may function as a means for mediating sustained release of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) by providing one or more barrier layers, which may be located entirely surrounding the active compound, between layers of a multi-layer solid dosage form (see below), and/or on one or more outer surfaces of one or multiple layers of a multi-layer solid dosage form (e.g., on end faces of layers of a substantially cylindrical tablet). Such barrier layers may, for example, be composed of polymers which are either substantially or completely impermeable to water or aqueous media, or are slowly erodible in water or aqueous media or biological liquids and/or which swell in contact with water or aqueous media. Suitable polymers for use as a barrier layer include acrylates, methacrylates, copolymers of acrylic acid, celluloses and derivatives thereof such as ethylcelluloses, cellulose acetate propionate, polyethylenes and polyvinyl alcohols etc. Barrier layers comprising polymers which swell in contact with water or aqueous media may swell to such an extent that the swollen layer forms a relatively large swollen mass, the size of which delays its immediate discharge from the stomach into the intestine. The barrier layer may itself contain active material content, for example the barrier layer may be a slow or delayed release layer. Barrier layers may typically have an individual thickness of 10 microns up to 2 mm. Suitable polymers for barrier layers which are relatively impermeable to water include the Methocel™ series of polymers, used singly or combined, and Ethocel™ polymers. Such polymers may suitably be used in combination with a plasticizer such as hydrogenated castor oil. The barrier layer may also include conventional binders, fillers, lubricants and compression acids etc. such as Polyvidon K30 (trade mark), magnesium stearate, and silicon dioxide.

Additional enteric coating materials for mediating sustained release of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) include coatings in the form of polymeric membranes, which may be semipermeable, porous, or asymmetric membranes (see, e.g., U.S. Pat. No. 6,706,283, incorporated herein by reference). Coatings of these and other types for use within the invention may also comprise at least one delivery port, or pores, in the coating, e.g., formed by laser drilling or erosion of a plug of water-soluble material. Other useful coatings within the invention include coatings that rupture in the environment of use (e.g., a gastrointestinal compartment) to form a site of release or delivery port. Exemplary coatings within these and other embodiments of the invention include poly(acrylic) acids and esters; poly(methacrylic) acids and esters; copolymers of poly(acrylic) and poly(methacrylic) acids and esters; cellulose esters; cellulose ethers; and cellulose ester/ethers.

Additional coating materials for use in constructing solid dosage forms to mediate sustained release of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) include, but are not limited to, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmethylcellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxylated ethylene-vinylacetate, cellulose acetate butyrate, polyalkenes, polyethers, polysulfones, polyethersulfones, polystyrenes, polyvinyl halides, polyvinyl esters and ethers, natural waxes and synthetic waxes.

In additional embodiments of the invention, sustained release of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is provided by formulating the active compound in a dosage form comprising a multi-layer tablet or other multi-layer or multi-component dosage form. In exemplary embodiments, the active compound is formulated in layered tablets, for example having a first layer which is an immediate release layer and a second layer which is a slow release layer. Other multi-layered dosage forms of the invention may comprise a plurality of layers of compressed active ingredient having variable (i.e., selectable) release properties selected from immediate, extended and/or delayed release mechanisms. Multi-layered tablet technologies useful to produce sustained release dosage forms of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) are described, for example, in International Publications WO 95/20946; WO 94/06416; and WO 98/05305 (each incorporated herein by reference). Other multi-component dosage forms for providing sustained delivery of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) include tablet formulations having a core containing the active compound coated with a release retarding agent and surrounded by an outer casing layer (optionally containing the active compound) (see, e.g., International Publication WO 95/28148, incorporated herein by reference). The release retarding agent is an enteric coating, so that there is an immediate release of the contents of the outer core, followed by a second phase from the core which is delayed until the core reaches the intestine. Additionally, International Publication WO 96/04908 (incorporated herein by reference) describes tablet formulations which comprise an active agent in a matrix, for immediate release, and granules in a delayed release form comprising the active agent. Such granules are coated with an enteric coating, so release is delayed until the granules reach the intestine. International Publication WO 96/04908 (incorporated herein by reference) describes delayed or sustained release formulations formed from granules which have a core comprising an active agent, surrounded by a layer comprising the active agent.

Another useful multi-component (bi-layer tablet) dosage form for sustained delivery of additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is described in U.S. Pat. No. 6,878,386 (incorporated herein by reference). Briefly, the bilayer tablet comprises an immediate release and a slow release layer, optionally with a coating layer. The immediate release layer may be, for example, a layer which disintegrates immediately or rapidly and has a composition similar to that of known tablets which disintegrate immediately or rapidly. An alternative type of immediate release layer may be a swellable layer having a composition which incorporates polymeric materials which swell immediately and extensively in contact with water or aqueous media, to form a water permeable but relatively large swollen mass. Active material content may be immediately leached out of this mass. The slow release layer may have a composition comprising the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) with a release retarding vehicle, matrix, binder, coating, or excipient which allows for slow release of the active compound. Suitable release retarding excipients include pH sensitive polymers, for instance polymers based upon methacrylic acid copolymers, which may be used either alone or with a plasticiser; release-retarding polymers which have a high degree of swelling in contact with water or aqueous media such as the stomach contents; polymeric materials which form a gel on contact with water or aqueous media; and polymeric materials which have both swelling and gelling characteristics in contact with water or aqueous media. Release retarding polymers which have a high degree of swelling include, inter alia, cross-linked sodium carboxymethylcellulose, cross-linked hydroxypropylcellulose, high-molecular weight hydroxypropylmethylcellulose, carboxymethylamide, potassium methacrylatedivinylbenzene co-polymer, polymethylmethacrylate, cross-linked polyvinylpyrrolidone, high-molecular weight polyvinylalcohols etc. Release retarding gellable polymers include methylcellulose, carboxymethylcellulose, low-molecular weight hydroxypropylmethylcellulose, low-molecular weight polyvinylalcohols, polyoxyethyleneglycols, non-cross linked polyvinylpyrrolidone, xanthan gum etc. Release retarding polymers simultaneously possessing swelling and gelling properties include medium-viscosity hydroxypropylmethylcellulose and medium-viscosity polyvinylalcohols. An exemplary release-retarding polymer is xanthan gum, in particular a fine mesh grade of xanthan gum, preferably pharmaceutical grade xanthan gum, 200 mesh, for instance the product Xantural 75 (also known as Keltrol CR™ Monsanto, 800 N Lindbergh Blvd, St Louis, Mo. 63167, USA). Xanthan gum is a polysaccharide which upon hydration forms a viscous gel layer around the tablet through which the active has to diffuse. It has been shown that the smaller the particle size, the slower the release rate. In addition, the rate of release of active compound is dependent upon the amount of xanthan gum used and can be adjusted to give the desired profile. Examples of other polymers which may be used within these aspects of the invention include Methocel K4M™, Methocel E5™, Methocel E5O™ Methocel E4M™, Methocel K15M™ and Methocel K100M™. Other known release-retarding polymers which may be incorporated within this and other embodiments of the invention to provide a sustained release composition or dosage form of an additional psychotherapeutic compound and/or (+)-1-(3,4-dichlorophenyl)-3-azabicyclo[3.1.0]hexane agents include, hydrocolloids such as natural or synthetic gums, cellulose derivatives other than those listed above, carbohydrate-based substances such as acacia, gum tragacanth, locust bean gum, guar gum, agar, pectin, carrageenan, soluble and insoluble alginates, carboxypolymethylene, casein, zein, and the like, and proteinaceous substances such as gelatin.

Within other embodiments of the invention, a sustained release delivery device or system is placed in the subject in proximity of the target of the active compound, thus requiring only a fraction of the systemic dose (see, e.g., Goodson, in “Medical Applications of Controlled Release,” supra, vol. 2, pp. 115-138, 1984; and Langer, 1990, Science 249:1527-1533, each incorporated herein by reference). In other embodiments, an oral sustained release pump may be used (see, e.g., Langer, supra; Sefton, 1987, CRC Crit. Ref. Biomed. Eng. 14:201; and Saudek et al., 1989, N. Engl. J. Med. 321:574, each incorporated herein by reference).

The pharmaceutical compositions and dosage forms of the current invention will typically be provided for administration in a sterile or readily sterilizable, biologically inert, and easily administered form.

In other embodiments the invention provides pharmaceutical kits for reducing symptoms in a human subject suffering from a disorder affected by monoamine neurotransmitters, including depression. The kits comprise the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) therapeutic agent in an effective amount, and a container means for containing the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) for coordinate administration to the said subject (for example a container, divided bottle, or divided foil pack). The container means can include a package bearing a label or insert that provides instructions for multiple uses of the kit contents to treat the disorder and reduce symptoms in the subject. In more detailed embodiments, the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) are admixed or co-formulated in a single, combined dosage form, for example a liquid or solid oral dosage form. In alternate embodiments, the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) are contained in the kit in separate dosage forms for coordinate administration. An example of such a kit is a so-called blister pack. Blister packs are well-known in the packaging industry and are widely used for the packaging of pharmaceutical dosage forms (tablets, capsules and the like).

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The following examples illustrate certain aspects of the invention, but are not intended to limit in any manner the scope of the invention.

### Example I

### Preparation of 1-(3,4-dichlorophenyl)-3-azabicyclo[3.1.0]hexane

As described in U.S. Pat. No. 4,231,935, a solution of 59.5 g of 3,4-dichlorophenylacetic acid in 500 ml of absolute ethanol is saturated with anhydrous hydrogen chloride and then heated at reflux for 2 hours. The mixture is concentrated under reduced pressure to 200 ml, diluted with 200 ml of water and neutralized with concentrated ammonium hydroxide. This aqueous mixture is extracted 3 times with chloroform. Concentration and decolorization of the chloroform extracts gives ethyl 3,4-dichlorophenylacetate as a yellow oil.

In a three-necked flask fitted with a Nichrome stirrer and a reflux condenser is placed 7.0 g of ethyl 3,4-dichlorophenylacetate, 5.9 g of N-bromosuccinimide, 0.1 g of benzoyl peroxide and 150 ml of carbon tetrachloride. The reaction mixture is heated at reflux for 18 hours, cooled and filtered. The carbon tetrachloride filtrate is concentrated under reduced pressure to give a deep orange liquid. Vacuum distillation at 115°-120° C. (0.5 mm) gives ethyl α-bromo-3,4-dichlorophenylacetate as a pale yellow liquid.

This product is converted to diethyl cis-1-(3,4-dichlorophenyl)-1,2-cyclopropanedicarboxylate by the method of L. L. McCoy, J.A.C.S., 80, 6568 (1958).

A mixture of 150 g of this diester and 66 g of 85% KOH in 500 ml of water and 500 ml of ethanol is refluxed for 6 hours and then chilled in ice. The oily material is extracted into ether and the aqueous layer is made acidic with 100 ml of 12 N hydrochloric acid. The oily lower layer crystallizes slowly to give a colorless crystalline cake. This is recrystallized from a mixture of ethanol and ethyl acetate to give colorless crystals of 1-(3,4-dichlorophenyl)-1,2-cyclopropanedicarboxylic acid.

A mixture of 30.3 g of this diacid and 12.6 g of urea in one liter of xylene is refluxed for 6 hours. The solvent is stripped under reduced pressure and the crystalline residue is slurried with water. The colorless crystals are collected by filtration, washed with water and air dried to give 1-(3,4-dichlorophenyl)-1,2-cyclopropanedicarboximide.

To 40 ml of 1 molar borane-tetrahydrofuran is added with stirring under nitrogen at 0° C. a solution of 2.56 g of this imide in 50 ml of tetrahydrofuran during 15 minutes. The solution is warmed in a steam bath for 1 hour and is then cooled in ice, and then 20 ml of 6 N hydrochloric acid is added, and the tetrahydrofuran is removed under reduced pressure. The residue is made basic with 75 ml of 5 N sodium hydroxide and this is extracted with ether. The extract is dried over magnesium sulfate, filtered, and the filtrate is saturated with hydrogen chloride. The precipitated crystals are collected by filtration and are recrystallized from isopropyl alcohol to give 1.70 g of 1-(3,4-dichlorophenyl)-3-azabicyclo[3.1.0]hexane hydrochloride as colorless crystals, m.p. 180°-181° C.

### Example II

### (+)-1-(3,4-dichlorophenyl)-3-azabicyclo[3.1.0]hexane hydrochloride

To 279 mg of (±)-1-(3,4-dichlorophenyl)-3-azabicyclo[3.1.0]hexane hydrochloride obtained using the methods described above or in Epstein et al., J. Med. Chem., 24:481-490 (1981) was added 7 mL of 9:1 hexane:isopropyl alcohol, followed by 8 drops of diethylamine. To the resulting mixture was added isopropyl alcohol, dropwise, until a solution was obtained. The solution was concentrated to a volume of 6 mL using a stream of helium gas, and six 1-mL portions of the concentrate were subjected to high-performance liquid chromatography using an HPLC instrument equipped with a 1 cm×25 cm Daicel CHIRALPAK AD column (Chiral Technologies, Inc., Exton, Pa.). Elution was carried out at ambient temperature using 95:5 (v/v) hexane:isopropyl alcohol solution containing 0.05% diethylamine as a mobile phase at a flow rate of 6 mL/min. The fraction eluting at about 21.5 to 26 minutes was collected and concentrated to provide a first residue, which was dissolved in a minimal amount of ethyl acetate. Using a stream of nitrogen, the ethyl acetate solution was evaporated to provide a second residue, which was dissolved in 1 mL of diethyl ether. To the diethyl ether solution was added 1 mL diethyl ether saturated with gaseous hydrochloric acid. A colorless precipitate formed, which was filtered, washed with 2 mL of diethyl ether and dried to provide 73.4 mg of (+)-1-(3,4-dichlorophenyl)-3-azabicyclo[3.1.0]hexane hydrochloride: optical rotation [α]²⁵D=+60° in methanol at 2 mg/mL; 99.7% enantiomeric excess. (See, U.S. Pat. No. 6,372,919)

### Example III

### Preparation of (1R,5S)-(+)-1-(3,4-dichlorophenyl)-3-azabicyclo[3,10]hexane

To a solution of 3,4-dichlorophenylacetonitrile (3.50 kg) and S-(+)-epichlorohydrin (2.22 kg) in THF (18.5 L) at −15° C. under atmosphere of N₂ was added NaHMDS (16.5 L, 2M in THF) dropwise over 3 h. The reaction mixture was stirred for 3 h at −15° C., then, overnight at −5° C. BH₃-Me_{2S} (neat, 10M, 4.4 L) was added over 2 h. The reaction mixture was then gradually warmed to 40° C. over 3 h. After aging 1.5 h at 40° C., the reaction mixture was cooled to 20-25° C. and slowly quenched into a 2N HCl solution (27.7 L). The quenched mixture was then aged for 1 h at 40° C. Concentrated NH₄OH (6.3 L) was added and the aqueous layer was discarded. i-PrOAc (18.5 L) and 5% dibasic sodium phosphate (18.5 L) were charged. The organic phase was then washed with saturated brine (18.5 L), azeotropically dried and solvent-switched to i-PrOAc (ca. 24.5 L) in vacuum.

The above crude amino alcohol solution in i-PrOAc was slowly subsurface-added to a solution of SOCl₂ (22.1 mol, 1.61 L) in i-PrOAc (17.5 L) at ambient temperature over 2 h. After aging additional 1-5 h, 5.0 N NaOH (16.4 L) was added over 1 h while the batch temperature was maintained at <30° C. with external cooling. The two-phase reaction mixture was stirred for 1 h at ambient temperature to allow pH to stabilize (usually to 8.5-9.0) with NaOH pH titration. The organic phase was washed with 40% aqueous i-PrOH (21 L) followed by water (10.5 L). Conc. HCl (1.69 L) was added. The aqueous i-PrOAc was azeotropically concentrated in vacuum to ca. 24.5 L. Methylcyclohexane (17.5 L) was added dropwise over 2 h. The wet cake was displacement-washed with 7 L of 40% methylcyclohexane/1-PrOAc followed by a slurry wash (7 L, i-PrOAc) and a displacement wash (7 L, i-PrOAc). Typical isolated yield: 57-60% corrected with wt %: 87-99.5% (based on HCl salt).

(1R,5S)-(+)-1-(3,4-dichlorophenyl)-3-azabicyclo[3,10]hexane HCl salt (5.0 kg) was dissolved in i-PrOH (14.25 L) and water (0.75 L) at 55° C. Seeds (50 g) were added at 48-50° C. The batch was allowed to cool to ambient temperature (20° C.) over 2-4 h. MeOBu-t (37 L) was added dropwise over 2 h. After aging 1 h at 20° C., the batch was filtered. The wet cake was displacement-washed with 10 L of 30% i-PrOH in MeOBu-t followed by 2×7.5 L 10% i-PrOH in MeOBu-t (slurry wash, then displacement wash). The wet cake was suction dried under N₂ (10-50 RH %) at ambient temperature to give the hemihydrate HCl salt of (1R,5S)-(+)-1-(3,4-dichlorophenyl)-3-azabicyclo[3,10]hexane. Typical yield: 92%. ¹H-NMR (400 MHz, d₄-MeOH): Δ 7.52 (d, J=2.2 Hz, 1H), 7.49 (d, J=8.4 Hz, 1H), 7.26 (dd, J=2.1, 8.4 Hz, 1H), 3.78 (d, J=11.4 Hz, 1H), 3.69 (dd, J=3.9, 11.3 Hz, 1H), 3.62 (dd, J=1.4, 11.3 Hz, 1H), 3.53 (d, J=11.4 Hz, 1H), 2.21 (m, 1H), 1.29 (t, J=7.5 Hz, 1H), 1.23 (dd, J=4.9, 6.5 Hz, 1H). ¹³C-NMR (100 MHz, d₄-MeOH): Δ 141.0, 133.7, 132.2, 132.0, 130.6, 128.4, 51.7, 49.1, 31.8, 24.9, 16.5. Anal. Calcd for C₁₁H₁₃Cl₃NO_{0.5}: C, 48.29; H, 4.79; N, 5.12; Cl, 38.88. Found: C, 48.35; H, 4.87; N, 5.07; 38.55. (See U.S. patent application Ser. No. 11/740,667)

### Example IV

### Method of Manufacture of (+)-1-(3,4-dichlorophenyl)-3-azabicyclo[3.1.0]hexane hydrochloride

(+)-1-(3,4-dichlorophenyl)-3-azabicyclo[3.1.0]hexane hydrochloride may also be manufactured according to the procedure described in U.S. patent application Ser. No. 12/428,399 as follows:

### Step 1: Synthesis of α-bromo-3.4-dichlorophenylacetic acid methyl ester

100 kg 3,4-dichlorophenylacetonitrile was added in portions over 1.25 hours to a mixture of 12 kg water and 140 kg 98% sulfuric acid. Exotherm was allowed to 65° C. maximum, and the reaction mix was maintained at 60-65° C. for 30 minutes. After cooling to 50° C., 80 kg methanol was slowly added over 25-30 minutes. The mixture was warmed to 92-98° C., and maintained at this temperature for an additional three hours. After cooling to 35° C., the reaction mixture was quenched into an agitated mixture (precooled to 0-5° C.) of 150 L ethylene dichloride and 250 L water. The reactor and lines were washed with water into the quench mix, which was agitated 5 minutes and allowed to stratify. The lower organic phase was separated, and the aqueous phase washed with 2×150 L ethylene dichloride. The combined organic phases were washed with 100 L water and then with aqueous sodium carbonate (3 kg sodium carbonate in 100 L water). The solution of crude ester was azeotropically “dried” in vacuo at 60-62° C., resulting in the collection of 100 L ethylene dichloride. A theoretical yield was assumed without isolation and the solution was used “as is” in the following bromination reaction.

A mixture of the solution (line-filtered) of crude methyl 3,4-dichlorophenylacetate (from above) and 88 kg 1,3-dibromo-1,3-dlmethylhydantoin (DBDMH) was warmed to 80° C., and a solution of 2.5 kg VAZO 52 in 15 L ethylene dichloride was added portion wise over a 5 hour period, maintaining 85-90° C. (under reflux). An additional 8.8 kg DBDMH was then added, and a solution of 0.5 kg VAZO 52 in 4 L ethylene dichloride was added portion wise over a 2.5 hour period, maintaining 85-90° C. (under reflux). Heating was then discontinued, and 350 L water was added with agitation. The mixture was allowed to stratify, the lower organic phase was separated and the aqueous phase was washed with 50 L ethylene dichloride. The combined organic phases were washed with aqueous thiosulfate (5.0 kg sodium thiosulfate in 150 L water), aqueous sodium carbonate (2.5 kg sodium carbonate in 150 L water), and dilute hydrochloric acid (5.4 L 32% HCl in 100 L water). The organic phase was line-filtered and distilled in vacuo to “dryness” (full vacuum to 83° C.). Residual ethylene dichloride was chased with 20 kg toluene (full vacuum at 83° C.). The crude α-bromo-3,4-dichlorophenylacetic acid methyl ester was taken up in 82 kg toluene, cooled to 40° C., and discharged to steel drums. The product was not isolated, and was used “as is” in Step 2. A theoretical yield was assumed for calculation purposes.

### Step 2: Synthesis of 1-(3,4-dichlorophenyl-1,2-cyclopropane-dicarboxylic acid dimethyl ester

The crude α-bromo-3,4-dichlorophenylacetic acid methyl ester from Step 1 was mixed well with 55.6 kg methyl acrylate, and then the mixture was added to a precooled (−2° C.) mixture of 54.4 kg potassium methoxide in 500 L toluene (argon blanket) over 5.5 hours with good agitation and maintained at <+10° C. After standing overnight (5 psig argon) with brine cooling (−5° C.), the cold reaction mixture was quenched into a mix of 250 L water and 30 kg 32% hydrochloric acid with good agitation. 200 L water and 2.5 kg potassium carbonate were added to the mixture with good agitation for an additional 30 minutes. After stratification, the lower aqueous phase was separated, and 150 L water and 1.0 kg potassium carbonate were added to the organic phase. The mixture was agitated 5 minutes and stratified. The lower aqueous phase was separated and discarded, as well as the interfacial emulsion, and the organic phase was washed with 100 L water containing 1 L 32% hydrochloric acid. After stratification and separation of the lower aqueous phase, the organic phase was line-filtered and distilled in vacuo to “dryness” (full vacuum at 65° C.). To the hot residue was added 70 kg methanol with agitation. The mix was cooled (seeding at +10° C.) to −5° C. and maintained at this temperature overnight. The cold thick suspension was suction-filtered (Nutsche), and the cake of 1-(3,4-dichlorophenyl)-1,2-cyclopropane-dicarboxylic acid dimethyl ester was suction dried, washed with 2×20 L hexane, suction dried for 30 minutes and air-dried on paper (racks) for 2 days at ambient conditions.

To the methanolic liquors was added 50 kg caustic soda flake portion wise over 8 hours with good agitation. After gassing and the slow exotherm (60° C. maximum) ceased, the heavy suspension was held at 50° C. for 1 hour. 100 L isopropanol was slowly added over 10 minutes, and then the mixture was agitated slowly overnight at ambient conditions. The solids were suction-filtered (Nutsche) and reslurried with 80 L methanol. The resulting 1-(3,4-dichlorophenyl)-1,2-cyclopropane-dicarboxylic acid disodium salt was suctioned-filtered (Nutsche), washed with methanol (40 L), suction dried for 1 hour and air-dried on paper (racks).

### Step 3: Synthesis of 1-(3.4-dichlorophenyl)-1,2-cyclopropane-dicarboxylic acid

A suspension of 42.0 kg 1-(3,4-dichlorophenyl)-1,2-cyclopropane-dicarboxylic acid disodium salt (from Step 2) and 120 L deionized water was warmed to 30-35° C., and the solution was line-filtered and neutralized with 30 kg 32% hydrochloric acid to precipitate the free dicarboxylic acid. 120 kg ethyl acetate was added, and the mix warmed to 40-50° C. to effect solution. The lower aqueous phase was separated and washed with 20 kg ethyl acetate. The combined organic extracts were washed with saturated sodium chloride (3 kg in 30 L water) and then distilled in vacuo to “dryness” (full vacuum to 70° C.). 60 kg ethylene dichloride was added to the warm residue, and the solution cooled with slow agitation at −5° C. overnight. Residual ethyl acetate was distilled (full vacuum to 43° C.) to yield a thick suspension, which was then cooled with full vacuum to −5° C. over a 2.5 hour period and then suction-filtered (Nutsche). The 1-(3,4-dichlorophenyl)-1,2-cyclopropane-dicarboxylic acid cake was washed with cold ethylene dichloride (2×5 L), followed by ambient ethylene dichloride (4×5 L). The dicarboxylic acid product was suction dried for 15 minutes and air-dried on paper (racks).

A mixture of 31.0 kg 1-(3,4-dichlorophenyl)-1,2-cyclopropane-dicarboxylic acid dimethyl ester (from Step 2), 40 L water, 35 kg methanol and 18.0 kg 50% caustic soda was warmed to 70-75° C. (under reflux) and maintained at 70-75° C. for 1.5 hours. 10 L water was then added, and the mixture was kept at 75-77° C. for an additional 2 hours. Methanol was slowly distilled off in vacuo to 70° C. to give a heavy suspension, which was then mixed with 80 L water to effect solution. The free dicarboxylic acid was precipitated with 31 kg of 32% hydrochloric acid and extracted with 100 kg ethyl acetate. The lower aqueous phase was separated and washed with 20 kg ethyl acetate. The combined organic phases were washed with 50 L water, and then saturated aqueous sodium chloride. Distillation in vacuo to 80° C. with full vacuum yielded a concentrate of 1-(3,4-dichlorophenyl)-1,2-cyclopropane-dicarboxylic acid, which was used “as is” for the next step, cyclization to the imide. A quantitative yield from the diester was assumed for calculation purposes.

### Step 4: Synthesis and Recrystallization of 1-(3,4-dichlorophenyl)-3-azabicyclo[3.1.0]hexane-2,4-dione

The slurry of 1-(3,4-dichlorophenyl)-1,2-cyclopropane-dicarboxylic acid (from Step 3) was added to 45.6 kg warm (68° C.) formamide, and residual ethyl acetate was distilled with full vacuum at 68-73° C. An additional 14.4 kg formamide was added to the mixture, followed by 11.2 kg of the dicarboxylic acid (derived from the disodium salt, Step 3). An argon blanket on the mixture was maintained for the following operation. The mixture was agitated 15 minutes at 73-75° C. to effect a complete solution, and then heated over a 1 hour period to 140-145° C. and maintained at this temperature for an additional 2.25 hours. Heating was discontinued, and the mixture was cooled to 70° C. and 10 L water containing 20 ml 32% HCl was slowly added over 30 minutes. The mixture was seeded and crystallization commenced. An additional 20 L water was slowly added to the heavy suspension over a 2 hour period. After standing overnight at ambient conditions, the mixture was agitated for 1.25 hours at ambient temperature and then suction-filtered (Nutsche). The cake of crude 1-(3,4-dichlorophenyl)-3-azabicyclo-[3.1.0]hexane-2,4-dione was washed with water (3×20 L), suction dried for 30 minutes and air-dried on paper (racks) for 2 days under ambient conditions.

A mixture of 37 kg crude, damp 1-(3,4-dichlorophenyl)-3-azabicyclo-[3.1.0]hexane-2,4-dione (from Step 4, above) and 120 L toluene was warmed to 75-80° C. to effect solution. After stratification and separation of the residual water (3.3 kg), 1 kg Darco G-60 activated carbon (American Norit Co.) (suspended in 5 L toluene) was added. The mixture was agitated at 80° C. for 30 minutes and then pressure filtered through a preheated Sparkler (precoated with filteraid), polishing with a 10 μm in-line filter. The clear light yellow solution was concentrated in vacuo at 75-80° C. to 100 L final volume and slowly cooled, with seeding at 70° C. The heavy crystalline suspension was cooled to −5° C., held 30 minutes at this temperature and suction-filtered (Nutsche). The cake of purified 1-(3,4-dichlorophenyl)-3-azabicyclo-[3.1.0]hexane-2,4-dione was washed with 2×10 L cold (−10° C.) toluene, and then 2×20 L hexane. After suction drying for 30 minutes, the 2,4-dione product was dried in vacuo (≦62° C.).

### Step 5: Synthesis and Purification of (±)-1-(3.4-Dichlorophenyl)-3-azabicyclo [3.1.0]hexane hydrochloride

BH3-THF complex is charged into a 2 L addition funnel (9×2 L, then 1×1.5 L) and drained into a 50 L flask.

1000 g of (±)-1-(3,4 dichlorophenyl)-3-azabicyclo[3.1.0]-hexane-2,4-dione is dissolved in 2 L of THF and added to the BH3-THF dropwise over a period of 2 hours. The reaction mixture is heated to reflux and held at this temperature overnight. The mixture is then cooled to <10° C., adjusted to pH 2 with the addition of 1200 mL of 6N HCl dropwise at <20° C., and stirred for a minimum of 1 hour.

The reaction mixture is then transferred to a 10 L Buchi flask, concentrated to a milky white paste, and transferred again to a 5-gallon container. The mixture is diluted with 4 L of cold water and adjusted to pH 10 with 2000 mL of a 25% sodium hydroxide solution. A temperature of <20° C. is maintained. Following this, 4.5 L of ethyl acetate is added and the mixture is stirred for 15 minutes. The solution is then filtered through a 10 inch funnel with a filter cloth and washed with ethyl acetate (2×250 mL).

The filtrate is then transferred into a 40 L separatory funnel and the phases are allowed to separate. Each phase is then drained into separate 5-gallon containers. The aqueous layer is returned to the 40 L separatory funnel and extracted with ethyl acetate (2×2 L). The organic phases are combined. The aqueous layer is discarded.

250 g of magnesium sulfate and 250 g of charcoal are added to the combined organics and the mixture is stirred well. The solution is then filtered through an 18.5 cm funnel using a filter pad and washed with ethyl acetate (2×250 mL). The filtrate is then transferred to a 10 L Buchi flask and concentrated to dryness. The resulting yellowish oil is diluted with ethyl acetate (2.25 mL/g).

HCl gas is bubbled through a 12 L flask containing 10 L of ethyl acetate to make an approximately 2.3 M solution of HCl/ethyl acetate. This HCl/ethyl acetate solution is added to the oil dropwise at a rate that maintains a temperature of <20° C. using an ice/water bath. The solution is then stirred at <10° C. for a minimum of 2 hours in the ice/water bath. The material is chilled in a cold room overnight.

The resulting solids are then filtered through a 10 inch funnel utilizing a filter cloth and washed with ethyl acetate (2×200 mL) and ethyl ether (3×500 mL). The product, crude (±)-1-(3,4-Dichlorophenyl)-3-azabicyclo[3.1.0]-hexane hydrochloride, is then transferred to Pyrex drying trays and dried for 4 hours.

1900 g of crude (±)-1-(3,4-dichlorophenyl)-3-azabicyclo[3.1.0]hexane hydrochloride from above, and 15.2 L of isopropyl alcohol are charged to a 22 L flask. The mixture is heated to dissolve all material.

The material is then filtered through a 18.5 cm funnel utilizing a filter pad and transferred to a 22 L flask. The solution is then stirred at room temperature for 1 hour. After stirring, the solution is chilled to 4° C. with an ice/water bath and stirred for 3.75 hours. The product is then placed in a cold room overnight.

The solids are then filtered through a 13 inch filter using a filter cloth and washed with ethyl ether (3×633 mL). The product is then air dried for 2 hours.

The product, pure (±)-1-(3,4-dichlorophenyl)-3-azabicyclo[3.1.0]hexane hydrochloride, is transferred to clean Pyrex drying trays and dried to constant weight.

### Step 6: Resolution of (±)-1-(S3,4-dichlorophenl)-3-azabicyclo[3.1.0]hexane hydrochloride into (+)-1-(3,4-dichlorophenyl)-3-azabicyclo[3.1.0]hexane hydrochloride

In a 50 gallon reactor containing 60 L of 15% NaOH, 13.6 kg of pure (±)-1-(3,4dichlorophenyl)-3-azabicyclo[3.1.0]hexane hydrochloride (from Step 5, above) is added while keeping the temperature constant at approximately 20° C. Once the addition of (±)-1-(3,4-dichlorophenyl)-3-azabicyclo[3.1.0]hexane hydrochloride is complete, the reaction mixture is allowed to stir at room temperature for a minimum of 8 hours.

40 L of ethyl acetate is added to the reactor and the two phase mixture is stirred until a clear solution is obtained (approximately 2 hours). The phases are allowed to separate and the organic layer is transferred to another 50 gallon reactor. The remaining aqueous layer is extracted with ethyl acetate (6×6 L). All organic phases are combined into the 50-gallon reactor. The organic phase is dried and decolorized by the addition of 4000 g magnesium sulfate and 250 g of charcoal. The mixture is then filtered through an in-line filter. The filtrate is transferred via in-line filter to a 50-gallon reactor.

In a separate 50-gallon reactor, 23,230 g of L-(−)-dibenzoyl tartaric acid is dissolved with stirring (approximately 30 minutes) in 71 L of methanol. The dissolution is assisted with heating if necessary.

The L-(−)-dibenzoyl tartaric acid solution in methanol is added via addition funnel to the reactor containing the filtrate, over a period of approximately 1 hour, maintaining the temperature at 15-25° C. After the addition is complete the mixture is stirred for approximately 16 hours at 15-25° C. Following stirring, 50 L of methanol is added to the mixture and it is stirred again for 30 additional minutes. The resulting solids are filtered onto a plate filter. The solids are then washed with methanol (3×5 L) and pressed dry. The crude solids are weighed and transferred to a 50-gallon reactor to which 80 L of methanol is added. The mixture is heated to reflux and stirred at reflux for approximately 30 minutes. The mixture is then cooled to 15-20° C. and stirred at this temperature for approximately 2 hours. The resulting solids are filtered onto a plate filter using a polypropylene filter cloth. The cake is washed with methanol (3×5 L) and pressed dry. The solids are transferred to a tarred 5-gallon container and weighed (yield ˜20 kg).

The solids are then added (over a period of approximately 1 hour) to a 50 gallon reactor vessel containing 60 L of 15% NaOH while maintaining the temperature at approximately 20° C. Once the addition of the solids is complete the reaction mixture is stirred for approximately 19 hours.

40 L of ethyl acetate is charged to the reactor, while maintaining the temperature at ≦35° C. and the two phase mixture is stirred until a clear solution is obtained (approximately 2 hours). The phases are allowed to separate and the organic layer is transferred to another 50 gallon reactor. The remaining aqueous layer is extracted with ethyl acetate (6×6 L). All organic phases are combined into the 50-gallon reactor. 5000 g of magnesium sulfate is then added to the organic phase. The mixture is then filtered through an in-line filter. The filtrate is transferred via in-line filter to a 50-gallon reactor. The filtrate is concentrated to a total volume of 20-30 L.

In a 22 L three neck round bottom flask, HCl gas is bubbled through 12 L of ethyl acetate to make an approximately 2.3 M solution of HCl/ethyl acetate. After titration assay, the solution is adjusted to exactly 2.3 M by adding either ethyl acetate or HCl gas.

8.2 L of the 2.3 M solution of HCl/ethyl acetate is added (over a period of approx. 1.5 hours) to the filtrate (above), maintaining the temperature at ≦20° C. and ensuring that a pH of 2 is obtained. Once the addition is complete, the mixture is stirred at 0 to −5° C. for a period of 16 hours.

The resulting solids, crude (+)-1-(3,4-dichlorophenyl)-3-azabicyclo[3.1.0]hexane hydrochloride, are filtered onto a plate filter using a polypropylene filter cloth. The solids are then washed with ethyl acetate (2×2 L), acetone (2×2 L) and ethyl ether (2×2 L) and dried under vacuum. The material is transferred to a tarred 5-gallon polyethylene container and weighed.

### Step 6a: Recrystallization of (+)-1-(3.4-dichloroPhenyl)-3-azabicyclo[3.1.0]hexane hydrochloride from isopropanol

The solids (from Step 6, above) are transferred to a 50-gallon reactor and isopropanol is added (8-10 mL/g of solid). The mixture is heated to reflux. The solution is filtered through an in-line filter into another 50 gallon reactor. The solution is cooled to 0 to −5° C. and maintained at this temperature with stirring for approximately 2 hours. The resulting solids are filtered onto a plate filter using a polypropylene filter cloth. The solids are then washed with ethyl acetate (2×2 L), acetone (2×2 L) and ethyl ether (2×2 L). The solids are dried under vacuum.

The product, (+)-1-(3,4-dichlorophenyl)-3-azabicyclo[3.1.0]hexane hydrochloride, is transferred into clean, tarred drying tray(s). The tray(s) are placed in a clean, vacuum drying oven. The product is dried at 50° C. to constant weight. The material is dried for a minimum of 12 hours at <10 mm Hg. This product was a mixture of polymorph form A and polymorph form B, with each polymorph present in the mixture in an amount of about 50% by weight. This product was used as the starting material for Examples V, VI, and VII below.

### Example V

### Preparation of Polymorph Form A of (+)-1-(3,4-dichlorophenyl)-3-azabicyclo[3.1.0]hexane

As in U.S. patent application Ser. No. 12/428,399, 20 mg samples of the 50% by weight mixture of polymorph form A and polymorph form B of the hydrochloride salt of (+)-1-(3,4-dichlorophenyl)-3-azabicyclo[3.1.0]hexane from Example IV were dissolved in 0.5 ml of aqueous ethanol. Other samples were prepared by dissolving 20 mg of this mixture in 0.5 mL of water. Both solutions were filtered through a 0.2 micron nylon filter. Both filtered solutions were then allowed to evaporate under ambient conditions, some samples partially covered and other samples completely uncovered. After 6 days, both the uncovered and partially covered ethanol solution samples evaporated. After 7 days, the uncovered water solutions evaporated. After 15 days, the partially covered water solutions evaporated. For each sample, after the solvent (either aqueous ethanol or water) evaporated completely, 20 mg of dry solid residue was left. The solid in all samples thus produced was the pure polymorph form A crystals of the hydrochloride salt of (+)-1-(3,4-dichlorophenyl)-3-azabicyclo[3.1.0]hexane as demonstrated by Raman spectroscopy and XRPD analysis as described above.

### Example VI

### Preparation of Polymorph Form B of (+)-1-(3,4-dichlorophenyl)-3-azabicyclo[3.1.0]hexane

As in U.S. patent application Ser. No. 12/428,399, 40 mg samples of the 50% by weight mixture of polymorph form A and polymorph form B of the hydrochloride salt of (+)-1-(3,4-dichlorophenyl)-3-azabicyclo[3.1.0]hexane from Example IV were mixed with 0.5 mL of anhydrous acetonitrile to produce a concentration of about 80-100 mg/mL and the resulting samples were stirred at various temperatures between 50° C. and 80° C. for various periods of time (some for 4 days and 6 days at about 50° C. and some for 1 day at about 80° C.). The resulting samples were each mixtures of a clear liquid and some solid. The clear liquid was decanted off, and the remaining solid was vacuum dried at ambient temperature for 1 hour to 2 days (50° C. sample), or 6 days (80° C. sample) to afford pure crystalline polymorph form B. All samples produced the pure polymorph form B crystals of the hydrochloride salt of (+)-1-(3, 4-dichlorophenyl)-3-azabicyclo[3.1.0]hexane as demonstrated by Raman spectroscopy and XRPD analysis as described above.

### Example VII

### Preparation of Polymorph Form C of (+)-1-(3,4-dichlorophenyl)-3-azabicyclo[3.1.0]hexane

51 mg of the 50% by weight mixture of polymorph form A and polymorph form B of the hydrochloride salt of (+)-1-(3,4-dichlorophenyl)-3-azabicyclo[3.1.0]hexane prepared in Example IV was weighed into a vial. The vial was covered with aluminum foil perforated with pinholes and placed in an oven at 80° C. for 4 days to produce the pure polymorph C crystals of the hydrochloride salt of (+)-1-(3, 4-dichlorophenyl)-3-azabicyclo[3.1.0]hexane as demonstrated by Raman spectroscopy and XRPD analysis as described above.

### Example VIII

### Activity Comparison of (+)-1-(3,4-dichlorophenyl)-3-Azabicyclo[3.1.0]Hexane and (±)-1-(3,4-dichlorophenyl)-3-Azabicyclo[3.1.0]Hexane

### Norepinephrine Transporter Binding Assay

The norepinephrine binding assay was performed according to the methods described in Raisman et al., Eur. J. Pharmacol. 78:345-351 (1982) and Langer et al., Eur. J. Pharmacol. 72:423 (1981). The receptor source was rat forebrain membranes; the radioligand was [³H]-nisoxetine (60-85 Ci/mmol) at a final ligand concentration of 1.0 nM; the non-specific determinant [1.0 μm]; reference compound and positive control were (±)-desmethylimipramine HCl. (+)-1-(3,4-dichlorophenyl)-3-azabicyclo[3.1.0]hexane HCl was obtained according to the method of Example II, above. Reactions were carried out in 50 mM TRIS-HCl (pH 7.4), containing 300 mM NaCl and 5 mM KCl at 0° C. to 4° C. for 4 hours. The reaction was terminated by rapid vacuum filtration onto glass fiber filters. Radioactivity trapped in the filters was determined and compared to control values in order to ascertain the interactions of the test compound with the norepinephrine uptake site. The data are reported in Table 5 below.

TABLE 5
Norepinephrine Transporter Binding Assay

| Compound | Ki |
|---|---|
| (±)-1-(3,4-dichlorophenyl)-3-Azabicyclo[3.1.0]Hexane | 1.42 × 10⁻⁷ |
| (+)-1-(3,4-dichlorophenyl)-3-azabicyclo[3.1.0]hexane HCl | 8.20 × 10⁻⁸ |
| (±)-desmethylimiprimine HCl | 1.13 × 10⁻⁹ |

The data in Table 5 show that (+)-1-(3,4-dichlorophenyl)-3-azabicyclo[3.1.0]hexane HCl has a significantly greater affinity for the norepinephrine uptake site than does the (±)-1-(3,4-dichlorophenyl)-3-azabicyclo[3.1.0]hexane HCl. Successful inhibition of norepinephrine reuptake has been has been associated with the treatment of one or more of the symptoms of depression (R. J. Baldessarini, Drugs and the Treatment of Psychiatric Disorders: Depression and Mania, in Goodman& Gilman's The Pharmacological Basis of Therapeutics 431-459 (9^{th} ed. 1996)). Therefore, (+)-1-(3,4-dichlorophenyl)-3-azabicyclo[3.1.0]hexane or a pharmaceutically acceptable salt thereof will be significantly more active than (±)-1-(3,4-dichlorophenyl)-3-azabicyclo[3.1.0]hexane or a pharmaceutically acceptable salt thereof for treating or preventing depression in a patient.

### Serotonin Transporter Binding Assay

The serotonin binding assay was performed according to the methods described in D'Amato et al., J. Pharmacol. Exp. Ther. 242:364-371 (1987) and Brown et al., Eur. J. Pharmac. 123:161-165 (1986). The receptor source was rat forebrain membranes; the radioligand was [³H]-citalopram (70-87 Ci/mmol) with a final ligand concentration of 0.7 nM; the non-specific determinant was clomipramine [10 μm]; and the reference compound and positive control were (±)-desmethylimipramine. (+)-1-(3,4-dichlorophenyl)-3-azabicyclo[3.1.0]hexane HCl was obtained according to the method of Example II, above. Reactions were carried out in 50 mM TRIS-HCl (pH 7.4) containing 120 mM NaCl and 5 mM KCl at 25° C. for 60 minutes. The reaction was terminated by rapid vacuum filtration onto glass fiber filters. Radioactivity trapped in the filters was determined using liquid scintillation spectrometry and compared to control values in order to ascertain any interactions of test compound with the serotonin transporter binding site. The data are reported in Table 6 below.

TABLE 6
Serotonin Transporter Binding Assay

| Compound | Ki |
|---|---|
| (±)-1-(3,4-dichlorophenyl)-3-Azabicyclo[3.1.0]Hexane | 1.18 × 10⁻⁷ |
| (+)-1-(3,4-dichlorophenyl)-3-azabicyclo[3.1.0]hexane HCl | 5.08 × 10⁻⁸ |
| (±)-desmethylimiprimine HCl | 2.64 × 10⁻⁸ |

The data in Table 6 show that (+)-1-(3,4-dichlorophenyl)-3-azabicyclo[3.1.0]hexane HCl has a significantly greater affinity for the serotonin uptake site than does (±)-1-(3,4-dichlorophenyl)-3-azabicyclo[3.1.0]hexane HCl. Successful inhibition of serotonin reuptake has been has been associated with the treatment of one or more of the symptoms of depression (R. J. Baldessarini, Drugs and the Treatment of Psychiatric Disorders: Depression and Mania, in Goodman& Gilman's The Pharmacological Basis of Therapeutics 431-459 (9^{th} ed. 1996)). Therefore, (+)-1-(3,4-dichlorophenyl)-3-azabicyclo[3.1.0]hexane or a pharmaceutically acceptable salt thereof will be significantly more active than (±)-1-(3,4-dichlorophenyl)-3-azabicyclo[3.1.0]hexane or a pharmaceutical salt thereof for treating or preventing depression in a patient. (See U.S. Pat. No. 6,372,919)

### Example IX

### Acute Intravenous or Oral Rat Study of Amitifadine (EB-1010)

In a rat bioavailability study, blood samples were collected at 0.25, 0.5, 1, 2, 4, 6, 8, 12, and 24 hours following a single oral (10 mg/kg) or IV (5 mg/kg) dose. Plasma samples were assayed using a validated LC/MS/MS assay. Systemic exposure to amitifadine (EB-1010) was greater in females than in males, due to a lower rate of clearance and slightly lower volume of distribution. The absolute oral bioavailability of amitifadine (EB-1010) was high in both sexes (≧77%).

### Example X

### Acute Intravenous or Oral Mouse Study of Amitifadine (EB-1010)

In a mouse bioavailability study, blood samples were collected at 0.25, 0.5, 1, 2, 4, 6, 8, 10, and 24 hours following a single oral dose of 6 or 20 mg/kg or an IV dose of 5 mg/kg. Fasted male mice were used in this study. Concentrations were determined using an LC/MS/MS assay following liquid-liquid extraction of plasma samples. Bioavailability was calculated using the mean normalized AUC_{0-∞} values obtained following oral administration of 6 mg/kg relative to the mean normalized AUC_{0-∞} at 5 mg/kg IV. Mean oral bioavailability of amitifadine (EB-1010) in male mice was ˜16%.

### Example XI

### Anesthetized Dog Intravenous Cardiovascular Study of EB-1010

This study was performed in 4 sodium pentobarbital-anesthetized, ventilated, vagotomized, and conditioned mixed-breed dogs to assess effects of amitifadine (EB-1010) on blood pressure, heart rate, blood flow, and ECG activity following rising IV femoral infusions. Doses of 2, 7, and 20 mg/kg in 10 mL deionized water were infused over 3 consecutive 30-minute periods using a programmable syringe infusion pump yielding cumulative doses of 2, 9, and 29 mg/kg. Whole blood samples were collected via a cannulated femoral vein at 0, 10, 20, and 29 minutes (timed from the start of each dose administration). Derived plasma fractions were frozen until analyzed for amitifadine (EB-1010) concentrations via LC/MS/MS.

Peak amitifadine (EB-1010) plasma concentrations (mean±SD) measured during each consecutive 30-minute IV infusion were 35±16 μM (7980±3650 ng/mL), 120±45 μM (27400±10300 ng/mL), and 236±105 μM (53800±23900 ng/mL) at cumulative doses of 2, 9, and 29 mg/kg, respectively. Dose-related hemolysis was apparent in derived plasma fractions.

### Example XII

### Comparative In Vitro Metabolism of amitifadine (EB-1010) in Hepatocytes from Mice, Rats, Dogs, and Humans

To identify metabolites, amitifadine (EB-1010) was incubated at a concentration of 87 μM (20 μg/mL) with mouse, rat, dog, and human hepatocytes for 4 hours. Six different metabolites were identified using LC/MS and LC/MS/MS analyses. Not all of the metabolites were present in incubations from the various species. One of the prominent metabolites, the 5-lactam (5-(3,4-dichloro-phenyl)-3-aza-bicyclo[3.1.0]hexan-2-one), was formed due to oxidation of a carbon atom adjacent to the nitrogen to form a ketone derivative. Another metabolite was due to the addition of CO₂ to the nitrogen to form a carbamate analog, which was subsequently conjugated to the glucuronide. The other 4 metabolites were minor; their structures were not determined.

### Example XIII

### In Vitro Plasma Protein Binding Study of EB-1010

The objective of this study was to determine the extent of binding of amitifadine (EB-1010) to human plasma proteins. Samples of human plasma were fortified with 250, 500, 1,000, and 1,500 ng (free base)/mL. The concentration in both the unfiltered plasma and the plasma ultrafiltrate were analyzed by an HPLC/MS/MS method.

Amitifadine (EB-1010) in human plasma was nearly completely bound (mean, 99%) over the concentration range evaluated; nonspecific binding over the same concentration range was negligible.

### Example XIV

### 2 Week Rat Toxicology and Toxokinetic Study of EB-1010

In two experiments, groups of Crl:CD® (SD)IGS BR rats (Charles River, Wilmington, Mass.) received oral (gavage) administration of amitifadine (EB-1010) for 14 days at dose levels of 0, 10, 20, or 40 mg/kg/day. Blood samples were collected on Day 0 and Day 13 of the study from up to 3 animals/gender/group.

For the toxicology assessment, five mice of each gender were in each group. Assessment included clinical observations, body weight and food consumptions measures were noted periodically during the study. At termination, clinical pathology evaluations (hematology and serum chemistry) were performed, necropsies conducted, selected organs were weighed, and selected tissues examined microscopically.

For the toxicokinetics study, a control group was not used, but three groups of 10 mice of each gender were given doses of amitifadine (EB-1010) and additional blood samples were taken pre-dose and 1, 2, 4, 8, and 24 hours. The toxicokinetic results are summarized in Table 7.

TABLE 7
Toxicokinetics of EB-1010 in the 2-Week Rat Study

| Gender/ | AUC₀₋₂₄ | | Cₘₐₓ | | Tₘₐₓ | |
|---|---|---|---|---|---|---|
| EB-1010 | (ng · h/mL) | | (ng/mL) | | (h) | |
| (mg/kg/day) | Day 0 | Day 13 | Day 0 | Day 13 | Day 0 | Day 13 |
| Males | | | | | | |
| 10 | 7878 | 6306 | 1432 | 878 | 1 | 2 |
| 20 | 17424 | 12974 | 2999 | 1448 | 1 | 2 |
| 40 | 34843 | 25627 | 3037 | 2730 | 1 | 1 |
| Females | | | | | | |
| 10 | 11649 | 8193 | 1830 | 1150 | 2 | 2 |
| 20 | 28607 | 22013 | 2803 | 2301 | 1 | 1 |
| 40 | 50609 | 32926 | 4438 | 3103 | 4 | 2 |

There were no compound-related deaths, clinical findings, or effects on body weight for male or female rats in this study. No compound-related effects were noted at 10 mg/kg/day. Food consumption was reduced slightly only in females at ≧20 mg/kg/day. The liver was the primary target system in this study. A dose-related increase in cholesterol levels occurred in both sexes and was significantly increased in females at 40 mg/kg/day. No other treatment-related effects on serum chemistry and no effects on hematology were noted. A dose-related increase in mean absolute and relative (to body weight) liver weights occurred; increases of 20% and 21%, respectively for the males and 18% and 23%, respectively, for the females were noted at 40 mg/kg/day. Minimal centrilobular hepatocellular hypertrophy accompanied liver weight changes. These hepatic findings were considered adaptive changes following repeated dosing with amitifadine (EB-1010) and were not considered adverse. The no observed adverse effect level (NOAEL) for systemic toxicity was 40 mg/kg/day.

### Example XV

### 13-Week Rat Toxicity Study of EB-1010

In two separate experiments, toxicology and toxicokinetic studies were performed. Groups of twenty Crl:CD®(SD)IGS BR rats (Charles River, Wilmington, Mass.) (ten of each gender per group) received daily oral (gavage) administration of amitifadine (EB-1010) for approximately 13 weeks at dose levels of 0, 10, 25, or 60 mg/kg/day. Blood samples were collected on Day 0 and Day 87 of the study from up to 3 animals/gender/group.

For toxicology assessment, clinical observations, body weight and food consumptions measures were noted periodically during the study. At termination, clinical pathology evaluations (hematology and serum chemistry) were performed, necropsies conducted, selected organs were weighed, and selected tissues examined microscopically.

In the toxicokinetic study, there was no control group, but three groups given EB-1010. Blood samples were additionally taken pre-dose and 1, 2, 4, 8, and 24 hours. The toxicokinetic results are summarized in Table 8.

TABLE 8
Toxicokinetics of EB-1010 in the 13-Week Rat Study

| Gender/ | AUC₀₋₂₄ | | Cₘₐₓ | | Tₘₐₓ | |
|---|---|---|---|---|---|---|
| EB-1010 | (ng · h/mL) | | (ng/mL) | | (h) | |
| (mg/kg/day) | Day 0 | Day 87 | Day 0 | Day 87 | Day 0 | Day 87 |
| Males | | | | | | |
| 10 | 5940 | 8133 | 863 | 1116 | 2 | 1 |
| 25 | 17401 | 21975 | 2355 | 2486 | 2 | 2 |
| 60 | 36257 | 60093 | 3685 | 5013 | 1 | 1 |
| Females | | | | | | |
| 10 | 10920 | 14350 | 1392 | 1848 | 1 | 1 |
| 25 | 30969 | 30205 | 2811 | 3702 | 2 | 2 |
| 60 | 54368 | 88137 | 6596 | 6529 | 2 | 2 |

Exposures to amitifadine (EB-1010) increased in a generally dose-proportional manner over the dose range of 10 to 60 mg/kg/day. Exposure tended to increase slightly with 13 weeks of repeated dosing. Gender differences in exposure were observed for EB-1010; female rats had slightly higher AUC₀₋₂₄ and Cₘₐₓ values than male rats (differences up to 85%) in all dose groups.

Three females in the 60 mg/kg/day group died during the first week of the study as a result of test article administration, and 1 female died as a result of suspected gavage accident. Clinical observations of clear material around the mouth were commonly observed following dose administration at 25 and 60 mg/kg/day. Other treatment-related clinical signs consisted of red material around the mouth at 25 and 60 mg/kg/day, and yellow staining of the urogenital area at 60 mg/kg/day. Decreased body weight and food consumption parameters were noted at 25 and 60 mg/kg/day. By the end of the study, mean cumulative body weight gains were decreased compared to control values by 30% and 13% at 60 mg/kg/day in males and females, respectively, and by 16% and 13% at 25 mg/kg/day in males and females, respectively.

Treatment-related changes noted in clinical chemistry parameters included higher alanine aminotransferase (ALT) and cholesterol levels and higher urine volume in both males and females, higher ALP in males, and higher bilirubin in females at 60 mg/kg/day. Changes in cholesterol (both sexes) and urine volume (males only) extended to the 25 mg/kg/day group. The ALT, cholesterol, ALP, and bilirubin levels were 35% to 55% higher than controls at 60 mg/kg/day and occurred in the presence of microscopic observations of hepatocellular hypertrophy and vacuolation. Urine volume was approximately 90% to 170% higher than controls at 60 mg/kg/day in males and females and 25 mg/kg/day in males. Higher mean urea nitrogen in females at 60 mg/kg/day was associated with higher urine volume and kidney weights. Dose-related changes in organ weights (expressed as absolute, relative to final body weight, or relative to brain weight) consisted of higher liver, kidney, and thyroid weights, and lower epididymis and uterine weights. Relative liver weights were 7%, 22%, and 43% higher than controls in 10, 25, and 60 mg/kg/day males, respectively, and 6%, 19%, and 51% higher at 10, 25, and 60 mg/kg/day in females, respectively. Relative epididymis weights were 11% and 13% lower than controls at 25 and 60 mg/kg/day, respectively. Higher relative kidney weights were observed at 60 mg/kg/day in males and females (24% and 22% higher, respectively) and were associated with higher urea nitrogen (females) and higher urine volume (both sexes), but no histologic changes. Absolute and relative thyroid weights ranged from 24% to 27% and 37% to 45% higher than control values in the 25 and 60 mg/kg/day group females, respectively, in the absence of any histopathologic findings. Relative uterine weights ranged from 23% to 50% lower than controls in all treated groups, but there were no associated histopathologic findings.

Treatment-related microscopic changes occurred in a dose-related manner and were limited to the liver (all EB-1010-treated groups) and epididymis (at 25 and 60 mg/kg/day). Minimal to mild hepatocellular vacuolation was noted in males from all EB-1010-treated groups and in females at 60 mg/kg/day. Minimal to mild hypertrophy was noted in all EB-1010-treated groups (both sexes). Epididymal changes consisted of minimal to mild inflammation, minimal to moderate edema, and minimal to mild epithelial degeneration at 60 mg/kg/day, as well as minimal epithelial degeneration at 25 mg/kg/day.

In summary, body weight and food consumption parameters were decreased and treatment-related liver (weight and histopathology), epididymal (weight and histopathology), and kidney, thyroid, and uterine effects occurred at doses of 25 mg/kg/day and above. Dose-related hepatocellular vacuolation and hypertrophy were noted in all dose groups. However, the minimal hepatic findings at 10 mg/kg/day were not accompanied by changes in measured indicators of hepatic damage, other histopathologic changes, or general measures of toxicity. Therefore, the NOAEL for oral (gavage) administration of amitifadine (EB-1010) to rats for 13 weeks was 10 mg/kg/day. Corresponding Day 87 AUC₀₋₂₄ values for the 10 mg/kg/day group males and females were 8,133 and 14,350 ng·h/mL, respectively.

### Example XVI

### 26-Week Rat Toxicity Study of EB-1010

Groups of Crl:CD®(SD)IGS BR rats (Charles River, Wilmington, Mass.) received daily oral (gavage) administration of amitifadine (EB-1010) for approximately 26 weeks at dose levels of 0, 2.5, 8, or 25 mg/kg/day. A portion of the animals from each group was maintained for an additional 4-week post-treatment recovery period. Blood samples were collected on Day 0 and Day 177 of the study from up to 3 animals/gender/group.

For toxicology assessment, groups comprised thirty animals of each gender. Assessments included clinical observations, body weight, and food consumption measures noted periodically during the study. Clinical pathology evaluations (hematology, serum chemistry, and urinalysis) were conducted on all surviving animals at the scheduled necropsies. Ophthalmic examinations were performed during Weeks 1 and 13. Complete necropsies were conducted on all animals, selected organs were weighed, and selected tissues were examined microscopically from all animals in the control and 25 mg/kg/day groups. In addition, the liver, vagina, and ovaries were identified as potential target tissues and were examined microscopically for all animals from all groups at the primary and recovery necropsies. Sections of liver from rats in the control and 25 mg/kg/day groups were collected at both the primary and recovery necropsies for assessment of drug metabolism enzyme (microsomal cytochrome P450) activity. 10 animals of each gender from each group were held to week 30 for a 4-week post-treatment recovery period.

For the toxicokinetic studies, there were ten animals of each gender in each group. There were no control groups, but three groups given the dosages of amitifadine (EB-1010) listed above. Additionally, blood samples were taken pre-dose and 1, 2, 4, 8 and 24 hours after dosing. The toxicokinetic results for parent drug and the lactam metabolite 5-(3,4-dichloro-phenyl)-3-aza-bicyclo[3.1.0]hexan-2-one (EB-10101) are summarized in Table 9.

TABLE 9
Toxicokinetics of EB-1010 and EB-10101* in the
26-Week Rat Study

| | EB-1010 (free base) | | | | | |
|---|---|---|---|---|---|---|
| Gender/ | AUC₀₋₂₄ | | | | Tₘₐₓ (h) | |
| EB-1010 | (ng · h/mL) | | Cₘₐₓ (ng/mL) | | | Day |
| (mg/kg/day) | Day 0 | Day 177 | Day 0 | Day 177 | Day 0 | 177 |
| Males | | | | | | |
| 2.5 | 1506 | 2381 | 260 | 313 | 1 | 1 |
| 8 | 5379 | 7129 | 1040 | 954 | 1 | 1 |
| 25 | 18098 | 17821 | 2600 | 2600 | 1 | 1 |
| Females | | | | | | |
| 2.5 | 3216 | 3120 | 370 | 425 | 2 | 1 |
| 8 | 9970 | 11133 | 1430 | 1417 | 1 | 2 |
| 25 | 23845 | 29743 | 2633 | 3500 | 1 | 1 |
| | EB-10101* | | | | | |
| Gender/ | AUC₀₋₂₄ | | | | Tₘₐₓ (h) | |
| EB-1010 | (ng · h/mL) | | Cₘₐₓ (ng/mL) | | | Day |
| (mg/kg/day) | Day 0 | Day 177 | Day 0 | Day 177 | Day 0 | 177 |
| Males | | | | | | |
| 2.5 | 9558 | 20435 | 656 | 1084 | 4 | 4 |
| 8 | 36816 | 80438 | 2372 | 3838 | 8 | 8 |
| 25 | 133779 | 253936 | 6725 | 13110 | 8 | 4 |
| Females | | | | | | |
| 2.5 | 8984 | 15709 | 676 | 1018 | 8 | 8 |
| 8 | 31457 | 88098 | 1921 | 4513 | 8 | 8 |
| 25 | 107007 | 366055 | 5588 | 22020 | 24 | 4 |

*Formerly identified as DOV 216.298

There were no treatment-related deaths. One male each in the 8 and 25 mg/kg/day groups was found dead on Days 151 and 46, respectively. The cause of death was undetermined. One male in the 2.5 mg/kg/day group was euthanized in extremis on Day 98 due to clinical observations of prostration, unkempt appearance, impaired muscle coordination, thinness, dermal atonia, extremities pale and cool to touch, pale body, red material on various body surfaces (nose, mouth, urogenital area, ventral trunk, and hind/forelimbs), and decreased defecation. There were no EB-1010-related effects on hematology parameters on any assessment day; on estrous stage at termination; no treatment-related ophthalmic findings at any assessment period; no macroscopic findings at the scheduled necropsies; and no toxicologically relevant findings in any assessments for animals receiving 2.5 mg/kg/day.

Clinical findings consisted of limited incidences of clear and/or red material around the nose and/or mouth and yellow material on the urogenital area (females only) for animals from the 8 and 25 mg/kg/day groups, primarily at the 1-hour post-dosing observation period. These findings generally did not persist to the following day nor were they observed during the post-treatment recovery period.

Significantly lower mean body weight gains, accompanied by only occasionally lower food consumption, persisted throughout the treatment period in animals receiving 25 mg/kg/day. Mean body weights of animals in this group were reduced significantly compared to the controls throughout the 26-week treatment period. By the end of the treatment period, mean cumulative body weight gains were 18% and 14% lower in males and females, respectively, from the 25 mg/kg/day group. Somewhat higher mean body weight gains and food consumption occurred in this group once treatment was terminated, but mean body weights remained 12% and 10% lower than control values in males and females, respectively, at the end of the 4-week recovery period. The lower body weight gains and cumulative body weight gains at 25 mg/kg/day were considered to be adverse due to the magnitude of change and the persistence of these changes during the 4-week recovery period.

At Week 13 and/or 26, EB-1010-related findings for clinical pathology parameters that were noted in a dose-related manner included 20% to 55% higher mean serum cholesterol levels for males and females in the 8 and 25 mg/kg/day groups and 3% to 10% higher mean total serum protein and albumin levels for females from these groups. The serum globulin level was also increased in females in the 25 mg/kg/day group (4%) but this change was not observed in a dose-related manner. In addition, 6% to 19% higher mean phosphorus levels were noted for males in the 8 and 25 mg/kg/day groups and females in the 25 mg/kg/day group. Higher mean total urine volume (approximately 60% to 75%) and lower mean specific gravity were observed for males in the 8 and 25 mg/kg/day groups. These changes were not considered to be adverse due to the magnitude of the changes and because none of the changes persisted to the end of the 4-week recovery period.

At the primary necropsy, mean liver weights were 7% to 27% higher in animals of both sexes from the 8 and 25 mg/kg/day groups. Higher liver weights occurred in conjunction with hepatocellular hypertrophy and vacuolation, as well as liver enzyme induction (25 mg/kg/day only). Centrilobular hepatocellular hypertrophy occurred with increased incidence and severity in all groups and centrilobular hepatocellular vacuolation also occurred in males from the 8 and 25 mg/kg/day groups and females from the 25 mg/kg/day group. Induction of multiple P450 isoenzymes also occurred in animals receiving 25 mg/kg/day. Together these findings were considered adaptive liver changes and not toxicological changes. Higher (12%) mean ovary/oviduct weights were observed in females from the 25 mg/kg/day group and higher (9.5%) mean testes weights were noted in males from the 25 mg/kg/day group. The toxicologic relevance of the higher mean ovary/oviduct and testes weights is uncertain in the absence of correlating macroscopic or microscopic changes. Following the 4-week recovery period, there was evidence of recovery in these organ weight changes, and no treatment-related microscopic effects were noted.

Significantly lower mean body weight gains, accompanied by only occasionally lower food consumption, persisted throughout the treatment period in animals receiving 25 mg/kg/day.

The exposures to both amitifadine (EB-1010) and 5-(3,4-dichloro-phenyl)-3-aza-bicyclo[3.1.0]hexan-2-one (EB-10101) appeared to be proportional to the dosage of amitifadine (EB-1010) over the range of 2.5 to 25 mg/kg/day. Exposures to 5-(3,4-dichloro-phenyl)-3-aza-bicyclo[3.1.0]hexan-2-one were higher than to amitifadine (EB-1010), and AUC₀₋₂₄ values for the metabolite relative to the parent drug increased from approximately 7-fold higher on Day 0 to as much as 14-fold higher on Day 177. Exposure patterns to amitifadine (EB-1010) were similar on Days 0 and 177. However, exposures to 5-(3,4-dichloro-phenyl)-3-aza-bicyclo[3.1.0]hexan-2-one were higher on Study Day 177, suggesting that 5-(3,4-dichloro-phenyl)-3-aza-bicyclo[3.1.0]hexan-2-one accumulates in the plasma. At equivalent dosages of amitifadine, exposures to amitifadine (EB-1010) were up to approximately 2-fold higher for female than for male rats, while exposures to 5-(3,4-dichloro-phenyl)-3-aza-bicyclo[3.1.0]hexan-2-one were typically similar between the genders.

Following 26 weeks of treatment at 25 mg/kg/day, liver metabolic enzyme analysis suggested that amitifadine (EB-1010) is an inducer of CYP1A, CYP2B, and CYP2E in male rats and an inducer of CYP1A, CYP2B, CYP2E, CYP3A, and CYP4A in female rats. However, the degrees of induction were not as great as the degrees produced by the prototypical inducers. These effects were largely reversed following the 4-week recovery period.

In summary, increased salivation, lower body weight gains and terminal body weights, clinical pathology changes, higher ovary/oviduct and testes weights in the absence of adverse histologic changes, adaptive hepatic changes, and evidence of enzyme induction occurred at 25 mg/kg/day. The lower body weight gains and terminal body weights were considered adverse due to the magnitude of the changes and the persistence of these changes during the 4-week recovery period. At 8 mg/kg/day, only the clinical signs, slight clinical pathology changes, and adaptive hepatic changes occurred. There were no remarkable effects in any assessments at 2.5 mg/kg/day. Therefore, the no observer effect level (NOEL) for oral (gavage) administration of amitifadine (EB-1010) to rats for 26 consecutive weeks was considered to be 2.5 mg/kg/day, and the NOAEL was considered to be 8 mg/kg/day. Corresponding Day 177 AUC₀₋₂₄ values for males and females receiving 8 mg/kg/day were 7,129 and 11,133 ng·h/mL, respectively, for amitifadine (EB-1010) (free base) and 80,438 and 88,096 ng·h/mL, respectively, for the lactam metabolite (5-(3,4-dichloro-phenyl)-3-aza-bicyclo[3.1.0]hexan-2-one (EB-10101)).

### Example XVII

### 2-Week Dog Toxicity Study of EB-1010

Groups of beagle dogs (2 of each gender per group) received daily oral (capsule) administration of amitifadine (EB-1010) for a period of 14 days at dose levels of 0, 5, 10, or 20 mg/kg/day; an additional group of dogs received empty capsules. Blood samples were collected on Day 0 and Day 13 of the study from up to 2 animals/gender/group. Samples were taken pre-dose, and 1, 2, 4, 8 and 24 hours.

For toxicology assessment, clinical observations, body weight, and food consumption measures were noted periodically during the study. Clinical pathology evaluations (hematology and serum chemistry) were performed prior to the initiation of treatment (Week −1) and just prior to the scheduled necropsy. At termination, necropsies were conducted and selected organs were weighed. Selected tissues were examined microscopically for animals in the control and high-dose groups. The toxicokinetic results are summarized in Table 10.

TABLE 10
Toxicokinetics of EB-1010 in the 2-Week Dog Study

| Gender/ | AUC₀₋₂₄ | | Cₘₐₓ | | Tₘₐₓ | |
|---|---|---|---|---|---|---|
| EB-1010 | (ng · h/mL) | | (ng/mL) | | (h) | |
| (mg/kg/day) | Day 0 | Day 13 | Day 0 | Day 13 | Day 0 | Day 13 |
| Males | | | | | | |
| 5 | 7365 | 7552 | 1578 | 1836 | 1 | 2 |
| 10 | 13791 | 3173 | 3826 | 1276 | 1 | 1 |
| 20 | 41685 | 32765 | 11624 | 9279 | 1 | 1 |
| Females | | | | | | |
| 5 | 5567 | 5610 | 1648 | 2044 | 2 | 1 |
| 10 | 15037 | 11180 | 3452 | 2805 | 1 | 2 |
| 20 | 57102 | 11499 | 9948 | 3531 | 1 | 2 |

All animals survived to the scheduled necropsy. The following dose-related clinical signs, attributed to the extended pharmacology of EB-1010, were noted during the post-treatment period: pupillary dilation and emesis at ≧5 mg/kg/day; injected sclera at ≧10 mg/kg/day; and clear or green ocular discharge, visual impairment, and head movements at ≧20 mg/kg/day. Visual impairment was indicated by the following observations: animals did not seem to be aware of their location when walking around the room, were not able to focus on moving objects (loss of tracking), and were noted to have excessive back and forth movement of the head. These observations did not persist to the following day.

Amitifadine (EB-1010) was not detected in any of the samples from the group receiving empty capsules. The exposure to amitifadine (EB-1010) generally increased in a dose-proportional manner over the range of 5 to 20 mg/kg/day; apparent departures from proportionality were typically due to large differences between the 2 replicate animals. There was no evidence of accumulation of amitifadine (EB-1010) with repeated dosing for 2 weeks. No clear gender differences in exposure were observed; apparent differences in male and female exposures were due to large differences between the 2 replicate animals. In addition, there was no evidence of (+) to (−) stereoisomer conversion in the dog.

Treatment-related mean body weight losses and mean cumulative body weight losses were noted at ≧10 mg/kg/day. Mean food consumption was 16% to 28% and 15% to 20% lower than control values in the 20 mg/kg/day group males and females, respectively. Hematology and serum chemistry parameters were unaffected by treatment with EB-1010. No treatment-related gross or histopathologic findings or changes in organ weights were observed at the scheduled necropsy. The NOAEL for systemic toxicity was 10 mg/kg/day. Corresponding Day 13 AUC₀₋₂₄ values for the 10 mg/kg/day group males and females were 3,173 and 11,180 ng·h/mL, respectively.

### Example XVIII

### 13-Week Dog Toxicity Study of EB-1010

Groups of beagle dogs (four of each gender per group) received daily oral (capsule) administration of amitifadine (EB-1010) for approximately 13 weeks at dose levels of 0, 2, 6, or 20 mg/kg/day; an additional group of dogs received empty capsules. Blood samples were collected on Day 0 and Day 88 of the study from up to 4 animals/gender/group. For toxicology assessment, clinical observations, body weight, and food consumption measures were recorded periodically during the study. Ophthalmic examinations were performed and ECGs recorded during Weeks −1 and 12. Clinical pathology evaluations (hematology, serum chemistry, and urinalysis) were performed prior to the initiation of dose administration (Week −1) and prior to the scheduled necropsy (Week 13). At termination, complete necropsies were performed on all dogs and selected organs were weighed. Selected tissues from the control and 20 mg/kg/day group animals and animals euthanized in extremis were examined microscopically. Gross lesions from the 2 and 6 mg/kg/day groups were also examined. The toxicokinetic results are summarized in Table 11.

TABLE 11
Toxicokinetics of EB-1010 in the 13-Week Dog Study

| Gender/ | AUC₀₋₂₄ | | Cₘₐₓ | | Tₘₐₓ | |
|---|---|---|---|---|---|---|
| EB-1010 | (ng · h/mL) | | (ng/mL) | | (h) | |
| (mg/kg/day) | Day 0 | Day 86 | Day 0 | Day 86 | Day 0 | Day 86 |
| Males | | | | | | |
| 2 | 1570 | 2275 | 507 | 753 | 1 | 1 |
| 6 | 12836 | 13121 | 4637 | 4143 | 1 | 1 |
| 20 | 47843 | 44011 | 11392 | 7229 | 1 | 1 |
| Females | | | | | | |
| 2 | 1476 | 2154 | 635 | 663 | 1 | 1 |
| 6 | 13568 | 15908 | 5119 | 4847 | 1 | 1 |
| 20 | 63,607 | 29557 | 9278 | 8434 | 2 | 1 |

Amitifadine (EB-1010) was detected at a low, but quantifiable level in a single sample collected from an animal in the control group. The exposure to amitifadine (EB-1010) in the treated dogs increased in a generally dose-proportional manner over the dose range of 2 to 20 mg/kg/day. There was no evidence of accumulation of amitifadine (EB-1010) with repeated dosing for 13 weeks. No gender differences in exposure were observed.

There were no test article-related deaths during the study. One 2 mg/kg/day group female was euthanized in extremis as a result of Canine Juvenile Polyarteritis Syndrome (Beagle Pain Syndrome). Treatment-related clinical observations consisted primarily of dilated pupils following dose administration in all EB-1010-treated groups. Additional test article-related clinical findings at ≧6 mg/kg/day included reddened ears, emesis, wet clear material around the mouth, and partial eyelid closure. These findings were attributed to the extended pharmacology of EB-1010. Increased post-dosing incidences of soft feces occurred primarily in the females at 20 mg/kg/day. Treatment-related reduced body weight gain and food consumption occurred at 20 mg/kg/day throughout the study. Control males and females gained 1.5±0.87 and 1.7±0.65 kg, respectively, during the study, whereas the 20 mg/kg group gained −0.3±0.58 and 0.4±0.79 kg, respectively. By the end of the study, mean body weights of males and females were 13% and 9% lower, respectively, compared to the control group. These body weight decreases were accompanied by reduced food consumption (generally ≧10% less than control values in males) throughout the study.

Treatment-related increased relative liver weights occurred at 20 mg/kg/day in males and females (19% and 27% higher, respectively). No macroscopic or microscopic changes accompanied higher liver weights. No treatment-related changes in hematology, serum chemistry, urinalysis, ophthalmic, or ECG parameters were noted. Based on body weight loss and/or smaller body weight gains, reduced food consumption and increased relative liver weights at 20 mg/kg/day, the NOAEL for oral (capsule) administration of amitifadine (EB-1010) to dogs for 13 weeks was 6 mg/kg/day. Corresponding Day 88 AUC₀₋₂₄ values for the 6 mg/kg/day group males and females were 13,121 and 15,908 ng·h/mL, respectively.

### Example XIX

### 12-Month Dog Study of Amitifadine (EB-1010)

Groups of beagle dogs received a daily oral dose (capsule) of amitifadine (EB-1010) for approximately 52 weeks at dose levels of 0, 2, 6, or, 20 (15) mg/kg/day; an additional group of dogs received empty capsules (the 20 mg/kg/day dosage was lowered to 15 mg/kg/day on Day 84). Blood samples were collected on Days 0, 91, 181, 268 and 358 from up to 4 animals/gender/group. Plasma samples collected on Days 91, 181, 268, as well as all plasma samples collected from the control group were not evaluated.

For toxicology assessment four dogs of each gender were included in each group. Assessments included clinical observations, body weight, and food consumption measures which were recorded periodically during the study. Ophthalmic examinations were performed during Weeks 1, 12, 25, 38, 51, and 55 (recovery period). Electrocardiograms were recorded during Weeks 2, 12, 25, 38, and 51. Clinical pathology evaluations (hematology, serum chemistry, and urinalysis) were performed during Weeks 1, 12, 25, 38, 52, and 56 (recovery period). At termination, complete necropsies were performed on all dogs and selected organs were weighed. Selected tissues from the control and 20 (15) mg/kg/day group animals and animals euthanized in extremis were examined microscopically. Sections of livers from dogs in the control and 20 (15) mg/kg/day groups were collected at both the primary and 4-week post-treatment recovery necropsies for assessment of drug metabolism enzyme (microsomal cytochrome P450) activity.

The toxicokinetic results for parent drug and the lactam metabolite, 5-(3,4-dichloro-phenyl)-3-aza-bicyclo[3.1.0]hexan-2-one (EB-10101), are summarized in Table 12. The results of drug metabolism enzyme analyses are summarized in Table 13.

TABLE 12
Toxicokinetics of EB-1010 and EB-10101** in the 12-Month Dog Study

| | EB-1010 (free base) | | | | | |
|---|---|---|---|---|---|---|
| Gender/ | AUC₀₋₂₄ | | | | Tₘₐₓ (h) | |
| EB-1010 | (ng · h/mL) | | Cₘₐₓ (ng/mL) | | | Day |
| (mg/kg/day) | Day 0 | Day 358 | Day 0 | Day 358 | Day 0 | 358 |
| Males | | | | | | |
| 2 | 2127 | 2222 | 1043 | 844 | 1.3 | 1.0 |
| 6 | 10532 | 15811 | 3251 | 2780 | 1.3 | 1.3 |
| 20 (15)* | 29624 | 18078 | 7980 | 4832 | 1.0 | 1.2 |
| Females | | | | | | |
| 2 | 1581 | 3991 | 615 | 1151 | 1.0 | 1.0 |
| 6 | 10797 | 11113 | 3679 | 2421 | 1.3 | 1.0 |
| 20 (15)* | 24086 | 19608 | 6879 | 5797 | 1.0 | 1.2 |
| | EB-10101** | | | | | |
| Gender/ | AUC₀₋₂₄ | | | | Tₘₐₓ (h) | |
| EB-1010 | (ng · h/mL) | | Cₘₐₓ (ng/mL) | | | Day |
| (mg/kg/day) | Day 0 | Day 358 | Day 0 | Day 358 | Day 0 | 358 |
| Males | | | | | | |
| 2 | 2023 | 2334 | 243 | 309 | 2.0 | 4.0 |
| 6 | 13496 | 22501 | 1535 | 1699 | 3.3 | 7.0 |
| 20 (15)* | 51189 | 45524 | 4116 | 3329 | 7.1 | 7.3 |
| Females | | | | | | |
| 2 | 2219 | 7984 | 286 | 377 | 1.8 | 4.0 |
| 6 | 11686 | 18455 | 889 | 1360 | 3.8 | 7.0 |
| 20 (15)* | 50085 | 50832 | 3942 | 3809 | 7.1 | 7.3 |

*Dosage decreased to 15 mg/kg/day beginning on Day 84.
**Formerly identified as DOV 216,298

The exposures to amitifadine (EB-1010) and 5-(3,4-dichloro-phenyl)-3-aza-bicyclo[3.1.0]hexan-2-one (EB-10101) appeared to be proportional to the dosage of amitifadine (EB-1010) over the range of 2 to 20 (15) mg/kg/day, although emesis on Day 0 may have contributed to inter-animal variability. Exposures to 5-(3,4-dichloro-phenyl)-3-aza-bicyclo[3.1.0]hexan-2-one (EB-10101) were typically higher than the exposures to amitifadine (EB-1010) in terms of the AUC_{0-24′} but typically lower than to amitifadine (EB-1010) in terms of the Cₘₐₓ. Exposure to the metabolite relative to the parent drug typically increased with increasing dosage, but the AUC₀₋₂₄ was never more than about 3.5-fold higher for any individual animal. Respective exposures to parent drug and to metabolite were typically similar for each on Days 0 and 358, suggesting that neither accumulates in plasma. At equivalent dosages of EB-1010, exposure to either parent drug or metabolite was similar between the genders.

No deaths were attributed to amitifadine (EB-1010) treatment. No treatment-related effects were observed in any of the clinical pathology parameters assessed or on organ weights, nor were there any treatment-related ECG, ophthalmic, macroscopic, or microscopic findings in this study. Clinical findings consisted of thinness in males from the 20 (15) mg/kg/day group, emesis, abnormal excreta, and dilated pupils in animals of both sexes from the 20 (15) mg/kg/day group. Abnormal excreta and dilated pupils also occurred in females from the 6 mg/kg/day group. Dilated pupils and emesis were considered extended pharmacologic effects of EB-1010. None of these observations persisted into the recovery period.

Dose-related lower mean body weights occurred in the 6 and 20 (15) mg/kg/day groups throughout the treatment period (Weeks 1-52). By Week 52, male weights were 5% and 15% lower and female weights were 12% and 14% lower in the 6 and 20 (15) mg/kg/day groups, respectively, than in the control group. Although individual males in the 6 and 20 (15) mg/kg/day groups and individual females in the 20 (15) mg/kg/day group received supplemented feed and/or subcutaneous fluid support periodically throughout the study, mean food consumption was somewhat lower in these groups only during the first 12 weeks of the treatment period (during administration of the 20 mg/kg/day dose). Thereafter, the animals from the 20 (15) mg/kg/day group were found to have generally similar or higher mean food consumption values than those in the control group. Three dogs of each gender given placebo or 20 (15) mg/kg/day were given a 4 week post-treatment recovery period. During the 4-week post-treatment (recovery) period, mean weight gains and food consumption for animals in the 20 (15) mg/kg/day group were generally similar to or slightly higher than the control values indicating partial to substantial body weight recovery after treatment with amitifadine (EB-1010) was terminated.

In summary, dose-related clinical signs, including emesis and dilated pupils, as well as effects on mean body weight parameters and individual food consumption patterns occurred in dogs receiving 6 and/or 20 (15) mg EB-1010/kg/day. The clinical findings of emesis and dilated pupils were considered to be extended pharmacologic effects of EB-1010. Inappetence and body weight loss in 2 males receiving 20 mg/kg/day resulted in lowering this dosage to 15 mg/kg/day on Day 84. No other treatment-related effects occurred in any of the additional parameters assessed in this study. During the 4-week post-treatment (recovery) period, no relevant clinical signs were noted and slight to substantial recovery in body weight gains occurred in animals from the 20 (15) mg/kg/day group. The NOAEL for oral (capsule) administration of amitifadine (EB-1010) to beagle dogs for 52 consecutive weeks was considered to be 6 mg/kg/day. Corresponding Day 358 AUC₀₋₂₄ values for males and females at the NOAEL of 6 mg/kg/day were 15,811 and 11,113 ng·h/mL, respectively, for amitifadine (EB-1010) (free base) and 22,501 and 18,455 ng·h/mL, respectively for the lactam metabolite 5-(3,4-dichloro-phenyl)-3-aza-bicyclo[3.1.0]hexan-2-one.

TABLE 13
Liver Metabolism Enzyme Changes (fold change vs controls)
in the 12-Month Dog Study

| | Study Week 52 | | Study Week 56 | |
|---|---|---|---|---|
| | Males | Females | Males | Females |
| 7-ethoxyresorufin O-dealkylation | 5.1 | 5.9* | 1.4 | 1.0 |
| 7-benzylocyresorufin O-dealkylation | 0.5 | 0.4 | 1.6 | 0.7 |

*Statistically significant vs. control value

Drug metabolic enzyme analysis suggested that amitifadine (EB-1010) was a reversible inducer of CYP1A (7-ethoxyresorufin O-dealkylation) and a reversible suppressor or inhibitor of CYP2B11 (7-benzyloxyresorufin O-dealkylation). As an inducer of CYP1A (7-ethoxyresoufin O-dealkylation) activity, amitifadine (EB-1010) was approximately ⅓ to ½ as effective as the prototypical CYP1A inducer, β-naphthoflavone. It is not clear why amitifadine (EB-1010) suppressed or inhibited 7-benzyloxyresorufin O-dealkylation but not testosterone 16α-hydroxylase activity, both of which are markers of CYP2B11 activity.

### Example XX

### 14-Day Mouse Study of EB-1010

Groups of Crl:CD-1(ICR) BR mice (Charles River, Wilmington, Mass.) received daily oral (gavage) administration of amitifadine (EB-1010) for 14 days at dose levels of 0, 10, 30, 100, or 150 mg/kg/day. Blood samples were collected on Days 0 and 13 from up to 3 satellite animals/gender/group pre-dose, and 1, 2, 4, 8, and 24 hours post-dose. For toxicology assessment ten mice of each gender per group were assessed. Clinical observations, body weight, and food consumption measures were noted periodically during the study. Clinical pathology evaluations (hematology, serum chemistry, and urinalysis) were conducted on all surviving animals at the scheduled necropsy. Complete necropsies were conducted on all animals, selected organs were weighed, and selected tissues were examined microscopically from all animals in the control, 100, and 150 mg/kg/day groups.

In the toxicokinetic assessment, 38 mice of each gender were given amitifadine (EB-1010) in the dosages described above. The toxicokinetic results for parent drug and the lactam metabolite 5-(3,4-dichloro-phenyl)-3-aza-bicyclo[3.1.0]hexan-2-one (EB-10101) are summarized in Table 14.

TABLE 14
Toxicokinetics of EB-1010 and EB-10101* in the 14-Day Mouse
Study

| | EB-1010 (free base) | | | | | |
|---|---|---|---|---|---|---|
| Gender/ | AUC₀₋₂₄ | | | | Tₘₐₓ (h) | |
| EB-1010 | (ng · h/mL) | | Cₘₐₓ (ng/mL) | | | Day |
| (mg/kg/day) | Day 0 | Day 13 | Day 0 | Day 13 | Day 0 | 13 |
| Males | | | | | | |
| 10 | 1974 | 1907 | 261 | 300 | 1 | 1 |
| 30 | 12780 | 7250 | 1059 | 1017 | 1 | 1 |
| 100 | 40306 | 22274 | 3898 | 3081 | 2 | 2 |
| 150 | 71098 | 40248 | 5609 | 4500 | 1 | 2 |
| Females | | | | | | |
| 10 | 2486 | 2086 | 323 | 258 | 2 | 1 |
| 30 | 9916 | 6474 | 1140 | 726 | 2 | 1 |
| 100 | 26436 | 20235 | 4247 | 2051 | 2 | 1 |
| 150 | 48325 | 23799 | 4938 | 3185 | 2 | 1 |
| | EB-10101* | | | | | |
| Gender/ | AUC₀₋₂₄ | | | | Tₘₐₓ (h) | |
| EB-1010 | (ng · h/mL) | | Cₘₐₓ (ng/mL) | | | Day |
| (mg/kg/day) | Day 0 | Day 13 | Day 0 | Day 13 | Day 0 | 13 |
| Males | | | | | | |
| 10 | 5584 | 4269 | 583 | 401 | 2 | 1 |
| 30 | 40764 | 16269 | 2955 | 1415 | 8 | 1 |
| 100 | 135809 | 155532 | 10069 | 12292 | 4 | 8 |
| 150 | 236733 | 267444 | 17548 | 20656 | 8 | 8 |
| Females | | | | | | |
| 10 | 4883 | 3111 | 476 | 368 | 2 | 2 |
| 30 | 34323 | 13880 | 2571 | 1081 | 4 | 1 |
| 100 | 123552 | 111657 | 8491 | 9177 | 8 | 4 |
| 150 | 238332 | 232113 | 18010 | 17637 | 8 | 8 |

*Formerly identified as DOV 216,298

The exposures to both amitifadine (EB-1010) and 5-(3,4-dichloro-phenyl)-3-aza-bicyclo[3.1.0]hexan-2-one (EB-10101) appeared to be proportional to the dosage of amitifadine (EB-1010) over the range of 10 to 150 mg/kg/day, although exposures to the metabolite were higher than to parent drug. In terms of AUC₀₋₂₄ values, exposure to the metabolite relative to the parent drug increased from approximately 2- to 5-fold on Day 0 to as much as 10-fold higher on Day 13 at 150 mg/kg/day. Exposure to amitifadine (EB-1010) was typically lower on Day 13 than on Day 0, suggesting a change in the absorption or metabolism of amitifadine (EB-1010) upon repeated dosing. Exposure to 5-(3,4-dichloro-phenyl)-3-aza-bicyclo[3.1.0]hexan-2-one (EB-10101), however, was similar on Study Days 0 and 13, suggesting that the metabolite does not accumulate in the plasma. At equivalent dosages of EB-1010, relative exposure to parent drug versus metabolite was slightly higher for male than for female mice, increasing by approximately 50% to 70% as dosage increased to 150 mg/kg/day. Exposures to 5-(3,4-dichloro-phenyl)-3-aza-bicyclo[3.1.0]hexan-2-one (EB-10101) were typically similar between the genders.

EB-1010-related deaths were observed in males and females receiving 150 mg/kg/day. Two toxicology group males receiving 100 and 150 mg/kg/day, respectively, were found dead on Days 8 and 1, respectively. There were no macroscopic or microscopic findings for these animals from which the cause of death could be identified. In addition, 1 toxicokinetic group male and 1 toxicokinetic group female receiving 100 mg/kg/day and 7 toxicokinetic group males and 4 toxicokinetic group females receiving 150 mg/kg/day were found dead or euthanized in extremis between Days 1 and 10. The cause of death was determined to be esophageal perforation as a result of intubation difficulty for both animals receiving 100 mg/kg/day and 1 male receiving 150 mg/kg/day, but was undetermined for the remaining animals receiving 150 mg/kg/day. Clinical findings prior to euthanasia for the toxicokinetic group animals receiving 150 mg/kg/day included body and extremities cool to touch, shallow and labored respiration, gasping, twitching, tremors (continuous or intermittent), ptosis, prostration, and/or yellow material on various body surfaces. Macroscopic examinations were only conducted in an attempt to identify potential intubation error in the toxicokinetic group animals; microscopic examinations were not conducted.

There were no effects on food consumption or serum chemistry parameters related to treatment with EB-1010. There were no macroscopic findings or differences in the stage of estrous at necropsy (as determined by vaginal lavage) related to treatment with EB-1010. Clinical observations in the toxicology group males and females receiving 100 and 150 mg/kg/day consisted of yellow material on various body surfaces. These observations were noted primarily at the 1- and 3-hour post-dosing observation periods and were generally resolved by the time of dosing on the following day. Twitching was also observed in 3/10 males receiving 150 mg/kg/day on Day 0.

Transient body weight losses were noted in males receiving 30, 100, and 150 mg/kg/day and females receiving 150 mg/kg/day and lower body weight gains were noted in females receiving 100 mg/kg/day during Days 0 to 7. Correspondingly larger body weight gains were noted in these groups during Days 7 to 14 when compared to the control group values. Overall there was a slight body weight decrease in males receiving 100 and 150 mg/kg/day at the end of the dosing period.

A slight (not statistically significant) dose-related trend in lower white blood cell (WBC) counts was observed in males and females receiving EB-1010. This was accompanied by higher percent neutrophil counts (183%) and lower absolute and percentage lymphocyte counts (54% and 31%, respectively) in males receiving 150 mg/kg/day. The observed changes in these parameters in the context of the observed body weight loss, higher adrenal gland weights, and clinical observations in this group were consistent with a stress response and were not considered target organ toxicity. Additionally, absolute and percent reticulocyte counts in males receiving 150 mg/kg/day and females receiving 100 or 150 mg/kg/day were 29% to 53% higher compared to the respective control group. Increased reticulocytes occurred in conjunction with microscopic observations of minimal to mild erythroid hyperplasia in the spleen (discussed below), but as there were no differences in red blood cell (RBC) counts or microscopic observations of hemosiderin deposition indicative of RBC consumption, the toxicologic relevance of this finding is uncertain.

Higher liver weights were noted in males receiving 150 mg/kg/day and females receiving 30, 100, or 150 mg/kg/day. Absolute liver weights were 22% higher in males and 14% to 28% higher in females; relative (to final body weight) liver weights were 26% higher in males and 11% to 26% higher in females; and relative (to brain weight) liver weights were 25% higher in males and 15% to 31% higher in females. Increased mean liver weights and centrilobular hepatocellular hypertrophy present in both males and females are consistent with hepatic enzyme induction and are considered to be an adaptive response and not adverse. Higher adrenal gland weights were noted in males receiving 100 or 150 mg/kg/day. Absolute adrenal gland weights were 46% to 50% higher; relative (to final body weight) adrenal gland weights were 43% to 50% higher; and relative (to brain weight) adrenal gland weights were 50% higher in males receiving 100 or 150 mg/kg/day. Higher adrenal weights noted in males in the context of the observed WBC and body weight changes, as well as clinical observations, likely reflect a stress response.

Histologic changes were also noted in the spleen (erythroid hyperplasia) of males receiving 100 or 150 mg/kg/day and females receiving 30, 100, or 150 mg/kg/day and epididymides (cellular luminal debris) of males receiving 150 mg/kg/day.

Splenic erythroid hyperplasia suggests a regenerative response, although there was no evidence of anemia or hemorrhage. The toxicologic relevance of luminal cellular debris in the epididymides at 150 mg/kg/day is uncertain in the absence of testicular findings, since primary epididymal lesions are believed to be relatively rare in mouse toxicity studies.

In summary, systemic toxicity of amitifadine (EB-1010) administered orally to Crl:CD1(ICR) mice for 14 days occurred at a dose level of 150 mg/kg/day. Mortality, clinical signs, slight body weight losses, adaptive hepatic effects, increased reticulocyte counts, erythroid hyperplasia, and a series of indicators attributed to generalized stress occurred at this dose level. At a dose level of 100 mg/kg/day, clinical signs, transient body weight effects, adaptive liver effects, and slight indicators attributed to generalized stress occurred. Only minimal body weight and/or adaptive hepatic effects were noted at dose levels of 10 and 30 mg/kg/day. In the absence of a high mortality rate or definitive adverse EB-1010-related target organ toxicity, the NOAEL for oral (gavage) administration of amitifadine (EB-1010) to Crl:CD1(ICR) mice for 14 consecutive days was considered to be 100 mg/kg/day. This level was considered appropriate for use as a high dose in a subsequent repeated-dose toxicity study. Corresponding Day 13 AUC₀₋₂₄ values for males and females at the NOAEL of 100 mg/kg/day were 22,274 and 20,235 ng·h/mL, respectively, for amitifadine (EB-1010) (free base) and 155,532 and 111,657 ng·h/mL, respectively, for the lactam metabolite, 5-(3,4-dichloro-phenyl)-3-aza-bicyclo[3.1.0]hexan-2-one (EB-10101).

### Example XXI

### 13-Week Mouse Study of EB-1010

Groups of Crl:CD-1(ICR) BR mice (Charles River, Wilmington, Mass.) received daily oral (gavage) administration of amitifadine (EB-1010) for 13 weeks at dose levels of 0, 10, 30, or 100 mg/kg/day. Blood samples were collected on Days 0 and 84 from up to 3 animals/gender/group pre-dose, and 1, 2, 4, 8 and 24 hours. For toxicology assessment ten mice of each gender were placed in each group. Clinical observations, body weight, and food consumption measures were noted periodically during the study.

For the toxicokinetic assessment, thirty-eight mice of each gender were given the dosages of amitifadine (EB-1010) noted above. The toxicokinetic results for parent drug and the lactam metabolite, 5-(3,4-dichloro-phenyl)-3-aza-bicyclo[3.1.0]hexan-2-one (EB-10101) are summarized in Table 15. Clinical pathology evaluations (hematology, serum chemistry, and urinalysis) were conducted on all surviving animals at the scheduled necropsy. Ophthalmic examinations were performed during Weeks −1 and 12. Complete necropsies were conducted on all animals, selected organs were weighed, and selected tissues were examined microscopically from all animals in the control and 100 mg/kg/day groups; liver, ovaries, and vagina were examined microscopically from animals in the 10 and 30 mg/kg/day groups.

TABLE 15
Toxicokinetics of EB-1010 and EB-10101* in the 13-Week Mouse
Study

| | EB-1010 (free base) | | | | | |
|---|---|---|---|---|---|---|
| Gender/ | AUC₀₋₂₄ | | | | Tₘₐₓ (h) | |
| EB-1010 | (ng · h/mL) | | Cₘₐₓ (ng/mL) | | | Day |
| (mg/kg/day) | Day 0 | Day 84 | Day 0 | Day 84 | Day 0 | 84 |
| Males | | | | | | |
| 10 | 2052 | 2141 | 288 | 350 | 1 | 1 |
| 30 | 6659 | 7289 | 704 | 863 | 1 | 2 |
| 100 | 28952 | 24068 | 2846 | 2559 | 1 | 1 |
| Females | | | | | | |
| 10 | 2540 | 1980 | 296 | 229 | 1 | 2 |
| 30 | 7629 | 7115 | 972 | 825 | 2 | 2 |
| 100 | 17431 | 19995 | 2449 | 2081 | 1 | 1 |
| | EB-10101* | | | | | |
| Gender/ | AUC₀₋₂₄ | | | | Tₘₐₓ (h) | |
| EB-1010 | (ng · h/mL) | | Cₘₐₓ (ng/mL) | | | Day |
| (mg/kg/day) | Day 0 | Day 84 | Day 0 | Day 84 | Day 0 | 84 |
| Males | | | | | | |
| 10 | 8064 | 8006 | 872 | 1099 | 2 | 1 |
| 30 | 24279 | 30039 | 1632 | 2186 | 2 | 4 |
| 100 | 146423 | 117819 | 11131 | 9212 | 8 | 4 |
| Females | | | | | | |
| 10 | 9390 | 6979 | 765 | 960 | 4 | 2 |
| 30 | 27953 | 36395 | 1958 | 3104 | 2 | 4 |
| 100 | 93984 | 144151 | 8257 | 13046 | 4 | 4 |

*Formerly identified as DOV 216,296

The exposures to both amitifadine (EB-1010) and 5-(3,4-dichloro-phenyl)-3-aza-bicyclo[3.1.0]hexan-2-one (EB-10101) appeared to be proportional to the dosage of amitifadine (EB-1010) over the range of 10 to 100 mg/kg/day; exposures to metabolite were higher than to parent drug. Exposure to amitifadine (EB-1010) was similar on Days 0 and 84, although variable, suggesting that neither amitifadine (EB-1010) nor 5-(3,4-dichloro-phenyl)-3-aza-bicyclo[3.1.0]hexan-2-one (EB-10101) accumulates in the plasma upon repeated oral dosing with EB-1010. Exposures to 5-(3,4-dichloro-phenyl)-3-aza-bicyclo[3.1.0]hexan-2-one (EB-10101) were approximately 2- to 5-fold higher than the exposures to amitifadine (EB-1010) on Day 0, and this relationship remained relatively constant for male mice on Day 84. However, following repeated dosing, exposure to the metabolite relative to the parent drug was as much as 7-fold higher for female mice at the 100 mg/kg dose.

There were no amitifadine (EB-1010)-related deaths, ophthalmic, hematology, or macroscopic findings in this study. Treatment-related post-dosing clinical signs consisted of yellow material on various body surfaces (urogenital area, trunk, neck, and/or mouth) in the males and females from the 100 mg/kg/day group. These signs were noted sporadically throughout the treatment period and were more commonly observed in the males.

Treatment-related, transient body weight loss and slightly lower food consumption were noted for the males and females from the 100 mg/kg/day group during week 0 to 1. These losses were more pronounced in males than females. Throughout the remainder of the treatment period, mean body weight gains and food consumption in animals from the 100 mg/kg/day group were generally similar or slightly higher than the control group values.

The only treatment-related serum chemistry alteration obtained was lower total cholesterol levels in males from all treated groups and in females from the 100 mg/kg/day group at the end of the treatment period. Treatment-related effects on organ weights consisted of higher absolute and relative liver weights in both sexes receiving 100 mg/kg/day (17% to 18% in males and 22% to 32% in females) and lower ovary/oviduct weights (23% to 28%) in females from the 100 mg/kg/day group. In addition, higher thyroid/parathyroid gland weights (36% to 38%, respectively) were noted in males administered 100 mg/kg/day and may have been directly or indirectly related to treatment with amitifadine (EB-1010) via potential compound-related hepatic microsomal enzyme induction and/or disruption of the hypothalamic-pituitary-thyroid axis. Since serum thyroid hormones were not evaluated in this study, and in the absence of histological changes, the toxicologic relevance of the higher thyroid/parathyroid gland weights in males is unclear.

Histologic changes attributed to treatment with amitifadine (EB-1010) occurred in the livers of both sexes and in the female reproductive tract. Centrilobular hepatocellular hypertrophy was seen in males from all dose groups and in females from the 30 and 100 mg/kg/day groups. These findings were accompanied by increased liver weights in both sexes only in animals from the 100 mg/kg/day group and were considered an adaptive, not an adverse, response to repeated treatment. In the ovary, anovulatory status, as evidenced by decreased numbers of corpora lutea and a lack of histologic evidence of recent ovulation, was noted in 5/10 females from the 100 mg/kg/day group. This finding was consistent with the observed lower mean ovary/oviduct weights.

In summary, dose-related systemic effects of amitifadine (EB-1010) were noted following repeated-dose oral administration to Crl:CD-1 (ICR) mice (Charles River, Wilmington, Mass.) for 13 weeks. Clinical signs were observed at dosage levels of 10 (males only), 30, and 100 mg/kg/day throughout the study, while body weight losses and lower mean food consumption were noted at 100 mg/kg/day only during the first week of amitifadine (EB-1010) administration. Centrilobular hepatocellular hypertrophy occurred in all treated males and in females receiving ≧30 mg/kg/day, and was accompanied by lower cholesterol levels, higher absolute and relative liver weights (at 100 mg/kg/day), and higher thyroid/parathyroid gland weights in males receiving 100 mg/kg/day. Centrilobular hepatocellular hypertrophy and increased liver weights were considered an adaptive response to treatment and not adverse findings. Although higher thyroid/parathyroid weights in the absence of any histologic changes occurred in males from the 100 mg/kg/day group, the toxicologic relevance of this change is unclear. Lower mean ovary/oviduct weights in conjunction with anovulatory status, evidenced by decreased numbers of corpora lutea and lack of histologic evidence of recent ovulation, occurred in females from the 100 mg/kg/day group. While considered to be adverse, the ovarian changes are not considered dose-limiting findings. Therefore, the MTD for oral administration of amitifadine (EB-1010) to Crl:CD-1 (ICR) mice (Charles River, Wilmington, Mass.) for 13 weeks was considered to be 100 mg/kg/day, and the NOAEL was considered to be 30 mg/kg/day. Corresponding Day 84 AUC₀₋₂₄ values for males and females receiving 100 mg/kg/day were 24,068 and 19,995 ng·h/mL, respectively, for amitifadine (EB-1010) and 117,819 and 144,151 ng·h/mL, respectively, for the lactam metabolite, 5-(3,4-dichloro-phenyl)-3-aza-bicyclo[3.1.0]hexan-2-one (EB-10101); corresponding Day 84 AUC₀₋₂₄ values for males and females receiving 30 mg/kg/day were 7,289 and 7,115 ng·h/mL, respectively, for amitifadine (EB-1010) and 30,039 and 36,395 ng·h/mL, respectively, for the lactam metabolite, 5-(3,4-dichloro-phenyl)-3-aza-bicyclo[3.1.0]hexan-2-one (EB-10101).

### Example XXII

### Rat Pilot Developmental Toxicity Study of EB-1010

Amitifadine (EB-1010) was administered orally on GDs 6 through 17 at daily doses of 0, 5, 12.5, 25, or 50 mg/kg/day to mated female Crl:CD®(SD)IGS BR rats (Charles River, Wilmington, Mass.) (5 per group). Clinical observations, body weights, and food consumption were recorded periodically. On GD 20, a laparohysterectomy was performed on each surviving female. The uteri, placentas, and ovaries were examined; gravid uterine weights, the numbers of fetuses, early and late resorptions, total implantations, and corpora lutea were recorded. The fetuses were weighed, sexed, and examined for external malformations and developmental variations.

Due to neuromotor and respiratory signs as well as soft stools and/or decreased defecation and urogenital staining, body weight losses, and loss of appetite, all females in the 50 mg/kg/day group were euthanized in extremis on GD 8, 9, or 10, precluding any assessment of reproductive outcome. All other animals survived to the scheduled laparohysterectomy. Body weight losses or reduced body weight gains, accompanied by decreased food consumption, occurred from GD 6 to 9 in females at doses ≧12.5 mg/kg/day. Soft stools and/or decreased defecation were noted occasionally in all amitifadine (EB-1010) dose groups, and occurred for all females in the 50 mg/kg/day group. A statistically significant reduction in mean fetal weight occurred at 25 mg/kg/day. No other differences were noted in intrauterine parameters that could be attributed to maternal treatment. Based on the results of this study, dosage levels of 3, 10, and 20 mg/kg/day were selected for the definitive embryo/fetal development study of amitifadine (EB-1010) in rats.

### Example XXIII

### Rat Developmental Toxicity Study of EB-1010

Groups of mated Crl:CD®(SD)IGS BR rats (Charles River, Wilmington, Mass.) received daily oral (gavage) administration of amitifadine (EB-1010) at doses of 0, 3, 10, or 20 mg/kg on gestation days (GD) 6 through 17. For the toxicology assessment, there were twenty-five mated females/group. Clinical observations, body weights, and food consumption were recorded periodically. Blood samples were collected from up to 4 pregnant dams/group following the first and last dose pre-dose and 1, 2, 4, 8 and 24 hours.

For the toxicokinetic assessment, eight mated females per group were given the doses of amitifadine (EB-1010) listed above. The toxicokinetic results are summarized in Table 16. Females that delivered early were necropsied on the day of delivery. On GD 20, a laparohysterectomy was performed on each surviving female. The uteri, placentas, and ovaries were examined; gravid uterine weights, the numbers of fetuses, early and late resorptions, total implantations, and corpora lutea were recorded. The fetuses were weighed, sexed, and examined for external, visceral, and skeletal malformations and developmental variations.

TABLE 16
Toxicokinetics of EB-1010 in the Pregnant Rat

| | AUC₀₋₂₄ | | Cₘₐₓ | | Tₘₐₓ | |
|---|---|---|---|---|---|---|
| EB-1010 | (ng · h/mL) | | (ng/mL) | | (h) | |
| (mg/kg/day) | GD 6 | GD 17 | GD 6 | GD 17 | GD 6 | GD 17 |
| 3 | 2568 | 2623 | 387 | 397 | 2 | 2 |
| 10 | 10898 | 11244 | 1393 | 1304 | 2 | 2 |
| 20 | 19467 | 19709 | 1968 | 1889 | 2 | 2 |

The exposures to amitifadine (EB-1010) increased in a generally dose-proportional manner over the dose range of 3 to 20 mg/kg/day and were similar on GD 6 and GD 17. Additionally, repeated-dose exposure values (GD 17) in the 10 and 20 mg/kg groups were similar to those values obtained after 14 daily doses to nonpregnant female rats in the 2-week study.

Three females in the 20 mg/kg/day group delivered litters prior to GD 20 laparohysterectomy due to errors in detection of mating. All other animals survived to the GD 20 examination. Treatment-related clinical findings (hair loss) occurred at 20 mg/kg/day. Dose-related initial body weight losses and decreases in maternal body weight gains and food consumption occurred at 10 and 20 mg/kg/day. Increases in food consumption during the later phase of the treatment period likely were related to the pharmacology of amitifadine (EB-1010) and were not considered toxicologically meaningful. No maternal effects were observed at 3 mg/kg/day. Decreased fetal body weights accompanied by occurrences of variations indicative of developmental delay (e.g., increased litter proportions of unossified sternebra[e], cervical centrum no. 1, and renal papilla[e], or undeveloped and/or distended ureters) were observed at 20 mg/kg/day. All other intrauterine parameters were similar to control values. No indications of teratogenesis, selective developmental toxicity, or maternal reproductive disruption were noted in this study. Therefore, 3 mg/kg/day was considered to be the NOEL for maternal toxicity and 10 mg/kg/day was considered the NOEL for developmental toxicity. The corresponding maternal GD 17 AUC₀₋₂₄ value at the 10 mg/kg/day group was 11,244 ng·h/mL.

### Example XXIV

### Rabbit Pilot Developmental Toxicity Study of EB-1010

Amitifadine (EB-1010) was administered orally on GDs 7 through 20 at daily doses of 0, 3, 10, 20, 25, or 30 mg/kg/day to inseminated female rabbits (6 per group). Clinical observations, body weights, and food consumption were recorded periodically. On GD 29, a laparohysterectomy was performed on each surviving female. The uteri, placentas, and ovaries were examined, and gravid uterine weights, the numbers of fetuses, early and late resorptions, total implantations, and corpora lutea were recorded. The fetuses were weighed, sexed, and examined for external malformations and developmental variations.

Because of inappetence for 4 consecutive days, all females in the 30 mg/kg/day group were euthanized on GD 14. All other animals survived to the scheduled laparohysterectomy. Females receiving doses ≧20 mg/kg/day exhibited body weight losses or reduced body weight gains accompanied by decreased food consumption and defecation throughout the treatment period. Soft stool was also noted for rabbits in the 20 and 25 mg/kg/day groups. Because rabbits in the 30 mg/kg/day group were euthanized prior to the scheduled laparohysterectomy, no evaluation of treatment-related developmental effects could be performed. At 25 mg/kg/day, slight increases in the mean litter proportions of early resorptions and post-implantation loss were noted as a result of increases in these parameters in 2 of 6 doses in this group. These effects were not statistically significantly different from the control group but were outside the range of the WIL historical control data for definitive studies. Other maternal reproductive parameters and all fetal measurements were similar to the control group values. Based on the results of this study, dose levels of 0, 3, 10, and 20 mg/kg/day were selected for a definitive embryo/fetal development study of amitifadine (EB-1010) administered orally by gavage in rabbits.

### Example XXV

### Rabbit Developmental Toxicity Study of EB-1010

Groups of pregnant New Zealand white rabbits received daily oral (gavage) administration of amitifadine (EB-1010) at doses of 0, 3, 10, or 20 mg/kg on GD (gestational day) 7 through 20 to inseminated female rabbits in 2 replicates, due to <20 pregnancies in all groups in the initial, standard study. Blood samples were collected from up to 4 pregnant does/group following the last dose administered on GD20 were taken pre-dose, and 1, 2, 4, 8, and 24 hours. For the toxicology assessment, 27-33 per rabbits per group were dosed. Clinical observations, body weights, and food consumption were recorded periodically. For the toxicokinetic study, 4 inseminated rabbits per group were given the dosages of amitifadine (EB-1010) listed above. The toxicokinetic results are summarized in Table 17.

TABLE 17
Toxicokinetics of EB-1010 in the Pregnant Rabbit

| | AUC₀₋₂₄ | Cₘₐₓ | Tₘₐₓ |
|---|---|---|---|
| EB-1010 | (ng · h/mL) | (ng/mL) | (h) |
| (mg/kg/day) | GD 20 | GD 20 | GD 20 |
| 3 | 1324 | 309 | 1 |
| 10 | 5859 | 1092 | 1 |
| 20 | 19158 | 3197 | 1 |

The repeated-dose exposures to amitifadine (EB-1010) determined on the final day of treatment increased in a generally dose-proportional manner over the dose range of 3 to 20 mg/kg/day.

On GD 29, a laparohysterectomy was performed on each surviving female. The uteri, placentas, and ovaries were examined, and gravid uterine weights, the numbers of fetuses, early and late resorptions, total implantations, and corpora lutea were recorded. The fetuses were weighed, sexed, and examined for external malformations and developmental variations.

One female each in the control and 3 mg/kg/day groups died during the study, and 1 female in the 20 mg/kg/day group aborted; no relationship to amitifadine (EB-1010) administration was evident. There were no test article-related maternal clinical observations or effects on mean body weights or net body weights at any dose level. Body weight losses or reduced weight gains and decreased food consumption occurred during the treatment period at 20 mg/kg/day. Mean gravid uterine weights, numbers, and litter proportions of viable fetuses and post-implantation losses, as well as mean fetal sex ratios and fetal weights were unaffected by treatment. A slight increase in 27 pre-sacral vertebrae occurred at 20 mg/kg/day. This is a frequently occurring developmental variation in rabbit fetuses and, in the absence of any other fetal effects, was not considered an adverse finding. No indications of treatment-related malformations, other developmental variations, or fetal effects were observed. Therefore, 10 mg/kg/day was considered the NOAEL for maternal toxicity, and 20 mg/kg/day was considered the NOAEL for developmental toxicity. The corresponding maternal GD 20 AUC₀₋₂₄ value, at 20 mg/kg/day, was 19,158 ng·h/mL.

### Example XXVI

### Rat CNS Study of EB-1010

Amitifadine (EB-1010) was administered orally by gavage as a single dose on Day 0 to Crl:CD® (SD)IGS BR rats at doses of 0, 10, 30, or 100 mg/kg. There were eight animals of each gender per group. Functional observational battery (FOB) assessments and automated 1-hour locomotor activity data were recorded for all animals 7 days prior to dose administration (pre-test), and again beginning 1 hour following dosing on Day 0. Following completion of the FOB and locomotor activity evaluations, all animals were euthanized and discarded.

There were no deaths or test article-related effects on home cage observations, handling observations, neuromuscular observations, or sensory observations noted. In the 30 mg/kg group females and in the 100 mg/kg group males and females, mean rearing counts in the 2-minute open field test were significantly lower (67.1%, 56.5%, and 78.6%, respectively) than in respective control groups. Hunched body posture was observed in ⅜ animals of each gender in the 100 mg/kg group during the open-field assessment, and in ⅜ females in the 100 mg/kg group were noted as having low arousal. No other treatment-related changes were observed in rotarod performance, time-to-first-step, gait, or mobility scores in the FOB. Mean urination values in the 10, 30, and 100 mg/kg group males were significantly higher (approximately 3-, 3-, and 5-fold higher, respectively) than in the control group. This might represent a pharmacologic effect of the test article. In an automated measure of spontaneous locomotor activity that followed the FOB evaluation, less habituation was noted in the treated than in the control groups, but there were no significant test article-related effects on cumulative mean total or cumulative mean ambulatory motor activity counts during the 1-hour assessment. Therefore, the NOAEL for CNS toxicity following a single oral (gavage) administration of amitifadine (EB-1010) to rats was 100 mg/kg.

### Example XXVII

### Rat Pulmonary Function Study of EB-1010

Amitifadine (EB-1010) was administered orally by gavage as a single dose on Day 0 to male Crl:CD® (SD)IGS BR rats (Charles River, Wilmington, Mass.) at doses of 0, 10, 30, or 100 mg/kg. There were eight male rats per group. Clinical examinations were performed daily, and detailed physical examinations were performed prior to dosing and again following pulmonary functional assessments. Four replicates of 2 animals from each treatment group were evaluated over a 2-week period. Following dose administration, the rats were placed in a head-out neck-sealed plethysmograph for measurement of pulmonary function parameters (respiratory rate, tidal volume, and calculated minute volume). Pulmonary function data were collected for at least 60 minutes prior to dosing (Day −1) and for at least 6 hours post-dosing on Day 0.

All animals survived to scheduled termination. Upon removal from the plethysmograph (6 hours post-dosing), test article-related clinical observations were noted in ⅜ animals in the 30 mg/kg group and 6/8 eight animals in the 100 mg/kg group. These CNS and respiratory observations included high stepping of hind limbs, hunched appearance, hypoactivity, hypothermia, increased shallow respiration rate, Straub tail, and/or walking on tiptoes. The more extensive CNS signs observed in this study compared to the CNS study described above were attributed to several differences in testing conditions between the 2 studies. There were statistically significant and dose-dependent increases in respiratory frequency accompanied by inversely proportional decreases in tidal volume following the 10-, 30, and 100 mg/kg doses of EB-1010. Therefore, the minute volume remained relatively unchanged throughout the assessment period. Increased respiratory frequency was observed as early as 16 to 30 minutes post-dosing (23%) in the 100 mg/kg group and remained elevated for the duration of the 6-hour assessment. At later post-treatment intervals, an elevated respiratory frequency of ≧33% was observed (with no evidence of recovery) in the 100 mg/kg group. In contrast, neither this degree nor duration of increased respiratory frequency was observed in the 10 or 30 mg/kg group. Therefore, the NOAEL for pulmonary function following single oral (gavage) administration of amitifadine (EB-1010) to rats was 30 mg/kg.

### Example XXVIII

### Dog Oral Cardiovascular Study of Amitifadine (EB-1010)

Amitifadine (EB-1010) was administered orally by capsule as a single dose on Day 0 to male and female beagle dogs at doses of 0 (empty capsule), 1, 3, or 10 mg/kg. There were four male and four female dogs per group. Surgical implantation of radiotelemetry devices occurred under anesthesia approximately 1 week prior to study initiation. The test article was administered according to a Latin square design such that each radiotelemetry-implanted dog received each treatment once, with a 7-day washout period between doses. Clinical examinations and detailed physical examinations were performed, and individual body weights were recorded on dosing days. Heart rate (derived from arterial waveforms), arterial blood pressure (systolic, diastolic, and calculated mean), body temperature, and ECG waveform intervals (PR, QRS, RR, QT, and QTcV) were collected for a 30-second period every 10 minutes for approximately 24 hours following dosing.

All animals survived to scheduled termination. There were no obvious treatment-related clinical observations, or physiologically meaningful and statistically relevant changes in blood pressure (systolic, diastolic, or calculated mean), heart rate, body temperature, or PR, QRS, RR, or QT/QTcV intervals indicative of a test article-related effect when compared to values obtained following administration of empty capsules. Although there was a slight, but significant, increase in the absolute QT interval at 10 mg/kg, there was <2% change when this value was corrected for heart rate (QTcV interval). Therefore, the NOEL for oral (capsule) administration of amitifadine (EB-1010) in dogs was 10 mg/kg.

### Example XXIX

### Anesthetized Dog Intravenous Cardiovascular Study of Amitifadine (EB-1010)

This study was performed in 4 sodium pentobarbital-anesthetized, ventilated, vagotomized, and conditioned mixed-breed dogs to assess cardiovascular effects of amitifadine (EB-1010) following rising IV femoral infusions. Doses of 2, 7, and 20 mg/kg in 10 mL deionized water were infused over 3 consecutive 30-minute periods using a programmable syringe infusion pump yielding cumulative doses of 2, 9, and 29 mg/kg. A separate group of 4 dogs was infused with vehicle only (30-60 mL over 90 minutes) for comparison. Mean arterial pressure (MAP), heart rate (HR), femoral arterial blood flow (BF), and ECG activity were recorded prior to (baseline) and 5, 10, 15, 20, and 29 minutes after the start of each 30-minute IV infusion of amitifadine (EB-1010) via femoral vein. Whole blood samples were collected for determination of peak plasma concentrations (see Section 5.3.3) following infusion of each dose.

Dose-related increases in MAP at the 2, 9, and 29 mg/kg cumulative doses were +10 (8%), +27 (22%), and 55 mm Hg (44%), respectively. A modest increase in HR (+10% change) was observed at the top dose. The increase in MAP was attributed to elevated arterial vascular resistance since a dose-dependent marked increase in femoral vascular resistance (+354% change) and a decrease in BF (−57% change) occurred in conjunction with the increases in MAP at the top dose. Little or no change was seen in the PR (−9%), QRS (+2%), or QTC (+2%) cardiac intervals of the ECG compared to baseline values at the top dose. Increased bleeding was noted at surgical incision sites in the dogs dosed with EB-1010, most likely a consequence of the elevated blood pressure. Dose-related hemolysis was apparent in derived plasma fractions. Peak amitifadine (EB-1010) plasma concentrations (mean±SD) measured during each 30-minute IV infusion were 35±16, 120±45, and 236±105 μM at cumulative doses of 2, 9, and 29 mg/kg, respectively.

### Example XXX

### Cloned hERG Channel Study of Amitifadine (EB-1010)

The objective of this study was to examine the in vitro effects of amitifadine (EB-1010) on the hERG channel current (Iₖᵣ, the rapidly activating, delayed rectifier cardiac potassium current).

Amitifadine (EB-1010) produced a concentration-dependent inhibition of the hERG channel current, ranging from 16.5% at 1 μM to 88.4% at 30 μM. The estimated IC₅₀ value was 4.6 μM (1,054 ng free base/mL) with a Hill coefficient of 1.1. The positive control (60 nM terfenadine) produced an 88% block of hERG current. Based on the hERG IC₅₀ value of EB-1010, the mean Cₘₐₓ value of amitifadine (EB-1010) measured in a human rising single dose study (1,420 ng/mL), and the free plasma concentration of the compound (1%), a provisional safety margin for amitifadine (EB-1010) was calculated to be 74.

### Example XXXI

### Single-Dose Rate Toxicology Study of Amitifadine (EB-1010)

Amitifadine (EB-1010) in 0.9% sodium chloride for injection, USP, was administered as a single bolus injection to Crl:CD®(SD)IGS BR rats (Charles River, Wilmington, Mass.) at doses of 0, 0.5, 1.5, and 5 mg/kg. Clinical observations, body weights, and food consumption were noted periodically. All animals were euthanized following a minimum 13-day non-dosing (recovery) period. Clinical pathology evaluations (hematology and serum chemistry) were performed on all rats at the time of necropsy and selected organs were weighed. No microscopic examinations were performed.

There were no treatment-related deaths or clinical signs. Body weights, food consumption, hematology parameters, macroscopic pathology, and organ weights were unaffected by administration of EB-1010. Higher mean ALP occurred only in males that received 5 mg/kg and was considered a treatment-related effect. Therefore, the NOAEL for a single IV administration of amitifadine (EB-1010) to male rats was 1.5 mg/kg; the NOAEL for female rats was 5 mg/kg.

### Example XXXII

### Male Rat Fertility Study of Amitifadine (EB-1010)

Amitifadine (EB-1010) was administered orally to male Crl:CD®(SD) IGS BR rats (24 per group) daily at 0, 4, 13, or 39 mg/kg/day for approximately 10 weeks prior to cohabitation, during cohabitation, and until the day prior to scheduled sacrifice (approximately 14 weeks total) to evaluate potential effects on mating and fertility. Physical signs, body weight, and food consumption were recorded periodically. Males were housed for up to 10 nights 1:1 with untreated females following approximately 10 weeks of treatment. Mating was confirmed by daily examination of females for the presence of a vaginal copulatory plug or sperm in a vaginal lavage. The day of observing a seminal plug and/or sperm was considered GD 0. Females were euthanized on GD 15, 16, or 17 and the uterus of each female rat was examined to determine pregnancy status. Corpora lutea per female were counted. Uterine implantations were counted, and each was classified as a live fetus, dead fetus, or a resorption. At termination, the left cauda epididymis from males were weighed, and frozen. Subsequently, these tissues were prepared and analyzed for sperm number. At necropsy, a sperm suspension from the mid-region of the left vas deferens of each male was prepared and evaluated for percent motile sperm (from ≧150 cells per male). Sections of both testes and the right epididymis from rats in the control and 39 mg/kg/day groups, and the epididymis from rats in the 13 mg/kg/day group were evaluated histologically.

There were no treatment-related deaths or physical signs during the study. All males in this study mated with untreated females. Treatment-related effects in the 39 mg/kg/day group included mean body weight loss (−24 g during the initial week of treatment) and decreased mean body weight gain (9%); decreased mean food consumption (−60%) only during the first week; decreased sperm count/g cauda epididymis (−44%); a decrease in percent motile sperm (−33%); slight decreases in the fecundity and fertility indices (−9%); and histologic changes in the epididymis. Slight to moderate vacuolation of the epididymal epithelium was noted only in the 39 mg/kg/day group. This change was characterized by epididymal cells with large, clear, cytoplasmic vacuoles and was accompanied by rare single cell necrosis of the epididymal epithelium. Treatment-related effects in the 13 mg/kg/day group included decreased mean body weight gain (7%); a decrease in sperm count/g cauda epididymis (−18%); and slight decreases in the fecundity and fertility indices (−9%); epididymal morphology was unaffected. There were no treatment-related effects on testicular weights or morphology. No treatment-related effects were noted on any parameters measured in males from the 4 mg/kg/day group. Based on these results, the NOEL for both general and reproductive toxicity parameters in male rats for amitifadine (EB-1010) was 4 mg/kg/day.

### Example XXXIII

### Female Rat Fertility Study of Amitifadine (EB-1010)

Amitifadine (EB-1010) was administered orally to female Crl:CD®(SD) rats (24 per group) at 0, 2.6, 9, or 34 mg/kg/day for 21 days prior to cohabitation, during cohabitation, and through GD 7. Physical signs, body weight, and food consumption were recorded periodically. Females were housed for up to 20 nights 1:1 with untreated males following 3 weeks of treatment. During cohabitation, vaginal lavages were performed daily. The presence of sperm in the lavage or a vaginal plug was considered positive evidence of mating. Following 12 nights of cohabitation, 3 apparently not bred females (2 in the control group and 1 female in the 34 mg/kg/day group) were re-housed with untreated proven breeder males. The day of confirmed mating was considered GD 0. Females with a confirmed mating were euthanized between GD 15 and 17. The uterus of each female was examined to determine pregnancy status. If there was no gross evidence of pregnancy, the uterus was stained with ammonium sulfide to visualize any early implantation sites. Corpora lutea per female were counted. Uterine implantations were counted, and each was classified as a live fetus, dead fetus, or a resorption. Fetuses were euthanized by rapid induction of hypothermia and discarded without further examination.

Treatment-related findings were limited to the 34 mg/kg/day group and consisted of body weight loss (−26 g) and decreased food consumption (−50%) during the first 4 days of dosing, and physical signs (red nasal discharge, unkempt appearance, urine-stained fur, and alopecia). All cohabited females showed signs of mating. No effects on fertility or on numbers of corpora lutea, implantations, live fetuses, or resorptions were attributed to treatment with EB-1010. Based on these results, the NOEL for effects of amitifadine (EB-1010) on general toxicity parameters was 9 mg/kg/day; for reproductive performance of female rats, the NOEL was ≧34 mg/kg/day.

### Example XXXIV

### Mammalian Erythrocyte Micronucleus Test of Amitifadine (EB-1010)

Amitifadine (EB-1010) was evaluated for its potential to increase the incidence of micronucleated polychromatic erythrocytes in bone marrow of male and female ICR mice following a single intraperitoneal injection. Based on mortality in 2 initial mouse-screening studies at doses of amitifadine (EB-1010) ranging from 1 to 2000 mg/kg, a maximum dose of 60 mg/kg was selected for the gene toxicity study. Male and female mice received amitifadine (EB-1010) at 15, 30, or 60 mg/kg, vehicle, or positive control (50 mg/kg cyclophosphamide monohydrate). Evaluations were conducted 24 hours later for all groups, as well as 48 hours later for the 60 mg/kg and vehicle groups. One female mouse in the 60 mg/kg group died, and post-dosing hyperactivity occurred at 60 mg/kg. All other mice survived treatment and appeared normal.

Bone marrow cells, including polychromatic erythrocytes (PCEs) and normochromatic erythrocytes (NCEs), collected 24 and 48 hours after treatment were examined microscopically for the presence of micronuclei. Reductions of 9% to 14% in the ratio of polychromatic erythrocytes to total erythrocytes were observed in the groups treated with EB-1010, suggesting that the compound did not inhibit erythropoiesis. No significant increases in micronucleated PCEs were observed in compound-treated groups at 24 or 48 hours after dose administration. The concurrent negative and positive control data were consistent with historical control values, indicating that there was no problem with the test system or the quality of the test. It was concluded that amitifadine (EB-1010) had no effects in the mouse micronucleus assay.

### Example XXXV

### Bacterial Reverse Mutation Assay of Amitifadine (EB-1010)

Amitifadine (EB-1010) was evaluated in the Ames/Salmonella plate incorporation assay to determine its ability to induce reverse mutations at selected histidine loci in 4 tester strains of Salmonella typhimurium and in an Escherichia coli tester strain in the presence and absence of an exogenous metabolic activation system (Aroclor-induced rat liver S9). Amitifadine (EB-1010) was evaluated in cultures at concentrations up to 5000 μg/plate. In the initial toxicity-mutation assay, toxicity was observed at doses of 600, 1800, and 5000 μg/plate and no positive mutagenic response was observed. In the confirmatory mutagenicity assay, toxicity was observed at 1500 and 5000 μg/plate and no positive mutagenic response was observed. Under the conditions of this study, it was concluded that amitifadine (EB-1010) was negative in the bacterial reverse mutation assay.

### Example XXXVI

### Mammalian Chromosome Aberration Test of Amitifadine (EB-1010)

Amitifadine (EB-1010) was evaluated in Chinese hamster ovary (CHO) cells to determine its ability to induce chromosome aberrations in the presence and absence of an exogenous metabolic activation system, Aroclor-induced S9. Cells were treated for 4 hours or 20 hours in the nonactivated system and for 4 hours in the S9-activated system. The results of the prescreen showed toxicity (i.e., at least 50% cell growth inhibition relative to the solvent control) at levels ≧50 μg/mL in both the nonactivated and the S9-activated 4-hour exposure groups and at levels ≧15 μg/mL in the nonactivated 20-hour continuous-exposure group. The concentrations used in the chromosome aberration assay ranged from 2.5 to 40 μg/mL for the nonactivated 4-hour exposure group, from 1.25 to 40 μg/mL for the S9-activated 4-hour exposure group, and from 0.625 to 25 μg/mL for the nonactivated 20-hour continuous exposure group.

In the nonactivated 4-hour and 20-hour exposure groups, no significant increase in the percentage of cells with structural or numerical aberrations above that of the solvent control was observed at any concentration of EB-1010. In the S9-activated 4-hour exposure group, the percentage of cells with structural aberrations was statistically increased at 40 μg/mL. However, the percentage of cells with structural aberrations at this level (3.5%) was within the range for the historical solvent control (0-6.5%) and was not considered to be biologically meaningful. There was no significant increase in the percentage of cells with numerical aberrations. Based upon the findings in this study, it was concluded that amitifadine (EB-1010) was negative for the induction of structural and numerical chromosome aberrations in CHO cells.

### Example XXXVII

### Assessment of NADPH Dependent Metabolism of Amitifadine

Amitifadine (EB-1010) (10 μM) was incubated in pooled male human liver microsome (HLM) and rat liver microsome (RLM) (XenoTech, LLC, Lenexa, Kans.) in the presence and absence of NADPH. The microsomal incubation mixtures (ca. 0.2 mL) were prepared in triplicate in 0.1 M potassium phosphate buffer (pH 7.4) containing 0.25 mg/mL microsomal protein (RLM and HLM), 1 mM EDTA, 4 mM MgCL₂ (Sigma-Aldrich, St. Louis, Mo.), and 10 μM amitifadine (EB-1010) in the presence and absence of NADPH spiking solution (final concentration of 1 mM). Incubation mixtures were pre-incubated aerobically in a shaking water bath at 37° C. for ca. 5 min. The reaction was initiated by the addition of NADPH (final concentration of 1 mM) or phosphate buffer. After 30 min. incubation, the reaction was stopped by mixing the incubation samples with two volumes of ice-cold quench solution (150 nM bicifadine-d5 prepared in acetonitrile (375 μL of bicifadine-d5 spiking solution (200 μM) to 500 mL of acetonitrile) by dilution of the internal standard spiking solution which was 200 μM bicifadine-d5 prepared in methanol by diluting the stock solution). In the positive controls, bicifadine was incubated in the microsomes instead of EB-1010. In the negative controls, amitifadine (EB-1010) and bicifadine were incubated with water instead of microsomal protein. The mixtures were kept at ca. −20° C. before analysis by LC-MS/MS.

The formation of 5-(3,4-dichloro-phenyl)-3-aza-bicyclo[3.1.0]hexan-2-one (EB-10101) was monitored using LC-MS/MS. Bicifadine (10 μM) was used as the positive control and the formation of the lactam metabolites of bicifadine (M-12) was monitored.

It was determined that the formation of 5-(3,4-dichloro-phenyl)-3-aza-bicyclo[3.1.0]hexan-2-one (EB-10101) from amitifadine (EB-1010) was partially NADPH-dependent. As shown in Table 18 and FIG. 10, the amount of 5-(3,4-dichloro-phenyl)-3-aza-bicyclo[3.1.0]hexan-2-one (EB-10101) formed in the HLM in the presence of NADPH was ˜2-fold higher than that in the absence of NADPH. In the RLM, 5-(3,4-dichloro-phenyl)-3-aza-bicyclo[3.1.0]hexan-2-one formed in the presence of NADPH was ˜5 fold higher than 5-(3,4-dichloro-phenyl)-3-aza-bicyclo[3.1.0]hexan-2-one formed in the absence of NADPH. The y-axis in FIG. 10 is the area ratio of analyte to the internal standard (bicifadine-d5 chromatograph).

TABLE 18
Formation of lactam metabolites in the liver microsome incubation
of EB-1010 and bicifadine^{a}

| | EB-1010 | | Bicifadine | |
|---|---|---|---|---|
| | | EB-10101 | | M-12 |
| | EB-10101 | isomer^{b} | M-12 | isomer^{c} |
| HLM with NADPH | 0.08 ± 0.01 | 2.02 ± 0.04 | 0.39 ± 0.02 | 0.06 ± 0.01 |
| HLM without NADPH | 0.04 ± 0.00 | ND | 0.35 ± 0.02 | ND |
| RLM with NADPH | 0.05 ± 0.01 | 6.86 ± 0.33 | 0.24 ± 0.03 | 0.57 ± 0.05 |
| RLM without NADPH | 0.01 ± 0.00 | ND | 0.10 ± 0.00 | ND |
| Negative Control^{d} | ND | ND | ND | ND |

^{a}EB-1010 and bicifadine (10 μM) were incubated with liver microsomes (0.25 mg/mL) for 30 min in the presence and absence of NADPH (1 mM). The lactam metabolites were monitored using LC-MS/MS method developed based on reference standards (EB-10101 and M-12). The numbers are mean ± standard deviation of the area under the curve (AUC) ratio of analyte to the internal standard (bicifadine-d5) chromatograph.
^{b}Isomers with identical MRM transitions with (EB-10101) but different retention times from the EB-10101 were detected in the liver microsome incubation systems.
^{c}Isomers with identical MRM transitions with (M-12) but different retention times from the M-12 were detected in the liver microsome incubation systems.
^{d}Negative controls were incubation of EB-1010 and bicifadine in the absence of liver microsomes
ND: not detected

The liver microsome incubation result of the positive control, bicifadine, is consistent with the previous study using radiolabeled bicifadine, indicating that the microsome incubation systems were properly prepared.

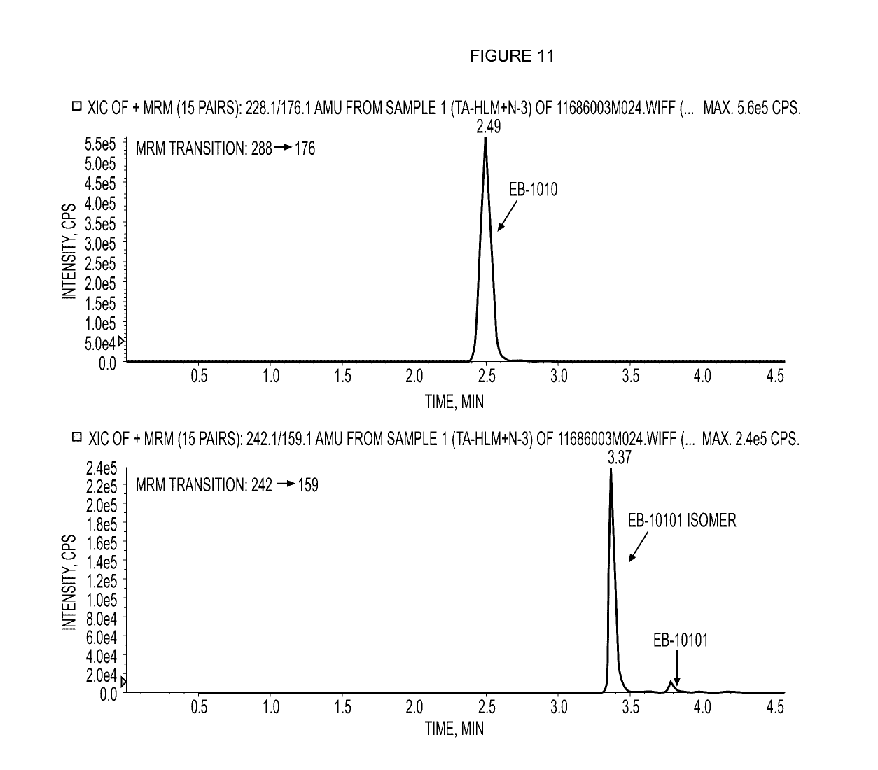

Additional metabolites of amitifadine (EB-1010) and bicifadine were observed in both HLM and RLM incubations in the presence of NADPH. In the LC-MS/MS chromatographs, the additional metabolites of amitifadine (EB-1010) and bicifadine and MRM transitions identical to those of EB-10101 and M-12, respectively, but appeared at different retention times in the chromatographs as seen in FIG. 11. The additional metabolite of amitifadine (EB-1010) was further analyzed using high-resolution mass spectrometer (Thermo LTQ Orbitrap XL, ThermoFisher Scientific, Waltham, Mass.), and was confirmed to be an isomer of 5-(3,4-dichloro-phenyl)-3-aza-bicyclo[3.1.0]hexan-2-one (EB-10101) and identified as (1S,5R)-5-(3,4-dichlorophenyl)-3-azabicyclo[3.1.0]hexan-4-one. The formation rates of the 5-(3,4-dichloro-phenyl)-3-aza-bicyclo[3.1.0]hexan-2-one (EB-10101) isomer appeared to be greater than those of 5-(3,4-dichloro-phenyl)-3-aza-bicyclo[3.1.0]hexan-2-one in both HLM and RLM systems (area ratios were ca. 25-fold and 140-fold, respectively) and as shown in Table 18 and FIG. 10, the formation of both 5-(3,4-dichloro-phenyl)-3-aza-bicyclo[3.1.0]hexan-2-one and M-12 isomers were NADPH dependent.

### Example XXXVIII

### Assessment of FMO Dependent Metabolism

Amitifadine (EB-1010) (10 μM) and bicifadine (10 μM) were incubated separately in pooled male human liver microsome (HLM) and rat liver microsome (RLM) (XenoTech, LLC, Lenexa, Kans.), which were pretreated at 37° C. and 45° C. for ca. 5 min, in the presence of NADPH. The microsomal incubation mixtures (ca. 0.2 mL) were prepared in triplicate in 0.1M potassium phosphate buffer (pH 7.4) containing 0.25 mg/mL microsomal protein (RLM and HLM), 1 mM EDTA, 4 mM MgCl₂, and 1 mM NADPH (Sigma-Aldrich, St. Louis, Mo.). Incubation mixtures were pre-incubated aerobically in a shaking water bath at 37° C. and 45° C. respectively for ca. 5 minutes. The reaction was initiated by the addition of NADPH (final concentration of 1 mM). After 30 min. incubation, the reaction was stopped by mixing the incubation samples with two volumes ice-cold quench solution (150 nM bicifadine-d5 prepared in acetonitrile by dilution of the internal standard spiking solution which was 200 μM bicifadine-d5 prepared in methanol by diluting the stock solution). For the positive controls, benzydamine (Sigma-Aldrich, St. Louis, Mo.) (final concentration 200 μM) was used in the incubation mixture. For the negative controls, water was used in the incubation mixtures instead of microsome protein. The mixtures were kept at ca. −20° C. before analysis by LC-MS/MS.

TABLE 19
Effect of pre-incubation temperature on the formation of metabolites of
EB-1010, bicifadine, and benzydamine in the liver microsome incubation^{a}

| | | EB-1010 | | Bicifadine | | Benzydamine |
|---|---|---|---|---|---|---|
| | Pre-treatment | | EB-10101 | | M-12 | Benzydamine |
| | temperature | EB-10101 | isomer^{b} | M-12 | isomer^{c} | N-oxide |
| HLM | 37° C. | 0.048 ± 0.007 | 1.69 ± 0.16 | 0.50 ± 0.02 | 0.07 ± 0.01 | 35.70 ± 0.86 |
| | 45° C. | 0.055 ± 0.002 | 2.02 ± 0.07 | 0.51 ± 0.01 | 0.07 ± 0.00 | 7.68 ± 1.51 |
| RLM | 37° C. | 0.021 ± 0.001 | 3.89 ± 0.36 | 0.21 ± 0.01 | 0.55 ± 0.04 | 75.35 ± 0.75 |
| | 45° C. | 0.023 ± 0.001 | 0.53 ± 0.05 | 0.20 ± 0.01 | 0.12 ± 0.01 | 15.86 ± 1.31 |
| NC^{d} | 37° C. | ND | ND | ND | ND | 1.40 ± 0.03 |
| | 45° C. | ND | ND | ND | ND | 1.46 ± 0.05 |

^{a}EB-1010, bicifadine (10 μM) and benzydamine (200 μM, positive control) were incubated with liver microsomes (0.25 mg/mL) at ca. 37° C. or 45° C. for 5 min before the addition of NADPH (1 mM). The incubation mixtures were further incubated at ca. 37° C for 30 min. The EB-10101, M-12 and benzydamine N-oxide were monitored using LC-MS/MS method developed based on reference standards. The numbers are mean ± standard deviation of the area under the curve (AUC) ratio of analyte to the internal standard (bicifadine-d5) chromatograph.
^{b}Isomers with identical MRM transitions with (EB-10101) but different retention times from the EB-10101 were detected in the liver microsome incubation systems.
^{c}Isomers with identical MRM transitions with (M-12) but different retention times from the M-12 were detected in the liver microsome incubation systems.
^{d}Negative controls were incubation of EB-1010, bicifadine, and benzydamine in the absence of liver microsomes
ND: not detected

The formation of 5-(3,4-dichloro-phenyl)-3-aza-bicyclo[3.1.0]hexan-2-one (EB-10101) and M-12 in the liver microsome incubation systems was monitored by LC-MS/MS. The incubation of benzydamine (positive control, 200 μM) was carried out concurrently and the formation of benzydamine N-oxide was monitored by LC-MS/MS.

As shown in Table 19 and FIG. 12, the formation of 5-(3,4-dichloro-phenyl)-3-aza-bicyclo[3.1.0]hexan-2-one (EB-10101) was independent from pre-incubation temperatures in humans.

The amount of 5-(3,4-dichloro-phenyl)-3-aza-bicyclo[3.1.0]hexan-2-one (EB-10101) formed in the HLM and RLM with 37° C. pre-incubation was similar to the amount of 5-(3,4-dichloro-phenyl)-3-aza-bicyclo[3.1.0]hexan-2-one (EB-10101) formed with 45° C. pre-incubation. The results indicated that the formation of 5-(3,4-dichloro-phenyl)-3-aza-bicyclo[3.1.0]hexan-2-one (EB-10101) was not FMO-mediated in humans. However, the formation of 5-(3,4-dichloro-phenyl)-3-aza-bicyclo[3.1.0]hexan-2-one (EB-10101) isomer was dependent on pre-incubation temperatures in rats with the amount of 5-(3,4-dichloro-phenyl)-3-aza-bicyclo[3.1.0]hexan-2-one (EB-10101) and M-12 isomers formed in the RLM with 37° C. pre-treatment 7.3- and 4.6-fold of those formed with 45° C. pre-treatment respectively.

### Example XXXIX

### MAO Reaction Phenotyping

Incubations of amitifadine (EB-1010) (1 μM) and bicifadine (1 μM) in HLM (XenoTech, LLC, Lenexa, Kans.) and human liver mitochondria (HLmito) in the absence and presence of various concentrations of MAO-A and MAO-B inhibitors were performed. In addition, amitifadine (EB-1010) and bicifadine were incubated with recombinant human MAO enzymes individually. The formation of EB-10101 and M-12 in the incubation systems was monitored by LC-MS/MS.

The microsomal and mitochondria incubation mixtures (ca. 0.2 mL) were prepared in triplicate in 0.1 M potassium phosphate buffer (pH 7.4) (BD Biosciences, Woburn, Mass.), 4 mM MgCl₂, 0.5 mg/mL HLM and 1 mg/mL HLmito, respectively, in the absence and presence of different concentrations of MAO inhibitors. Incubation mixtures were pre-incubated aerobically in a shaking water bath at 37° C. for ca. 5 min. The reaction was initiated by the addition of amitifadine (EB-1010) (final concentration of 1 μM) working solutions (working solutions were prepared in incubation buffer by dilutions of respective stock solutions (10 μM, 10×)). After 30 min incubation, the reaction was stopped by mixing the incubation samples with two volumes of ice-cold quench solution (150 nM bicifadine-d5 prepared in acetonitrile by dilution of the internal standard spiking solution which was 200 μM bicifadine-d5 prepared in methanol by diluting the stock solution). The mixtures were maintained at ca. −20° C. before analysis by LC-MS/MS. The positive control incubations were carried out concurrently under the same conditions as described above with the exception that bicifadine was used instead of EB-1010. Negative control samples were prepared as described above with the exception that instead of HLM or HLmito, water was added to the incubation mixture.

As shown in Table 20 and FIG. 13, the formation of 5-(3,4-dichloro-phenyl)-3-aza-bicyclo[3.1.0]hexan-2-one (EB-10101) in the HLM and HLmito incubations was inhibited by the MAO-A inhibitor, clorgyline (Regis Technologies, Inc., Morton Grove, Ill.) the formation rates of 5-(3,4-dichloro-phenyl)-3-aza-bicyclo[3.1.0]hexan-2-one (EB-10101) decreased with increasing clorgyline concentrations. In addition, the formation of 5-(3,4-dichloro-phenyl)-3-aza-bicyclo[3.1.0]hexan-2-one (EB-10101) in the human recombinant MAO-A enzyme incubations confirmed that the formation of 5-(3,4-dichloro-phenyl)-3-aza-bicyclo[3.1.0]hexan-2-one (EB-10101) was MAO-A mediated.

TABLE 20
Effect of clorgryline and selegiline on the formation of EB-10101 and
M-12 from EB-1010 and bicifadine, respectively, in human liver
microsomal and mitochondrial incubations^{a}

| | | EB-1010 formation rate | | M-12 formation rate | |
|---|---|---|---|---|---|
| | | (mean ± SD in pmol/min/mg protein) | | (mean ± SD in pmol/min/mg protein) | |
| Inhibitor | Conc. (μM) | Microsomes | Mitochondria | Microsomes | Mitochondria |
| Clorgyline | 0 | 31.2 ± 2.6 | 16.9 ± 0.5 | 41.1 ± 0.7 | 19.9 ± 2.2 |
| | 0.001 | 24.4 ± 1.7 | 17.5 ± 3.5 | 39.3 ± 3.6 | 20.3 ± 2.1 |
| | 0.01 | 26.5 ± 5.8 | 15.3 ± 2.8 | 49.5 ± 2.3 | 20.3 ± 1.6 |
| | 0.1 | 0.9 ± 0.3 | 4.4 ± 1.3 | 39.6 ± 4.5 | 19.7 ± 3.5 |
| | 1 | 0.0 ± 0.1 | 0.1 ± 0.0 | 38.1 ± 2.1 | 17.0 ± 0.8 |
| Selegiline | 0 | 25.8 ± 9.1 | 15.9 ± 4.7 | 46.5 ± 3.2 | 20.7 ± 1.9 |
| | 0.001 | 24.0 ± 3.6 | 17.3 ± 2.0 | 49.6 ± 0.6 | 21.9 ± 2.9 |
| | 0.01 | 24.2 ± 1.1 | 18.5 ± 5.7 | 50.2 ± 2.6 | 23.1 ± 2.7 |
| | 0.1 | 28.5 ± 4.9 | 19.3 ± 5.6 | 22.0 ± 2.7 | 16.3 ± 1.5 |
| | 1 | 25.0 ± 4.1 | 16.1 ± 2.7 | 9.7 ± 0.2 | 11.4 ± 0.5 |

^{a}Pooled human liver microsomes (0.5 mg/mL) and mitochondria (1 mg/mL) were incubated with various concentrations of clorgyline and selegiline separately at ca. 37° C. for 5 min before the addition of EB-1010 and bicifadine (1 μM). The incubation mixtures were further incubated at ca. 37° C. for 30 min. The lactam metabolites of EB-1010 and bicifadine, were monitored using LC-MS/MS.
SD: standard deviation

The formation of 5-(3,4-dichloro-phenyl)-3-aza-bicyclo[3.1.0]hexan-2-one (EB-10101) in the HLM and HLmito incubations was not inhibited by the MAO-B inhibitor, selegiline (Santa Cruz Biotechnology, Inc., Santa Cruz, Calif.). The formation rates of EB-10101 remained unchanged with increasing selegiline concentrations. In addition, as shown in Table 20 and FIG. 13, the absence of the formation of 5-(3,4-dichloro-phenyl)-3-aza-bicyclo[3.1.0]hexan-2-one (EB-10101) in the human recombinant MAO-B enzyme incubations confirmed that the formation of 5-(3,4-dichloro-phenyl)-3-aza-bicyclo[3.1.0]hexan-2-one (EB-10101) was not MAO-B mediated.

### Example XL

### Metabolism by Recombinant Enzymes

Incubation systems (ca. 0.2 mL) of human recombinant MAO-A (1.0 mg/mL) and MAO-B (1.0 mg/mL) (BD Biosciences, Woburn, Mass.) were prepared in triplicated 0.1 M potassium phosphate buffer (pH 7.4) (BD Biosciences, Woburn, Mass.). Incubation systems were pre-incubated aerobically in a shaking water bath at 37° C., the reaction was stopped by mixing the incubation samples with two volumes of ice-cold quench solution (150 nM bicifadine-d5 prepared in acetonitrile by dilution of the internal standard spiking solution which was 200 μM bicifadine-d5 prepared in methanol by diluting the stock solution). The mixtures were maintained at ca. −20° C. before analysis by LC-MS/MS. The positive control incubations were carried out concurrently under the same conditions as described above with the exception that bicifadine was used instead of EB-1010. The negative samples were prepared as described above, with the exception that instead of HLM, HLmito, or recombinant enzymes, water was added to the incubation mixture.

As shown in Table 21, the formation of 5-(3,4-dichloro-phenyl)-3-aza-bicyclo[3.1.0]hexan-2-one (EB-10101) in the human recombinant MAO-A enzyme incubations confirmed that the formation of 5-(3,4-dichloro-phenyl)-3-aza-bicyclo[3.1.0]hexan-2-one (EB-10101) was MAO-A mediated. However, the formation rate of 5-(3,4-dichloro-phenyl)-3-aza-bicyclo[3.1.0]hexan-2-one (EB-10101) in the human recombinant MAO-A enzyme incubations system shown in Table 21 was probably underestimated due to limited substrate in the incubation.

TABLE 21
Formation rates of EB-10101 and M-12 from EB-1010 and bicifadine,
respectively, in the human recombinant MAO-A and MAO-B incubations^{a}

| | EB-10101 formation rate | M-12 formation rate |
|---|---|---|
| | (mean ± SD in | (mean ± SD in |
| Enzyme | pmol/min/mg protein) | pmol/min/mg protein) |
| MAO-A | 7.0 ± 2.7^{b} | 2.8 ± 0.6 |
| MAO-B | ND | 0.5 ± 0.1 |
| NC | ND | ND |

^{a}EB-1010 and bicifadine (1 μM) were incubated separately with 1 mg/ML recombinant MAO-A, MAO-B and MAO insect control (as negative control) at ca. 37° C. for 30 min. The lactam metabolites of EB-1010 and bicifadine were monitored using LC-MS/MS.
^{b}The formation rate of EB-10101 was underestimated because the substrate (EB-1010) was limited under the incubation condition.
SD: standard deviation
NC: negative controls
ND: not detected

### Example XLI

### Safety, Tolerance, and Pharmacokinetics of Amitifadine in Humans

The pharmacokinetic parameters of amitifadine, (Dov 21, 947) in healthy male volunteers following a single oral dose of amitifadine HCL (10-150 mg immediate release formulation) were calculated using plasma concentrations determined using a validated LC/MS/MS assay.

Single oral doses of amitifadine HCl immediate release (IR) formulation were administered to five groups of healthy adult male volunteers. Blood samples were collected from pre-dose to 24 hour for the determination of plasma amitifadine concentrations.

Fifty (50) healthy male adult volunteers, between the ages of 19 and 43 years (mean±SD: 28±6 years) were enrolled in the study and randomized to the six different treatments. All volunteers met the criteria for inclusion in study as detailed in the protocol and signed an informed consent form. The subjects were divided into panels of 10 subjects/dose group. Seven subjects in each group received the test drug, while three received an identical-looking placebo. Amitifadine was administered in capsules; the placebo group received an equal number of matching capsules. The five treatments are outlined below:

Treatment A: 1 capsule of amitifadine IR 10 mg (10 mg total), PO

Treatment B: 1 capsule of amitifadine IR 25 mg (25 mg total), PO

Treatment C: 2 capsules of amitifadine IR 25 mg (50 mg total), PO

Treatment D: 4 capsules of amitifadine IR 25 mg (100 mg total), PO

Treatment E: 6 capsules of amitifadine IR 25 mg (150 mg total), PO

The subjects were admitted to the clinic the night before dose administration and fasted from approximately 10 hour before to 4 hour after ingestion of the study capsules. Treatments were administered with 240 mL of mineral water between 08:00 and 09:00 hours. Venous blood samples (approximately 10 mL) were obtained via an indwelling cannula or by direct venipuncture of the antecubital veins at the following times following drug administration: 0 (pre-dose), 0.25, 0.5, 0.75, 1, 1.5, 2, 2.5, 3, 4, 6, 8, 10, 12, and 24 hours. Samples were collected into lithium heparin-containing Vacutainers and centrifuged at 3,000 rpm for a period of 15 min at 2° C. within 1 hour of sample collection. The plasma was pipetted into prelabeled polypropylene tubes, stored at approximately −20° C., and sent to WIL Research Laboratories (Ashland, Ohio) for the analysis of amitifadine by a validated LC/MS/MS assay. The assay calibration range was 5 to 2,000 ng/mL.

The values of Cₘₐₓ and Tₘₐₓ were the observed value for each subject. The terminal elimination rate constant λz was derived from the log-linear disposition phase of the concentration-time curve using linear least-squares regression for which the program determined the appropriate number of terminal points to calculate λz. At least three terminal plasma concentrations were used for estimating λz. The t_{1/2} (HL_Lambda_z) was calculated as ln 2/λz. AUC₀₋ₜ and AUMC₀₋ₜ were determined using the linear trapezoidal rule for each increasing concentration and the log-trapezoidal rule for descending concentrations. The AUC values from the last quantifiable time point to infinity was calculated as Ct/λz. This value was added to AUC₀₋ₜ to obtain AUC_{0-∞}. The mean residence time from time zero to infinity (MRT_{0-∞}) was calculated as AUMC_{0-∞}/AUC_{0-∞}. Oral clearance was calculated as Dose/AUC_{0-∞}. The 90% confidence intervals were calculated for all of the variables.

An analysis of variance (ANOVA) to test for differences among pharmacokinetic parameters and the dose levels of amitifadine was performed using the general linear model. SAS, version 8.2, was used for the analysis.

TABLE 22
Plasma Concentrations (ng/mL) of DOV 21,947 in Healthy Male Volunteers
Following a Single Oral Dose of DOV 21,947-HCl 10 mg IR Formulation

| | Hour | | | | | | | | | | | | | | |
|---|---|---|---|---|---|---|---|---|---|---|---|---|---|---|---|
| Subject | 0.00 | 0.25 | 0.50 | 0.75 | 1.00 | 1.50 | 2.00 | 2.50 | 3.00 | 4.00 | 6.00 | 8.00 | 10.00 | 12.00 | 24.00 |
| 1 | 0.00^{a} | 0.00 | 40.5 | 67.5 | 79.0 | 71.8 | 60.2 | 61.6 | 60.4 | 44.8 | 30.0 | 23.7 | 13.4 | 11.2 | 0.00 |
| 2 | 0.00 | 9.06 | 70.7 | 82.3 | 73.9 | 60.9 | 52.7 | 49.9 | 48.9 | 33.0 | 20.3 | 15.7 | 8.28 | 6.30 | 0.00 |
| 3 | 0.00 | 27.9 | 123 | 113 | 103 | 87.0 | 74.4 | 65.0 | 64.3 | 54.6 | 30.7 | 23.4 | 13.8 | 11.9 | 0.00 |
| 4 | 0.00 | 43.4 | 91.0 | 87.4 | 88.3 | 65.9 | 57.9 | 53.4 | 48.0 | 43.3 | 30.3 | 17.7 | 13.1 | 8.79 | 0.00 |
| 5 | 0.00 | 0.00 | 53.9 | 67.9 | 64.3 | 42.3 | 38.5 | 29.0 | 24.2 | 21.2 | 15.5 | 9.08 | 7.47 | 5.28 | 0.00 |
| 7 | 0.00 | 16.6 | 103 | 112 | 104 | 71.1 | 75.6 | 47.3 | 61.0 | 44.0 | 26.3 | 20.4 | 11.1 | 7.82 | 0.00 |
| 10 | 0.00 | 0.00 | 45.3 | 113 | 109 | 87.6 | 71.9 | 61.4 | 61.7 | 45.2 | 28.9 | 16.3 | 12.1 | 7.26 | 0.00 |
| Mean | 0.00 | 13.8 | 75.4 | 91.7 | 88.8 | 69.5 | 61.6 | 52.5 | 52.7 | 40.9 | 26.0 | 18.0 | 11.3 | 8.37 | 0.00 |
| SD | 0.00 | 16.7 | 31.3 | 20.6 | 17.1 | 15.6 | 13.5 | 12.3 | 14.1 | 10.7 | 5.88 | 5.08 | 2.53 | 2.47 | 0.00 |
| Minimum | 0.00 | 0.00 | 40.5 | 67.5 | 64.3 | 42.3 | 38.5 | 29.0 | 24.2 | 21.2 | 15.5 | 9.08 | 7.47 | 5.28 | 0.00 |
| Median | 0.00 | 9.06 | 70.7 | 87.4 | 88.3 | 71.1 | 60.2 | 53.4 | 60.4 | 44.0 | 28.9 | 17.7 | 12.1 | 7.82 | 0.00 |
| Maximum | 0.00 | 43.4 | 123 | 113 | 109 | 87.6 | 75.6 | 65.0 | 64.3 | 54.6 | 30.7 | 23.7 | 13.8 | 11.9 | 0.00 |
| CV % | ND | 121 | 42 | 22 | 19 | 22 | 22 | 23 | 27 | 26 | 23 | 28 | 22 | 29 | ND |

^{a}Less than LLOQ (5 ng/mL)
ND: Not determined when mean = 0

TABLE 23
Plasma Concentrations (ng/mL) of DOV 21,947 in Healthy Male Volunteers
Following a Single Oral Dose of DOV 21,947-HCl 25 mg IR Formulation

| | Hour | | | | | | | | | | | | | | |
|---|---|---|---|---|---|---|---|---|---|---|---|---|---|---|---|
| Subject | 0.00 | 0.25 | 0.50 | 0.75 | 1.00 | 1.50 | 2.00 | 2.50 | 3.00 | 4.00 | 6.00 | 8.00 | 10.00 | 12.00 | 24.00 |
| 12 | 0.00^{a} | 37.4 | 468 | 669 | 510 | 424 | 430 | 345 | 340 | 202 | 160 | 81.5 | 43.9 | 33.1 | 0.00 |
| 13 | 0.00 | 15.5 | 313 | 519 | 370 | 256 | 281 | 162 | 175 | 116 | 154 | 78.2 | 21.5 | 37.3 | 0.00 |
| 15 | 0.00 | 76.4 | 325 | 368 | 235 | 212 | 198 | 175 | 135 | 108 | 100 | 48.1 | 24.7 | 23.9 | 0.00 |
| 16 | 0.00 | 61.7 | 253 | 337 | 256 | 218 | 227 | 172 | 157 | 101 | 59.1 | 40.8 | 22.9 | 16.1 | 0.00 |
| 18 | 0.00 | 101 | 258 | 294 | 291 | 229 | 205 | 172 | 154 | 126 | 92.0 | 61.3 | 44.1 | 30.1 | 6.62 |
| 19 | 0.00 | 0.00 | 65.5 | 210 | 362 | 300 | 228 | 198 | 187 | 127 | 73.4 | 38.4 | 25.6 | 17.9 | 0.00 |
| 20 | 0.00 | 21.1 | 104 | 186 | 190 | 195 | 186 | 164 | 157 | 106 | 73.4 | 43.2 | 25.7 | 19.8 | 0.00 |
| Mean | 0.00 | 44.7 | 255 | 369 | 316 | 262 | 251 | 199 | 186 | 127 | 102 | 56.0 | 29.8 | 25.5 | 0.946 |
| SD | 0.00 | 36.2 | 137 | 172 | 108 | 79.3 | 84.8 | 65.8 | 69.7 | 34.4 | 40.1 | 18.0 | 9.83 | 8.15 | 2.50 |
| Minimum | 0.00 | 0.00 | 65.5 | 186 | 190 | 195 | 186 | 162 | 135 | 101 | 59.1 | 38.4 | 21.5 | 16.1 | 0.00 |
| Median | 0.00 | 37.4 | 258 | 337 | 291 | 229 | 227 | 172 | 157 | 116 | 92.0 | 48.1 | 25.6 | 23.9 | 0.00 |
| Maximum | 0.00 | 101 | 468 | 669 | 510 | 424 | 430 | 345 | 340 | 202 | 160 | 81.5 | 44.1 | 37.3 | 6.62 |
| CV % | ND | 81 | 54 | 47 | 34 | 30 | 34 | 33 | 37 | 27 | 39 | 32 | 33 | 32 | 265 |

b. Less than LLOQ (5 ng/mL)
ND: Not determined when mean = 0

TABLE 24
Plasma Concentrations (ng/mL) of DOV 21,947 in Healthy Male Volunteers
Following a Single Oral Dose of DOV 21,947-HCl 50 mg IR Formulation

| | Hour | | | | | | | | | | | | | | |
|---|---|---|---|---|---|---|---|---|---|---|---|---|---|---|---|
| Subject | 0.00 | 0.25 | 0.50 | 0.75 | 1.00 | 1.50 | 2.00 | 2.50 | 3.00 | 4.00 | 6.00 | 8.00 | 10.00 | 12.00 | 24.00 |
| 21 | 0.00 a | 56.5 | 160 | 421 | 457 | 383 | 302 | 257 | 244 | 171 | 141 | 84.9 | 55.1 | 37.5 | 5.23 |
| 25 | 0.00 | 29.8 | 188 | 451 | 503 | 423 | 319 | 222 | 217 | 167 | 163 | 93.2 | 58.5 | 41.0 | 5.38 |
| 26 | 0.00 | 17.3 | 107 | 206 | 284 | 381 | 318 | 295 | 224 | 158 | 106 | 62.3 | 42.0 | 33.5 | 0.00 |
| 27 | 0.00 | 64.3 | 357 | 715 | 736 | 594 | 416 | 372 | 362 | 298 | 179 | 88.3 | 63.3 | 38.7 | 0.00 |
| 28 | 0.00 | 40.9 | 306 | 499 | 499 | 387 | 338 | 252 | 244 | 178 | 139 | 112 | 60.2 | 41.5 | 0.00 |
| 29 | 0.00 | 40.6 | 158 | 695 | 796 | 573 | 547 | 457 | 374 | 293 | 229 | 139 | 74.8 | 51.0 | 7.96 |
| 30 | 0.00 | 124 | 461 | 573 | 705 | 596 | 536 | 377 | 376 | 322 | 214 | 128 | 90.1 | 48.8 | 7.09 |
| Mean | 0.00 | 53.4 | 248 | 509 | 569 | 477 | 397 | 319 | 292 | 227 | 167 | 101 | 63.4 | 41.7 | 3.67 |
| SD | 0.00 | 34.9 | 129 | 175 | 183 | 105 | 106 | 85.3 | 74.6 | 73.5 | 43.7 | 26.6 | 15.3 | 6.21 | 3.56 |
| Minimum | 0.00 | 17.3 | 107 | 206 | 284 | 381 | 302 | 222 | 217 | 156 | 106 | 62.3 | 42.0 | 33.5 | 0.00 |
| Median | 0.00 | 40.9 | 188 | 499 | 503 | 423 | 338 | 295 | 244 | 178 | 163 | 93.2 | 60.2 | 41.0 | 5.23 |
| Maximum | 0.00 | 124 | 461 | 715 | 796 | 596 | 547 | 457 | 376 | 322 | 229 | 139 | 90.1 | 51.0 | 7.98 |
| CV % | ND | 65 | 52 | 34 | 32 | 22 | 27 | 27 | 26 | 32 | 26 | 26 | 24 | 15 | 97 |

a. Less than LLOQ (5 ng/mL)
ND: Not determined when mean = 0

TABLE 25
Plasma Concentrations (ng/mL) of DOV 21,947 in Healthy Male Volunteers
Following a Single Oral Dose of DOV 21,947-HCl 100 mg IR Formulation

| | Hour | | | | | | | | | | | | | | |
|---|---|---|---|---|---|---|---|---|---|---|---|---|---|---|---|
| Subject | 0.00 | 0.25 | 0.50 | 0.75 | 1.00 | 1.50 | 2.00 | 2.50 | 3.00 | 4.00 | 6.00 | 8.00 | 10.00 | 12.00 | 24.00 |
| 33 | 0.00 a | 255 | 936 | 1660 | 1530 | 1160 | 981 | 834 | 748 | 475 | 330 | 208 | 126 | 97.6 | 11.0 |
| 34 | 0.00 | 43.2 | 224 | 348 | 530 | 776 | 904 | 670 | 639 | 478 | 349 | 205 | 105 | 71.2 | 11.0 |
| 36 | 0.00 | 160 | 365 | 703 | 971 | 1080 | 883 | 826 | 686 | 510 | 354 | 173 | 90.8 | 72.4 | 8.11 |
| 37 | 0.00 | 120 | 362 | 377 | 369 | 332 | 622 | 823 | 673 | 534 | 361 | 213 | 132 | 100 | 13.9 |
| 38 | 0.00 | 47.5 | 111 | 149 | 176 | 249 | 374 | 491 | 518 | 572 | 352 | 219 | 141 | 104 | 12.8 |
| 39 | 0.00 | 75.3 | 558 | 1250 | 909 | 859 | 717 | 592 | 525 | 418 | 325 | 184 | 115 | 94.9 | 13.6 |
| 40 | 0.00 | 79.0 | 623 | 791 | 1000 | 924 | 773 | 730 | 556 | 484 | 366 | 231 | 139 | 87.9 | 13.2 |
| Mean | 0.00 | 111 | 454 | 753 | 783 | 769 | 750 | 709 | 621 | 496 | 348 | 205 | 121 | 89.7 | 12.0 |
| SD | 0.00 | 75.4 | 277 | 538 | 457 | 352 | 206 | 133 | 89.1 | 49.0 | 15.3 | 20.1 | 18.5 | 13.2 | 2.06 |
| Minimum | 0.00 | 43.2 | 111 | 149 | 176 | 249 | 374 | 491 | 518 | 418 | 325 | 173 | 90.8 | 71.2 | 8.11 |
| Median | 0.00 | 79.2 | 365 | 703 | 909 | 859 | 773 | 730 | 639 | 484 | 352 | 208 | 126 | 94.9 | 12.8 |
| Maximum | 0.00 | 255 | 936 | 1660 | 1530 | 1160 | 981 | 834 | 748 | 572 | 366 | 231 | 141 | 104 | 13.9 |
| CV % | ND | 68 | 61 | 71 | 58 | 46 | 27 | 19 | 14 | 10 | 4 | 10 | 15 | 15 | 17 |

a. Less than LLOQ (5 ng/mL)
ND: Not determined when mean = 0

TABLE 26
Plasma Concentrations (ng/mL) of DOV 21,947 in Healthy Male Volunteers
Following a Single Oral Dose of DOV 21,947-HCl 150 mg IR Formulation

| | Hour | | | | | | | | | | | | | | |
|---|---|---|---|---|---|---|---|---|---|---|---|---|---|---|---|
| Subject | 0.00 | 0.25 | 0.50 | 0.75 | 1.00 | 1.50 | 2.00 | 2.50 | 3.00 | 4.00 | 6.00 | 8.00 | 10.00 | 12.00 | 24.00 |
| 41 | 0.00a | 284 | 284 | 511 | 658 | 817 | 1080 | 971 | 825 | 736 | 609 | 419 | 289 | 188 | 32.2 |
| 42 | 0.00 | 73.6 | 718 | 1480 | 1250 | 1410 | 1420 | 1160 | 1080 | 901 | 509 | 331 | 208 | 119 | 15.0 |
| 44 | 0.00 | 91.9 | 429 | 897 | 1010 | 1590 | 1320 | 1140 | 843 | 751 | 446 | 299 | 161 | 149 | 17.6 |
| 46 | 0.00 | 38.4 | 270 | 575 | 895 | 1010 | 869 | 713 | 743 | 622 | 499 | 303 | 202 | 160 | 28.1 |
| 48 | 0.00 | 146 | 473 | 994 | 1060 | 973 | 1450 | 1420 | 1300 | 1040 | 983 | 626 | 418 | 291 | 30.4 |
| 49 | 0.00 | 212 | 291 | 553 | 1160 | 1240 | 1280 | 1230 | 1370 | 1170 | 904 | 615 | 378 | 293 | 6.72 |
| 50 | 0.00 | 351 | 1630 | 1510 | 1880 | 1950 | 1760 | 1420 | 1300 | 917 | 538 | 305 | 203 | 136 | 12.4 |
| Mean | 0.00 | 171 | 584 | 931 | 1130 | 1280 | 1310 | 1150 | 1070 | 877 | 642 | 414 | 269 | 191 | 20.3 |
| SD | 0.00 | 116 | 485 | 426 | 381 | 396 | 283 | 250 | 263 | 189 | 213 | 147 | 95.5 | 72.2 | 9.88 |
| Minimum | 0.00 | 38.4 | 270 | 511 | 658 | 817 | 869 | 713 | 743 | 622 | 448 | 299 | 181 | 119 | 6.72 |
| Median | 0.00 | 146 | 429 | 897 | 1060 | 1240 | 1320 | 1160 | 1060 | 901 | 538 | 331 | 208 | 160 | 17.6 |
| Maximum | 0.00 | 351 | 1630 | 1510 | 1880 | 1950 | 1760 | 1420 | 1370 | 1170 | 983 | 626 | 418 | 293 | 32.2 |
| CV % | ND | 68 | 83 | 48 | 34 | 31 | 22 | 22 | 25 | 22 | 33 | 36 | 36 | 38 | 49 |

aLess than LLOQ (5 ng/mL)
ND: Not determined when mean = 0

TABLE 27
Mean Pharmacokinetic Parameters (SD) of DOV 21,947 in
Healthy Male Volunteers Following a Single Oral Dose
of DOV 21,947 HCl IR-Formulation

| | Dose DOV 21,947-HCl^{a} | | | | | | | | | |
|---|---|---|---|---|---|---|---|---|---|---|
| | 10 mg | | 25 mg | | 50 mg | | 100 mg | | 150 mg | |
| Cₘₐₓ (ng/mL) | 95.4 | (20.6) | 392 | (156) | 583 | (160) | 1040 | (344) | 1420 | (316) |
| Tₘₐₓ (hr) | 0.714 | (0.173) | 0.893 | (0.283) | 1.04 | (0.23) | 1.79 | (1.18) | 1.75 | (0.69) |
| AUC₀₋ₜ (ng · hr/mL) | 387 | (85) | 1389 | (418) | 2390 | (652) | 4810 | (609) | 8510 | (1690) |
| AUC_{0-∞} (ng · hr/mL) | 429 | (95) | 1470 | (431) | 2490 | (623) | 4880 | (604) | 8620 | (1660) |
| λ_{z} (hr⁻¹) | 0.205 | (0.022) | 0.251 | (0.114) | 0.192 | (0.306) | 0.167 | (0.008) | 0.195 | (0.047) |
| t_{1/2} (hr) | 3.42 | (0.37) | 3.15 | (1.15) | 3.68 | (0.55) | 4.15 | (0.19) | 3.71 | (0.79) |
| Oral Clearance (L/hr) | 21.2 | (6.0) | 15.6 | (3.6) | 18.3 | (4.6) | 17.9 | (2.2) | 15.5 | (2.9) |
| MRT_{0-∞} (hr) | 5.23 | (0.49) | 4.83 | (0.77) | 5.25 | (0.43) | 5.75 | (0.77) | 6.04 | (0.05) |

^{a}n = 7/dosing groups

TABLE 28
Individual and Mean Pharmacokinetic Parameters of DOV 21,947 in Healthy Male Volunteers
Following a Single Oral Dose of DOV 21,947-HCl 10 mg IR Formulation

| | | | | | | | Oral | |
|---|---|---|---|---|---|---|---|---|
| | Cₘₐₓ | Tₘₐₓ | AUC₀₋ₜ | AUC_{0-∞} | λ₂ | t_{1/2} | Clearance | MRT_{0-∞} |
| Subject | (ng/mL) | (hr) | (ng · hr/mL) | (ng · hr/mL) | (hr⁻¹) | (hr) | (L/hr) | (hr) |
| 1 | 79.0 | 1.00 | 409 | 470 | 0.183 | 3.79 | 18.3 | 6.09 |
| 2 | 82.3 | 0.750 | 328 | 356 | 0.225 | 3.08 | 24.2 | 4.84 |
| 3 | 123 | 0.500 | 492 | 551 | 0.200 | 3.47 | 15.6 | 5.36 |
| 4 | 91.0 | 0.500 | 406 | 452 | 0.191 | 3.63 | 19.1 | 5.35 |
| 5 | 67.9 | 0.750 | 227 | 257 | 0.178 | 3.90 | 33.6 | 5.47 |
| 7 | 112 | 0.750 | 424 | 459 | 0.225 | 3.09 | 18.8 | 4.79 |
| 10 | 113 | 0.750 | 423 | 454 | 0.233 | 2.98 | 19.0 | 4.72 |
| Mean | 95.4 | 0.714 | 387 | 429 | 0.205 | 3.42 | 21.2 | 5.23 |
| SD | 20.6 | 0.173 | 85.1 | 94.6 | 0.0224 | 0.374 | 6.00 | 0.491 |
| Minimum | 67.9 | 0.500 | 227 | 257 | 0.178 | 2.98 | 15.6 | 4.72 |
| Median | 91.0 | 0.750 | 409 | 454 | 0.200 | 3.47 | 19.0 | 5.35 |
| Maximum | 123 | 1.00 | 492 | 551 | 0.233 | 3.90 | 33.6 | 6.09 |
| CV % | 22 | 24 | 22 | 22 | 11 | 11 | 28 | 9 |
| Geometric Mean | 93.5 | 0.696 | 377 | 418 | 0.204 | 3.40 | 20.6 | 5.21 |
| 90% CI Lower Mean | 80.2 | 0.588 | 324 | 359 | 0.188 | 3.14 | 16.8 | 4.87 |
| 90% CI Upper Mean | 111 | 0.841 | 450 | 498 | 0.221 | 3.69 | 25.6 | 5.59 |

TABLE 29
Individual and Mean Pharmacokinetic Parameters of DOV 21,947 in Healthy Male Volunteers
Following a Single Oral Dose of DOV 21,947-HCl 25 mg IR Formulation

| | | | | | | | Oral | |
|---|---|---|---|---|---|---|---|---|
| | Cₘₐₓ | Tₘₐₓ | AUC₀₋ₜ | AUC_{0-∞} | λ₂ | t_{1/2} | Clearance | MRT_{0-∞} |
| Subject | (ng/mL) | (hr) | (ng · hr/mL) | (ng · hr/mL) | (hr⁻¹) | (hr) | (L/hr) | (hr) |
| 12 | 669 | 0.750 | 2220 | 2350 | 0.258 | 2.69 | 9.19 | 4.29 |
| 13 | 519 | 0.750 | 1520 | 1600 | 0.493 | 1.41 | 13.5 | 4.39 |
| 15 | 368 | 0.750 | 1190 | 1300 | 0.222 | 3.13 | 16.6 | 4.81 |
| 16 | 337 | 0.750 | 1090 | 1160 | 0.231 | 3.00 | 18.7 | 4.26 |
| 18 | 294 | 0.750 | 1480 | 1530 | 0.132 | 5.25 | 14.1 | 6.46 |
| 19 | 382 | 1.00 | 1180 | 1270 | 0.191 | 3.62 | 17.0 | 4.65 |
| 20 | 195 | 1.50 | 995 | 1080 | 0.231 | 3.00 | 20.0 | 4.98 |
| Mean | 392 | 0.893 | 1380 | 1470 | 0.251 | 3.15 | 15.6 | 4.83 |
| SD | 158 | 0.283 | 418 | 431 | 0.114 | 1.15 | 3.64 | 0.768 |
| Minimum | 195 | 0.750 | 995 | 1080 | 0.132 | 1.41 | 9.19 | 4.26 |
| Median | 362 | 0.750 | 1190 | 1300 | 0.231 | 3.00 | 16.6 | 4.65 |
| Maximum | 669 | 1.50 | 2220 | 2350 | 0.493 | 5.25 | 20.0 | 6.46 |
| CV % | 40 | 32 | 30 | 29 | 45 | 36 | 23 | 15 |
| Geometric Mean | 367 | 0.863 | 1340 | 1430 | 0.234 | 2.97 | 15.2 | 4.79 |
| 90% CI Lower Mean | 277 | 0.685 | 1080 | 1150 | 0.168 | 2.31 | 12.9 | 4.27 |
| 90% CI Upper Mean | 507 | 1.10 | 1690 | 1790 | 0.335 | 4.00 | 18.2 | 5.40 |

TABLE 30
Individual and Mean Pharmacokinetic Parameters of DOV 21,947 in Healthy Male Volunteers
Following a Single Oral Dose of DOV 21,947-HCl 50 mg IR Formulation

| | | | | | | | Oral | |
|---|---|---|---|---|---|---|---|---|
| | Cₘₐₓ | Tₘₐₓ | AUC₀₋ₜ | AUC_{0-∞} | λ₂ | t_{1/2} | Clearance | MRT_{0-∞} |
| Subject | (ng/mL) | (hr) | (ng · hr/mL) | (ng · hr/mL) | (hr⁻¹) | (hr) | (L/hr) | (hr) |
| 21 | 457 | 1.00 | 2020 | 2060 | 0.167 | 4.16 | 21.0 | 5.60 |
| 25 | 503 | 1.00 | 2120 | 2150 | 0.170 | 4.08 | 20.1 | 5.66 |
| 26 | 381 | 1.50 | 1530 | 1690 | 0.213 | 3.25 | 25.5 | 5.18 |
| 27 | 736 | 1.00 | 2640 | 2800 | 0.245 | 2.82 | 15.4 | 4.38 |
| 28 | 499 | 0.750 | 1990 | 2200 | 0.199 | 3.48 | 19.6 | 5.29 |
| 29 | 796 | 1.00 | 3220 | 3270 | 0.158 | 4.39 | 13.2 | 5.43 |
| 30 | 705 | 1.00 | 3220 | 3260 | 0.191 | 3.62 | 13.2 | 5.22 |
| Mean | 563 | 1.04 | 2390 | 2490 | 0.192 | 3.68 | 16.3 | 5.25 |
| SD | 160 | 0.225 | 652 | 623 | 0.0306 | 0.554 | 4.56 | 0.427 |
| Minimum | 361 | 0.750 | 1530 | 1690 | 0.158 | 2.62 | 13.2 | 4.38 |
| Median | 503 | 1.00 | 2120 | 2200 | 0.191 | 3.62 | 19.6 | 5.29 |
| Maximum | 796 | 1.50 | 3220 | 3270 | 0.245 | 4.39 | 25.5 | 5.66 |
| CV % | 27 | 22 | 27 | 25 | 16 | 15 | 25 | 8 |
| Geometric Mean | 564 | 1.02 | 2320 | 2420 | 0.190 | 3.65 | 17.8 | 5.23 |
| 90% CI Lower Mean | 465 | 0.871 | 1910 | 2030 | 0.170 | 3.28 | 14.9 | 4.94 |
| 90% CI Upper Mean | 700 | 1.20 | 2870 | 2950 | 0.215 | 4.09 | 21.6 | 5.56 |

TABLE 31
Individual and Mean Pharmacokinetic Parameters of DOV 21,947 in Healthy Male Volunteers
Following a Single Oral Dose of DOV 21,947-HCl 100 mg IR Formulation

| | | | | | | | Oral | |
|---|---|---|---|---|---|---|---|---|
| | Cₘₐₓ | Tₘₐₓ | AUC₀₋ₜ | AUC_{0-∞} | λ₂ | t_{1/2} | Clearance | MRT_{0-∞} |
| Subject | (ng/mL) | (hr) | (ng · hr/mL) | (ng · hr/mL) | (hr⁻¹) | (hr) | (L/hr) | (hr) |
| 33 | 1660 | 0.750 | 5900 | 5960 | 0.177 | 3.92 | 14.5 | 4.85 |
| 34 | 904 | 2.00 | 4460 | 4530 | 0.159 | 4.35 | 19.0 | 5.69 |
| 36 | 1080 | 1.50 | 4960 | 5010 | 0.176 | 3.94 | 17.2 | 4.92 |
| 37 | 823 | 2.50 | 4550 | 4630 | 0.162 | 4.28 | 18.6 | 6.39 |
| 38 | 572 | 4.00 | 3960 | 4030 | 0.173 | 4.02 | 21.4 | 7.03 |
| 39 | 1250 | 0.750 | 4730 | 4820 | 0.159 | 4.35 | 17.9 | 5.71 |
| 40 | 1000 | 1.00 | 5110 | 5190 | 0.164 | 4.22 | 16.6 | 5.63 |
| Mean | 1040 | 1.79 | 4810 | 4880 | 0.167 | 4.15 | 17.9 | 5.75 |
| SD | 344 | 1.18 | 609 | 604 | 0.00780 | 0.192 | 2.15 | 0.769 |
| Minimum | 572 | 0.750 | 3960 | 4030 | 0.159 | 3.92 | 14.5 | 4.85 |
| Median | 1000 | 1.50 | 4730 | 4820 | 0.164 | 4.22 | 17.9 | 5.69 |
| Maximum | 1660 | 4.00 | 5900 | 5960 | 0.177 | 4.35 | 21.4 | 7.03 |
| CV % | 33 | 66 | 13 | 12 | 5 | 5 | 12 | 13 |
| Geometric Mean | 993 | 1.50 | 4780 | 4850 | 0.167 | 4.15 | 178 | 5.70 |
| 90% CI Lower Mean | 788 | 0.922 | 4360 | 4440 | 0.161 | 4.01 | 16.3 | 5.18 |
| 90% CI Upper Mean | 1290 | 2.65 | 5260 | 5320 | 0.173 | 4.29 | 19.5 | 6.31 |

TABLE 32
Individual and Mean Pharmacokinetic Parameters of DOV 21,947 in Healthy Male Volunteers
Following a Single Oral Dose of DOV 21,947-HCl 150 mg IR Formulation

| | | | | | | | Oral | |
|---|---|---|---|---|---|---|---|---|
| | Cₘₐₓ | Tₘₐₓ | AUC₀₋ₜ | AUC_{0-∞} | λ₂ | t_{1/2} | Clearance | MRT_{0-∞} |
| Subject | (ng/mL) | (hr) | (ng · hr/mL) | (ng · hr/mL) | (hr⁻¹) | (hr) | (L/hr) | (hr) |
| 41 | 1080 | 2.00 | 7520 | 7720 | 0.159 | 4.35 | 16.7 | 7.14 |
| 42 | 1480 | 0.750 | 7940 | 8010 | 0.209 | 3.31 | 16.1 | 5.18 |
| 44 | 1590 | 1.50 | 7210 | 7300 | 0.177 | 3.91 | 17.7 | 5.67 |
| 46 | 1010 | 1.50 | 6390 | 6590 | 0.142 | 4.87 | 19.6 | 7.03 |
| 48 | 1450 | 2.00 | 10900 | 11100 | 0.189 | 3.68 | 11.7 | 6.70 |
| 49 | 1370 | 3.00 | 10300 | 10300 | 0.288 | 2.41 | 12.5 | 5.87 |
| 50 | 1950 | 1.50 | 9250 | 9310 | 0.200 | 3.47 | 13.9 | 4.66 |
| Mean | 1420 | 1.75 | 8510 | 8620 | 0.195 | 3.91 | 15.5 | 6.04 |
| SD | 316 | 0.692 | 1690 | 1660 | 0.0470 | 0.785 | 2.89 | 0.953 |
| Minimum | 1010 | 0.750 | 6390 | 6590 | 0.142 | 2.41 | 11.7 | 4.66 |
| Median | 1450 | 1.50 | 7940 | 8010 | 0.189 | 3.68 | 16.1 | 5.87 |
| Maximum | 1950 | 300 | 10900 | 11100 | 0.288 | 4.87 | 19.6 | 7.14 |
| CV % | 22 | 40 | 20 | 19 | 24 | 21 | 19 | 16 |
| Geometric Mean | 1390 | 1.63 | 8360 | 8490 | 0.191 | 3.64 | 15.2 | 5.97 |
| 90% CI Lower Mean | 1190 | 1.24 | 7260 | 7400 | 0.160 | 3.14 | 13.3 | 5.34 |
| 90% CI Upper Mean | 1650 | 2.26 | 9750 | 9840 | 0.229 | 4.29 | 17.6 | 6.74 |

As can be seen in Tables 27-32, both Cₘₐₓ and AUC_{0-∞} of amitifadine increased in a dose-dependent manner as the dose increased from 10 mg to 150 mg. Oral clearance remained at approximately 15 to 21 L/hr over the dose range; the mean residence time was approximately 5 to 6 hours. The mean Tₘₐₓ increased from approximately 0.7 to 1.75 hr as the dose increased, but the elimination half-life remained at 3 to 4 hours. The interindividual variability of both AUC and Cₘₐₓ, as measured by the CV %, was generally less than 30% at all dose levels.

The association between the dose level of amitifadine and Cₘₐₓ, AUC₀₋ₜ, and AUC_{0-∞} was statistically significant; the linear association accounted for 83%, 93%, and 93% of the total variance for Cₘₐₓ, AUC₀₋ₜ, and AUC_{0-∞}, respectively. The linear relationship for t_{1/2} and oral clearance did not reach statistical significance.

### Example XLII

### Double-Blind, Randomized, Placebo-Controlled, Single Dose Pharmacokinetic, Safety, and Tolerability Study of Amitifadine in Healthy Adult Male and Female Human Subjects

Fourteen subjects, divided equally between males and females, were enrolled in the study and randomized between test article (5/sex) and placebo (2/sex). Demographics of the persons in Cohort 1 are listed below in Table 33. Cohort 1 was composed of 1 Asian, 6 black/African-American, 1 Caucasian, and 6 Hispanic/Latin subjects. All subjects met the inclusion and exclusion criteria for entry into the study as detailed in the protocol and signed an informed consent form.

TABLE 33

| | | | | |
|---|---|---|---|---|
| | Age (yr) | Weight (kg) | Height (cm) | BMI (kg/m²) |
| Number of | Mean ± SD | Mean ± SD | Mean ± SD | Mean ± SD |
| Subjects | (Range) | (Range) | (Range) | (Range) |

All Subjects

| | | | | |
|---|---|---|---|---|
| 14 | 25.9 ± 5.4 | 73.2 ± 16.0 | 172 ± 11 | 24.6 ± 3.9 |
| | (19.9-35.1) | (51.8-95.3) | (153-188) | (18.1-30.1) |

Males

| | | | | |
|---|---|---|---|---|
| 7 | 25.7 ± 5.7 | 83.5 ± 11.5 | 176 ± 7 | 26.9 ± 3.7 |
| | (20.3-35.1) | (65.0-95.3) | (164-188) | (19.8-30.1) |

Females

| | | | | |
|---|---|---|---|---|
| 7 | 26.1 ± 5.6 | 62.8 ± 13.2 | 167 ± 12 | 22.4 ± 2.8 |
| | (19.9-34.2) | (51.8-83.6) | (153-188) | (18.1-25.9) |

The subjects were confined to the clinical study site from Day −1 to the morning of Day 2, when they underwent a brief physical examination, clinical laboratory tests, and a physician visit before they were discharged. On Day 7, they reported to the clinical site again and were confined to the site until the morning of Day 9. At that time, they underwent a complete physical examination, clinical laboratory tests, and a physician's visit before they were discharged from the study. All subjects were fasted overnight on Days −1 and 7 from approximately 22:00 to approximately 4 hours post-dose on Days 1 and 8, at which time they received a standard lunch.

On Day 1, ten subjects (5/sex) received 300 mg of amitifadine HCl IR formulation in a total of six capsules (50 mg/capsule). Subjects that received the placebo (2/sex) ingested six identical looking capsules with no drug. On Day 8, the test subjects received 450 mg amitifadine HCl IR formulation in nine capsules (50 mg/capsule) and placebo subjects ingested nine identical looking capsules with no drug. Capsules on both days were ingested with 240 mL water. On both Day 1 and Day 8, venous blood samples (ca. 7 mL) for the collection of plasma were obtained at 0 (pre-dose), 0.25, 0.5, 0.75, 1, 1.25, 1.5, 2, 3, 4, 6, 8, 10, 12, 18, and 24 hr post-dose. Samples were collected into lithium heparin-containing Vacutainers® and centrifuged at approximately 3000 rpm to obtain the plasma fraction. The plasma was split into two polypropylene tubes and stored at −20° C. Resting 12-lead ECG and vital signs were collected at 0 (pre-dose), 0.5 (vital signs only), 1, 2, 3, 4, and 24 hr post-dose. Urine samples were collected on Days 1 and 8 at pre-dose and at intervals of 0-1, 1-2, 2-4, 4-8, 8-12, and 12-24 hr post-dose. The urine volumes were recorded and an aliquot was stored for drug concentration measurements.

Plasma and urine samples were sent on dry ice to WIL Research Laboratories (Ashland, Ohio) for the determination of amitifadine and the lactam metabolite DOV 216,298 (EB-10101). Validated LC/MS/MS assays were used for analyses; the assay range for both analytes in plasma and urine were 5.00 to 2000 ng/mL.

The following pharmacokinetic parameters for amitifadine were derived from the plasma concentration data for the subjects: area under the plasma concentration-time curve from time zero to the last quantifiable concentration (AUC₀₋ₜ), AUC from time zero to infinity (AUC_{0-∞}), the maximum observed plasma concentration (Cₘₐₓ), the time to Cₘₐₓ (Tₘₐₓ), the terminal phase rate constant (λ), the arithmetic and harmonic means of the half-life (t½), the apparent oral clearance (CL/F), the apparent oral volume of distribution (Vz/F), and mean residence time to infinity (MRT_{0-∞}). The same parameters were calculated for DOV 216,298 with the exception of CL/F, Vz/F, and MRT_{0-∞}. In addition, AUC_{0-∞} of DOV 216,298 is not reported in the tables since greater than 25% of AUC_{0-∞} was extrapolated from the time of the last sample collected to infinity for Cohort 1 (Days 1 and 8) and Cohort 2 (Day 1). Plasma concentration-time profiles for amitifadine and DOV 216,298 were summarized using descriptive statistics and graphical displays. Plasma concentration-time data for each subject were analyzed with WinNonlin, version 4.1 (Pharsight Corporation, Mountain View, Calif.) using a non-compartmental model. Since all PK samples were collected within 10% of their scheduled times, calculations were based on nominal sampling times, with the exception of two samples [Subject No. 1004, Day 8 (nominal 0.25 hr; actual 0.32 hr); Subject 2011, Day 1 (nominal 0.5 hr; actual 0.63 hr)] where the actual time was used. Plasma concentrations listed as not detected (ND) or below the lower limit of quantitation (LLOQ) were set to zero. The values of Cₘₐₓ and Tₘₐₓ were the observed values for each subject. AUC₀₋ₜ was determined using the linear trapezoidal rule whenever the concentration data were increasing and the logarithmic trapezoidal rule any time that the concentration data were decreasing. The apparent λz was calculated as the negative slope of the log-linear terminal portion of the blood concentration-time curve using linear regression. To allow for accurate calculation of λz, a minimum of three observations, selected by WinNonlin, was used. The t½ was calculated as ln 2/λz. AUC₀₋ₜ was extrapolated to infinity (AUC_{0-∞}) as follows: AUC_{0-∞}=AUC₀₋ₜ+Clast/λz where Clast is the last quantifiable concentration. The apparent oral clearance (CL/F) was calculated as Dose/AUC_{0-∞}. The apparent oral volume of distribution (Vz/F) was calculated as Dose/λz×AUC_{0-∞}. The mean residence time from time zero to infinity (MRT_{0-∞}) was calculated as AUMC_{0-∞}/AUC_{0-∞}. The urinary concentrations of amitifadine and DOV 216,298 from subjects in Cohort 1 were multiplied by the volume of urine collected during the period to obtain the amount of both analytes excreted during the interval (ARt). The total amounts of amitifadine and DOV 216,298 excreted in 24 hr were calculated for each subject. The renal clearance (CLr) of amitifadine was calculated using the equation CLr=Amount of amitifadine in the 0-24 hr urine collections divided by plasma AUC₀₋₂₄ for amitifadine.

Mean plasma concentrations of amitifadine (Dov 21,947) and DOV 216,298 following single oral doses of either 300 mg or 450 mg amitifadine HCl are shown in Tables 34 and 35.

TABLE 34
Mean Plasma Concentrations (SD) of DOV 21,947 and the
Lactam Metabolite DOV 216,298 in Healthy Adult Subjects
Following a Single Oral Dose of 300 mg or 450 mg DOV 21,947
HCl Immediate Release Formulation (Cohort 1)

| | 300 mg^{a} | | | | 450 mg^{a} | | | |
|---|---|---|---|---|---|---|---|---|
| Hour | DOV 21,947 | | DOV 216,298 | | DOV 21,947 | | DOV 216,298 | |
| 0.00 | 0.00^{b} | (0.00) | 0.00^{b} | (0.00) | 0.00 | (0.00) | 0.00 | (0.00) |
| 0.25 | 54.4 | (88.0) | 1.52 | (4.79) | 39.9 | (47.0) | 0.00 | (0.00) |
| 0.50 | 431 | (331) | 42.8 | (44.7) | 728 | (694) | 71.0 | (111) |
| 0.75 | 1240 | (907) | 175 | (93.1) | 1460 | (893) | 184 | (140) |
| 1.00 | 1830 | (1050) | 296 | (162) | 2260 | (1080) | 337 | (189) |
| 1.25 | 2470 | (986) | 473 | (208) | 3480 | (1400) | 646 | (300) |
| 1.50 | 2860 | (1020) | 589 | (221) | 3860 | (1660) | 807 | (369) |
| 2.00 | 3230 | (992) | 900 | (285) | 4760 | (1650) | 1330 | (606) |
| 3.00 | 2760 | (714) | 1410 | (466) | 4290 | (1130) | 1820 | (656) |
| 4.00 | 2080 | (434) | 1540 | (400) | 3280 | (682) | 2160 | (519) |
| 6.00 | 1530 | (535) | 1690 | (719) | 2400 | (565) | 2520 | (829) |
| 8.00 | 980 | (214) | 1900 | (565) | 1510 | (455) | 2670 | (655) |
| 10.0 | 564 | (127) | 1830 | (485) | 982 | (360) | 2770 | (638) |
| 12.0 | 409 | (115) | 1580 | (424) | 770 | (285) | 2620 | (772) |
| 18.0 | 148 | (60.1) | 1390 | (639) | 213 | (122) | 2010 | (1060) |
| 24.0 | 59.5 | (15.4) | 1010 | (448) | 95.0 | (30.0) | 1920 | (1030) |

^{a}N = 10
^{b}<LLOQ (5.00 ng/mL)

TABLE 35
Mean Plasma Concentrations (SD) of DOV 21,947 and the Lactam
Metabolite DOV 216,298 in Healthy Adult Male and Female Subjects
Following a Single Oral Dose of 300 mg or 450 mg DOV 21,947
HCl Immediate Release Formulation (Cohort 1)

| | Male^{a} | | Female^{a} | |
|---|---|---|---|---|
| Hour | DOV 21,947 | DOV 216,298 | DOV 21,947 | DOV 216,298 |

300 mg

| | | | | | | | | |
|---|---|---|---|---|---|---|---|---|
| 0.00 | 0.00^{b} | (0.00) | 0.00^{b} | (0.00) | 0.00 | (0.00) | 0.00 | (0.00) |
| 0.25 | 79.6 | (119) | 3.03 | (6.78) | 29.3 | (41.1) | 0.00 | (0.0) |
| 0.50 | 599 | (342) | 62.9 | (53.9) | 264 | (243) | 22.8 | (24.3) |
| 0.75 | 1690 | (1000) | 238 | (55.2) | 787 | (582) | 111 | (80.8) |
| 1.00 | 2390 | (1060) | 404 | (107) | 1280 | (768) | 188 | (137) |
| 1.25 | 3120 | (780) | 629 | (68.7) | 1830 | (737) | 317 | (179) |
| 1.50 | 3400 | (695) | 753 | (111) | 2330 | (1070) | 426 | (175) |
| 2.00 | 3340 | (835) | 1040 | (298) | 3110 | (1220) | 760 | (212) |
| 3.00 | 2610 | (278) | 1490 | (480) | 2910 | (1010) | 1340 | (498) |
| 4.00 | 1750 | (243) | 1470 | (383) | 2410 | (303) | 1600 | (450) |
| 6.00 | 1280 | (330) | 1520 | (532) | 1780 | (617) | 1870 | (896) |
| 8.00 | 874 | (169) | 1590 | (337) | 1090 | (215) | 2210 | (603) |
| 10.0 | 535 | (103) | 1670 | (398) | 592 | (154) | 1980 | (557) |
| 12.0 | 395 | (124) | 1410 | (316) | 424 | (119) | 1760 | (479) |
| 18.0 | 134 | (38.9) | 1100 | (323) | 162 | (78.1) | 1690 | (771) |
| 24.0 | 56.5 | (17.1) | 820 | (294) | 62.5 | (15.0) | 1190 | (527) |

450 mg

| | | | | | | | | |
|---|---|---|---|---|---|---|---|---|
| 0.00 | 0.00 | (0.00) | 0.00 | (0.00) | 0.00 | (0.00) | 0.00 | (0.00) |
| 0.25 | 55.3 | (61.4) | 0.00 | (0.00) | 24.4 | (24.5) | 0.00 | (0.00) |
| 0.50 | 923 | (906) | 107 | (155) | 532 | (407) | 35.2 | (25.4) |
| 0.75 | 1550 | (759) | 214 | (170) | 1380 | (1100) | 155 | (112) |
| 1.00 | 2510 | (939) | 388 | (203) | 2020 | (1270) | 285 | (180) |
| 1.25 | 4080 | (1540) | 752 | (345) | 2880 | (1080) | 539 | (235) |
| 1.50 | 4520 | (1950) | 954 | (434) | 3200 | (1130) | 660 | (254) |
| 2.00 | 5560 | (1260) | 1620 | (645) | 3950 | (1710) | 1030 | (438) |
| 3.00 | 4350 | (677) | 1980 | (520) | 4220 | (1550) | 1650 | (794) |
| 4.00 | 3180 | (504) | 2150 | (454) | 3390 | (876) | 2180 | (632) |
| 6.00 | 2300 | (319) | 2320 | (462) | 2500 | (768) | 2710 | (1110) |
| 8.00 | 1420 | (308) | 2360 | (459) | 1590 | (593) | 2990 | (712) |
| 10.0 | 895 | (122) | 2520 | (444) | 1070 | (507) | 3030 | (743) |
| 12.0 | 714 | (178) | 2310 | (476) | 826 | (379) | 2930 | (937) |
| 18.0 | 238 | (87.6) | 1880 | (537) | 187 | (156) | 2140 | (1480) |
| 24.0 | 103 | (31.8) | 1450 | (524) | 87.4 | (29.6) | 2390 | (1250) |

^{a}N = 5
^{b}<LLOQ (5.00 ng/mL)

The mean pharmacokinetic parameters are shown in Tables 36 through 38, below. The mean Cmax of amitifadine following the 300 mg dose was 3372 ng/mL (Table 38). This increased proportionately to 5110 ng/mL when the dose increased to 450 mg. Similarly, exposure to amitifadine, based on either AUC₀₋ₜ or AUC_{0-∞}, increased proportionately. Tₘₐₓ occurred at 2.15 hr and 2.43 hr at the 300 mg and 450 mg dose levels, respectively. The elimination half-life was approximately 4 hr at the two dose levels, while the mean residence time was 6.3-6.4 hr. Oral clearance was also similar at the 300 mg and 450 mg dose levels (13.2-13.6 L/hr). The mean apparent oral volume of distribution was 82.6 L at the lower dose level and 72.6 L at the higher dose. Variability of the pharmacokinetic parameters was low: the CV % values of Cₘₐₓ, AUC_{0-∞}, t½, and MRT_{0-∞} at the two dose levels were 28-29%, 18-21%, 10-13%, and 9-12%, respectively.

The mean Cₘₐₓ of the lactam, DOV 216,298, was lower (2031 ng/mL) compared to the parent drug at the 300 mg dose level and increased proportionately to 2954 ng/mL after the 450 mg dose. Tₘₐₓ was longer (7.8-9.0 hr) compared to amitifadine. The harmonic mean of the half-life was 14.3 hr and 19.8 hr with the low and high doses, respectively. These values should be viewed as less reliable than those for amitifadine since the decline in the concentrations of DOV 216,298 was not as great and the correlation coefficients for the best fit of the data were not as high. While the concentration of amitifadine at 24 hr was approximately 2% of its Cₘₐₓ, the concentration of DOV 216,298 at 24 hr post-dose was 50% and 65% of its Cₘₐₓ at the 300 mg and 450 mg dose levels, respectively. Exposure to the lactam was approximately 70% greater than for the parent drug over the 24-hr period, but it is most likely greater due to its higher concentration at 24 hr post-dose. AUC_{0-∞} for DOV 216,298 is not reported since the calculated values of AUC_{24-∞} accounted for 38% and 54% of the total exposure. Accurate calculation of the lactam's t½ and AUC_{0-∞} will require the collection of samples at time points greater than 24 hr.

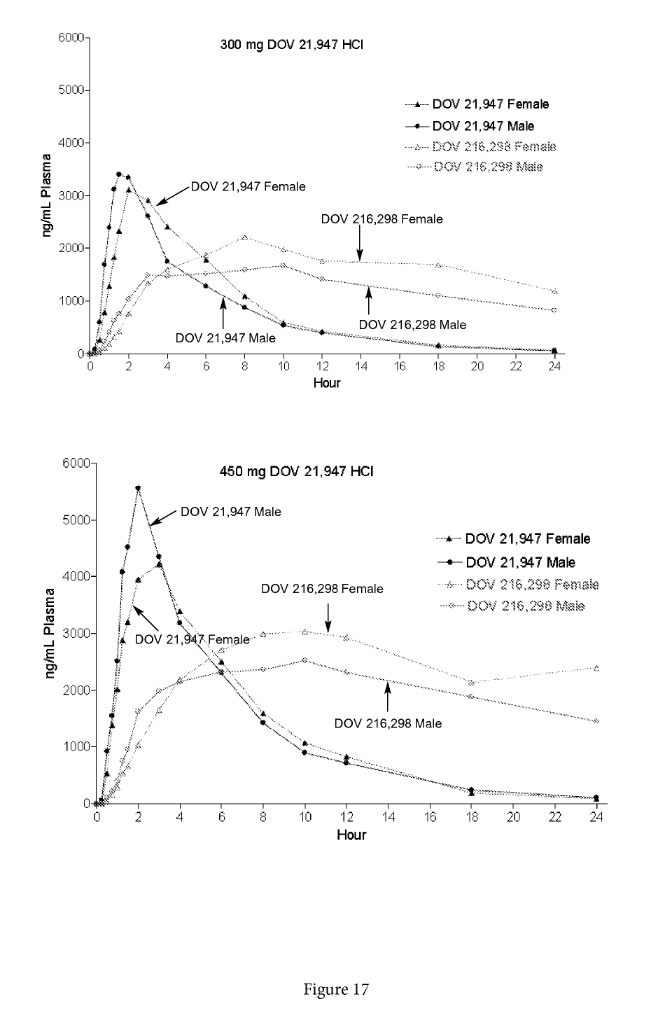

There were few differences in the pharmacokinetic parameters of either the parent drug or the lactam after either dose when the results were compared according to the sex of the subjects (Tables 39 and 40, FIG. 17). Cₘₐₓ, AUC, t_{1/2}, and CL/F were comparable between the two sexes. Tₘₐₓ for amitifadine was slightly longer in females than males at both dose levels. There were no obvious differences in the parameters after a single oral dose of amitifadine, but the low number of subjects per sex (N=5) precluded a robust statistical comparison.

TABLE 36
Mean Pharmacokinetic Parameters (SD) of DOV 21,947
and the Lactam Metabolite DOV 216,298 in Healthy Adult
Subjects Following a Single Oral Dose of 300 mg
or 450 mg DOV 21,947 HCl Immediate Release Formulation
(Cohort 1)

| | | |
|---|---|---|
| Parameter (units) | 300 mg^{a} | 450 mg^{a} |

DOV 21,947

| | | | | |
|---|---|---|---|---|
| Cₘₐₓ (ng/mL) | 3372 | (970) | 5110 | (1410) |
| Tₘₐₓ (hr) | 2.15 | (0.91) | 2.43 | (0.80) |
| AUC₀₋ₜ (ng · hr/mL) | 19,368 | (3549) | 29,991 | (6148) |
| AUC_{0-∞} (ng · hr/mL) | 19,735 | (3626) | 30,525 | (6306) |
| λ_{z} (hr⁻¹) | 0.165 | (0.018) | 0.184 | (0.021) |
| t½ (hr) | 4.24 | (0.43) | 3.82 | (0.51) |
| t½ (hr)^{c} | 4.20 | — | 3.77 | — |
| CL/F (L/hr) | 13.6 | (29.5) | 13.2 | (2.89) |
| V_{z}/F (L ) | 82.6 | (16.1) | 72.6 | (16.3) |
| MRT_{0-∞} (hr) | 6.29 | (0.58) | 6.39 | (0.74) |

DOV 216,298

| | | | | |
|---|---|---|---|---|
| Cₘₐₓ (ng/mL) | 2031 | (655) | 2954 | (874) |
| Tₘₐₓ (hr) | 7.80 | (4.34) | 9.00 | (1.94) |
| AUC₀₋ₜ (ng · hr/mL) | 33,268 | (10,306) | 50,718 | (15,713) |
| λ_{z} (hr⁻¹) | 0.049^{b} | (0.016) | 0.035 | (0.020) |
| t½ (hr) | 15.4^{b} | (4.04) | 32.6 | (29.8) |
| t½ (hr)^{c} | 14.3^{b} | — | 19.8 | — |

^{a}N = 10
^{b}N = 9, value not calculated for one subject
^{c}Harmonic mean

TABLE 37
Mean Pharmacokinetic Parameters (SD) of DOV 21,947 and the Lactam
Metabolite DOV 216,298 in Healthy Adult Male and Female Subjects
Following a Single Oral Dose of 300 mg DOV 21,947 HCl Immediate
Release Formulation (Cohort 1)

| | | |
|---|---|---|
| Parameter (units) | Male^{a} | Female^{a} |

DOV 21,947

| | | | | |
|---|---|---|---|---|
| Cₘₐₓ (ng/mL) | 3484 | (773) | 3261 | (1220) |
| Tₘₐₓ (hr) | 1.60 | (0.38) | 2.70 | (0.97) |
| AUC₀₋ₜ (ng · hr/mL) | 18,594 | (3603) | 20,143 | (3724) |
| AUC_{0-∞} (ng · hr/mL) | 18,951 | (3671) | 20,519 | (3818) |
| λ_{z} (hr⁻¹) | 0.161 | (0.015) | 0.169 | (0.021) |
| t½ (hr) | 4.34 | (0.40) | 4.14 | (0.48) |
| t½ (hr)^{c} | 4.31 | — | 4.10 | — |
| CL/F (L/hr) | 14.2 | (3.38) | 13.0 | (2.71) |
| V_{z}/F (L) | 88.6 | (20.0) | 76.7 | (9.88) |
| MRT_{0-∞} (hr) | 6.07 | (0.47) | 6.51 | (0.65) |

DOV 216,298

| | | | | |
|---|---|---|---|---|
| Cₘₐₓ (ng/mL) | 1737 | (440) | 2325 | (746) |
| Tₘₐₓ (hr) | 6.40 | (3.51) | 9.20 | (5.02) |
| AUC₀₋ₜ (ng · hr/mL) | 29,208 | (7186) | 37,327 | (12,089) |
| λ_{z} (hr⁻¹) | 0.051 | (0.020) | 0.046^{b} | (0.010) |
| t½ (hr) | 15.3 | (5.18) | 15.5^{b} | (2.78) |
| t½ (hr)^{c} | 13.7 | — | 15.0 | — |

^{a}N = 5
^{b}N = 4, value not calculated for one female subject.
^{c}Harmonic mean

TABLE 38
Mean Pharmacokinetic Parameters (SD) of DOV 21,947 and the Lactam
Metabolite DOV 216,298 in Healthy Adult Male and Female Subjects
Following a Single Oral Dose of 450 mg DOV 21,947 HCl Immediate
Release Formulation (Cohort 1)

| | | |
|---|---|---|
| Parameter (units) | Male^{a} | Female^{a} |

DOV 21,947

| | | | | |
|---|---|---|---|---|
| Cₘₐₓ (ng/mL) | 5675 | (1354) | 4544 | (1356) |
| Tₘₐₓ (hr) | 1.85 | (0.34) | 3.00 | (0.71) |
| AUC₀₋ₜ (ng · hr/mL) | 30,511 | (5047) | 29,470 | (7674) |
| AUC_{0-∞} (ng · hr/mL) | 31,128 | (5250) | 29,922 | (7810) |
| λ_{z} (hr⁻¹) | 0.173 | (0.024) | 0.194 | (0.012) |
| t½ (hr) | 4.06 | (0.53) | 3.58 | (0.22) |
| t½ (hr)^{b} | 4.00 | — | 3.57 | — |
| CL/F (L/hr) | 12.8 | (2.28) | 13.7 | (3.61) |
| V_{z}/F (L) | 74.3 | (13.5) | 70.9 | (20.1) |
| MRT_{0-∞} (hr) | 6.35 | (0.42) | 6.42 | (1.03) |

DOV 216,298

| | | | | |
|---|---|---|---|---|
| Cₘₐₓ (ng/mL) | 2550 | (468) | 3358 | (1044) |
| Tₘₐₓ (hr) | 9.20 | (1.79) | 8.80 | (2.28) |
| AUC₀₋ₜ (ng · hr/mL) | 46,146 | (8429) | 55,290 | (20,789) |
| λ_{z} (hr⁻¹) | 0.042 | (0.022) | 0.028 | (0.018) |
| t½ (hr) | 23.4 | (16.8) | 41.8 | (38.9) |
| t½ (hr)^{b} | 16.6 | — | 24.5 | — |

^{a}N = 5
^{b}Harmonic mean

The mean amounts and percentage of the dose excreted into urine as amitifadine and DOV 216,298 in the first 24 hr following administration of the dose are shown in Tables 39 and 40.

TABLE 39
Mean Amounts of DOV 21,947 and the Lactam Metabolite DOV 216,298 in
Urine (0-24 hr) of Healthy Adult Subjects Following a Single Oral Dose of
300 mg or 450 mg DOV 21,947 HCl Immediate Release Formulation
(Cohort 1)

| | | | | |
|---|---|---|---|---|
| Period (hr) | μg DOV 21,947 | (SD) | μg DOV 216,298 | (SD) |

300 mg

| | | | | |
|---|---|---|---|---|
| 0-1 | 17.0 | (11.2) | 0.0 | (0.0) |
| 1-2 | 115 | (125) | 2.7 | (4.9) |
| 2-4 | 279 | (160) | 52.5 | (42.9) |
| 4-8 | 371 | (134) | 56.2 | (22.3) |
| 8-12 | 137 | (68.7) | 34.8 | (23.0) |
| 12-24 | 115 | (61.4) | 47.8 | (21.6) |
| Total | 1064 | (313) | 194 | (61.5) |

450 mg

| | | | | |
|---|---|---|---|---|
| 0-1 | 9.4 | (10.0) | 0.0 | (0.0) |
| 1-2 | 66.3 | (53.3) | 2.1 | (4.2) |
| 2-4 | 322 | (183) | 45.2 | (43.1) |
| 4-8 | 402 | (192) | 89.3 | (76.9) |
| 8-12 | 195 | (233) | 61.2 | (43.7) |
| 12-24 | 164 | (84.9) | 63.5 | (50.8) |
| Total | 1159 | (553) | 261 | (134) |

TABLE 40
Mean Percent of Dose in Urine (0-24 hr) as DOV 21,947 and the
Lactam Metabolite DOV 216,298 in Healthy Adult Subjects
Following a Single Oral Dose of 300 mg or 450 mg
DOV 21,947 HCl Immediate Release Formulation (Cohort 1)

| | | | | |
|---|---|---|---|---|
| | | | | Ratio |
| Subject | | | | DOV 216,298/ |
| (Sex) | DOV 21,947 | DOV 216,298 | Combined | DOV 21,947 |

300 mg

| | | | | |
|---|---|---|---|---|
| 1001 (M) | 0.681 | 0.082 | 0.763 | 0.120 |
| 1002 (M) | 0.406 | 0.087 | 0.496 | 0.215 |
| 1003 (M) | 0.375 | 0.066 | 0.441 | 0.175 |
| 1004 (M) | 0.323 | 0.051 | 0.374 | 0.156 |
| 1006 (M) | 0.436 | 0.042 | 0.478 | 0.096 |
| 1008 (F) | 0.289 | 0.124 | 0.413 | 0.429 |
| 1010 (F) | 0.506 | 0.081 | 0.587 | 0.161 |
| 1011 (F) | 0.273 | 0.052 | 0.325 | 0.191 |
| 1012 (F) | 0.356 | 0.085 | 0.441 | 0.240 |
| 1014 (F) | 0.462 | 0.078 | 0.540 | 0.169 |
| Mean (SD) | 0.411 (0.121) | 0.075 (0.024) | 0.486 (0.124) | 0.195 (0.092) |

450 mg

| | | | | |
|---|---|---|---|---|
| 1001 (M) | 0.669 | 0.064 | 0.733 | 0.096 |
| 1002 (M) | 0.314 | 0.087 | 0.401 | 0.278 |
| 1003 (M) | 0.294 | 0.037 | 0.331 | 0.176 |
| 1004 (M) | 0.362 | 0.052 | 0.414 | 0.145 |
| 1006 (M) | 0.217 | 0.028 | 0.245 | 0.129 |
| 1008 (F) | 0.096 | 0.121 | 0.317 | 0.615 |
| 1010 (F) | 0.229 | 0.108 | 0.338 | 0.473 |
| 1011 (F) | 0.242 | 0.048 | 0.290 | 0.197 |
| 1012 (F) | 0.293 | 0.101 | 0.394 | 0.345 |
| 1014 (F) | 0.172 | 0.027 | 0.199 | 0.158 |
| Mean (SD) | 0.299 (0.143) | 0.067 (0.035) | 0.366 (0.146) | 0.256 (0.173) |

M = Male, F = Female

Less than 0.5% of the administered drug was excreted into urine as either the unchanged drug or the lactam within 24 hr of ingesting either 300 mg or 450 mg amitifadine (Tables 39 and 40). Urinary amitifadine accounted for 0.4% and 0.3% of the ingested dose at the 300 mg and 450 mg dose level, respectively, and the lactam accounted for less (0.075% and 0.067%, respectively). Based on its plasma concentrations at 24 hr, it would be expected that urinary excretion of the lactam would be appreciably greater if urine was collected for a longer time period. Renal clearance of amitifadine was 56.6 mL/hr at the 300 mg dose level and 38.8 mL/hr at the 450 mg dose level as shown in Table 41.

TABLE 41
Renal Clearance (mL/hr) of DOV 21,947 in Healthy Adult Subjects
Following a Single Oral Dose of 300 mg or 450 mg DOV 21,947
HCl Immediate Release Formulation (Cohort 1)

| Subject (Sex) | 300 mg | 450 mg |
|---|---|---|
| 1001 (M) | 91.7 | 79.2 |
| 1002 (M) | 47.3 | 33.7 |
| 1003 (M) | 76.4 | 47.3 |
| 1004 (M) | 40.6 | 42.2 |
| 1006 (M) | 62.1 | 31.9 |
| 1008 (F) | 32.1 | 25.4 |
| 1010 (F) | 62.1 | 22.3 |
| 1011 (F) | 47.7 | 40.1 |
| 1012 (F) | 39.2 | 34.1 |
| 1014 (F) | 66.6 | 32.1 |
| Mean (SD) | 56.6 (18.6) | 38.8 (16.0) |

### Example XLIII

### Single Dose Study of Amitifadine (EB-1010) in 24 Men

This was a single-dose (25 mg), open-label, randomized, 2-period, crossover study to assess the effect of food (fed vs. fasted state) on the PK profile of amitifadine (EB-1010) in 24 young, healthy men. As seen in Table 42, Cₘₐₓ was slightly lower and delayed when amitifadine (EB-1010) was given with food. Both AUC₀₋ₜ and AUC_{0-∞} were within the 90% confidence limits (80-125%), indicating no effect of food on exposure to EB-1010.

TABLE 42
Mean (±SD) Pharmacokinetic Parameters of EB-1010 in
24 Healthy Male Subjects following a Single Oral Dose (25 mg)
under Fed and Fasted Conditions: Study DOV 947-003

| Parameter | Fasted | | Fed | | 90% Cl |
|---|---|---|---|---|---|
| Cₘₐₓ (ng/mL) | 272 | (85) | 224 | (79) | 72-92 |
| Tₘₐₓ (h) | 0.98 | (0.54) | 2.57 | (0.97) | 220-213 |
| AUC₀₋ₜ (ng · hr/mL) | 1195 | (321) | 1316 | (382) | 104-119 |
| AUC_{0-∞} (ng · hr/mL) | 1292 | (311) | 1425 | (358) | 105-115 |
| t_{1/2} (h) | 3.42 | (0.57) | 3.71 | (0.76) | 100-118 |

There were no deaths or SAEs in this study. One subject withdrew from the study due to AEs; 5 AEs in 3 subjects were reported. One subject had 2 episodes of dysphoria and 1 episode of an unpleasant dream that developed more than 12 hours. Two subjects had insomnia; 1 subject each had nausea, headache, and redness at the catheter insertion site. All AEs were mild, except for 1 episode of dysphoria (associated with nightmares), which was moderate in severity and which occurred approximately 12 hours after administration of the study drug. The subject discontinued the study due to the dysphoria and nightmares. All AEs resolved without treatment. There were no clinically significant laboratory, vital sign, or ECG abnormalities.

### Example XLIV

### Single-Dose Study of Amitifadine (EB-1010) in Males and Females

This study was an open-label, single-dose (25 mg) gender study of the safety, tolerability, and pharmacokinetics of amitifadine (EB-1010) in 70 healthy, young male and female subjects. There was no difference in either Cₘₐₓ or AUC values between the two sexes based on the 90% CI (80-125%).

TABLE 43
Mean (±SD) Pharmacokinetic Parameters of EB-1010 in
Healthy Male and Female Subjects Following a Single
Oral Dose (25 mg): Study DOV-947-005

| | Males | | Females | | |
|---|---|---|---|---|---|
| Parameter | (N = 32) | | (N = 38) | | 90% Cl |
| Cₘₐₓ (ng/mL) | 273 | (66) | 291 | (67) | 97-118 |
| Tₘₐₓ (h) | 1.27 | (0.39) | 1.38 | (0.48) | 95-123 |
| AUC₀₋ₜ (ng · hr/mL) | 1357 | (320) | 1491 | (389) | 99-121 |
| AUC_{0-∞} (ng · hr/mL) | 1442 | (323) | 1589 | (377) | 100-121 |
| t_{1/2} (h) | 3.41 | (0.76) | 3.73 | (0.75) | 101-119 |

There were no deaths or SAEs in this study. There was 1 mild episode of gastroesophageal reflux, 1 episode of mild abdominal bloating, and 1 transient, moderate vasovagal episode. The vasovagal episode was judged by the investigator as definitely not study drug-related because it occurred before study medication dosing. There were no clinically significant abnormal laboratory test values, vital sign changes, or ECG abnormalities.

A formal analysis of QTc at the protocol-specified time points was performed in this study, and no sustained or significant prolongation of QTc either in individual subjects or overall was noted.

Orthostatic hypotension (defined as a decrease of 20 mmHg in systolic or 10 mmHg in diastolic pressure upon moving from a supine to a standing position), was observed in a total of 21 (30%) subjects [10 (31.3%) males and 11 (28.9%) females]. Supine and standing blood pressures were obtained at baseline (pre-dose) and at 0.5, 1, and 2 hours. Orthostatic changes in blood pressure for these subjects occurred at only 1 time point, either at baseline or at one of the time points, except in 1 male subject who had occurrences at pre-dose and 1 hour. Of the 21 subjects, 16 (9 males and 7 females) had orthostatic changes at only 1 time point (at 0.5 or 1 hour in the male subjects, and at 0.5 or 2 hours in the female subjects). Four of the 21 subjects had orthostatic changes only at baseline (pre-dose). The fasting and hydration status of the subjects may have been a contributing factor in these observed changes. The significance of this finding is not clear. Orthostatic hypotension was not observed in other completed studies.

### Example XLV

### Open Label Study Using PET

This was a Phase 1, single-dose, randomized, open-label study using positron emission tomography (PET) and [11C]DASB ([11C]N,N-dimethyl-2-(2-amino-4-cyanophenylthio) benzylamine) as a PET tracer in 3 healthy, young, adult male volunteers to determine the level of occupancy of serotonin transporters (SERT) in the human brain following administration of EB-1010. With PET imaging, uptake inhibitor effects may be measured based on the proportion of SERT sites blocked in the brain. (Only the first phase of the study was conducted, because the criteria for the subsequent 2 phases were not met [receptor occupancy of ≧75% at trough plasma levels].) PET scans were done at baseline and at 2 and 7-hour via measurement of [¹¹C]DASB tracer binding. Periodic blood samples were collected for evaluation of amitifadine (EB-1010) PK. Safety was monitored by clinical and laboratory evaluations, including vital signs, physical examination, clinical chemistry, hematology, urinalysis, and ECG.

It was concluded that 1) brain SERT occupancy after administration of a single oral dose of amitifadine (EB-1010) 150 mg, as assessed by PET at approximately 2 and 7 hours after dosing, is approximately 48% and 32%, respectively; 2) mean plasma concentrations of amitifadine (EB-1010) at 2 and 7 hours after administration of a single oral dose 150 mg is approximately 1,107 ng/mL and 405 ng/mL, respectively.

TABLE 44
Preliminary Mean Plasma Concentration and
Mean SERT Occupancy at about 2 and 7 hours
after Administration of EB-1010 150 mg: Study DOV 947-007

| | | Mean (Range) | Mean (Range) | | |
|---|---|---|---|---|---|
| PET | | Plasma | | | |
| Scan | | Concentration | SERT Occupancy (%) (N = 3) | | |
| Time | | (ng/mL) | Thalamus | Striatum | Mean |
| ~2 | h | 1107 (1044-1192) | 48 (41-55) | 48 (44-53) | 48 (43-54) |
| ~7 | h | 405 (361-483) | 33 (22-44) | 32 (23-37) | 32 (23-41) |

As shown in Table 44, at approximately 2 hours after dosing, the mean SERT occupancy was approximately 48%. At approximately 7 hours after dosing, the mean SERT occupancy was approximately 32%. No serious AEs were reported, and no subjects were discontinued from the study due to a clinical or laboratory adverse experience. One subject reported a total of 5 AEs, all of which were rated by the investigator as definitely not related to study drug. Other safety evaluations, such as physical examinations and ECGs, revealed no clinically meaningful changes from pre-dose evaluations. No dose-related changes in vital sign measurements (semi-recumbent blood pressure, pulse rate, respiratory rate, and oral temperature) were noted. There were no clinically meaningful changes in the laboratory safety tests.

There were no deaths, SAEs, or dropouts due to AEs. No treatment-emergent adverse events (TEAEs) were reported. There were no clinically significant changes in clinical laboratory parameters, vital signs, physical examination, or ECG.

### Example XLVI

### Multi-Dose Study Evaluating the Safety and PK of EB-1010

This was an escalating, multiple-dose study designed to evaluate the multiple-dose safety and PK of amitifadine (EB-1010) 25 mg BID and 50 mg BID for 10 days in healthy, young men. Twenty-two male subjects were randomized for the study and received study medication. A total of 20 subjects completed the study according to the Protocol.

The drug accumulation at 25 and 50 mg doses was 1.27 and 1.44, respectively (Table 43). The t_{1/2} at both doses remained constant over the 10 days.

TABLE 45
Mean (±SD) Pharmacokinetic Parameters of EB-1010
in Healthy Male Subjects on a Multiple Dose
Regimen: Study DOV-941-002

| | 25 mg BID | | 50 mg BID | |
|---|---|---|---|---|
| Parameter | Day 1 | Day 10 | Day 1 | Day 10 |
| Cₘₐₓ (ng/mL) | 327 (104) | 388 (94) | 496 (62) | 744 (223) |
| Tₘₐₓ (h) | 0.90 (0.28) | 1.07 (0.31) | 1.04 (0.34) | 1.18 (0.51) |
| AUC₀₋₁₂ | 1361 (255) | 1700 (206) | 2235 (355) | 3197 (477) |
| (ng · h/mL) | | | | |
| t_{1/2} (h) | 3.27 (0.40) | 3.34 (0.29) | 3.19 (0.40) | 2.97 (0.68) |
| R (AUC₀₋ₜ | — | 1.27 (0.15) | — | 1.44 (0.12) |
| Day10/Day 1) | | | | |

There were no deaths or SAEs. Two subjects discontinued prematurely: one subject (25 mg BID) discontinued after receiving 10 doses of study medication due to an erythematous maculopapular rash associated with pruritus over most of his body. This was thought to be an allergic reaction to study drug; the second subject (50 mg BID) withdrew by his own request after receiving 3 doses of study drug. There were a total of 12 AEs in 7 subjects dosed with 25 mg BID; 3 AEs in 3 subjects dosed with 50 mg BID; and 3 AEs in 1 subject who received placebo. All events were mild to moderate in severity. The most common AEs were vasovagal events (3 subjects, all in the 25 mg BID group) and diarrhea (3 subjects, all in the 25 mg BID group). Other AEs included 2 subjects with headache; 1 with erythematous maculopapular rash, pruritus, and lightheadedness; 1 with mouth ulcer and rhinitis; and 1 each with postural dizziness, venipuncture hematoma, epistaxis, and pharyngitis. All but 1 AE (mouth ulcers) resolved without treatment. There were no clinically significant laboratory, vital sign, or ECG abnormalities.

### XLVII

### Safety Study of Amitifadine (EB-1010) in Humans

This was a multiple-dose, randomized placebo-controlled, double-blind study to assess the safety of amitifadine (EB-1010) in healthy adult male and female human subjects with BMI between 25 and 35. This Phase 1b study enrolled 46 male and female subjects. Following a 1-week placebo run-in, subjects received 8 weeks of treatment of either placebo (15 subjects) or amitifadine (EB-1010) (31 subjects, 25 mg BID for 2 weeks, then 50 mg BID for 2 weeks and 75 mg BID for 4 weeks). The study demonstrated that amitifadine (EB-1010) was safe and well-tolerated in this dose range with no reported SAEs. The proportion of subjects with TEAEs was similar in the 2 treatment groups, with 36% and 47% in the amitifadine (EB-1010) and the placebo treated group, respectively (see Table 46). Reported AEs with greater than 3% incidence in both the amitifadine (EB-1010) and placebo-treated arms included headache, nausea, diarrhea, and dizziness. No other reported AE with a greater than 5% incidence was observed in the EB-1010-treated subjects.

TABLE 46
Adverse Events by Treatment Group and
Preferred Term (Safety Population)

| | Total Number of Subjects with at Least One AE | | |
|---|---|---|---|
| MedDRA | EB-1010 | Placebo | Total |
| Preferred Term | (N = 31) | (N = 15) | (N = 46) |
| | 11 (35.5%) | 7 (46.7%) | 18 (39.1%) |
| Headache | 7 (22.6%) | 1 (6.7%) | 8 (17.4%) |
| Nausea | 5 (16.1%) | 1 (6.7%) | 6 (13.0%) |
| Diarrhea | 2 (6.5%) | 2 (13.3%) | 4 (8.7%) |
| Dizziness | 2 (6.5%) | 2 (13.3%) | 4 (8.7%) |
| Abdominal Pain Lower | 0 (0.0%) | 1 (6.7%) | 1 (2.2%) |
| Abdomibal Pain Upper | 0 (0.0%) | 1 (6.7%) | 1 (2.2%) |
| Gastroesophageal | 0 (0.0%) | 1 (6.7%) | 1 (2.2%) |
| Reflux Disease | | | |
| Toothache | 1 (3.2%) | 0 (0.0%) | 1 (2.2%) |
| Increased Appetite | 1 (3.2%) | 0 (0.0%) | 1 (2.2%) |
| Syncope | 0 (0.0%) | 1 (6.7%) | 1 (2.2%) |
| Metrorrhagia | 1 (3.2%) | 0 (0.0%) | 1 (2.2%) |
| Rhinitis Allergic | 1 (3.2%) | 0 (0.0%) | 1 (2.2%) |
| Hyperhidrosis | 1 (3.2%) | 0 (0.0%) | 1 (2.2%) |
| Pruritus | 1 (3.2%) | 0 (0.0%) | 1 (2.2%) |
| Skin Rash | 1 (3.2%) | 0 (0.0%) | 1 (2.2%) |

* One subject who developed a skin reaction during the placebo run-in phase, before randomization, was not included in this table.
N = Number of subjects with AEs.

One subject developed skin rash during the placebo run-in week. The skin rash occurred on the 3^{rd} day after placebo treatment and was reported as mild, erythematous, macules throughout the body, especially on arms, legs, and chest. The skin reaction was resolved by oral Benadryl (12.5 mg PRN) treatment.

One subject in the amitifadine (EB-1010) group developed skin rash. The skin rash occurred on the 10^{th} day after amitifadine (EB-1010) treatment (at the 25 mg BID phase) and was reported as mild, erythematous macules throughout the body, especially on bilateral thighs that blanched, no warmth, no edema, and no petechiae. The skin reaction was treated with oral Benadryl (25 mg PRN) and hydrocortisone cream and was resolved in 7 days.

The incidence of subjects who dropped out from the study due to AEs was 3.2% and 13.3% in the amitifadine (EB-1010) and placebo-treated group, respectively (see Table 47).

TABLE 47
Premature Discontinuations
Randomized Population

| | EB-1010 | Placebo | Total |
|---|---|---|---|
| | (N = 31) | (N = 15) | (N = 46) |
| | n (%) | n (%) | n (%) |
| Completed Subjects | 23 (74.2%) | 11 (73.3%) | 34 (73.9%) |
| Withdrawal/Early | 8 (25.8%) | 4 (26.7%) | 12 (26.1%) |
| Termination | | | |
| Allergic Reaction to | 1 (3.2%) | 0 (0.0%) | 1 (2.2%) |
| Study Medication | | | |
| Adverse Event | 1 (3.2%) | 2 (13.3%) | 3 (6.5%) |
| Lost to Follow Up | 1 (3.2%) | 1 (6.7%) | 2 (4.3%) |
| Subject Requested | 2 (6.5%) | 1 (6.7%) | 3 (6.5%) |
| Withdrawal | | | |
| Other | 3 (9.7%) | 0 (0.0%) | 3 (6.5%) |

Note:
N = Number of Randomized subjects.
n = Number of subjects within a specific category.
In case of multiple reasons for discontinuation, the primary reason for discontinuation is established in the following order: Allegeric Reaction to Study Medication, Adverse Event, Clinically Significant Laboratory Abnormality, ECG Abnormalities, Intercurrent Illness, Lost to Follow Up, Subject Requested Withdrawal, Protocol Violation, Request of Investigator or Sponsor, Other (Specify).

In addition, preliminary analysis of the clinical chemistry laboratory data indicates that EB-1010-treated subjects had lowered plasma triglyceride levels compared to placebo-treated subjects (p<0.015). This reduction in mean triglyceride levels was noted following 2 weeks of treatment (˜23% reduction), was maintained at the end of the amitifadine (EB-1010) treatment period (˜29% reduction) and was reversed after the 1-week washout period at the end of the study.

### Example XLVIII

### Efficacy of (+)-1-(3,4-dichlorophenyl)-3-azabicyclo[3.1.0]hexane in the Treatment of patients with Major Depressive Disorder

Subjects were identified who were between the ages of 18-65 (inclusive), and met criteria for Major Depressive Disorder in accordance with the Diagnostic and Statistical manual of Mental Disorders-IV-TR and confirmed by the MINI International Neuropsychiatric Interview. At the screening visit, subjects had a baseline Hamilton Depression Rating Scale (HAMD-17)≧22 and a severity of ≧2 on item 1 and a rating on the Hamilton Anxiety Scale (HAM-A)<17. They were also required to have a BMI≦35 and body weight >45 kg at the Screening Visit.

They were excluded if they were judged to be a suicide risk, known to be antidepressant treatment resistant or had other major clinically significant medical and/or other psychiatric illnesses such as panic disorder, social phobia, generalized anxiety disorder, obsessive compulsive disorder, post-traumatic stress disorder, acute stress disorder, substance abuse, anorexia, bulimia, antisocial personality disorder or bipolar disorder. Additionally, subjects who had a HAMD-17 reduction in score of more than 15% between the Placebo run-in visit and the baseline visit were eliminated.

Subjects were required to refrain from taking antidepressants, anticonvulsants including gabapentin and pregabalin, neuroleptics, MAO inhibitors, barbiturates, benzodiazepines, stimulants, antipsychotics, lithium, anxiolytics and beta blockers starting two weeks prior to the study and continuing until after the follow-up visit.

Subjects were evaluated for safety parameters prior to and throughout the trial by a variety of measures including electrocardiogram, physical examination, vital signs and body weight, and clinical laboratory testing including a lipid panel, CBC with differential and urinalysis. Samples were drawn to assess total bilirubin, alkaline phosphatase, ALT (SGPT), AST (SGOT), blood urea nitrogen (BUN), creatinine, glucose, uric acid, calcium, phosphorus, total protein, albumin, total cholesterol, LDL, HDL, triglycerides, sodium, potassium, bicarbonate, chloride, GGT and creatine kinase, Hepatitis B, C and HIV serologies, TSH, drug screen and serum pregnancy test for females. Sixty-three eligible subjects were identified who were not eliminated by the safety parameters. These sixty-three subjects had the following combined (placebo and EB-1010) mean baseline scores on the main outcome measures: MADRS (31.4) (primary); HAMD-17 (29.6) (secondary); and DISF-SR (25.38). The sixty-three subjects were randomized to receive either 25 mg of (+)-1-(3,4-dichlorophenyl)-3-azabicyclo[3.1.0]hexane twice a day for two weeks and then 50 mg twice a day for four weeks or placebo according to the following schedule:

TABLE 48
Titration schedule

| | | Study Medication Dispense Visit | | | | | |
|---|---|---|---|---|---|---|---|
| | | | | | | Visit 7 | |
| | | Visit 3: | Visit 4 | Visit 5 | Visit 6 | (Day 29 ± 2)** | Visit 8/EOT |
| | | Baseline/Day 1 | (Day 8 ± 2) | (Day 15 ± 2) | (Day 22 ± 2) | (Visit 7-1 and | (Day 43 ± 2) |
| Study Groups | | (Visit 3 Blister) | (Visit 4 Blister) | (Visit 5 Blister) | (Visit 6 Blister) | Visit 7-2 Blisters) | (Visit 8 Blister) |
| Placebo | Morning | 2 Placebo | 2 Placebo | 2 Placebo | 2 Placebo | 2 Placebo | 2 Placebo |
| | Dose | Capsules | Capsules | Capsules | Capsules | Capsules | Capsules |
| | Evening | 2 Placebo | 2 Placebo | 2 Placebo | 2 Placebo | 2 Placebo | 2 Placebo |
| | Dose | Capsules | Capsules | Capsules | Capsules | Capsules | Capsules |
| Amitifadine | Morning | 25 mg | 25 mg | 25 mg | 25 mg | 25 mg | 2 Placebo |
| | Dose | Capsule: 1 | Capsule: 1 | Capsule: 2 | Capsule: 2 | Capsule: 2 | Capsules |
| | | Placebo | Placebo | | | | |
| | | Capsule: 1 | Capsule: 1 | | | | |
| | Evening | 25 mg | 25 mg | 25 mg | 25 mg | 25 mg | 2 Placebo |
| | Dose | Capsule: 1 | Capsule: 1 | Capsule: 2 | Capsule: 2 | Capsule: 2 | Capsules |
| | | Placebo | Placebo | | | | |
| | | Capsule: 1 | Capsule: 1 | | | | |

Visits and evaluations were performed according to the following schedule of events:

### Visit 1: Screening Visit:

The following was obtained/performed at the Screening Visit (Visit 1):

Written informed consent

Medical history including:
Relevant demographic information
Detailed medical and surgical history, including review of systems
   Whenever possible, the patient's medical history was confirmed by medical records.
Prior medication: Medication taken by the patients 30 days prior to the Screening Visit was recorded.
AE assessment
Height (cm)
Weight (kg); BMI was determined and was ≦35 for the patient to be randomized
Complete physical examination
MINI diagnostic exam
Vital signs (respiratory rate, oral temperature (° C.), blood pressure, pulse). Blood pressure and pulse was measured twice: supine, after resting supine for at least 5 min and then at least 2 min but less than 3 min after standing up.
Fasted clinical laboratory tests (chemistry, CBC with differential and urinalysis)
Hepatitis B, C and HIV serologies, TSH
Resting 12-lead ECG
Urine drug screen
Pregnancy test (females; serum)
Review of inclusion and exclusion criteria
HAM-A (a score <17 is required for enrollment)

### Visit 2: Placebo Run-in Visit:

The following procedures were performed:
Concomitant medication record
AE assessment
Review inclusion and exclusion criteria
HAMD-17: To be eligible for the study, the total HAMD-17 score must be ≧22 and the score on HAMD-17 item 1 must be ≧2.
Patients found to be eligible were dispensed a single blind placebo blister package (the Visit 2 blister). The capsules were taken for 7 days prior to the Baseline/Day 1 Visit (Visit 3). The first dose of placebo was taken at the clinic with 240 mL of water after a light meal.
Patients were provided with a diary to record the date, time and dosage of each dose.

Patient Medication Diary:

Patients were provided with a diary at the Placebo Run-In Visit (Visit 2) and at each subsequent visit except the last visit (the Follow-Up Visit, Visit 9). Patients recorded the date, time and dosage of each study medication dose using the diary. The diary was collected at the next scheduled visit, reviewed for dosing compliance, and a new diary dispensed.

TABLE 49
Schedule of Events After Screening

| | Visit 3/ | Visit 4/ | Visit 5/ | Visit 6/ | Visit 7/ | Visit 8/ | Visit 9/Post |
|---|---|---|---|---|---|---|---|
| Procedure | Baseline | Week 2 | Week 3 | Week 4 | Week 5 | Week 6 | Treatment |
| Day | | 8 ± 2 | 15 ± 2 | 22 ± 2 | 29 ± 2 | 43 ± 2 | 50 ± 2 |
| Vital Signs | X | | | | | | |
| Height | | | | | | X | |
| Weight | | | | | | X | |
| 12-lead ECG | X | | | | | | |
| Physical | X | X | X | X | X | X | X |
| Examination | | | | | | | |
| Concomitant | X | X | X | X | X | X | X |
| Medication | | | | | | | |
| Inclusion/ | X | | | | | | |
| Exclusion Criteria | | | | | | | |
| Fasted Lab | X (and lipid | | X | | X | X (and lipid | X |
| Work | profile) | | | | | profile) | |
| Collect blood | X | | X | | X | | |
| sample | | | | | | | |
| Collect Urine | X | X | | X | | X | |
| Sample | | | | | | | |
| Urine Drug | X | | | | | | |
| Screen | | | | | | | |
| Serum Pregnancy | X | | | | | X | X |
| (females only) | | | | | | | |
| HAMD-17 | X | | | | | | |
| MADRS | X | | | | | | |
| DISF-SR | X | | | | | | |
| CGI-S | X | | | | | | |
| Review Inclusion/ | X | | | | | | |
| Exclusion Criteria | | | | | | | |
| Adverse Event | X | X | X | X | X | X | |
| Assessment | | | | | | | |
| Medication | X | X | X | X | X | X | |
| Dispensed | | | | | | | |
| Collect Diary | X | X | X | X | X | X | |

Post Dose

| | | | | | | | |
|---|---|---|---|---|---|---|---|
| Vital Signs | X | X | X | X | X | X | X |
| (1.5 hours | | | | | | | |
| after dosing) | | | | | | | |
| ECG-12 Lead | X | | X | | X | X | X |
| HAMD-17 | | X | X | X | X | X | X |
| CGI-I | | X | X | X | X | X | X |
| CGI-S | | X | X | X | X | X | X |
| DISF-SR | | | X | | X | X | X |
| MADRAS | | X | X | X | X | X | X |

Efficacy was determined by measuring the change from baseline in the Montgomery-Asberg Depression Rating Scale (MADRS), the HAMD-17, the Clinical Global Impression Global Improvement Scale (CGI-I), the Clinical Global Impression-Severity scale (CGI-S) and the Derogatis Interview for Sexual Functioning Self-Report (DISF-SR). Two analysis populations were studied: Modified Intent to Treat (MITT, N=56), defined as all randomized subjects with any confirmed dosing and MADRS data from at least one post-baseline visit (30 EB-1010-treated patients and 26 placebo-treated patients); and Completers (N=39), defined as the subset of MITT subjects who completed 6 weeks of treatment (20 EB-1010-treated patients and 19 placebo-treated patients). Comparisons between treatment groups based on MADRS (the primary efficacy parameter), HAMD-17, Anhedonia, DISF-SR, CGI-I and CGI-S scores were analyzed using a mixed-repeated measures (MMRM) analysis model including factors for Subject, Visit, Treatment Arm and Baseline value as a covariate. Adjusted least-squares means from these models are presented. Comparisons between groups were made at each post-baseline visit using model-based contrasts and adjusted degrees of freedom. For these analyses no explicit data imputations were made prior to the analysis. Response and remission categorical data were analyzed using chi-square tests. Inferential analyses of safety data were conducted with ANOVA models or chi-square tests. Two-tail alpha was set to 0.05. All analyses were conducted using SAS version 9.2.

The intent-to-treat (ITT) population (n=56) showed the following combined (placebo and (+)-1-(3,4-dichlorophenyl)-3-azabicyclo[3.1.0]hexane) mean baseline scores on the main outcome measures: MADRS (31.4) (primary); HAMD-17 (29.5) (secondary); and DISF-SR (25.8). As shown in FIG. 1, at the end of the double-blind treatment (Week 6), the estimated LS mean change from baseline (MMRM or mixed model repeated measures) in the MADRS total scores was statistically significantly superior for (+)-1-(3,4-dichlorophenyl)-3-azabicyclo[3.1.0]hexane when compared to placebo (18.16 vs 21.99; p=0.028), with an overall statistical effect size of −0.63 (Cohen's d). As shown in Table 49, when assessed with the CGI-I, a global impression scale sensitive to clinically relevant changes in improvement status, treatment with (+)-1-(3,4-dichlorophenyl)-3-azabicyclo[3.1.0]hexane was also statistically significantly superior to placebo (p=0.03; Week 6; MMRM). As shown in FIG. 6, an anhedonia factor score grouping Items 1 (apparent sadness), 2 (reported sadness), 6 (concentration difficulties), 7 (lassitude), and 8 (inability to feel) of the MADRS (analyzed using the mixed model for repeated measures LS means) demonstrated a statistically significant difference in favor of (+)-1-(3,4-dichlorophenyl)-3-azabicyclo[3.1.0]hexane in comparison to placebo (p=0.049). (+)-1-(3,4-dichlorophenyl)-3-azabicyclo[3.1.0]hexane was relatively well tolerated. Two patients in each treatment group discontinued the study early due to AEs but no serious AEs were reported.

TABLE 50
Least Square Adjusted Means with differences in Primary and
Secondary Efficacy Measures at Visit 8 (MMRM, MITT)

| | | (+)-1-(3,4- | | |
|---|---|---|---|---|
| | | dichlorophenyl)-3- | | |
| | Placebo | azabicyclo[3.1.0] | Difference | |
| Outcome | (n = 26) | hexane (n = 30) | (95% CI) | P value |
| MADRS (LS Mean - SE) | 21.99 (1.24) | 18.16 (1.21) | 3.83 (0.41, 7.26) | P = 0.028 |
| HAMD-17 (LS Mean - SE) | 18.02 (1.46) | 14.90 (1.40) | 3.12 (−0.87, 7.12) | P = 0.125 |
| Anhedonia factor (LS Mean - SE) | 9.33 (0.50) | 7.92 (0.50) | 1.41 (0.01, 2.82) | P = 0.049 |
| CGI-I (LS Mean - SE) | 2.75 (0.20) | 2.13 (0.20) | 0.62 (0.06, 1.18) | P = 0.030 |
| CGI-S (LS Mean - SE) | 3.53 (0.15) | 3.31 (0.15) | 0.22 (−0.21, 0.66) | P = 0.306 |

Abbreviations: MADRS, Montgomery Åsberg Depression Rating Scale; HAMD-17, Hamilton Rating Scale for Depression; CGI-I, Clinical Global Impressions-Improvement; CGI-S, Clinical Global Impressions-Severity; MMRM, Mixed Effect Models for Repeated Measures; MITT, Modified Intent-to-treat; CI, Confidence Interval, SE, Standard Error.

As shown in Table 51 and FIG. 5 (data analyzed using the last observation carried forward method), treatment with 100 mg of (+)-1-(3,4-dichlorophenyl)-3-azabicyclo[3.1.0]hexane was associated with significantly greater remission rates, defined by achieving a CGI-S score of ≦2, compared to placebo.

TABLE 51
Response and Remission Rates (Visit 8, LOCF, Completers)

| | | | | |
|---|---|---|---|---|
| | (+)-1-(3,4- | | | |
| | dichlorophenyl)- | | | |
| | 3-azabicyclo | | | |
| | [3.1.0]hexane | Placebo | Odds Ratio | |
| Outcome | 100 mg [n/N] (%) | [n/N] (%) | (95% CI) | P value |

Response

| | | | | |
|---|---|---|---|---|
| MADRS | (8/20) 40.00% | (3/19) 15.79% | 0.281 | 0.093 |
| | | | (0.061, 1.290) | |
| HAMD-17 | (11/20) 55.00% | (7/19) 36.84% | 0.477 | 0.256 |
| | | | (0.132, 1.721) | |

Remission

| | | | | |
|---|---|---|---|---|
| MADRS | (6/20) 30.00% | (2/19) 10.53% | 0.275 | 0.132 |
| | | | (0.048, 1.579) | |
| HAMD-17 | (4/20) 20.00% | (3/19) 15.79% | 0.750 | 0.732 |
| | | | (0.144, 3.904) | |
| CGI-S | (7/20) 35.00% | (1/19) 5.26% | 0.103 | 0.022 |
| | | | (0.011, 0.944) | |

Abbreviations: MADRS, Montgomery Åsberg Depression Rating Scale; HAMD-17, Hamilton Rating Scale for Depression; CGI-I, Clinical Global Impressions-Improvement; LOCF, Last Observation Carried Forward; Response, 50% reduction or more of the baseline total score of MADRS or HAMD-17 at endpoint; Remission, MADRS ≦ 12 or HAMD-17 ≦ 7 or CGI-S ≦ 2.

Additionally, unlike many antidepressants, as shown in FIG. 7, the DISF-SR scores stratified by low mean baseline scores (<25, indicating poor sexual function at baseline) versus high mean baseline scores (≧25, indicating preserved sexual function at baseline). In both the low baseline and high baseline groups, there are no differences between (+)-1-(3,4-dichlorophenyl)-3-azabicyclo[3.1.0]hexane 100 mg and placebo, indicating that treatment with (+)-1-(3,4-dichlorophenyl)-3-azabicyclo[3.1.0]hexane is not associated with emergence of sexual dysfunction. The efficacy of treatment with (+)-1-(3,4-dichlorophenyl)-3-azabicyclo[3.1.0]hexane was observed on the primary and secondary standard validated depression outcome measures (MADRS; global severity and improvement) as well as on the anhedonia factor of the MADRS. Furthermore, as shown in Tables 52 and 53, treatment with (+)-1-(3,4-dichlorophenyl)-3-azabicyclo[3.1.0]hexane was well tolerated and did not result in significant increases in heart rate, systolic or diastolic blood pressure compared to placebo. The number and percentage of patients who reported an adverse treatment event was similar between the two treatment groups (10 or 30.30% for amitifadine (EB-1010) ((+)-1-(3,4-dichlorophenyl)-3-azabicyclo[3.1.0]hexane) versus 11 or 39.28% for placebo).

TABLE 52
Treatment-Emergent Adverse Events* (% of Patients)

| | amitifadine | |
|---|---|---|
| | (EB-1010) | Placebo |
| | (n = 33) | (n = 28) |
| Headache NOS | 3 (9.09%) | 3 (10.71%) |
| Abdominal Pain (NOS) | 2 (6.06%) | 1 (3.57%) |
| Anxiety | 2 (6.06%) | 1 (3.57%) |
| Diarrhea NOS | 2 (6.06%) | 1 (3.57%) |
| Irritability | 2 (6.06%) | 1 (3.57%) |
| Nausea | 2 (6.06%) | 1 (3.57%) |
| Rash NOS | 2 (6.06%) | 1 (3.57%) |
| Upper Respiratory Tract Infection NOS | 2 (6.06%) | 1 (3.57%) |
| Emotional Disturbance NOS | 2 (6.06%) | 0 (0.00%) |

*Treatment-emergent adverse events defined as events reported by at least 5% of EB-1010- treated patients and at least twice the rate of placebo

TABLE 53
Changes From Baseline in Selected Vital Signs and
Laboratory Values at Visit 8, Safety Population (n = 61)

| | Amitifadine | | |
|---|---|---|---|
| | (EB-1010) | | |
| | (n = 33) | Placebo | |
| | Mean | (n = 28) | P value vs. |
| Assessment [Units] | Change | Mean Change | placebo |
| Systolic BP - Supine [mm Hg] | 2.58 | 2.28 | 0.904 |
| Diastolic BP - Supine [mm Hg] | −0.38 | −0.48 | 0.961 |
| Systolic BP - Standing (mm Hg) | 0.069 | 2.12 | 0.509 |
| Diastolic BP - Standing (mm Hg) | −3.00 | 2.80 | 0.017 |
| Supine Pulse [beats per minute] | 1.55 | −1.68 | 0.145 |
| Weight [kg] | 0.078 | 0.04 | 0.965 |
| Total Cholesterol Fasting | −5.86 | −11.36 | 0.412 |
| [mg/dL] | | | |
| LDL Cholesterol Fasting | −4.29 | −9.96 | 0.374 |
| [mg/dL] | | | |
| Triglycerides Fasting [mg/dL] | −12.00 | −7.80 | 0.750 |

Abbreviations: BP blood pressure; HDL high density lipoprotein; LDL low density lipoprotein; Safety population: All randomized patients who received study drug; P values were calculated by using ANOVA with treatment group as main effect

Additionally, treatment with (+)-1-(3,4-dichlorophenyl)-3-azabicyclo[3.1.0]hexane was not associated with significant weight gain or sexual dysfunction (See, for example, FIG. 7).

The results of this Phase 2 study demonstrated that EB-1010, at a titrated dose of 50 mg/day then 100 mg/day, was effective for treatment of patients with MDD. Efficacy was observed on the primary and secondary standard validated depression outcome measures (MADRS; global severity and improvement) as well as on the anhedonia factor of the MADRS. Overall, treatment with amitifadine (EB-1010) was well tolerated. The discontinuation rate due to AE was similar to placebo and treatment with amitifadine (EB-1010) was not associated with weight gain or sexual dysfunction.

### Example XLIX

### Caco-2 Bi-Directional Permeability Assay

The Caco-2 permeability of amitifadine is conducted by ADMETRx (Kalamazoo, Mich.). Aliquots of DMSO stock solution of drug are dissolved in Hanks Balanced Salt Solution containing 25 mM HEPES, pH 7.4 buffer with 0.05% polysorbate 80 to give target stock drug concentrations of 250 μM and 2.5% DMSO. Caco-2 cells are obtained from American Type Culture Collection (ATCC, Manassas, Va.) and are grown to confluence for 14-21 days on 1 μm filters in 24 well plates. Aliquots of drug are diluted in buffer to give 10 μM final concentration and <1% DMSO. The solutions are then transferred to either the apical or basolateral chamber of the permeability diffusion apparatus for incubation in an atmosphere of 95% air and 5% CO₂, relative humidity of 95%, and temperature of 37° C. Receiver solutions consisted of buffer only. Sequential samples of transported solute are taken at 20 minute intervals in duplicate over 2 hours and the concentration of the transported compound was determined by HPLC-UV/MS. Test sample concentration is determined by LC/MS. Permeability coefficients are calculated for each sampling interval, and averages from the intervals were determined. The apparent permeability coefficient Pₐₚₚ is calculated from the equation

$${P}_{app} = \frac{d Q}{d T} \times \frac{1}{A \times {C}_{0}} ,$$

where dQ/dt is the rate of permeation of drug across the cells, C₀ is the original concentration in donor compartment at time zero, and A is the area of the cell monolayer. Mass balance in the system is ascertained by comparing the sum of transported solute and remaining donor solute to the starting solution concentration. The integrity of the monolayers is verified by monitoring Lucifer yellow permeation and the known P-glycoprotein substrate Ac-D-Phe-(N-Me-D-Phe)₂-NH₂ is used as a positive control.

The bidirectional permeability of amitifadine across the Caco-2 monolayer is determined from 40 to 120 minutes. The result shows amitifadine (10 μM) is highly permeable in both the absorptive apical to basolateral (AP→BL) and the secretory basolateral to apical (BL→AP) directions with average apparent permeability coefficients of 69.2×10⁻⁶ cm/s and of 127×10⁻⁶ cm/s, respectively (Table 54). However, mass balance (total recovery) is only 62% in the AP→BL direction, which contributes to the lower permeability in the absorptive direction. It is not clear what contributes to the decreased recovery and, without being bound by theory, deviations from 100% recovery may be due to solute adsorption to the apparatus or monolayer, or chemical or metabolic instability during the course of the experiment. The p-glycoprotein substrate Ac-D-Phe-(N-Me-D-Phe)₂-NH₂ had BL→AP permeability 4 fold greater than in the AP→BL direction. The transfer of Lucifer yellow is ≦0.05%, which confirmed the integrity of the Caco-2 monolayer.

The data suggests that amitifadine has little propensity for active secretion. Since amitifadine is highly permeable and soluble, the oral absorption may not be a rate-limiting step and, thus, the oral bioavailability may be unlikely to be affected by other co-administered drugs.

TABLE 54
Bidirectional Permeability of Amitifadine (10 μM)
and Ac-D-Phe-(N-Me-D-PHe)₂-NH₂ (10 μM)
in the Absorptive Apical to Basolateral (AP→BL)
and Secretory Basolateral to Apical (BL→AP) Phases

| | PE_{AP→BL} | PE_{BL→AP} | |
|---|---|---|---|
| | 1 × 10⁻⁶ cm/s | 1 × 10⁻⁶ cm/s | |
| Compound | (Mass balance %) | (Mass balance %) | |
| Amitifadine | 69.2 (62%) | 127 | (88%) |
| Ac-D-Phe-(N-Me-D-Phe)₂-NH₂ | 18.8 (96%) | 82.8 | (104%) |

Values are average transport expressed as apparent permeability coefficient (PE) (1 × 10⁻⁶ cm/s) and mass balance in percent of total drug on both sides of membrane.

### Example L

### In Vitro Metabolism in Human Hepatocytes

Metabolism of amitifadine in human hepatocytes and detection of metabolites formed is conducted by XenoBiotic Laboratories, Inc. (Plainsboro, N.J.). Cryopreserved human hepatocytes (male) that are prepared by XenoTech, LLC (Lenexa, Kans.), are thawed on the day of use and resuspended in Waymouth's incubation media. The enzymatic activity of hepatocytes is monitored using 7-ethoxycoumarin and 7-hydroxycoumarin as substrates. Amitifadine is dissolved in methanol and added to the hepatocyte suspension with a final concentration of 87 μM containing 0.25% methanol. The incubations are carried out for 4 hours with 1×10⁶ viable cells/ml. All incubations are conducted in an incubator maintained at 37° C. and in an atmosphere consisting of about 95% air and 5% CO₂; relative humidity is maintained at about 95%. Cell viability is checked using trypan blue exclusion method at 0, 1, and 4 hours of incubation, and 82% of the cells remained viable after 4 hour incubation. At 0 and 4 hour, cell suspensions are extracted using two volumes of ice cold methanol. After centrifugation, the extracts are stored at −20° C. until further sample processing. The solvent in the samples are dried under a stream of nitrogen, and resuspended in methanol:water (1:1) before analysis. The extracts of the 4 hour incubation mixture of amitifadine are analyzed by LC/electrospray ionization-mass spectrometry (LC/ESI-MS) using LCQ™ Ion Trap Mass Spectrometer in the positive and negative ion modes to screen for possible metabolites (see below for methodology). Because amitifadine contains two chlorines, the unique pattern of ³⁵Cl and ³⁷Cl ion clusters is used for the determination and confirmation of molecular and fragment ions of the metabolites. The disappearance of the parent drug is estimated by comparison of the mass ion intensity (peak height) of amitifadine in the 0 and 4 hour incubations (see below for methodology).

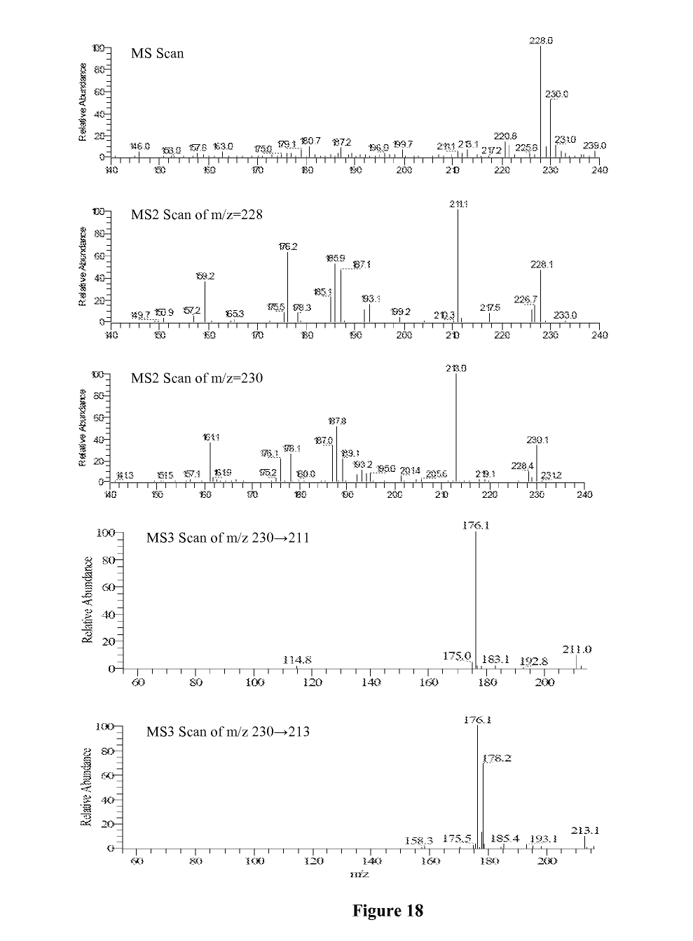

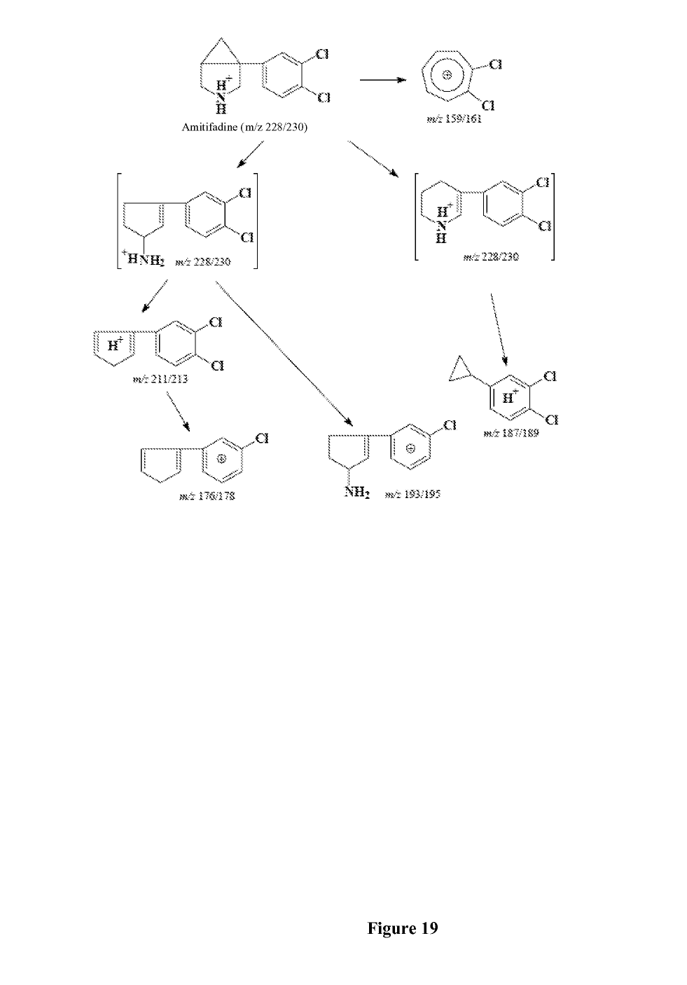

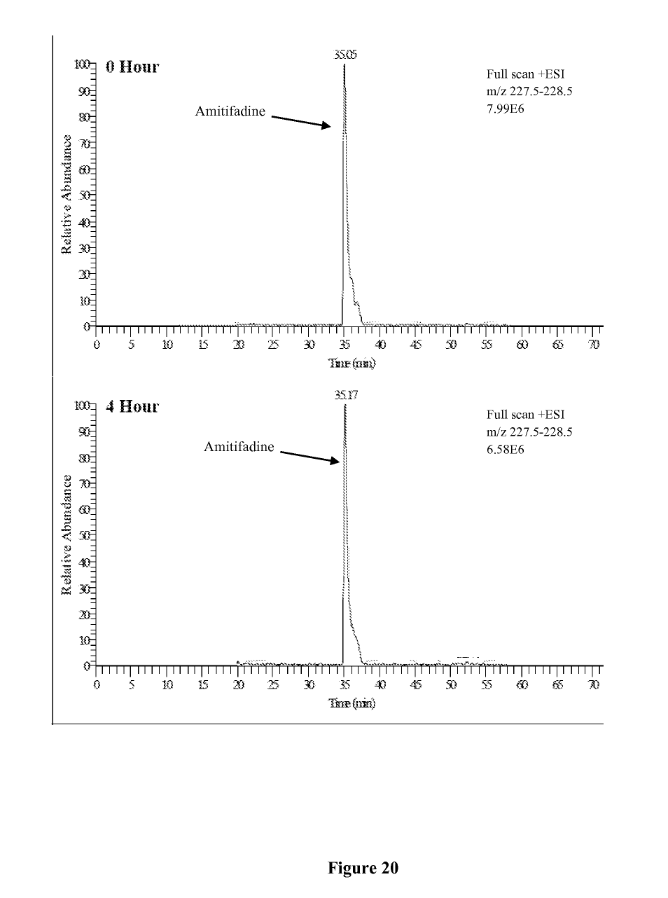

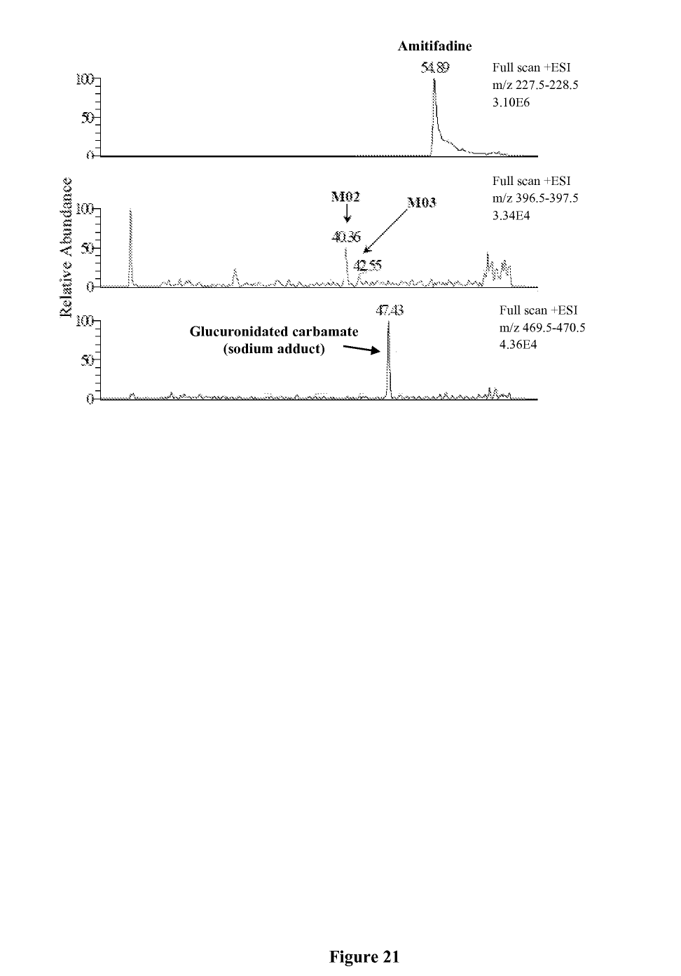

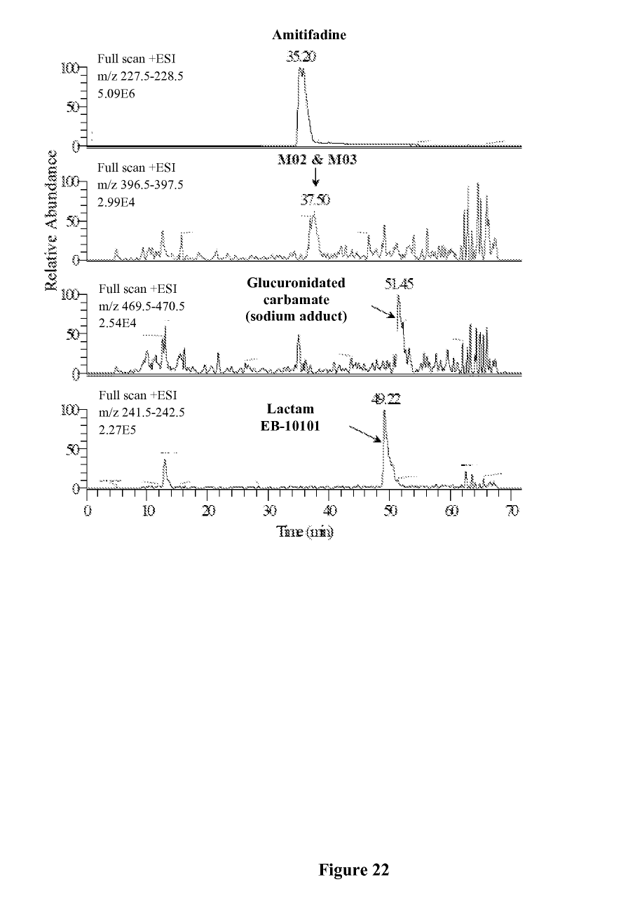

The amitifadine standard is analyzed using LC/MS and the mass ion patterns generated are shown in FIG. 18. Based on the MS2 and MS3 ion fragment patterns, the proposed ion fragmentation pathways of amitifadine are in FIG. 19. At completion of a 4 hour incubation, 82% human hepatocytes remain viable as determined by the trypan blue exclusion method (data not shown). After 4 hours incubation with the human hepatocytes, 68.9% of 7-ethoxycoumarin (100 μM), and 99.1% of 7-hydroxycoumarin (100 μM) are metabolized, indicating the hepatocytes are metabolically active. The majority of amitifadine (82.4%) from the 87 μM initial concentration remains unchanged after 4 hour incubation based on the parent mass ion intensity (FIG. 20). Two different LC-MS/MS methods are conducted to screen for prominent amitifadine metabolites formed in human hepatocytes incubation samples and 4 different metabolites are detected (FIGS. 21 and 22). The structures of the metabolites (Table 55) are proposed based on mass ion and fragment patterns (data not shown). One of the prominent metabolites is due to the oxidation of a carbon atom adjacent to the azabicyclo nitrogen to form the lactam EB-10101 and accounted for about 63% of the metabolites formed (data not shown). There is no appreciable formation of lactam isomer on the 5 position of the azabicyclo ring (data not shown). Another metabolite is due to the addition of CO₂ to the nitrogen to form a carbamate analog, which is subsequently conjugated to the glucuronide (Table 55). The other two metabolites (labeled M02 and M03) have a molecular weight of 396, are minor in quantity, and their structures are not determined.

TABLE 55
Proposed Structure, Molecular Weight, and Retention
Times of Putative Metabolites Formed from
Metabolism of Amitifadine by Human Hepatocytes

| | | | Reten- |
|---|---|---|---|
| | | | tion |
| | | | time, |
| Compound | Proposed structure | ³⁵Cl-MW | min |
| Amitifadine | 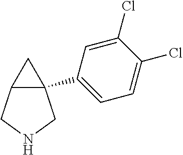 [SMILES: Clc1ccc([C@]23CNCC2C3)cc1Cl] | 227 | ~55.0^{a} ~35.8^{b} |
| Glucuronidated carbamate | 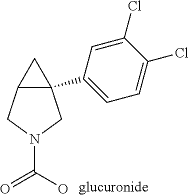 [SMILES: O=C(O)N1CC2C[C@@]2(c2ccc(Cl)c(Cl)c2)C1] | 447 | ~47.7^{a} ~51.4^{b} |
| Lactam EB-10101 | 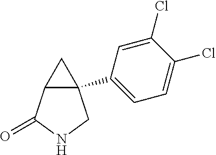 [SMILES: O=C1NC[C@]2(c3ccc(Cl)c(Cl)c3)CC12] | 241 | ~49.3^{b} |

^{a}Retention time when using HPLC Method 1.
^{b}Retention time when using HPLC Method 2.

The extracts of the 4 hour incubation mixture are analyzed by liquid chromatography/electrospray ionization-mass spectrometry (LC/ESI-MS) in the positive and negative ion modes to screen for possible metabolites. HPLC Methods 1 and 2 are used to find potential metabolites, and the HPLC and mass spectrometry systems are described in Table 56. The gradient for the HPLC system for Method 1 is shown in Table 57. The same instrumentation and methods in HPLC Method 1 are used in HPLC Method 2 with the exception that the mobile phases and gradient are changed to 0.4% HCOOH in H₂O for mobile A and CH₃OH for mobile phase B. The mass spectrometer is LCQ™ Ion Trap Mass Spectrometer and is in either the positive or negative electrospray mode with the ion spray set at 4.5 kV. The capillary temperature is 250° C., the nitrogen sheath gas flow rate is ˜80 unit, the auxiliary nitrogen gas flow rate is 20 unit, and the collision gas is helium.

TABLE 56
Mass Spectrometry Systems, Columns, and Parameters
Used for In vitro Metabolism Study in Hepatocytes

| Item | Type |
|---|---|
| HPLC system | Waters ® 2695 |
| | Separations Module |
| UV Detector | Waters ® 486 at 254 nm |
| Column | Ace 3, C18, 3 μm, 150 × 4.6 mm |
| Guard column | RP-18, 7 μm, 15 × 3.2 mm column |
| Temperature | 35° C. |
| Autosampler | 4° C. |
| temperature | |
| Mobile phase A | 0.01M NH₄OAc in H₂O (pH ca. 6.7) |
| Mobile phase B | CH₃CN |

TABLE 57
Gradient table for Method 1 in vitro
metabolism study in hepatocytes

| Time | Flow | | |
|---|---|---|---|
| (min) | (ml/min) | % A | % B |
| Initial | 0.7 | 100 | 0 |
| 3 | 0.7 | 100 | 0 |
| 8 | 0.7 | 90 | 10 |
| 58 | 0.7 | 65 | 35 |
| 65 | 0.7 | 0 | 100 |
| 70 | 0.7 | 0 | 100 |
| 72 | 0.7 | 100 | 0 |

TABLE 58
Gradient table for Method 2 in vitro
metabolism study in hepatocytes

| Time | Flow | % | % |
|---|---|---|---|
| (min) | (ml/min) | A | B |
| Initial | 0.7 | 100 | 0 |
| 3 | 0.7 | 100 | 0 |
| 8 | 0.7 | 80 | 20 |
| 58 | 0.7 | 30 | 70 |
| 65 | 0.7 | 0 | 100 |
| 70 | 0.7 | 0 | 100 |
| 72 | 0.7 | 100 | 0 |

### Example LI

### Inhibition of Human CYP Activity in Microsomes

The inhibitory potential of amitifadine towards seven human liver microsomal CYP activities is evaluated by Merck Research Laboratories (West Point, Pa.). Enzyme activities studied are CYP1A2-dependent phenacetin O-deethylation, CYP2B6-dependent bupropion hydroxylation, CYP2C8-dependent taxol 6α-hydroxylation, CYP2C9-dependent diclofenac 4′-hydroxylation, CYP2C19-dependent (S)-mephenytoin 4′-hydroxylation, CYP2D6-dependent bufuralol 1′-hydroxylation, and CYP3A4-dependent testosterone 6β-hydroxylation. The human liver microsomes are purchased from Tissue Transformation Technologies (Exton, Pa.) and from BD Biosciences (San Jose, Calif.) and are pooled from 10 individual samples.

To determine the CYP inhibitory potential, amitifadine at varying concentrations (0.05-100 μM) is incubated with liver microsomes in the presence of a single concentration of the enzyme substrate (approaching established Kₘ value). The substrates are phenactin, bupropion, taxol, diclofenac, (S)-mephenytoin, bufuralol, and testosterone for CYP 1A2, 2B6, 2C8, 2C9, 2C19, 2D6, and 3A4, respectively. The positive control inhibitors are fluvoxamine (0.005-10 μM), N-(α-methylbenzyl)-1-aminobenzotriazole (0.005-10 μM), quercetin (0.02-50 μM), sulfaphenazole (0.005-10 μM), (R)—N-3-benzylphenobarbital (0.005-10 μM), quinidine (0.005-10 μM), and ketoconazole (0.005-10 μM), respectively. The incubation mixtures (0.2 ml final volume) contain microsomal protein (0.25 or 0.5 mg/ml) and 100 mM potassium phosphate buffer (pH 7.4). The reaction is initiated by the addition of NADPH, and is allowed to proceed for 10 to 30 min at 37° C. in a shaking water bath. The reactions are terminated with the addition of two volumes of acetonitrile containing an appropriate internal standard, and the samples are mixed and centrifuged. Aliquots of the supernatant are analyzed with a LC-MS/MS method. All incubations are run in duplicate and the enzyme activity in the absence of amitifadine and positive control inhibitors is assigned as control value (100%).

The LC-MS/MS analysis used is a Perkin Elmer HPLC system, comprised of a series 200 LC pump and autosampler. The mass spectrometer unit is a API Triple Quadrupole LC-MS/MS Mass Spectrometer (Sciex API 2000) with a APCI ion source. Mobile Phase A (CYP1A2) consists of solvent A: 0.05% formic acid in water, and solvent B: 0.05% formic acid in acetonitrile. Mobile Phase B (CYP2C9, CYP2C19, CYP2D6, CYP3A4, CYP2C8, and CYP2B6) consists of solvent A: 90/10 water/methanol with 0.05% formic acid, and solvent B: 10/90 water/acetonitrile with 0.05% formic acid.

TABLE 59
HPLC and Mass Spectrometry Analysis Parameters
for In vitro Human CYP Inhibition

| | | | | Metabolite | Internal |
|---|---|---|---|---|---|
| Enzymatic | | Flow | | Q1/Q3 | standard |
| action | HPLC | rate | | mass | Q1/Q3 mass |
| (CYP) | column | (ml/min) | Gradient | (“m/z”)* | (“m/z”)* |
| Phenacetin | AquaSep, 5 μm, | 1.5 | 5 to 50% | Acetaminophen | 4-OH- |
| O-deethylation | 2.0 × 50 mm | | B 1.4 min | 151.9/110.1 | Butyranilide |
| (CYP1A2) | | | linear | | 180.2/71.0 |
| Diclofenac | BDS Hypersil | 1.5 | 10 to 70% | 4′OH- | Flufenamic |
| 4′hydroxylation | C8, 5 μm, | | B 1.9 min | Diclofenac | acid |
| (CYP2C9) | 2 × 50 mm | | linear | 312/231.1 | 272.1/264.1 |
| (S)-Mephenytoin | Zorbax SB-Aq, | 1.5 | 20 to 80% | 4′OH- | Phenytoin |
| 4-hyroxylation | 5 μm, | | B 1.9 min | Mephytoin | 253.2/182.2 |
| (CYP2C19) | 4.6 × 50 mm | | linear | 235.1/150.1 | |
| Bufurarol | BDS Hypersil | 1.5 | 0 to 50% | 1′OH- | DL-Propanolol |
| 1′hydroxylation | C8, 5 μm, | | B 1.9 min | Bufurarol | 260.2/155.1 |
| (CYP2D6) | 2 × 50 mm | | linear | 278.1/186.1 | |
| Testosterone | BDS Hypersil | 1.5 | 0 to 50% | 6β-OH- | Cortisone |
| 6β-hydroxylation | C8, 5 μm, | | B 1.9 min | Testosterone | 361.0/163.2 |
| (CYP3A4) | 2 × 50 mm | | linear | 305.5/269 | |
| Taxol | Zorbax | 1.5 | 20 to 80% | 6α-OH-taxol | Baccatin |
| 6α-hydroxylation | 300Extend | | B 2.0 min | 870.3/525.2 | 587.2/405.2 |
| (CYP2C8) | C18, 5 μm, | | linear | | |
| | 4.6 × 50 mm | | | | |
| Bupropion | BDS Hypersil | 1.5 | 20 to 80% | OH-Bupropion | DL-Propanolol |
| hydroxylation | C8, 5 μm, | | B 2.0 min | 256.2/184.2 | 260.2/155.1 |
| (CYP2B6) | 2 × 50 mm | | linear | | |

The direct inhibitory potential of amitifadine (0.05-100 μM) for the seven human CYP activities is evaluated in human liver microsomes. Enzyme activities studied are CYP 1A2, 2B6, 2C8, 2C9, 2C19, 2D6, and 3A4. In comparison to the positive control inhibitors in the instances where amitifadine had measurable activity, it is 19 to 198 times less potent than comparator inhibitors (Table 60). According to the ranking convention, amitifadine is considered a potent inhibitor of CYP2B6 activity (IC₅₀=1.8 μM), a moderate inhibitor of CYP 1A2, 2C19, 2D6, 2C9, 3A4 activities (IC₅₀=10-22 μM), and a weak inhibitor of CYP2C8 activity (IC₅₀>100 μM) in human liver microsomes.

TABLE 60
Inhibition of CYP Isoforms in Pooled Human Liver Microsomes
by Amitifadine and Comparators

| | | | IC₅₀, μM |
|---|---|---|---|
| | | | (ratio |
| CYP | | | Comparator/ |
| isoform | Reaction | Compound | amitifadine) |
| CYP1A2 | Phenacetin | Fluvoxamine | 0.5 |
| | O-deethylation | Amitifadine | 9.5 (19) |
| CYP2B6 | Bupropion | N-(α-methylbenzyl)-1-AB¹ | 0.06 |
| | hydroxylation | Amitifadine | 1.8 (30) |
| CYP2C8 | Taxol | Quercetin | 18.7 |
| | 6α-hydroxylation | Amitifadine | >100 (>5.3) |
| CYP2C9 | Diclofenac | Sulfaphenazole | 0.7 |
| | 4′hydroxylation | Amitifadine | 22.6 (32) |
| CYP2C19 | (S)-Mephenytoin | (R)-N-3-benzyl- | 0.3 |
| | 4′-hydroxylation | Phenobarb-ital | 21.6 (72) |
| | | Amitifadine | |
| CYP2D6 | Bufuralol | Quinidine | 0.1 |
| | 1′-hydroxylation | Amitifadine | 19.8 (198) |
| CYP3A4 | Testosterone | Ketoconazole | 0.02 |
| | 6β-hydroxylation | Amitifadine | 15.5 (77) |

Amitifadine (0.05 to 100 μM) or comparators are incubated for 10 to 30 min at 37° C. and the formation of respective metabolite is determined by LC-MS/MS techniques.
¹Abbreviation for N-(α-methylbenzyl)-1-aminobenzatriazole.

### Example LII

### Determination of Brain and Plasma Levels of Amitifadine and Lactam Metabolite EB-10101 in Rat

Brain and plasma levels of amitifadine are determined by Mithridion Inc. (Madison, Wis.). Adult male Sprague-Dawley rats weighing 229-291 g are obtained from Charles River Laboratories and acclimated for one week under standard housing conditions prior to use. The studies are conducted in accordance with the Institutional Animal Care and Use Committee guidelines. Rats in groups of three are food-deprived 16 hours prior to the oral administration of drug or vehicle (distilled water) with a dosing volume of 1 ml/kg. Amitifadine solution (10 mg/ml) in distilled water is freshly prepared on the day of dosing and administered to rats at 10 mg/kg. At 0.5, 1, 2, and 4 hour after dosage, animals are briefly anesthetized in a chamber containing 4% isoflurane and rapidly decapitated. Trunk blood is collected in heparinized tubes (9 units/ml whole blood), immediately chilled to 4° C., centrifuged, and the plasma is frozen at −80° C. until analyzed. Brains are quickly removed, bisected along the midline, and frozen at −80° C. until analyzed.

Plasma and brain samples are allowed to thaw on ice and are homogenized in a 20 ml glass vial in two volumes of ice cold water using a handheld homogenizer (VDI 12, VWR International). Standard curves are prepared in water, control plasma, and control brain homogenate. Equal volumes (100 μl) of plasma and brain homogenate samples, distilled water and internal standard are mixed in 1.5 ml polypropylene tubes and 300 μl of acetonitrile is added to each mixture. Samples are then mixed thoroughly by placing on a shaker at 200 RPM for 40 minutes at room temperature. The samples are then centrifuged at 10,000×g for 10 minutes at 4° C. and the supernatant (200 μl) is transferred to a tube containing 2 μl of 10% formic acid. The samples are subjected to a Zorbax SBC18 Solvent Saver Plus 3.0×150 mm 3.5-μm column on a Shimadzu Prominence LC. The compounds are eluted at 0.6 ml/min flow rate using a gradient of 40% to 100% mobile phase B in mobile phase A. Mobile phase A consists of water with 10 mM ammonium formate and 0.065% formic acid; and mobile phase B consists of 90% methanol with 10 mM ammonium formate and 0.065% formic acid. The concentration of compound in the column effluent is measured using an Applied Biosystems API-3200 triple quadrupole mass spectrometer.

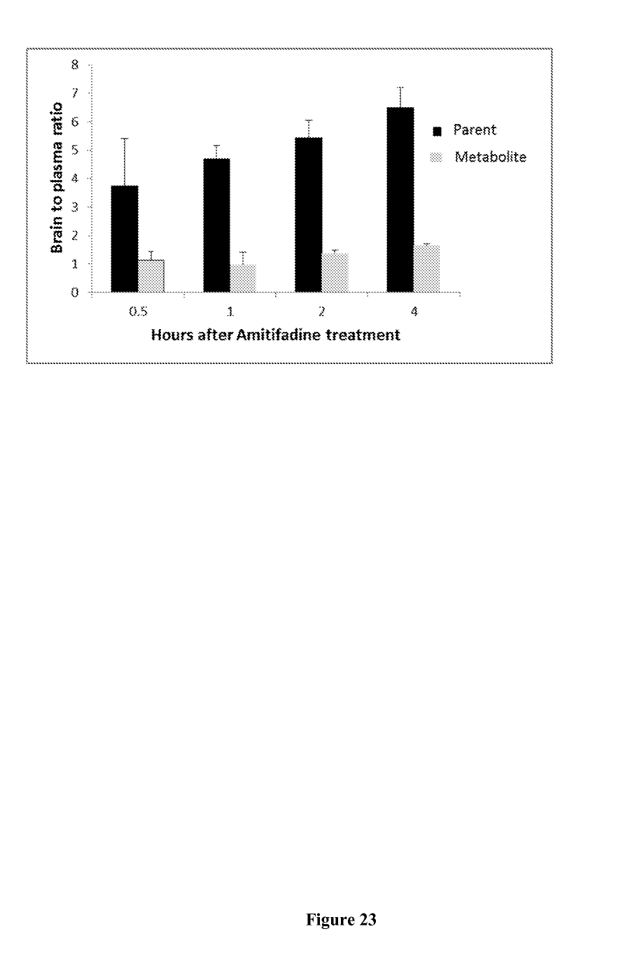

After oral administration of amitifadine (10 mg/kg) to rats, the concentrations of amitifadine and EB-10101 in rat brain (μg/g) and plasma (μg/ml) are determined by LC-MS/MS. The brain to plasma ratio for amitifadine is 3.7, 4.7, 5.4, and 6.5, at 0.5, 1, 2 and 4 hours after oral administration, respectively, indicating preferential penetration into the brain (FIG. 23). The brain to plasma ratio for EB-10101 is 1.1, 1.3, 1.4, and 1.7, respectively. Recovery for amitifadine is 128% in plasma and 67% in brain homogenate, and recovery of the lactam metabolite EB-10101 is 107% in plasma and 105% in brain.

TABLE 61
Mass spectrometry conditions

| | Amitifadine | EB-10101 |
|---|---|---|
| Parent > daughter (m/z) | 228.0 > 187.0 | 242.0 > 158.9 |
| Probe Temperature (° C.) | 400 | 400 |
| Ion Source Voltage (V) | 5500 | 5500 |
| Ion Source Gas 1 (psi) | 50 | 50 |
| Ion Source Gas 2 (psi) | 60 | 60 |
| Curtain Gas (psi) | 25 | 25 |
| Collision Gas (psi) | 5 | 5 |
| Declustering Potential (V) | 46 | 56 |
| Entrance Potential (V) | 9.5 | 10.5 |
| Collision Energy (V) | 31 | 35 |
| Collision Cell | 4 | 4 |
| Exit Potential (V) | | |

All publications and patents cited herein are incorporated herein by reference for the purpose of describing and disclosing, for example, the materials and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

“Depression”. World Health Organization. http://www.who.int/mental_health/management/depression/definition/en/ (2012).
Abraham B K, Adithan C. Genetic polymorphism of CYP2D6. Indian J Pharmacol. 2001; 33:147-69.
Basile, A. S., et al., J. Pharmacol. Exp. Ther., 321:1208-1225 (2007)
Basile, A.; Janowsky, A.; Golembiowska, K.; Kowalska, M.; Tam, E.; Benveniste, M.; Popik, P.; Nikiforuk, A. et al. (2007). “Characterization of the antinociceptive actions of bicifadine in models of acute, persistent, and chronic pain”. The Journal of pharmacology and experimental therapeutics 321 (3): 1208-1225.
Beck, A. T., R. A Steer (1988) Manual for Beck Hopelessness Scale. Psychological Corp., Harcourt Brace Jovanovich San Antonio, Tex.
Beck, A. T., R. A Steer (1991) Manual or Beck Scale For Suicide Ideation. San Antonio, Tex.: Psychological Corporation.
Bernard S, Neville K A, Nguyen A T, Flockhart D A. Inter-ethnic differences in genetic polymorphisms of CYP2D6 in the U.S. population: clinical implications. Oncologist. 2006; 11:126-35
Briley, M., Hum. Psychopharmacol. Clin. Exp. 19:S21-S25 (2004) [0449]Skolnick, P. in “Dopamine and glutamate in psychiatric disorders,” W. Schmidt, Editor; Humana Press, Totowa, Chapter 9, pp. 199-214 (2005) [0450]Atkinson, J. H. et al., Pain 83:137-145 (1999)
Byrne S E, Rothschild A J. Loss of antidepressant efficacy during maintenance therapy: possible mechanisms and treatments. J Clin Psychiatry. 1998 June; 59(6):279-88.
W-M Cai, D M Nikoloff, R-M Pan, J de Leon, P Fanti, M Fairchild, W H Koch and P J Wedlund. CYP2D6 genetic variation in healthy adults and psychiatric African-American subjects: implications for clinical practice and genetic testing The Pharmacogenomics Journal (2006) 6,343-350. doi:10.1038/sj.tpj.6500378; published online 21 Mar. 2006.
Chandler G M, Iosifescu D V, Pollack M H, Targum S D, Fava M Validation of the Massachusetts General Hospital Antidepressant Treatment History Questionnaire (ATRQ) CNS Neurosci Ther. 2009 Sep. 21. [Epub ahead of print]
Chong E, Ensom M H. Pharmacogenetics of the proton pump inhibitors: a systematic review. Pharmacotherapy. 2003; 23:460-71.
Coric, Vladimir•Stock, Elyse G•Pultz, Joseph•Marcus, Ronald•Sheehan, David V. Sheehan Suicidality Tracking Scale (Sheehan-STS): Preliminary Results from a Multicenter Clinical Trial in Generalized Anxiety Disorder. Psychiatry (Edgmont (Pa.: Township)) 2009 6 (1): 26-31.
Crews K R, Gaedigk A, Dunnenberger H M, Klein T E, Shen D D, Callaghan J T, Kharasch E D, Skaar T C; Clinical Pharmacogenetics Implementation Consortium. Clinical Pharmacogenetics Implementation Consortium (CPIC) guidelines for codeine therapy in the context of cytochrome P450 2D6 (CYP2D6) genotype. Clin Pharmacol Ther. 2012 February; 91(2):321-6.
deVane, Lindsay Antidepressant-Drug Interactions are Potentially but Rarely Clinically significant Neuropsychopharmacology (2006) 31, 1594-1604.
Di Chiara G, Bassareo V, Fenu S, De Luca M A, Spina L, Cadoni C, Acquas E, Carboni E, Valentini V, Lecca D (2004) Dopamine and drug addiction: The nucleus accumbens shell connection. Neuropharmacology 47 Suppl 1: 227-241.
Eshleman, A. J. et al., Journal of Pharmacology & Experimental Therapeutics 289:877-885 (1999)
Fava M, Davidson K G. Definition and epidemiology of treatment-resistant depression. Psychiatr Clin North Am. 1996; 19:179-200
Fava M, Evins A E, Dorer D J, Schoenfeld D A. The problem of the placebo response in clinical trials for psychiatric disorders: culprits, possible remedies, and a novel study design approach. Psychother Psychosom. 2003; 72(3):115-27
Fava M, Iosifescu D V, Pedrelli P, Baer L. Reliability and validity of the Massachusetts general hospital cognitive and physical functioning questionnaire. Psychother Psychosom. 2009; 78(2):91-7
Fava M, Rush A J, Thase M E, Clayton A, Stahl S M, Pradko J F, Johnston J A. 15 years of clinical experience with bupropion HCl: from bupropion to bupropion SR to bupropion XL. Prim Care Companion J Clin Psychiatry. 2005; 7(3):106-13
Fava M. Diagnosis and definition of treatment-resistant depression. Biol Psychiatry. 2003; 53(8):649-59
Fava M. Prospective studies of adverse events related to antidepressant discontinuation. J Clin Psychiatry. 2006; 67 Suppl 4:14-21
First, Michael B., Williams, Janet B. W., Spitzer, Robert L., and Gibbon, Miriam: Structured Clinical Interview for DSM-IV-TR Axis I Disorders, Clinical Trials Version (SCID-CT). New York: Biometrics Research, New York State Psychiatric Institute, 2007.
Forcehimes A A, Tonigan J S, Miller W R, Kenna G A, Baer J S. Psychometrics of the Drinker Inventory of Consequences (DrInC). Addict Behay. 2007 August; 32(8):1699-704. Epub 2006 Dec. 19.
Frantz S. Drug discovery: playing dirty. Nature. 2005; 437:942-3.
Gardner, E.; Liu, X.; Paredes, W.; Giordano, A.; Spector, J.; Lepore, M.; Wu, K.; Froimowitz, M. (2006). “A slow-onset, long-duration indanamine monoamine reuptake inhibitor as a potential maintenance pharmacotherapy for psychostimulant abuse: effects in laboratory rat models relating to addiction”. Neuropharmacology 51 (5): 993-1003.
Graff, Ole et al. Results of two double blind Placebo and Active-controlled Studies of GSK372475, a Triple Monoamine Reuptake Inhibitor, in the Treatment of Major Depressive Disorder. (ACNP 2009)
Green, T. W. and Wuts, P. G. M. in “Protective Groups in Organic Chemistry”, 3rd edition, John Wiley & Sons, New York, N.Y., 1999
Gu, H., et al. J. Biol. Chem. 269:7124-7130 (1994)
Guengerich F P (January 2008). “Cytochrome P450 and chemical toxicology”. Chem. Res. Toxicol. 21 (1): 70-83. DOI:10.1021/tx700079z. PMID 18052394.
Guy W. Clinician Global Impression (CGI). ECDEU Assessment Manual for Psychopharmacology. 1976. Rockville, Md., U.S. Department of Health, Education, and Welfare.
Hamilton M. A rating scale for depression. J. Neurol. Neurosurg. Psychiat., 1960, 23,56.
Hamilton M. Development of a rating scale for primary depressive illness. Br J Soc Clin Psychol. 1967; 6(4):278-96
Hamilton, M. (1959). The assessment of anxiety states by rating. British Journal of Medical Psychology, 32, 50-55.
Hollenberg, Paul F. Characteristics and common properties of inhibitors, inducers, and activators of cyp enzymes drug metabolism reviews, 34(1&2), 17-35 (2002).
Ingelman-Sundberg M. Pharmacogenetics of cytochrome P450 and its applications in drug therapy: the past, present and future. Trends Pharmacol Sci. 2004 April; 25(4):193-200.
Johansson, Inger and Magnus Ingleman-Sundberg. Genetic Polymorphism and Toxicology—With Emphasis on Cytochrome P450. Toxicol. Sci. (2011) 120 (1): 1-13
Judd L L, Akiskal H S, Maser J D, Zeller P J, Endicott J, Coryell W, Paulus M P, Kunovac J L, Leon A C, Mueller T I, Rice J A, Keller M B. Major depressive disorder: a prospective study of residual subthreshold depressive symptoms as predictor of rapid relapse. J Affect Disord. 1998; 50(2-3):97-108
Judd L L, Paulus M J, Schettler P J, Akiskal H S, Endicott J, Leon A C, Maser J D, Mueller T, Solomon D A, Keller M B. Does incomplete recovery from first lifetime major depressive episode herald a chronic course of illness? Am J Psychiatry. 2000; 157(9):1501-4.
Keller M B, Shapiro R W, Lavori P W, Wolfe N. Relapse in major depressive disorder: analysis with the life table. Arch Gen Psychiatry. 1982; 39(8):911-5
Labbate L A, Lare S B. Sexual dysfunction in male psychiatric outpatients: validity of the Massachusetts General Hospital Sexual Functioning Questionnaire. Psychother Psychosom. 2001; 70(4):221-5
Lobello K W, Preskorn S H, Guico-Pabia C J, Jiang Q, Paul J, Nichols A I, Patroneva A, Ninan P T. Cytochrome P450 2D6 phenotype predicts antidepressant efficacy of venlafaxine: a secondary analysis of 4 studies in major depressive disorder. J Clin Psychiatry. 2010 November; 71(11):1482-7.
Lynch Tom, PharmD, Price Amy, M D, The Effect of Cytochrome P450 Metabolism on Drug Response, Interactions, and Adverse Effects. Am Fam Physician. 2007 Aug. 1; 76(3):391-396.
L. L. McCoy, J. Am. Chem. Soc., 80, 6568 (1958).
Mcmillen, B.; Shank, J.; Jordan, K.; Williams, H.; Basile, A. (2007). “Effect of DOV 102,677 on the volitional consumption of ethanol by Myers' high ethanol-preferring rat” Alcoholism, clinical and experimental research 31 (11): 1866-1871.
Miller I W, Keitner G I, Schatzberg A F, Klein D N, Thase M E, Rush A J, Markowitz J C, Schlager D S, Kornstein S G, Davis S M, Harrison W M, Keller M B. The treatment of chronic depression, part 3: psychosocial functioning before and after treatment with sertraline or imipramine. J Clin Psychiatry. 1998; 59(11):608-19.
Montgomery S A, Åsberg M. A new depression scale designed to be sensitive to change. Br J Psychiatry 1979; 134:382-9.
Montgomery, S. A. & Åsberg, M. (1979) A New Depression Scale Designed To Be Sensitive To Change. British Journal of Psychiatry. Vol. 134, pp. 382-389.
Mrazek D A. Psychiatric pharmacogenomic testing in clinical practice. Dialogues Clin Neurosci. 2010; 12(1):69-76.
Meunier B. Mechanism of oxidation reactions catalyzed by cytochrome p450 enzymes. Chem Rev. 2004 September; 104(9):3947-80.
Nitrogen Protecting Groups in Organic Synthesis, John Wiley and sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry, 3rd edition” John Wiley & Sons, Inc. New York, N.Y., 1999.
Nyenhuis D L, Yamamoto C, Luchetta T, Terrien A, Parmentier A. Adult and geriatric normative data and validation of the profile of mood states. J Clin Psychol. 1999 January; 55(1):79-86.
Olsen, L. R., et al., The internal and external validity of the Major Depression Inventory in measuring severity of depressive states Psychological Medicine (2003), 33: 351-356 Cambridge University Press.
Papakostas G I, Fava M, Thase M E. Treatment of SSRI-resistant depression: a meta-analysis comparing within-versus across-class switches. Biol Psychiatry. 2008 Apr. 1; 63(7):699-704.
Parker G B, Brotchie H L, Hyett M. Tolerance to desvenlafaxine in rapid metabolizing depressed patients. Int Clin Psychopharmacol. 2011 March; 26(2):84-7.
Paykel E S, Ramana R, Cooper Z, Hayhurst H, Kerr J, Barocka A. Residual symptoms after partial remission: an important outcome in depression. Psychol Med. 1995 November; 25(6):1171-80.
Perovic, S. and Muller, W. E., Arzneimittelforschung 45: 1145-1148 (1995).
Polasek T M, Lin F P, Miners J O, Doogue M P. Perpetrators of pharmacokinetic drug-drug interactions arising from altered cytochrome P450 activity: a criteria-based assessment. Br J Clin Pharmacol. 2011 May; 71(5):727-36.
Porsolt R, Lenegre A. Behavioral models of depression. In: Elliott J, Heal D, Marsden C, eds. Experimental Approaches to Anxiety and Depression. London: Wiley; 1992:73-85.
Posner, K. (2008) Suicidality Across Drug Indications: Columbia Suicidal Adverse Event Identification and FDA Safety Concerns: The Issues and the Answers, From Outcomes to Feasibility. Invited presentation to the Center for Drug Evaluation and Research (CDER) at the Food and Drug Administration, Silver Spring, Md.
Posner K, Oquendo M, Gould M, et al. Columbia Classification Algorithm of Suicide Assessment (C CASA): classification of suicidal events in the FDA's pediatric suicidal risk analysis of antidepressants. Am J Psychiatry. 2007; 165:1035-1043.
Posner K, Oquendo M A, Gould M, Stanley B, Davies M (2007). Columbia Classification Algorithm of Suicide Assessment (C-CASA): Classification of Suicidal Events in the FDA's Pediatric Suicidal Risk Analysis of Antidepressants. Am J Psychiatry; 164:1035-1043.
Povlock, S. L. and Amara, S. G., in “Neurotransmitter transporters: structure, function, and regulation,” Reith M E A, Editor, Humana Press, Totowa, pp. 1-28 (1997).
Preskorn, Sheldon H., M.D. Clinical Pharmacology of SSRI's. Why are CYP Enzymes important when prescribing SSRIs? www.Preskorn.com (2010).
Preskorn S H, Do you feel lucky? J Prac Psycho Behav Hlth. 1998; 4:37-40.
Quick Reference to the Diagnostic Criteria From DSM-IV (Diagnostic and Statistical Manual of Mental Disorders, Fourth Edition), The American Psychiatric Association, Washington, D.C., 1994.
Roth, B.; Sheffler, D.; Kroeze, W. (2004). “Magic shotguns versus magic bullets: selectively non-selective drugs for mood disorders and schizophrenia”. Nature reviews. Drug discovery 3 (4): 353-359.
Rush A J, Trivedi M H, Wisniewski S R, Nierenberg A A, Stewart J W, Warden D, Niederehe G, Thase M E, Lavori P W, Lebowitz B D, McGrath P J, Rosenbaum J F, Sackeim H A, Kupfer D J, Luther J, Fava M. Acute and longer-term outcomes in depressed outpatients requiring one or several treatment steps: a STAR*D report. Am J Psychiatry. 2006 November; 163(11):1905-17.
Rush A J, Trivedi M H, Wisniewski S R, Stewart J W, Nierenberg A A, Thase M E, Ritz L, Biggs M M, Warden D, Luther J F, Shores-Wilson K, Niederehe G, Fava M; STAR*D Study Team. Bupropion-SR, sertraline, or venlafaxine-XR after failure of SSRIs for depression. N Engl J Med. 2006 Mar. 23; 354(12):1231-42.
Sachse, Christopher, Jurgen Brockmoller, Steffen Bauer, and Ivar Roots Cytochrome P450 2D6 Variants in a Caucasian Population: Allele Frequencies and Phenotypic Consequences. Am. J. Hum. Genet. 60:284-295, 1997.
Sayette M A, Shiffman S, Tiffany S T, et al. The measurement of drug craving. Addiction. 2000; 95:S189-S210.
Schrieber, R., R. Lew, L. Hardy, T. Cremers, K. Fang, U. Campbell; Poster 549.8/X2 Pharmacological characterization of the triple monoamine transporter uptake inhibitor SEP225289. Oct. 20, 2009.
Scott S A, Sangkuhl K, Gardner E E, Stein C M, Hulot J S, Johnson J A, Roden D M, Klein T E, Shuldiner A R; Clinical Pharmacogenetics Implementation Consortium. Clinical Pharmacogenetics Implementation Consortium guidelines for cytochrome P450-2C19 (CYP2C19) genotype and clopidogrel therapy. Clin Pharmacol Ther. 2011 August; 90(2):328-32.
Sepracor Press Release, Jul. 1, 2009 http://www.fiercebiotech.com/press-releases/sepracor-provides-update-clinical-trials-sep-225289-and-lunesta-r-pediatrics.
Sheehan D V, Y Lecrubier, K H Sheehan, P Amorim, J Janays, E Weiller, T Hergueta, R Baker, G C Dunbar. The Mini-International Neuropsychiatric Interview (M.I.N.I.): the development and validation of a structured diagnostic psychiatric interview for DSM-IV and ICD-10. J Clin Psychiatry. 1998; 59 Suppl. 20:22-33; quiz 34-57.
Skolnick P. Antidepressants beyond monoamine-based therapies: clues to new approaches. J Clin Psychiatry. 2002; 63:19-23.
Skolnick M, Popik P, Janowsky A, Beer B, Lippa A: Antidepressant-like actions of amitifadine: A triple reuptake inhibitor. European Journal of Pharmacology 2003 461:99-104.
Skolnick, P. et al., Life Sci. 73: 3175-3179 (2003).
Skolnick, P., et al., CNS Drug Reviews (2006).
Targum S D, Pollack M H, Fava M. Redefining affective disorders: relevance for drug development. CNS Neurosci Ther. 2008 Spring; 14(1):2-9.
Teh, L., et al., Pharmacogenetics of 2YPD6: Molecular Genetics, Interethnic Differences and Clinical Importance. Drug Metab. Pharmacokinet. 27 (1): 55-67 (2012).
Thase M E, Entsuah A R, Rudolph R L. Remission rates during treatment with venlafaxine or selective serotonin reuptake inhibitors. Br J Psychiatry. 2001; 178:234-41.
Thase M E, Simons A D, McGeary J, Cahalane J F, Hughes C, Harden T, Friedman E. Relapse after cognitive behavior therapy of depression: potential implications for longer courses of treatment. Am J Psychiatry. 1992; 149(8):1046-52.
Thase M E. Evaluating antidepressant therapies: remission as the optimal outcome. J Clin Psychiatry. 2003; 64(Suppl 13):18-25.
Tizzano, J.; Stribling, D.; Perez-Tilve, D.; Strack, A.; Frassetto, A.; Chen, R.; Fong, T.; Shearman, L. et al. (2008). “The triple uptake inhibitor (1R,5S)-(+)-1-(3,4-dichlorophenyl)-3-azabicyclo3.1.0 hexane hydrochloride (DOV 21947) reduces body weight and plasma triglycerides in rodent models of diet-induced obesity”. The Journal of pharmacology and experimental therapeutics 324 (3): 1111-1126.
Trivedi M H, Fava M, Wisniewski S R, Thase M E, Quitkin F, Warden D, Ritz L, Nierenberg A A, Lebowitz B D, Biggs M M, Luther J F, Shores-Wilson K, Rush A J; STAR*D Study Team. Medication augmentation after the failure of SSRIs for depression. N Engl J Med. 2006 Mar. 23; 354(12):1243-52.
Trivedi M H, Rush A J, Ibrahim H M et al. The Inventory of Depressive Symptomatology, Clinician Rating (IDS-C) and Self-Report (IDS-SR), and the Quick Inventory of Depressive Symptomatology, Clinician Rating (QIDS-C) and Self-Report (QIDS-SR) in public sector patients with mood disorders: a psychometric evaluation. Psychol Med 2004; 34(1):73-82.
U.S. Pat. No. 4,122,193; Scherm et al.; Oct. 24, 1978.
U.S. Pat. No. 6,132,724; Blum; Oct. 17, 2000.
U.S. patent application Ser. No. 11/371,178; Skolnick, et al.; filed Mar. 7, 2006.
van der Weide, Jan and John W J Hinrichs The Influence of Cytochrome P450 Pharmacogenetics on Disposition of Common Antidepressant and Antipsychotic Medications Clin Biochem Rev Vol 27 Feb. 2006.
Van Londen L, Molenaar R P, Goekoop J G, Zwinderman A H, Rooijmans H G. Three- to 5-year prospective follow-up of outcome in major depression. Psychol Med. 1998; 28(3):731-5.
Warren, W. L. Revised Hamilton Rating Scale for Depression (RHSD). (1994) Los Angeles, Western Psychological Services.
Zajecka J M. Treating depression to remission. J Clin Psychiatry. 2003; 64(Suppl 15):7-12.
\check{Z}ourková, A. and E. Hada\check{s}ová, Paroxetine-Induced Conversion of Cytochrome P450 2D6 Phenotype and Occurrence of Adverse Effects, Gen. Physiol. Biophys. (2003), 22, 103|113.

1-64. (canceled)

65. A method for treating depression in a human comprising administering to a human in need of treatment for depression a pharmaceutical composition comprising an effective amount of a (+)-1-(3,4-dichlorophenyl)-3-azabicyclo[3.1.0]hexane agent comprising (+)-1-(3,4-dichlorophenyl)-3-azabicyclo[3.1.0]hexane or a pharmaceutically acceptable active salt thereof, optionally in polymorphic form, wherein the (+)-1-(3,4-dichlorophenyl)-3-azabicyclo[3.1.0]hexane or a pharmaceutically acceptable active salt thereof optionally in polymorphic form, is substantially free of the corresponding (−) enantiomer and wherein the human in need of treatment for depression has previously been refractory to a prior course of treatment for depression due to a cytochrome P450 polymorphism.

66-69. (canceled)

70. The method of claim 65, wherein the (+)-1-(3,4-dichlorophenyl)-3-azabicyclo[3.1.0]hexane agent is Polymorph A of an acid addition salt of (+)-1-(3,4-dichlorophenyl)-3-azabicyclo[3.1.0]hexane substantially free of other geometric and optical isomers and polymorphic forms thereof.

71. The method of claim 70, wherein the acid addition salt is a hydrochloride salt.

72-84. (canceled)

85. A method for treating depression in a human comprising administering to a human in need of treatment for depression a pharmaceutical composition comprising an effective amount of a (+)-1-(3,4-dichlorophenyl)-3-azabicyclo[3.1.0]hexane agent comprising (+)-1-(3,4-dichlorophenyl)-3-azabicyclo[3.1.0]hexane or a pharmaceutically acceptable active salt thereof, optionally in polymorphic form, wherein the (+)-1-(3,4-dichlorophenyl)-3-azabicyclo[3.1.0]hexane or a pharmaceutically acceptable active salt thereof optionally in polymorphic form, is substantially free of the corresponding (−) enantiomer and wherein the human in need of treatment for depression is concurrently on a medication processed by a cytochrome P450 enzyme.

86-89. (canceled)

90. The method of claim 85, wherein the (+)-1-(3,4-dichlorophenyl)-3-azabicyclo[3.1.0]hexane agent is Polymorph A of an acid addition salt of (+)-1-(3,4-dichlorophenyl)-3-azabicyclo[3.1.0]hexane substantially free of other geometric and optical isomers and polymorphic forms thereof.

91. The method of claim 90, wherein the acid addition salt is a hydrochloride salt.

92-103. (canceled)

104. A method for treating depression in a human comprising administering to a human in need of treatment for depression a pharmaceutical composition comprising an effective amount of a (+)-1-(3,4-dichlorophenyl)-3-azabicyclo[3.1.0]hexane agent comprising (+)-1-(3,4-dichlorophenyl)-3-azabicyclo[3.1.0]hexane or a pharmaceutically acceptable active salt thereof, optionally in polymorphic form, wherein the (+)-1-(3,4-dichlorophenyl)-3-azabicyclo[3.1.0]hexane or a pharmaceutically acceptable active salt thereof optionally in polymorphic form, is substantially free of the corresponding (−) enantiomer and wherein the human in need of treatment for depression has a liver impairment.

105-108. (canceled)

109. The method of claim 104, wherein the (+)-1-(3,4-dichlorophenyl)-3-azabicyclo[3.1.0]hexane agent is Polymorph A of an acid addition salt of (+)-1-(3,4-dichlorophenyl)-3-azabicyclo[3.1.0]hexane substantially free of other geometric and optical isomers and polymorphic forms thereof.

110. The method of claim 109, wherein the acid addition salt is a hydrochloride salt.

111-122. (canceled)

123. The method of claim 104, wherein the liver impairment is due to alcoholism.

124. The method of claim 104, wherein the liver impairment is cirrhosis.

125. A method for treating depression in a human comprising administering to a human in need of treatment for depression a pharmaceutical composition comprising an effective amount of a (+)-1-(3,4-dichlorophenyl)-3-azabicyclo[3.1.0]hexane agent comprising (+)-1-(3,4-dichlorophenyl)-3-azabicyclo[3.1.0]hexane or a pharmaceutically acceptable active salt thereof, optionally in polymorphic form, wherein the (+)-1-(3,4-dichlorophenyl)-3-azabicyclo[3.1.0]hexane or a pharmaceutically acceptable active salt thereof, optionally in polymorphic form, is substantially free of the corresponding (−) enantiomer and wherein the human in need of treatment for depression has a mutant allele of CYP2D6.

126. The method of claim 125, wherein the mutant allele of CYP2D6 produces a phenotype that increases the metabolism of pharmaceutical agents metabolized by CYP2D6 in comparison to a wild type allele CYP2D6.

127. The method of claim 126, wherein the mutant allele CYP2D6 produces an ultra-rapid metabolizer phenotype.

128. The method of claim 126, wherein the mutant allele of CYP2D6 produces a rapid metabolizer phenotype.

129. The method of claim 125, wherein the mutant allele of CYP2D6 produces a phenotype that decreases the metabolism of pharmaceutical agents metabolized by CYP2D6 in comparison to a wild type CYP2D6 genotype.

130-133. (canceled)

134. The method of claim 125, wherein the (+)-1-(3,4-dichlorophenyl)-3-azabicyclo[3.1.0]hexane agent is Polymorph A of an acid addition salt of (+)-1-(3,4-dichlorophenyl)-3-azabicyclo[3.1.0]hexane substantially free of other geometric and optical isomers and polymorphic forms thereof.

135. The method of claim 134, wherein the acid addition salt is a hydrochloride salt.

136-147. (canceled)

148. A method for treating depression in a human comprising administering to a human in need of treatment for depression a pharmaceutical composition comprising an effective amount of a (+)-1-(3,4-dichlorophenyl)-3-azabicyclo[3.1.0]hexane agent comprising (+)-1-(3,4-dichlorophenyl)-3-azabicyclo[3.1.0]hexane or a pharmaceutically acceptable active salt thereof, optionally in polymorphic form, wherein the (+)-1-(3,4-dichlorophenyl)-3-azabicyclo[3.1.0]hexane or a pharmaceutically acceptable active salt thereof, optionally in polymorphic form, is substantially free of the corresponding (−) enantiomer and wherein the human in need of treatment for depression has a mutant allele of CYP2C19.

149. The method of claim 148, wherein the mutant allele of CYP2C19 produces a phenotype that increases the metabolism of pharmaceutical agents metabolized by CYP2C19 in comparison to a wild type allele CYP2C19.

150. The method of claim 149, wherein the mutant allele CYP2C19 produces an ultra-rapid metabolizer phenotype.

151. The method of claim 149, wherein the mutant allele CYP2C19 produces a rapid metabolizer phenotype.

152. The method of claim 148, wherein the mutant allele of CYP2C19 produces a phenotype that decreases the metabolism of pharmaceutical agents metabolized by CYP2C19 in comparison to a wild type CYP2C19 genotype.

153-156. (canceled)

157. The method of claim 148, wherein the (+)-1-(3,4-dichlorophenyl)-3-azabicyclo[3.1.0]hexane agent is Polymorph A of an acid addition salt of (+)-1-(3,4-dichlorophenyl)-3-azabicyclo[3.1.0]hexane substantially free of other geometric and optical isomers and polymorphic forms thereof.

158. The method of claim 157, wherein the acid addition salt is a hydrochloride salt.

159-170. (canceled)
